(19) United States
(12) Patent Application Publication
(10) Pub. No.: US 2024/0239801 A1
(43) Pub. Date: Jul. 18, 2024

(54) PYRIMIDO[5,4,D]PYRIMIDINE COMPOUNDS, COMPOSITIONS COMPRISING THEM AND USES THEREOF

(71) Applicant: Université de Montréal, Montréal (CA)

(72) Inventors: Pierre Louis BEAULIEU, Rosemère (CA); Eric BEAULIEU, Mercier (CA); Sasmita TRIPATHY, Pierrefonds (CA); Yannick ROSE, Montréal (CA); Michael DORE, Saint-Bruno-de-Montarville (CA)

(73) Assignee: Université de Montréal, Montréal, QC (CA)

(21) Appl. No.: 18/287,524

(22) PCT Filed: Apr. 19, 2022
(86) PCT No.: PCT/CA2022/050593

§ 371 Date: Oct. 19, 2023

Related U.S. Application Data
(60) Provisional application No. 63/201,222, filed on Apr. 19, 2021.

(51) Int. Cl. C07D 487/04; A61K 31/519; C07D 493/10; C07D 519/00

(52) U.S. Cl. CPC C07D 487/04; A61K 31/519; C07D 493/10; C07D 519/00

(57) ABSTRACT

Compounds, compositions and their use in the treatment of a proliferative disease or condition such as a said proliferative disease or disorder is associated with a RAF gene mutation and/or a RAS gene mutation. The compounds disclosed are of Formula I or a pharmaceutically acceptable salt or solvate thereof, wherein R¹, R², R³, X¹, X², X³, X⁴ and Y are as defined herein:

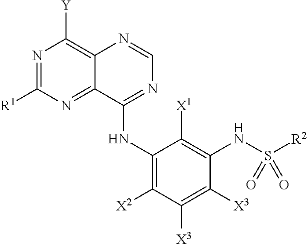

### RELATED APPLICATION

The present application claims priority under applicable law to U.S. provisional application No. 63/201,222 filed on Apr. 19, 2021, the content of which is incorporated herein by reference in its entirety for all purposes.

### TECHNICAL FIELD

This disclosure generally relates to pyrimido[5,4-d]pyrimidine compounds, pharmaceutical compositions comprising the same and their use in the treatment and prevention of diseases characterized by dysregulation of the RAS-ERK pathway (e.g. cancer, RASopathies).

### BACKGROUND

The RAS-RAF-MEK-ERK (RAS: rat sarcoma; RAF: rapidly accelerated fibrosarcoma; MEK: mitogen-activated protein kinase; ERK: extracellular signal-regulated kinase) signaling pathway (hereafter referred to as the RAS-ERK pathway) plays a critical role in transmitting proliferation signals generated by growth factor receptors from the plasma membrane to the nucleus. The pathway is dysregulated in a large proportion of cancers by activation of receptor tyrosine kinases (RTKs) (e.g. ERB1, ERBB2, FLT3, RET, KIT), activation or inactivation of RAS regulators (SOS1 and NF1) as well as constitutively activating mutations in RAS genes (H-, K- and NRAS; overall 30% of cancers) or in the BRAF gene (8% of cancers). The prevalence of KRAS mutations is especially high in pancreatic (>90%), colorectal (50%), and lung (30%) cancers. For their part, BRAF mutations are found with notably high frequencies in malignant melanoma (70%), thyroid cancer (40%) and colorectal cancer (10%) (mutation frequencies based on COSMIC (Catalogue Of Somatic Mutations In Cancer; Wellcome Trust Sanger Institute) release v95, Nov. 24, 2021).

RAS proteins are small GTPases that convey extracellular growth signals to intracellular effectors to control vital processes like cell differentiation, proliferation and survival (Nat. Rev. Cancer 2003, 3, 459). Physiological activation of RAS occurs at the plasma membrane after stimulation of RTKs, which leads to GTP loading of the GTPase and thus its activation. Activated RAS interacts and activates a battery of effector molecules, with the RAF kinases being the most critical RAS interactors in the context of cancer development (Nature Rev. Drug Discov. 2014, 13, 828). Oncogenic mutations at Glycine 12, Glycine 13 or Glutamine 61 in RAS isoforms lead to aberrant and constitutive signaling in human cancer (Nat. Rev. Cancer 2003, 3, 459) (COSMIC release v95, Nov. 24, 2021).

Downstream of RAS, mammalian cells express three RAF paralogs (ARAF, BRAF and CRAF) that share a conserved C-terminal kinase domain (KD) (Nat. Rev. Mol. Cell Biol. 2015, 16, 281) and an N-terminal regulatory region (NTR) comprising a RAS-binding domain (RBD). In unstimulated cells, RAF proteins are sequestered in the cytoplasm as monomers. Binding of GTP-bound activated RAS to the RBD induces membrane anchoring of RAF kinases (Nat. Rev. Mol. Cell Biol. 2015, 16, 281). Concomitantly, RAF proteins undergo kinase domain side-to-side dimerization and catalytic activation (Nature 2009, 461, 542). Activated RAF proteins convey signals through a phosphorylation cascade from RAF to MEK and then MEK to ERK, leading to phosphorylation by ERK of an array of substrates eliciting cell-specific responses (Nat. Rev. Mol. Cell Biol. 2020 Oct. 21(10), 607).

Activating mutations in RAF isoforms have so far been mostly restricted to the BRAF gene, although rare variants were observed in ARAF and CRAF, underlining the functional importance of this isoform (COSMIC release v95, Nov. 24, 2021). The most common cancer mutation in BRAF, a valine to glutamic acid substitution at position 600 (referred to as BRAF^{V600E}) enhances BRAF activity by stabilizing its active form (Cell 2004, 116, 855). Apart from the V600E allele, a diverse set of mutations occur at other residues (e.g. G466V, D594G, etc.) that lead to increased RAF signaling through a variety of mechanisms (Nat. Rev. Mol. Cell Biol. 2015, 16, 281). These have been grouped in three main classes (1 to 3) depending on the level of dependence to RAS activity and to RAF dimerization (Nature 2017 Aug. 10, 548(7666), 234-238). The key role of wild-type BRAF and CRAF in mediating RAS-driven oncogenesis by stimulating ERK signaling is extensively validated (Cancer Cell 2011, 19, 652; Cancer Discov. 2012, 2, 685; Nat. Commun. 2017, 8, 15262). Tumor cells thus rely on elevated and continued signaling of the RAS-ERK pathway through RAS and RAF activation, providing strong support for the concept of targeting RAF family kinases in cancers.

To address existing medical needs, the past decade has seen the development of a broad set of ATP-competitive RAF inhibitors (Nat. Rev. Cancer 2017, 17, 676). Efforts have focused mainly on the most common RAS-independent BRAF mutation (BRAFV^{600E}), leading to the development and FDA approval of sulfonamide derivatives such as vemurafenib and dabrafenib. Some of these RAF inhibitors have shown impressive efficacy against metastatic melanomas harboring the recurrent BRAF^{V600E} allele and have been approved for treating this patient population (N. Engl. J. Med. 2011, 364, 2507; Lancet 2012, 380, 358). The clinical responses against BRAF^{V600E}-dependent melanomas result from potent ATP-competitive inhibition of the monomeric form of this specific dimerization-independent BRAF mutant protein (Cancer Cell 2015, 28, 370). Unfortunately, acquired resistance to these agents invariably develops, which is mostly caused by re-activation of the RAS-ERK pathway in part through mechanisms that stimulate RAF dimerization. These include upregulation of RTK signaling, RAS mutations, and BRAF^{V600E} amplification or truncation (Sci. Signal. 2010, 3, ra84; Nature 2010, 468, 973; Nature 2011, 480, 387; Nature Commun. 2012, 3, 724).

Concurrently, tumors exhibiting RAS activity—owing to activating RAS mutations or elevated RTK signaling, but which are otherwise wild-type for BRAF—show primary resistance to BRAF^{V600E} inhibitors (Nature 2010, 464, 431). RAF inhibitors were oppositely found to induce ERK signaling in conditions where RAS activity is elevated and therefore enhances tumor cell proliferation (Nature 2010, 464, 431). This counterintuitive phenomenon, known as the paradoxical effect, was also observed in normal tissues relying on physiological RAS activity and is the basis for some of the adverse effects seen with RAF inhibitors in melanoma patients such as the development of new secondary tumors (e.g. squamous cell carcinomas and keratoacanthomas) (Nat. Rev. Cancer 2014, 14, 455). As a consequence, BRAF^{V600E} inhibitors are ineffective and even contraindicated against RAS-driven cancers. The underlying mechanism results from the compounds' ability to promote RAF kinase domain dimerization in the presence of active RAS (Nature 2010, 464, 431). This event is not restricted to BRAF, but also involves other RAF family members and is dictated by the compound binding mode and affinity (Nat. Chem. Biol. 2013, 9, 428).

Two strategies have recently been pursued to circumvent the limitation of first-generation RAF inhibitors in RAS-mutated cancers. The first one relies on the observation that paradoxical ERK activation is a dose-dependent phenomenon, i.e. induction occurs at sub-saturating inhibitor concentrations but the pathway is suppressed at saturating concentrations when the compound occupies both protomers of RAF dimers. The first strategy therefore focused on developing molecules with higher binding affinities to all RAF paralogs in order to saturate RAF proteins at lower drug concentration thereby reducing paradoxical pathway induction (Bioorg. Med. Chem. Lett. 2012, 22, 6237; Cancer Res. 2013, 73, 7043; J. Med. Chem. 2015, 58, 4165; Cancer Cell 2017, 31, 466; J Med Chem. 2020, 63, 2013; Clin Cancer Res. 2021, 27, 2061; Nature 2021, 594, 418). These compounds however retain strong RAF dimer induction capabilities and thus paradoxically stimulate RAS-ERK signaling, although with a lower amplitude than previous generations of RAF inhibitors. Although this class of compounds show improved properties, they were recently showed to mostly spare the ARAF isoform, which leads to paradoxical pathway activation and primary resistance as well as to the emergence of acquired resistance in vitro and in clinical settings (Clin Cancer Res. 2021, 27, 2061; Nature 2021, 594, 418). The second strategy consisted in designing compounds that conformationally bias the BRAF kinase domain in the inactive state and thus do not paradoxically induce ERK signaling. This has given rise to the “Paradox Breaker” (PB) molecule PLX8394, a derivative of PLX4032/vemurafenib (Nature 2015, 526, 583). These molecules retained high potency against BRAF^{V600E} and therefore should prove useful for treating BRAF^{V600E}-dependent melanomas. However, while PLX8394 does not induce ERK signaling in RAS-mutant cell lines that have been tested, it remains ineffective and is not useful against RAS-mutant tumors.

There remains a need for inhibitors that potently and consistently block RAS-ERK signaling and cellular proliferation in human tumor cells bearing a variety of RAS and RAF genotypes. The development of such inhibitors importantly being also devoid of paradoxical pathway induction in a variety of RAS-mutant tumor cell lines is highly desirable.

### SUMMARY

According to one aspect, the present technology relates to a compound of Formula I:

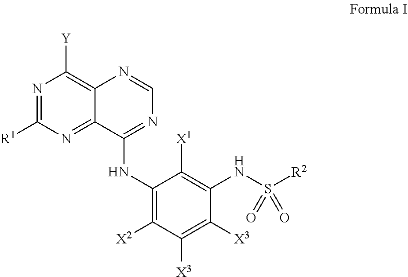

wherein:
R¹ is selected from substituted or unsubstituted OR³, SR³, NH₂, NHR³, N(R³)₂, C₃₋₈cycloalkyl, C₄₋₈heterocycloalkyl, C₆₋₁₀aryl and C₅₋₁₀heteroaryl;
R² is selected from substituted C₆aryl and C₅₋₁₀heteroaryl, substituted or unsubstituted C₄₋₈heterocycloalkyl and N(R³)₂;
R³ is independently in each occurrence selected from substituted or unsubstituted C₁₋₈alkyl, C₃₋₈cycloalkyl, C₄₋₈heterocycloalkyl, C₆₋₁₀aryl and C₅₋₁₀heteroaryl;
X¹ is halo or an electron-withdrawing group;
X² is selected from H, halo, and an electron-withdrawing group;
X³ and X⁴ are each selected from H, halo, an electron-withdrawing group, C₁₋₃alkyl, C₃₋₄cycloalkyl, and OC₁₋₃alkyl;
Y is selected from H, halo, CN, OH, OC₁₋₈alkyl, NH₂, NHC₁₋₈alkyl, N(C₁₋₈alkyl)₂, and a substituted or unsubstituted C₁₋₈alkyl;

or a pharmaceutically acceptable salt or solvate thereof;

provided that the compound is other than:

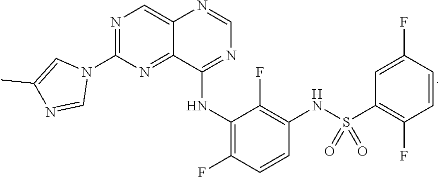 [SMILES: Cc1cn(-c2ncc3ncnc(Nc4c(F)ccc(NS(=O)(=O)c5cc(F)ccc5F)c4F)c3n2)cn1]

The compounds of Formula I are also defined according to any of the embodiments and examples described throughout the present document.

According to another aspect, the present technology relates to a pharmaceutical composition for a use as defined in any one of the aforementioned embodiments, the composition comprising a compound as herein defined together with a pharmaceutically acceptable carrier, diluent or excipient.

In a further aspect, the present technology relates to the use of a compound as herein defined for the treatment of a disease or disorder selected from a proliferative disease or disorder, a developmental anomaly caused by dysregulation of the RAS-ERK signaling cascade (RASopathies), or an inflammatory disease or an immune system disorder.

The present technology also further relates to a method for the treatment of a disease or disorder selected from a proliferative disease or disorder, a developmental anomaly caused by dysregulation of the RAS-ERK signaling cascade (RASopathies), or an inflammatory disease or an immune system disorder, comprising administering a compound as herein defined to a subject in need thereof. A method for inhibiting abnormal proliferation of cells, comprising contacting the cells with a compound as defined herein is also contemplated.

In one embodiment of the above uses and methods, the disease or disorder is selected from a neoplasm and a developmental anomaly, for instance, a disease or disorder associated with a RAF gene mutation (e.g. ARAF, BRAF or CRAF), a disease or disorder associated with a RAS gene mutation (e.g. KRAS), or a disease or disorder associated with both a RAF gene mutation and a RAS gene mutation. In one embodiment, the disease or disorder is associated with a receptor tyrosine kinase mutation or amplification (e.g. EGFR, HER2) or a mutation in a regulator of RAS downstream of the receptor (e.g. SOS1 gain of function, NF1 loss of function.

For instance, the disease or disorder is a neoplasm, such as those selected from melanoma, thyroid carcinoma (e.g. papillary thyroid carcinoma), colorectal, ovarian, breast cancer, endometrial cancer, liver cancer, sarcoma, stomach cancer, pancreatic carcinoma, Barret's adenocarcinoma, glioma (e.g. ependymoma), lung cancer (e.g. non-small cell lung cancer), head and neck cancer, acute lymphoblastic leukemia, acute myelogenous leukemia, non-Hodgkin's lymphoma, and hairy-cell leukemia. For instance, the neoplasm is selected from colon or colorectal cancer, lung cancer, pancreatic cancer, thyroid cancer, breast cancer and melanoma. For instance, any of the present uses and methods comprises inhibiting the RAS-ERK signaling pathway without substantial induction of a paradoxical pathway.

Additional objects and features of the present compound, compositions, methods and uses will become more apparent upon reading of the following non-restrictive description of exemplary embodiments and examples section, which should not be interpreted as limiting the scope of the invention.

### BRIEF DESCRIPTION OF THE DRAWINGS

FIG. 1 shows representative IC₅₀ inhibition dose response curves for compounds as described herein that do not induce paradoxical induction of pERK signaling (Y_{MIN}>−20%) in RAS-mutant HCT116 cells (Examples 80 and 81) and a compound (PLX4720; CAS #918505-84-7) that causes strong induction of the pathway in the same cell line (Y_{MIN}˜−600%).

FIG. 2 shows results of immunoblot analysis of RAS-mutant HCT-116 cells treated with a representative compound (Example 80; top panels) that does not induce paradoxical induction of pERK or pMEK signaling and by comparison, a compound (PLX4720; bottom panels) that induces the pathway in the same cell line.

### DETAILED DESCRIPTION

All technical and scientific terms and expressions used herein have the same definitions as those commonly understood by a person skilled in the art to which the present technology pertains. The definition of some terms and expressions used is nevertheless provided below. To the extent the definitions of terms in the publications, patents, and patent applications incorporated herein by reference are contrary to the definitions set forth in this specification, the definitions in this specification will control. The section headings used herein are for organizational purposes only and are not to be construed as limiting the subject matter disclosed.

### i. Definitions

Chemical structures described herein are drawn according to conventional standards. Also, when an atom, such as a carbon atom, as drawn seems to include an incomplete valency, then the valency is assumed to be satisfied by one or more hydrogen atoms even though these are not necessarily explicitly drawn. Hydrogen atoms should be inferred to be part of the compound.

The terminology used herein is for the purpose of describing particular embodiments only and is not intended to be limiting. It should be noted that, the singular forms “a”, “an”, and “the” include plural forms as well, unless the content clearly dictates otherwise. Thus, for example, reference to a composition containing “a compound” also contemplates a mixture of two or more compounds. It should also be noted that the term “or” is generally employed in its sense including “and/or” unless the context clearly dictates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the terms “compounds”, “compounds herein described”, “compounds of the present application”, “pyrimido[5,4-d]pyrimidine compounds”, “pyrimidopyrimidine compounds” and equivalent expressions refer to compounds described in the present application, e.g. those encompassed by structural Formula I, optionally with reference to any of the applicable embodiments, and also includes exemplary compounds, such as the compounds of Examples 1 to 114, as well as their pharmaceutically acceptable salts, solvates, esters, and prodrugs when applicable. When a zwitterionic form is possible, the compound may be drawn as its neutral form for practical purposes, but the compound is understood to also include its zwitterionic form. Embodiments herein may also exclude one or more of the compounds. Compounds may be identified either by their chemical structure or their chemical name. In a case where the chemical structure and chemical name would conflict, the chemical structure will prevail.

Unless otherwise stated, structures depicted herein are also meant to include all isomeric (e.g., enantiomeric, diastereomeric, and geometric (or conformational)) forms of the structure when applicable; for example, the R and S configurations for each asymmetric center. Therefore, single stereochemical isomers as well as enantiomeric, diastereomeric, and geometric (or conformational) mixtures of the present compounds are within the scope of the present description. The therapeutic compound unless otherwise noted, also encompasses all possible tautomeric forms of the illustrated compound, if any. The term also includes isotopically labeled compounds where one or more atoms have an atomic mass different from the atomic mass most abundantly found in nature. Examples of isotopes that may be incorporated into the present compounds include, but are not limited to, ²H (D), ³H (T), ¹¹C, ¹³C, ¹⁴C ¹⁵N, ¹⁸O, ¹⁷O, any one of the isotopes of sulfur, etc. The compound may also exist in unsolvated forms as well as solvated forms, including hydrated forms. The compound may exist in multiple crystalline or amorphous forms. In general, all physical forms are equivalent for the uses contemplated herein and are intended to be within the scope of the present invention.

Where a particular enantiomer is preferred, it may, in some embodiments be provided substantially free of the corresponding enantiomer and may also be enantiomerically enriched. “Enantiomerically enriched” means that the compound is made up of a significantly greater proportion of one enantiomer. In certain embodiments the compound is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including high-pressure liquid chromatography (HPLC) on chiral support and the formation and crystallization of chiral salts or be prepared by asymmetric syntheses.

The expression “pharmaceutically acceptable salt” refers to those salts of the compounds of the present description which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the present description, or separately by reacting a free base function of the compound with a suitable organic or inorganic acid (acid addition salts) or by reacting an acidic function of the compound with a suitable organic or inorganic base (base-addition salts).

The term “solvate” refers to a physical association of one of the present compound with one or more solvent molecules, including water and non-aqueous solvent molecules. This physical association may include hydrogen bonding. In certain instances, the solvate will be capable of isolation, for example when one or more solvent molecules are incorporated in the crystal lattice of a crystalline solid. The term “solvate” encompasses both solution-phase and isolable solvates. Exemplary solvates include, without limitation, hydrates, hemihydrates, ethanolates, hemiethanolates, n-propanolates, iso-propanolates, 1-butanolates, 2-butanolate, and solvates of other physiologically acceptable solvents, such as the Class 3 solvents described in the International Conference on Harmonization (ICH), Guide for Industry, Q3C Impurities: Residual Solvents (1997). Accordingly, the compound as herein described also includes each of its solvates and mixtures thereof.

As used herein, the expression “pharmaceutically acceptable ester” refers to esters of the compounds formed by the process of the present description which may hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates of hydroxyl groups, and alkyl esters of an acidic group. Other ester groups include sulfonate or sulfate esters.

The expression “pharmaceutically acceptable prodrugs” as used herein refers to those prodrugs of the compounds formed by the process of the present description which are, within the scope of sound medical judgment, suitable for use in contact with the tissues of humans and lower animals with undue toxicity, irritation, allergic response, and the like, commensurate with a reasonable benefit/risk ratio, and effective for their intended use. “Prodrug”, as used herein means a compound which is convertible in vivo by metabolic means (e.g. by hydrolysis) to afford any compound delineated by the formulae of the instant description.

Abbreviations may also be used throughout the application, unless otherwise noted, such abbreviations are intended to have the meaning generally understood by the field. Examples of such abbreviations include Me (methyl), Et (ethyl), Pr (propyl), i-Pr (isopropyl), Bu (butyl), t-Bu (tert-butyl), i-Bu (iso-butyl), s-Bu (sec-butyl), c-Bu (cyclobutyl), Ph (phenyl), Bn (benzyl), Bz (benzoyl), CBz or Cbz or Z (carbobenzyloxy), Boc or BOC (tert-butoxycarbonyl), and Su or Suc (succinimide). For more certainty, additional definitions of specific abbreviations are also included in the introduction of the Examples section.

The number of carbon atoms in a hydrocarbyl substituent can be indicated by the prefix “Cₓ-C_{y}” or “C_{x-y}” where x is the minimum and y is the maximum number of carbon atoms in the substituent. However, when the prefix “Cₓ-C_{y}” or “C_{x-y}” is associated with a group incorporating one or more heteroatom(s) by definition (e.g. heterocycloalkyl, heteroaryl, etc), then x and y define respectively the minimum and maximum number of atoms in the cycle, including carbon atoms as well as heteroatom(s).

The term “alkyl” as used herein, refers to a saturated, straight- or branched-chain hydrocarbon radical typically containing from 1 to 20 carbon atoms. For example, “C₁₋₈alkyl” contains from one to eight carbon atoms. Examples of alkyl radicals include, but are not limited to, methyl, ethyl, propyl, isopropyl, n-butyl, tert-butyl, neopentyl, n-hexyl, heptyl, octyl radicals and the like.

The term “alkenyl” as used herein, denotes a straight- or branched-chain hydrocarbon radical containing one or more double bonds and typically from 2 to 20 carbon atoms. For example, “C₂₋₈alkenyl” contains from two to eight carbon atoms. Alkenyl groups include, but are not limited to, for example, ethenyl, propenyl, butenyl, I-methyl-2-buten-1-yl, heptenyl, octenyl and the like.

The term “alkynyl” as used herein, denotes a straight- or branched-chain hydrocarbon radical containing one or more triple bonds and typically from 2 to 20 carbon atoms. For example, “C₂₋₈alkynyl” contains from two to eight carbon atoms. Representative alkynyl groups include, but are not limited to, for example, ethynyl, 1-propynyl, 1-butynyl, heptynyl, octynyl and the like.

The terms “cycloalkyl”, “alicyclic”, “carbocycle”, “carbocyclic” and equivalent expressions refer to a group comprising a saturated or partially unsaturated (non aromatic) carbocyclic ring in a monocyclic or polycyclic ring system, including spiro (sharing one atom), fused (sharing at least one bond) or bridged (sharing two or more bonds) carbocyclic ring systems, having from three to fifteen ring members. Examples of cycloalkyl groups include, without limitation, cyclopropyl, cyclobutyl, cyclopentyl, cyclopenten-1-yl, cyclopenten-2-yl, cyclopenten-3-yl, cyclohexyl, cyclohexen-1-yl, cyclohexen-2-yl, cyclohexen-3-yl, cycloheptyl, bicyclo[4,3,0]nonanyl, norbornyl, and the like. The term cycloalkyl includes both unsubstituted cycloalkyl groups and substituted cycloalkyl groups. For example, the term “C₃₋ₙcycloalkyl” refers to a cycloalkyl group having from 3 to the indicated “n” number of carbon atoms in the ring structure. Unless the number of carbons is otherwise specified, “lower cycloalkyl” groups as herein used, have at least 3 and equal or less than 8 carbon atoms in their ring structure.

As used herein, the terms “heterocycle”, “heterocycloalkyl”, “heterocyclyl”, “heterocyclic radical”, and “heterocyclic ring” are used interchangeably and refer to a chemically stable 3- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 1-3 heteroatoms selected from oxygen, sulfur or nitrogen, the nitrogen may be N (as in 3,4-dihydro-2H-pyrrolyl), NH (as in pyrrolidinyl), or NR (as in N-substituted pyrrolidinyl). A heterocyclic ring can be attached to its pendant group at any heteroatom or carbon atom that results in a chemically stable structure and any of the ring atoms can be optionally substituted. Examples of heterocycloalkyl groups include, but are not limited to, 1,3-dioxolanyl, pyrrolidinyl, pyrrolidonyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, tetrahydrofuranyl, tetrahydropyranyl, tetrahydrothiopyranyl, tetrahydrodithienyl, tetrahydrothienyl, thiomorpholino, thioxanyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, 2H-pyranyl, 4H-pyranyl, dioxanyl, dithianyl, dithiolanyl, dihydropyranyl, dihydrothienyl, dihydrofuranyl, 3-azabicyclo[3,1,0]hexanyl, 3-azabicyclo[4,1,0]heptanyl, quinolizinyl, quinuclidinyl, tetrahydroquinolinyl, tetrahydroisoquinolinyl, decahydroquinolinyl, and the like. Heterocyclic groups also include groups in which a heterocyclic ring is fused to one or more aryl, heteroaryl, or cycloaliphatic rings, such as indolinyl, 3H-indolyl, chromanyl, chromenyl, phenanthridinyl, 2-azabicyclo[2.2.1]heptanyl, octahydroindolyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted. For instance, the term “C₃₋ₙheterocycloalkyl” refers to a heterocycloalkyl group having from 3 to the indicated “n” number of atoms in the ring structure, including carbon atoms and heteroatoms.

As used herein, the term “partially unsaturated” refers to a ring moiety that includes at least one double or triple bond between ring atoms but is not aromatic. The term “partially unsaturated” is intended to encompass rings having multiple sites of unsaturation but is not intended to include aryl or heteroaryl moieties, as herein defined.

The term “aryl” used alone or as part of a larger moiety as in “aralkyl”, “aralkoxy”, “aryloxy”, or “aryloxyalkyl”, refers to aromatic groups having 4n+2 conjugated π(pi) electrons, wherein n is an integer from 1 to 3, in a monocyclic moiety or a bicyclic or tricyclic fused ring system having a total of six to 15 ring members, wherein at least one ring in the system is aromatic and wherein each ring in the system contains three to seven ring members. The term “aryl” may be used interchangeably with the term “aryl ring”. In certain embodiments of the present description, “aryl” refers to an aromatic ring system which includes, but not limited to, phenyl, biphenyl, naphthyl, azulenyl, anthracyl and the like, which may bear one or more substituents. The term “aralkyl” or “arylalkyl” refers to an alkyl residue attached to an aryl ring. Examples of aralkyl include, but are not limited to, benzyl, phenethyl, and the like. Also included within the scope of the term “aryl”, as it is used herein, is a group in which an aromatic ring is fused to one or more non-aromatic rings, such as indanyl, indenyl, phthalimidyl, naphthimidyl, fluorenyl, phenanthridinyl, or tetrahydronaphthyl, and the like. For example, the term “C₆₋ₙaryl” refers to an aryl group having from 6 to the indicated “n” number of atoms in the ring structure.

The term “heteroaryl”, used alone or as part of a larger moiety, e.g., “heteroaralkyl”, or “heteroaralkoxy”, refers to aromatic groups having 4n+2 conjugated π(pi) electrons, wherein n is an integer from 1 to 3 (e.g. having 5 to 18 ring atoms, preferably 5, 6, or 9 ring atoms; having 6, 10, or 14 π electrons shared in a cyclic array); and having, in addition to carbon atoms, from one to five heteroatoms. The term “heteroatom” includes but is not limited to nitrogen, oxygen, or sulfur, and includes any oxidized form of nitrogen or sulfur, and any quaternized form of a basic nitrogen. A heteroaryl may be a single ring, or two or more fused rings. The term “heteroaryl”, as used herein, also includes groups in which a heteroaromatic ring is fused to one or more aryl, cycloaliphatic, or heterocyclic rings, where the radical or point of attachment is on the heteroaromatic ring. Nonlimiting examples of heteroaryl groups include thienyl, furanyl (furyl), pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, triazinyl, indolyl, 3H-indolyl, isoindolyl, indolizinyl, benzothienyl (benzothiophenyl), benzofuranyl, dibenzofuranyl, indazolyl, benzimidazolyl, benzoxazolyl, benzothiazolyl, benzotriazolyl, pyrrolopyridinyl (e.g. pyrrolo[3,2-b]pyridinyl or pyrrolo[3,2-c]pyridinyl), pyrazolopyridinyl (e.g. pyrazolo[1,5-a]pyridinyl), furopyridinyl, purinyl, imidazopyrazinyl (e.g. imidazo[4,5-b]pyrazinyl), quinolyl (quinolinyl), isoquinolyl (isoquinolinyl), quinolonyl, isoquinolonyl, cinnolinyl, phthalazinyl, quinazolinyl, quinoxalinyl, 4H-quinolizinyl, naphthyridinyl, and pteridinyl carbazolyl, acridinyl, phenanthridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. Heteroaryl groups include rings that are optionally substituted. The term “heteroaralkyl” refers to an alkyl group substituted by a heteroaryl, wherein the alkyl and heteroaryl portions independently are optionally substituted. Examples include, but are not limited to, pyridinylmethyl, pyrimidinylethyl and the like. For instance, the term “C₅₋ₙheteroaryl” refers to a heteroaryl group having from 5 to the indicated “n” number of atoms in the ring structure, including carbon atoms and heteroatoms.

As described herein, compounds of the present description may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under the present description are preferably those that result in the formation of chemically stable or chemically feasible compounds. The term “chemically stable”, as used herein, refers to compounds that are not substantially altered when subjected to conditions to allow for their production, detection, and, in certain embodiments, their recovery, purification, and use for one or more of the purposes disclosed herein.

The term “halo” designates a halogen atom, i.e. a fluorine, chlorine, bromine or iodine atom, preferably fluorine or chlorine.

The term “optionally substituted” refers to groups that are substituted or unsubstituted by independent replacement of one, two, or three or more of the hydrogen atoms thereon with substituents including, but not limited to F, Cl, Br, I, OH, CO₂H, alkoxy, oxo, thiooxo, NO₂, CN, CF₃, NH₂, NHalkyl, NHalkenyl, NHalkynyl, NHcycloalkyl, NHaryl, NHheteroaryl, NHheterocyclic, dialkylamino, diarylamino, diheteroarylamino, O-alkyl, O-alkenyl, O-alkynyl, O-cycloalkyl, O-aryl, O-heteroaryl, O-haloalkyl, O-heterocyclic, C(O)alkyl, C(O)alkenyl, C(O)alkynyl, C(O)cycloalkyl, C(O)aryl, C(O)heteroaryl, C(O)heterocycloalkyl, CO₂alkyl, CO₂alkenyl, CO₂alkynyl, CO₂cycloalkyl, CO₂aryl, CO₂heteroaryl, CO₂heterocycloalkyl, OC(O)alkyl, OC(O)alkenyl, OC(O)alkynyl, OC(O)cycloalkyl, OC(O)aryl, OC(O)heteroaryl, OC(O)heterocycloalkyl, C(O)NH₂, C(O)NHalkyl, C(O)NHalkenyl, C(O)NHalkynyl, C(O)NHcycloalkyl, C(O)NHaryl, C(O)NHheteroaryl, C(O)NHheterocycloalkyl, OCO₂alkyl, OCO₂alkenyl, OCO₂alkynyl, OCO₂cycloalkyl, OCO₂aryl, OCO₂heteroaryl, OCO₂heterocycloalkyl, OC(O)NH₂, OC(O)NHalkyl, OC(O)NHalkenyl, OC(O)NHalkynyl, OC(O)NHcycloalkyl, OC(O)NHaryl, OC(O)NHheteroaryl, OC(O)NHheterocycloalkyl, NHC(O)alkyl, NHC(O)alkenyl, NHC(O)alkynyl, NHC(O)cycloalkyl, NHC(O)aryl, NHC(O)heteroaryl, NHC(O)heterocycloalkyl, NHCO₂alkyl, NHCO₂alkenyl, NHCO₂alkynyl, NHCO₂cycloalkyl, NHCO₂aryl, NHCO₂heteroaryl, NHCO₂heterocycloalkyl, NHC(O)NH₂, NHC(O)NHalkyl, NHC(O)NHalkenyl, NHC(O)NHalkenyl, NHC(O)NHcycloalkyl, NHC(O)NHaryl, NHC(O)NHheteroaryl, NHC(O)NHheterocycloalkyl, NHC(S)NH₂, NHC(S)NHalkyl, NHC(S)NHalkenyl, NHC(S)NHalkynyl, NHC(S)NHcycloalkyl, NHC(S)NHaryl, NHC(S)NHheteroaryl, NHC(S)NHheterocycloalkyl, NHC(NH)NH₂, NHC(NH)NHalkyl, NHC(NH)NHalkenyl, NHC(NH)NHalkenyl, NHC(NH)NHcycloalkyl, NHC(NH)NHaryl, NHC(NH)NHheteroaryl, NHC(NH)NHheterocycloalkyl, NHC(NH)alkyl, NHC(NH)alkenyl, NHC(NH)alkenyl, NHC(NH)cycloalkyl, NHC(NH)aryl, NHC(NH)heteroaryl, NHC(NH)heterocycloalkyl, C(NH)NHalkyl, C(NH)NHalkenyl, C(NH)NHalkynyl, C(NH)NHcycloalkyl, C(NH)NHaryl, C(NH)NHheteroaryl, C(NH)NHheterocycloalkyl, P(O)(alkyl)₂, P(O)(alkenyl)₂, P(O)(alkynyl)₂, P(O)(cycloalkyl)₂, P(O)(aryl)₂, P(O)(heteroaryl)₂, P(O)(heterocycloalkyl)₂, P(O)(Oalkyl)₂, P(O)(OH)₂, P(O)(Oalkenyl)₂, P(O)(Oalkynyl)₂, P(O)(Ocycloalkyl)₂, P(O)(Oaryl)₂, P(O)(Oheteroaryl)₂, P(O)(Oheterocycloalkyl)₂, S(O)alkyl, S(O)alkenyl, S(O)alkynyl, S(O)cycloalkyl, S(O)aryl, S(O)₂alkyl, S(O)₂alkenyl, S(O)₂alkynyl, S(O)₂cycloalkyl, S(O)₂aryl, S(O)heteroaryl, S(O)heterocycloalkyl, SO₂NH₂, SO₂NHalkyl, SO₂NHalkenyl, SO₂NHalkynyl, SO₂NHcycloalkyl, SO₂NHaryl, SO₂NHheteroaryl, SO₂NHheterocycloalkyl, NHSO₂alkyl, NHSO₂alkenyl, NHSO₂alkynyl, NHSO₂cycloalkyl, NHSO₂aryl, NHSO₂heteroaryl, NHSO₂heterocycloalkyl, CH₂NH₂, CH₂SO₂CH₃, alkyl, alkenyl, alkynyl, aryl, arylalkyl, heteroaryl, heteroarylalkyl, heterocycloalkyl, cycloalkyl, carbocyclic, heterocyclic, polyalkoxyalkyl, polyalkoxy, methoxymethoxy, methoxyethoxy, SH, S-alkyl, S-alkenyl, S-alkynyl, S-cycloalkyl, S-aryl, S-heteroaryl, S-heterocycloalkyl, or methylthiomethyl.

### ii. Compounds

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof. As such, the following embodiments are present alone or in combination if applicable.

The present compounds present a pyrimido[5,4-d]pyrimidine core structure to which is attached defined substituents to achieve the product's beneficial activity. Examples of pyrimidopyrimidine compounds as defined herein are illustrated by general Formula I:

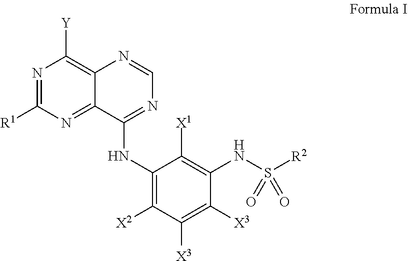

wherein:
R¹ is selected from substituted or unsubstituted OR³, SR³, NH₂, NHR³, N(R³)₂, C₃₋₈cycloalkyl, C₄₋₈heterocycloalkyl, C₆₋₁₀aryl and C₅₋₁₀heteroaryl;
R² is selected from substituted C₆aryl and C₅₋₁₀heteroaryl, substituted or unsubstituted C₄₋₈heterocycloalkyl and N(R³)₂;
R³ is independently in each occurrence selected from substituted or unsubstituted C₁₋₈alkyl, C₃₋₈cycloalkyl, C₄₋₈heterocycloalkyl, C₆₋₁₀aryl and C₅₋₁₀heteroaryl;
X¹ is halo or an electron-withdrawing group;
X² is selected from H, halo, and an electron-withdrawing group;
X³ and X⁴ are each selected from H, halo, an electron-withdrawing group, C₁₋₃alkyl, C₃₋₄cycloalkyl, and OC₁₋₃alkyl;
Y is selected from H, halo, CN, OH, OC₁₋₈alkyl, NH₂, NHC₁₋₈alkyl, N(C₁₋₈alkyl)₂, and a substituted or unsubstituted C₁₋₈alkyl;

or a pharmaceutically acceptable salt or solvate thereof;

provided that the compound is other than:

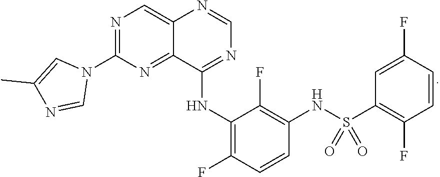 [SMILES: Cc1cn(-c2ncc3ncnc(Nc4c(F)ccc(NS(=O)(=O)c5cc(F)ccc5F)c4F)c3n2)cn1]

For example, the electron-withdrawing group is selected from perhaloalkyl (e.g. CF₃ or CCl₃), CN, NO₂, sulfonate, alkylsulfonyl (e.g. SO₂Me or SO₂CF₃), alkylcarbonyl (e.g. C(O)Me), carboxylate, alkoxycarbonyl (e.g. C(O)OMe), and aminocarbonyl (e.g. C(O)NH₂). In one embodiment, X¹ is Cl and X² is F, or X¹ is F and X² is H, or X¹ and X² are both F. In another embodiment, X³ and X⁴ are each H. In yet another embodiment, X³ is F and X⁴ is H.

According to one example, Y is H and all other groups are as herein defined. According to another example, Y is NH₂ and all other groups are as herein defined.

For instance the aminoarylsulfonamide moiety in Formula I is designated L and is selected from:

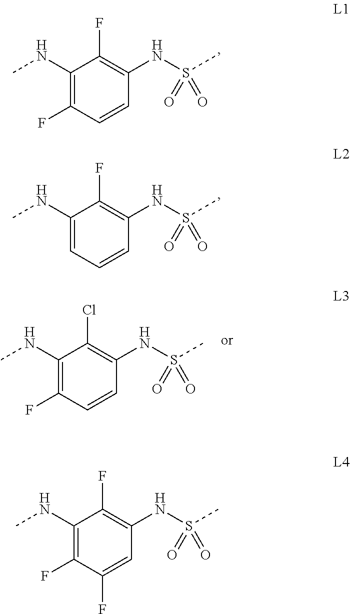

where the dashed line (---) represents a bond.

In a further embodiment, R² is a substituted C₆aryl or C₅₋₁₀heteroaryl, e.g. R² is a C₆aryl substituted with at least one group selected from F, CH, Br, CN, NO₂, and a substituted or unsubstituted C₁₋₃alkyl, C₃₋₄cycloalkyl or OC₁₋₃alkyl. For instance, R² is a group of the formula:

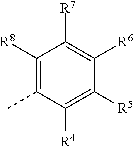

wherein:
R⁴ is selected from H, F, Cl, Br, CN, and a substituted or unsubstituted C₁₋₃alkyl, C₃₋₄cycloalkyl or OC₁₋₃alkyl, e.g. R⁴ is selected from H, F, Cl, Br, Me, Et, CN, CHF₂, and CF₃;
R⁵ is selected from H, F, Cl, CN, and a substituted or unsubstituted C₁₋₃alkyl, C₃₋₄cycloalkyl, or OC₁₋₃alkyl, e.g. R⁵ is selected from H, F, Me, CF₃, CN, and Cl;
R⁶ is selected from H, F, Cl, Br, NO₂, NH₂, and a substituted or unsubstituted C₁₋₃alkyl, C₃₋₄cycloalkyl or OC₁₋₃alkyl, e.g. R⁶ is selected from H, F, Cl, Br, and a substituted or unsubstituted C₁₋₃alkyl, C₃₋₄cycloalkyl or OC₁₋₃alkyl, or R⁶ is selected from H, F, Cl, Me, Et, and OMe;
R⁷ is selected from H, F, Cl, and a substituted or unsubstituted C₁₋₃alkyl, e.g. R⁷ is selected from H, Me, F, and Cl;
R⁸ is selected from H, F, and a substituted or unsubstituted C₁₋₃alkyl, e.g. R⁸ is selected from H, Me and F;
or R⁴ and R⁵ or R⁵ and R⁶ are taken together with their adjacent carbon atoms to form a substituted or unsubstituted carbocycle or heterocycle provided that the heterocycle (R²) is not a benzoxazolinone; and
(---) represents a bond;
wherein when R⁴ is H or F, then at least one of R⁵, R⁶, R⁷ or R⁸ is other than H or F; and
wherein when R⁵ is CN, then at least one of R⁴, R⁶, R⁷ or R⁸ is other than H.

In one embodiment, R⁸ is H. In another embodiment, R⁴ is selected from F, Cl, Et and Me, R⁵, R⁷, and R⁸ are each H, and R⁶ is selected from H, Cl, Me and OMe. In a further embodiment, R⁴ is selected from F, Cl, and Me, R⁶, R⁷ and R⁸ are each H, and R⁵ is selected from F and Cl.

In a further embodiment, R⁴ is selected from C and a substituted or unsubstituted C₁₋₃alkyl (e.g. Me); preferably R⁴ is C or Me; R⁵ is selected from H, F, Cl, and a substituted or unsubstituted C₁₋₃alkyl (e.g., Me); R⁶ is selected from H and a substituted or unsubstituted OC₁₋₃alkyl (e.g. OCH₃); and R⁷ and R⁸ are each H.

In yet another embodiment, R⁴ is selected from H, Cl, Br and methyl; R⁵ is selected from H, F, and Cl; R⁶ is selected from H, F, Cl, Me and OMe; and R⁷ and R⁸ are each H.

In a further embodiment, R⁴ is selected from Cl and a substituted or unsubstituted C₁₋₃alkyl (e.g. Me), preferably R⁴ is Cl or Me; R⁵ is selected from H, F, Cl, and a substituted or unsubstituted C₁₋₃alkyl (e.g., Me), preferably R⁵ is F, Cl or Me; R⁶ is selected from H, F, Cl, a substituted or unsubstituted C₁₋₃alkyl (e.g., Me), and a substituted or unsubstituted OC₁₋₃alkyl (e.g. OCH₃), preferably R⁶ is H or F, or R⁶ is Cl or a substituted or unsubstituted C₁₋₃alkyl or substituted or unsubstituted OC₁₋₃alkyl, or CH₃ or OCH₃; and R⁷ and R⁸ are each H. In yet another embodiment, R⁶ is a substituted C₁₋₃alkyl.

In another example, R² is a substituted C₅heteroaryl, such as a group of the formula:

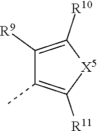

wherein:
X⁵ is selected from NH, NC₁₋₃alkyl, NC₃₋₄cycloalkyl, O and S;
R⁹, R¹⁰, R¹¹ are each independently selected from H, F, Cl, CN, and a substituted or unsubstituted C₁₋₃alkyl, C₃₋₄cycloalkyl, C(O)OC₁₋₃alkyl or OC₁₋₃alkyl, provided that one of R⁹ and R¹¹ is H and the other is not H; and
(---) represents a bond.

Alternatively, R² is a group of the formula:

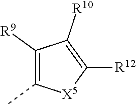

wherein:
X⁵ is selected from NH, NC₁₋₃alkyl, NC₃₋₄cycloalkyl, O and S;
R⁹ is selected from F, Cl, CN, and a substituted or unsubstituted C₁₋₃alkyl, C₃₋₄cycloalkyl, C(O)OC₁₋₃alkyl or OC₁₋₃alkyl;
R¹⁰ and R¹² are each independently selected from H, F, Cl, CN, and a substituted or unsubstituted C₁₋₃alkyl, C₃₋₄cycloalkyl, C(O)OC₁₋₃alkyl or OC₁₋₃alkyl; and
(---) represents a bond.

In a preferred embodiment, R⁹ and R¹⁰ are each independently selected from F, Cl, CN, and a substituted or unsubstituted C₁₋₃alkyl, C₃₋₄cycloalkyl, C(O)OC₁₋₃alkyl or OC₁₋₃alkyl, preferably Cl and a substituted or unsubstituted C₁₋₃alkyl, more preferably R⁹ and R¹⁰ are both Cl. In another embodiment, X⁵ is O or S, preferably S.

In another embodiment, R² is a substituted C₅₋₁₀heteroaryl, such as a group of the formula:

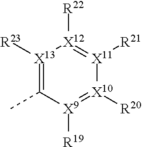

wherein:
X⁹, X¹⁰, X¹¹, X¹², and X¹³ are independently selected from N and C, wherein at least one and at most two of X⁹, X¹⁰, X¹¹, X¹², and X¹³ are N; and
R¹⁹, R²⁰, R²¹, R²² and R²³ are selected from H, F, Cl, Br, CN, NO₂, NH₂, and a substituted or unsubstituted C₁₋₃alkyl, C₃₋₄cycloalkyl or OC₁₋₃alkyl, or are absent when their attached X⁹, X¹⁰, X¹¹, X¹², or X¹³ is N;
wherein at least one of X⁹ and X¹³ is not N; and
wherein when one of X⁹ and X¹³ is N, then the other is not N or CH.

In another example, R² is a C₅heterocycloalkyl. For instance, R² is a group of the formula:

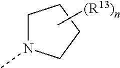

wherein:
R¹³ is independently in each occurrence selected from F, Cl, and a substituted or unsubstituted C₁₋₃alkyl, C₃₋₄cycloalkyl, or C₁₋₃alkoxy;
n is an integer selected from 0 to 8; or
n is between 2 and 8 and two R¹³ are taken together with their adjacent carbon atoms to form a C₃₋₄cycloalkyl; and
(---) represents a bond.

In one embodiment, R¹³ is in the 3-position. In another embodiment, R¹³ is selected from F, Me, OMe, and CH₂OMe, and n is 1 or 2. For instance, R¹³ is a methoxy group in the 3-position and n is 1.

In a further example, R² is N(R³)₂. For instance, R² is N(R³)₂ and R³ is selected from substituted or unsubstituted C₁₋₈alkyl or C₃₋₈cycloalkyl.

In yet another embodiment, the compound of Formula I is a compound of Formula II, or a pharmaceutically acceptable salt or solvate thereof:

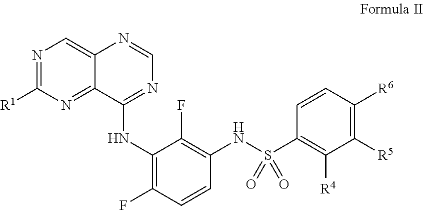

wherein R¹, R⁴, R⁵, and R⁶ are each independently as defined herein, preferably is selected from Cl, Br and methyl; R⁵ is selected from H, F, Cl and methyl; R⁶ is selected from H, F, Cl, Me and OMe.

In a further embodiment, the compound of Formula I is a compound of Formula III, or a pharmaceutically acceptable salt or solvate thereof:

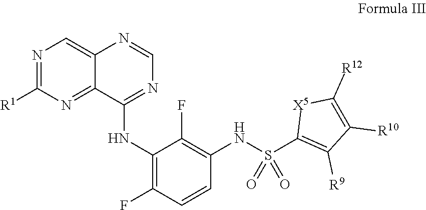

wherein R¹, R⁹, R¹⁰, R¹², and X⁵ are each independently as defined herein.

Exemplary R² groups are illustrated by groups 1 to B77 defined as follows:

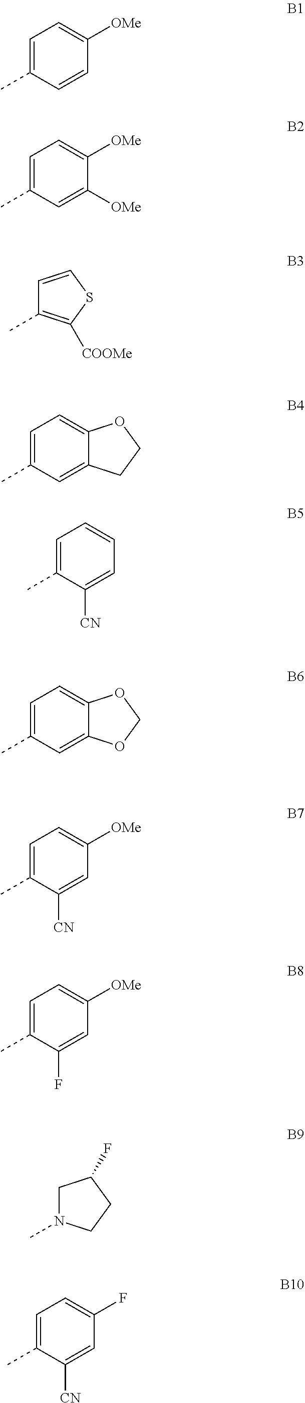

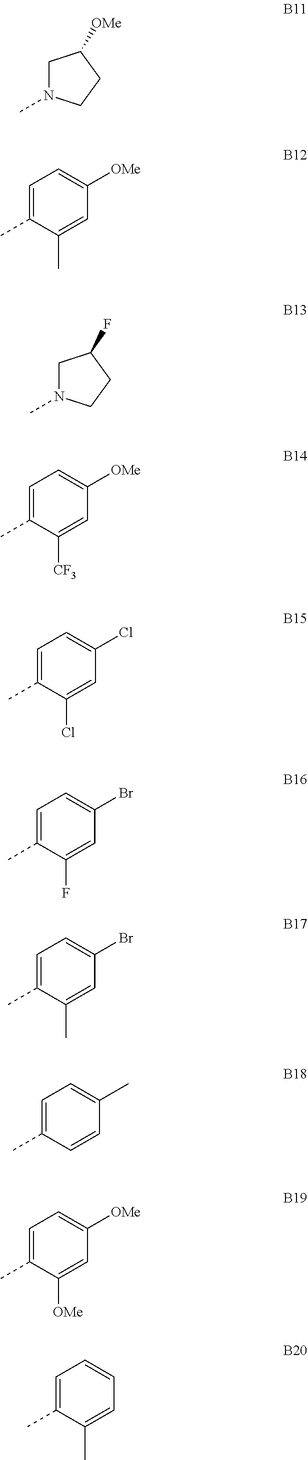

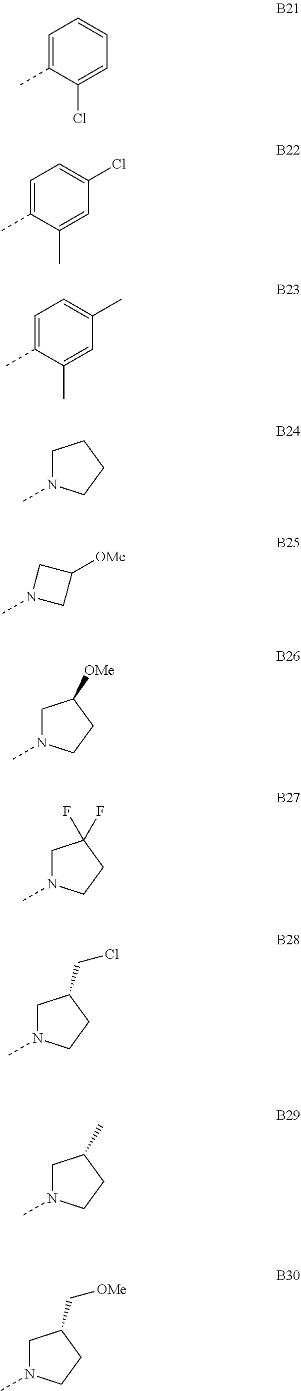

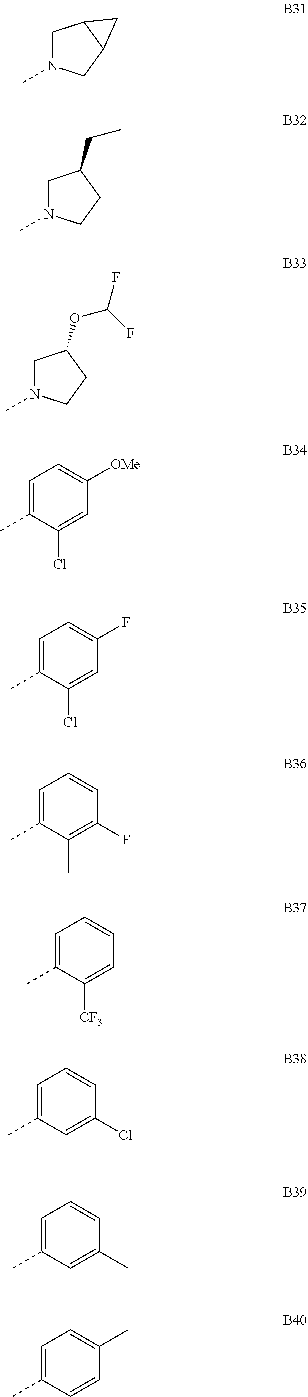

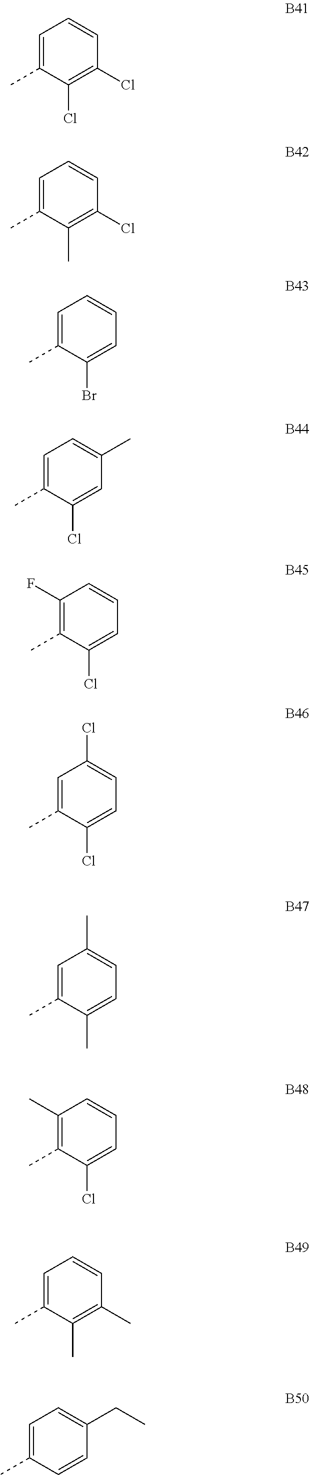

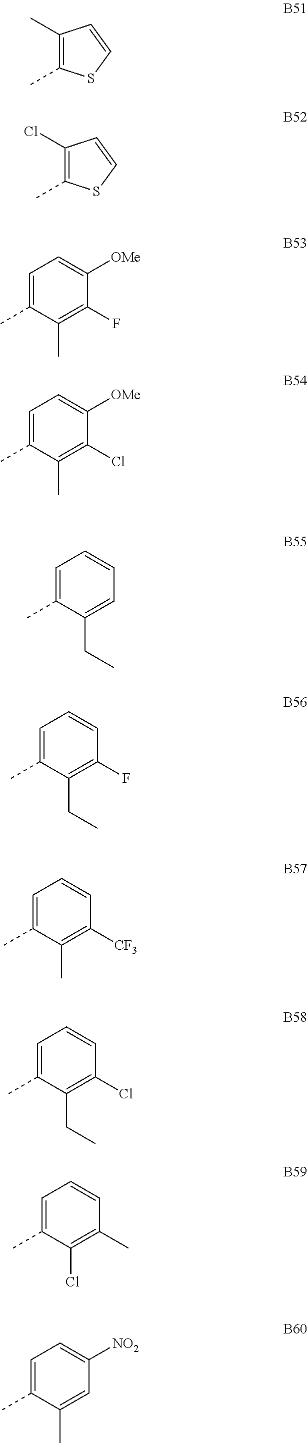

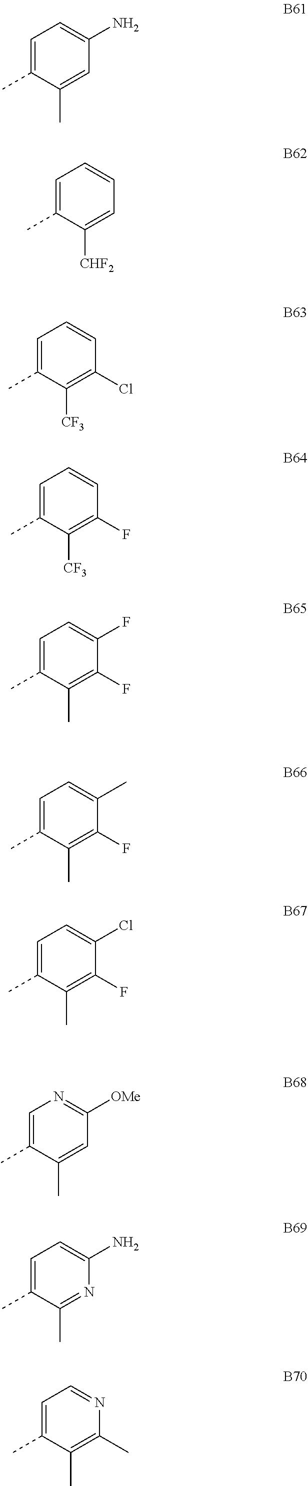

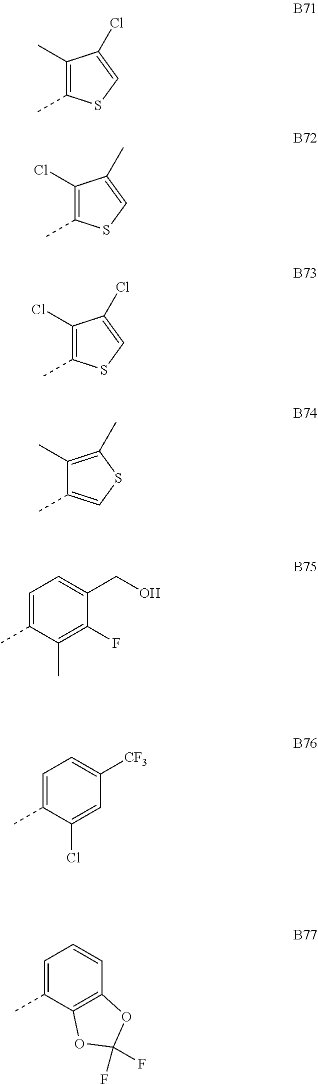

wherein (---) represents a bond.

In one embodiment, R² is selected from groups 1 to B37, B41 to B44, B49, B51 to B55, B57, B59, B62 to B67, B71 to B74, B76 and B77, or preferably R² is selected from groups B1-B33, B36, B41, B42, B51 to B54, B59, B65, B73 and B77, or more preferably R² is selected from groups 1, B2, B6, B8, B11, B12, B15, B20, B21, B36, B41, B42, B53, B54, B59, B65 and B73, or most preferably R² is selected from groups B21, B36, B41, B42, B52, B53, B54, B59, B65 and B73.

In one embodiment of the compound of Formula I, R¹ is OR³ or SR³, for instance, R¹ is SR³. In various embodiments, R³ is a substituted or unsubstituted C₁₋₈alkyl (e.g. C₁₋₃alkyl).

In another embodiment, R¹ is a substituted or unsubstituted C₆aryl group. In another embodiment, R¹ is a substituted or unsubstituted C₄₋₆heterocycloalkyl group. For instance, R¹ is a C₄₋₅heterocycloalkyl optionally substituted with one or two groups selected from halo, OH, C₁₋₆alkyl, and OC₁₋₆alkyl. For instance, R¹ is a N-pyrrolidinyl group substituted with one or two groups selected from F and OH.

In another embodiment, R¹ is a substituted or unsubstituted C₅₋₆heteroaryl group or a substituted or unsubstituted C₉heteroaryl group. In a further embodiment, R¹ is a substituted or unsubstituted group selected from thienyl, imidazolyl, pyrazolyl, triazolyl, thiazolyl, pyridyl, pyrimidinyl, indolyl, indazolyl, benzimidazolyl, benzotriazolyl, pyrrolopyridinyl (e.g. pyrrolo[3,2-b]pyridinyl or pyrrolo[3,2-c]pyridinyl), pyrazolopyridinyl (e.g. pyrazolo[1,5-a]pyridinyl), purinyl, imidazopyrazinyl (e.g. imidazo[4,5-b]pyrazinyl), and quinolyl (quinolinyl), preferably R¹ is a substituted or unsubstituted group selected from imidazolyl, pyrazolyl, triazolyl, indolyl, indazolyl, benzimidazolyl, benzotriazolyl, pyrrolopyridinyl (e.g. pyrrolo[3,2-b]pyridinyl or pyrrolo[3,2-c]pyridinyl), pyrazolopyridinyl (e.g. pyrazolo[1,5-a]pyridinyl), purinyl, and imidazopyrazinyl (e.g. imidazo[4,5-b]pyrazinyl), more preferably attached to the pyrimidopyrimidine core through a nitrogen atom.

Examples of R¹ include group selected from:

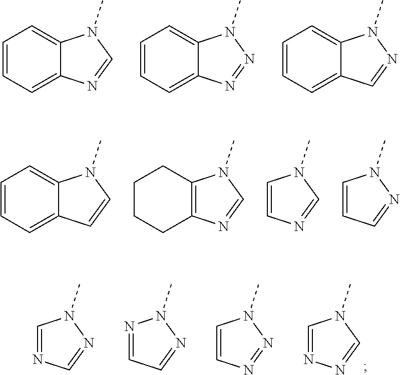

wherein (---) represents a bond, and wherein said group is optionally further substituted.

For instance, R¹ is a substituted or unsubstituted group selected from:

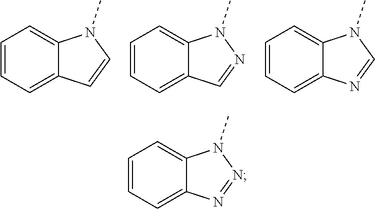

wherein (---) represents a bond.

In one embodiment, R¹ is one of the above groups further substituted with at least one substituent selected from OH, halo, CN, NO₂, C₁₋₆alkyl, C₂₋₆alkenyl, C₂₋₆alkynyl, OC₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂;
wherein:
   R¹⁴ is independently in each occurrence selected from H, C₁₋₆alkyl, C₂₋₆alkenyl, C₂₋₆alkynyl, C₃₋₁₀cycloalkyl, C₄₋₁₀heterocycloalkyl, C₆aryl, and C₅₋₁₀heteroaryl, or two R¹⁴ are taken together with their adjacent nitrogen atom to form a C₄₋₁₀heterocycloalkyl group;
   R¹⁵ is independently in each occurrence selected from C₁₋₆alkyl, C₂₋₆alkenyl, C₂₋₆alkynyl, C₃₋₁₀cycloalkyl, C₆aryl, and C₅₋₁₀heteroaryl; and
   R¹⁶ is independently in each occurrence selected from H, C₁₋₆alkyl, C₂₋₆alkenyl, C₂₋₆alkynyl, C₃₋₁₀cycloalkyl, C₆aryl, and C₅₋₁₀heteroaryl;
wherein said alkyl, alkenyl, alkynyl, cycloalkyl, heterocycloalkyl, aryl or heteroaryl group, included in R¹ (including in the definitions of R¹⁴, R¹⁵, and R¹⁶), is optionally further substituted.

In another embodiment, R¹ is a group of the formula:

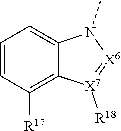

wherein:
R¹⁷ is selected from H, OH, halo, CN, NO₂, C₁₋₆alkyl, C₂₋₆alkenyl, C₂₋₆alkynyl, OC₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂;
X⁶ is N or CH; and
X⁷ is N and R¹⁸ is absent; or
X⁷ is C and R¹⁸ is selected from C₁₋₆alkyl, C₂₋₆alkenyl, C₂₋₆alkynyl, OC₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R⁴)₂;
wherein R¹⁴, R¹⁵, and R¹⁶ are as defined above;
wherein said alkyl, alkenyl, alkynyl, cycloalkyl, heterocycloalkyl, or heteroaryl, included in R¹ (including in the definitions of R¹, R¹⁵, R¹⁶, R¹⁷ and R¹⁸), is optionally further substituted; and
wherein (---) represents a bond.

In another embodiment, R¹ is a group of the formula:

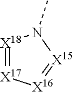

wherein:
X¹⁵, X¹⁶, X¹⁷, and X¹⁸ are independently selected from O, N, S, and CR¹⁷, wherein R¹⁷ is as previously defined;
wherein at most two of X¹⁵, X¹⁶, X¹⁷, and X¹⁸ are O, N, or S.

In one embodiment, the compound of Formula I is a compound of Formula IV or V, or a pharmaceutically acceptable salt or solvate thereof:

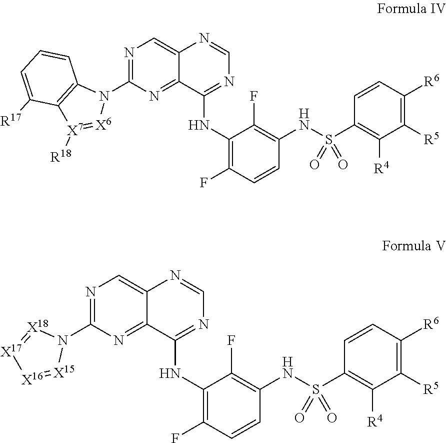

wherein R⁴, R⁵, R⁶, R¹⁷, R¹⁸, X⁶, X⁷, X¹⁵, X¹⁶, X¹⁷, and X¹⁸ are each independently as defined herein, preferably R⁴ is selected from Cl, Br and methyl; R⁵ is selected from H, F, Cl and methyl; R⁶ is selected from H, Cl, F, Me and OMe.

In a further embodiment, the compound of Formula I is a compound of Formula VI or VII, or a pharmaceutically acceptable salt or solvate thereof:

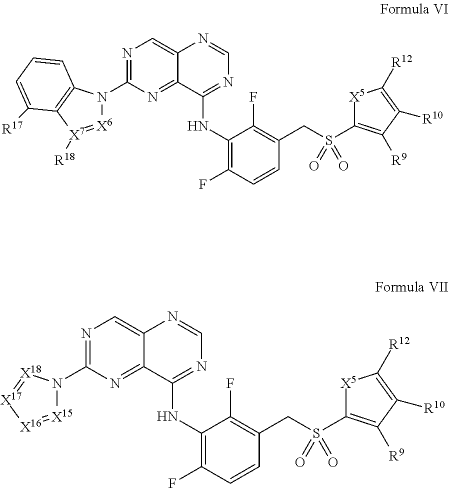

wherein R⁹, R¹⁰, R¹², R¹⁷, R¹⁸, X⁵, X⁶, X⁷, X¹⁵, X¹⁶, X¹⁷ and X¹⁸ are each independently as defined herein.

In one embodiment of the above formulae, X⁶ is N. In another embodiment, X⁶ is CH.

In another embodiment, X⁷ is N, R¹⁷ is selected from H, OH, CN, C₁₋₆alkyl, C₂₋₆alkenyl, C₂₋₆alkynyl, OC₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂, and R¹⁸ is absent, wherein said alkyl, alkenyl, alkynyl, cycloalkyl, heterocycloalkyl, or heteroaryl in R¹⁴, R¹⁵, R¹⁶, or R¹⁷, is optionally further substituted, preferably R¹⁷ is selected from C₁₋₆alkyl, C₅₋₁₀heteroaryl, C₄₋₁₀heterocycloalkyl, N(R¹⁴)₂, N(R¹⁶)C(O)R¹⁵, N(R¹⁶)SO₂R¹⁵, C(O)N(R¹⁴)₂, and SO₂N(R¹⁴)₂, wherein said alkyl, alkenyl, alkynyl, cycloalkyl, heterocycloalkyl, or heteroaryl in R¹⁴, R¹⁵, R¹⁶, or R¹⁷, is optionally further substituted. For instance, R¹⁷ is selected from R¹⁷ is H, NH₂, and an optionally substituted C₅₋₁₀heteroaryl or C₄₋₁₀heterocycloalkyl, preferably R¹⁷ is an optionally substituted C₅₋₁₀heteroaryl or C₄₋₁₀heterocycloalkyl.

In a further embodiment, R¹⁷ is an optionally substituted C₄₋₁₀heterocycloalkyl, wherein said heterocycloalkyl may be mono or bicyclic and include from 1 to 3 heteroatoms, preferably wherein X⁷ is N. In a preferred embodiment, the heterocycloalkyl is substituted, for instance, with at least one group selected from F, OH, oxo, CN, C₁₋₄alkyl and OC₁₋₄alkyl, wherein said C₁₋₄alkyl is optionally further substituted (e.g. with F, OH, OC₁₋₃alkyl, etc.). For instance, the heterocycloalkyl may be selected from optionally substituted piperidine, piperazine, thiomorpholine, and morpholine groups, or a bicyclic structure (bridged or spiro) containing a piperidine, piperazine, thiomorpholine, or morpholine ring.

In a further embodiment, X⁷ is C, for instance, X⁷ is C and R¹⁸ is selected from C₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂, wherein said alkyl, alkenyl, alkynyl, cycloalkyl, heterocycloalkyl, in R¹⁴, R¹⁵, R¹⁶, or R¹⁸, or heteroaryl is optionally further substituted, preferably R¹⁸ is selected from C(O)N(R¹⁴)₂, SO₂R¹⁵, and SO₂N(R¹⁴)₂. In a subclass of these embodiments, R¹⁷ is selected from H, OH, C₁₋₆alkyl, N(R¹⁴)₂, and an optionally substituted C₅₋₁₀heteroaryl. For instance, R¹⁷ is selected from H, NH₂, and an optionally substituted C₅₋₁₀heteroaryl, preferably H or NH₂.

In yet another embodiment, R¹⁴ is independently in each occurrence selected from H, optionally substituted C₁₋₆alkyl, optionally substituted C₃₋₁₀cycloalkyl, optionally substituted C₄₋₁₀heterocycloalkyl, and optionally substituted C₅₋₆heteroaryl, or two R¹⁴ are taken together with their adjacent nitrogen atom to form a C₄₋₁₀heterocycloalkyl group.

In another embodiment, R¹⁷ is N(R¹⁴)₂ wherein said R¹⁴ are taken together with their adjacent nitrogen atom to form a C₄₋₁₀heterocycloalkyl group, wherein said heterocycloalkyl may be mono or bicyclic and include from 1 to 3 heteroatoms, preferably wherein X⁷ is N. In a preferred embodiment, the heterocycloalkyl is substituted, for instance, with at least one group selected from F, OH, oxo, CN, C₁₋₄alkyl and OC₁₋₄alkyl, wherein said C₁₋₄alkyl is optionally further substituted (e.g. with F, OH, OC₁₋₃alkyl, etc.). For instance, the heterocycloalkyl may be selected from optionally substituted piperidine, piperazine, thiomorpholine, and morpholine groups, or a bicyclic structure (bridged or spiro) containing a piperidine, piperazine, thiomorpholine, or morpholine ring.

In another example, R¹ is selected from:

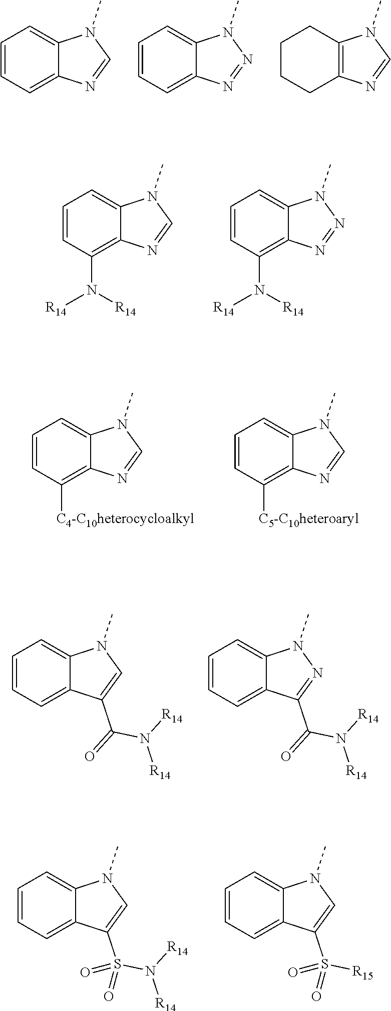

wherein R¹⁴ is as defined herein and (---) represents a bond.

In a further example, R¹ is selected from:

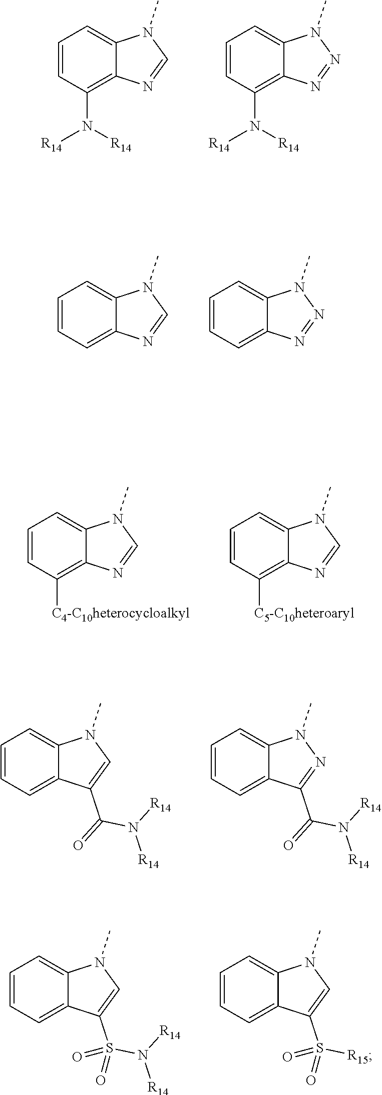

wherein R¹⁴ is as defined herein and (---) represents a bond.

Further subgeneric embodiments are also presented in the examples section, wherein each substituent group R¹ (C group), R² (B group), Y and L are defined. Examples of combinations are also set forth further below and in Tables 2 to 5. Representative preferred compounds of Examples 1 to 163 are also described herein.

More specifically, exemplary R¹ groups are illustrated as C1 to C493 defined as follows:

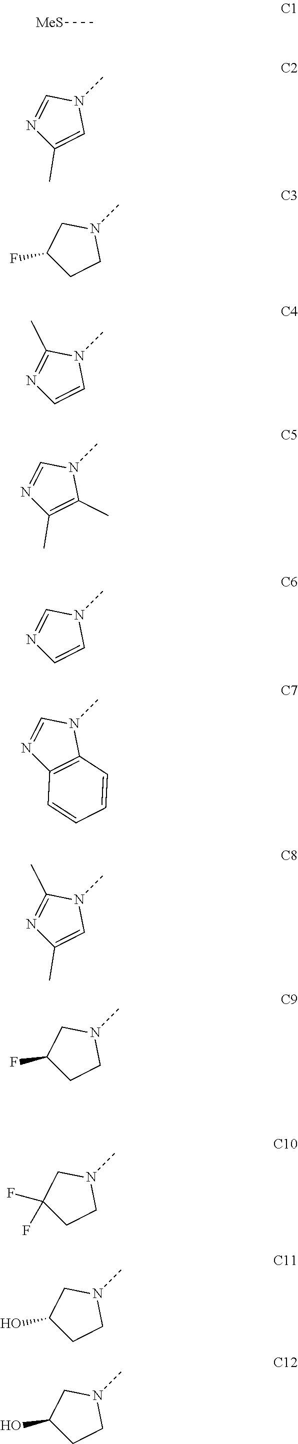

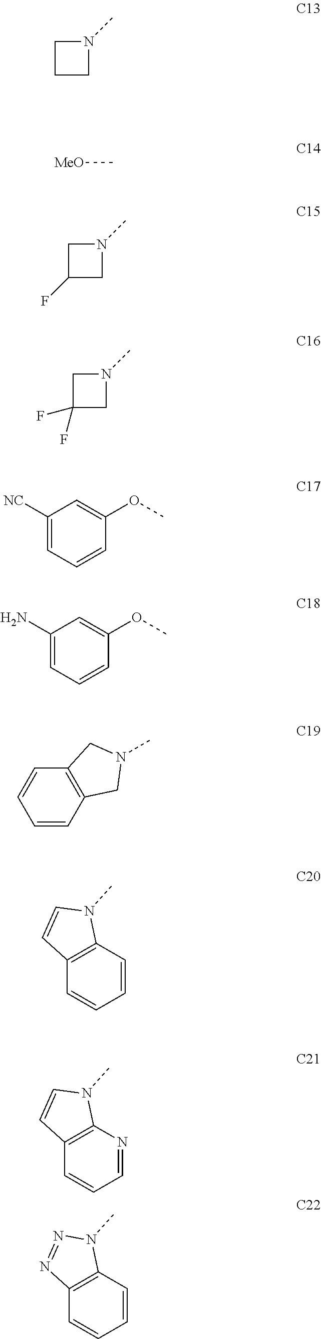

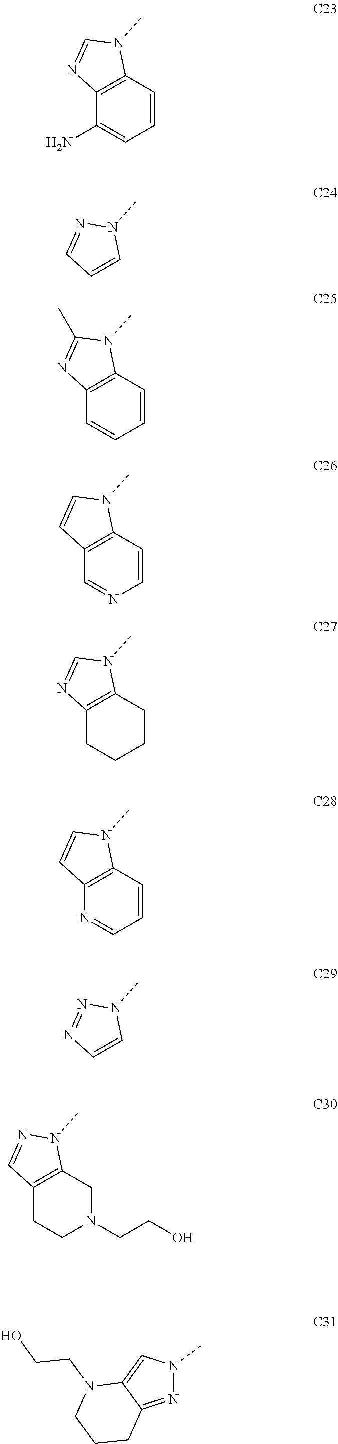

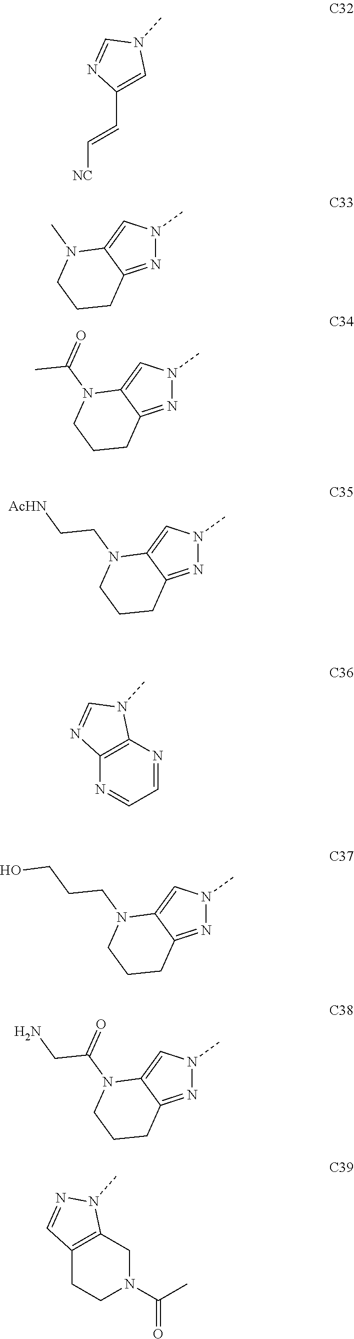

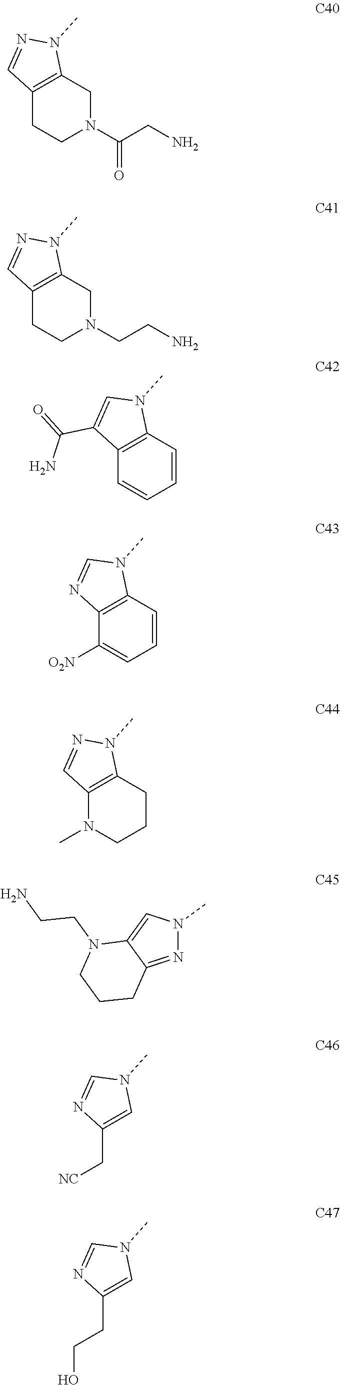

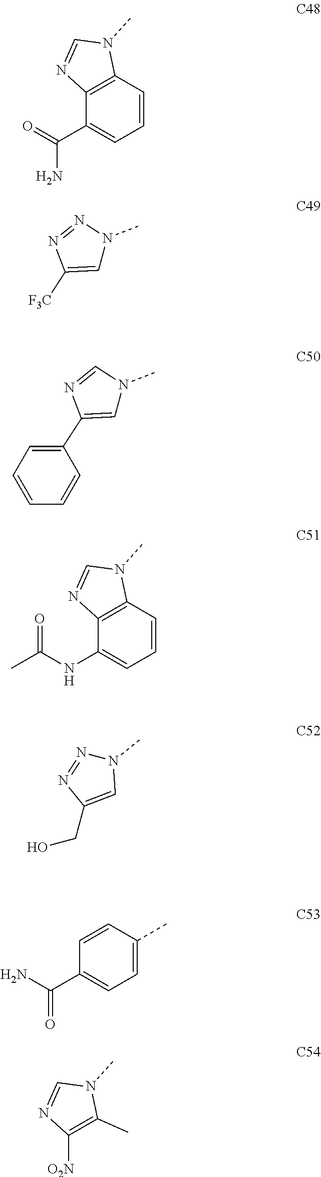

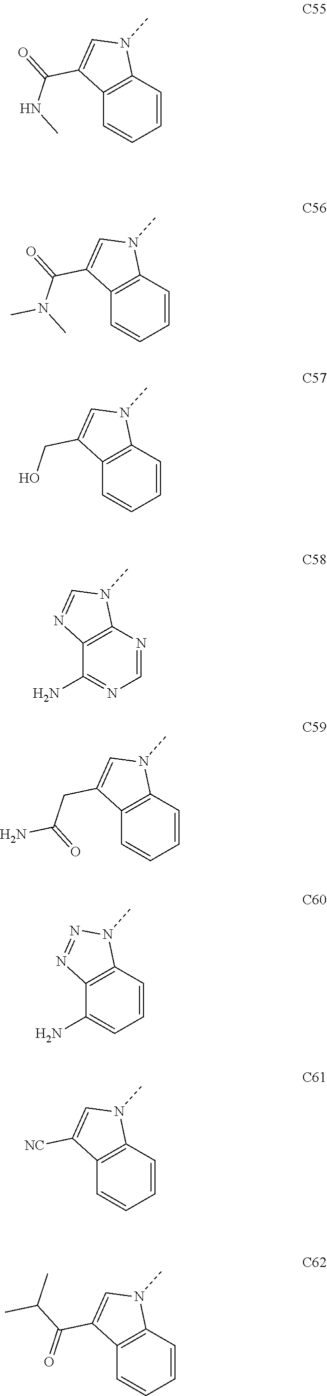

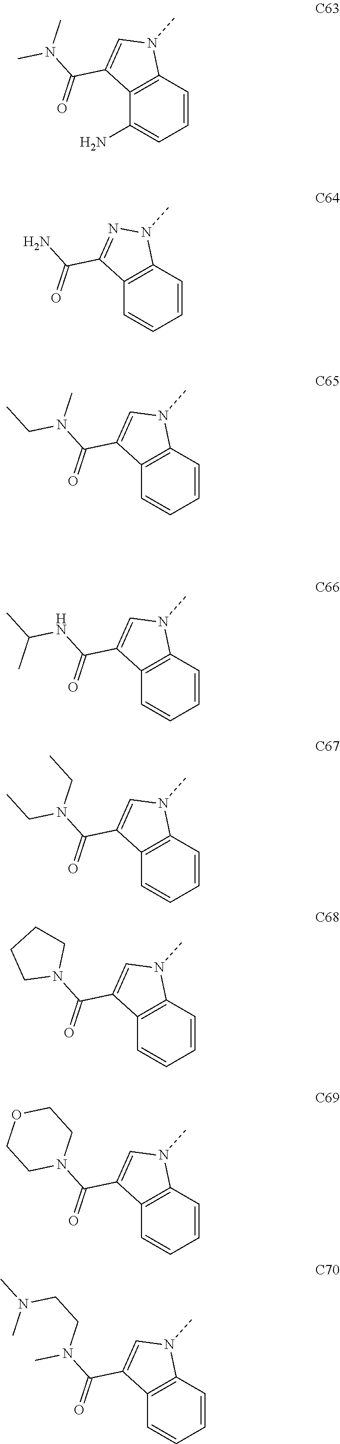

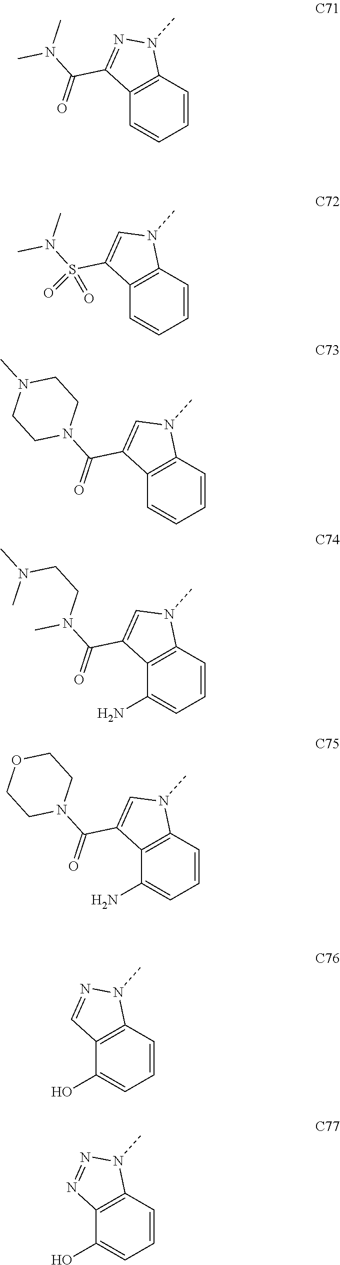

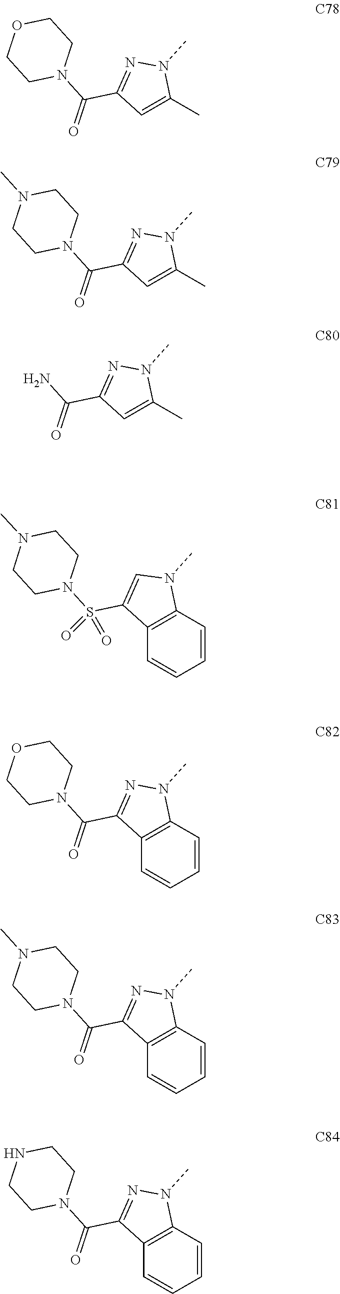

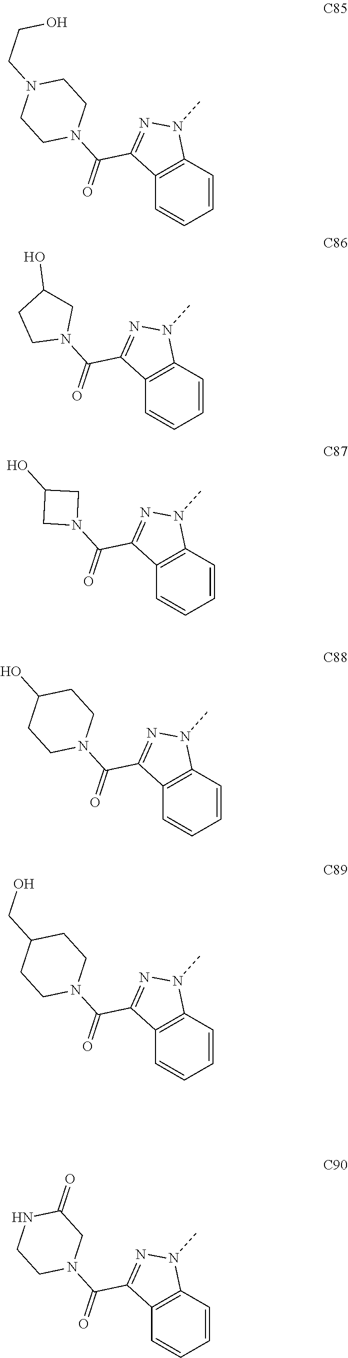

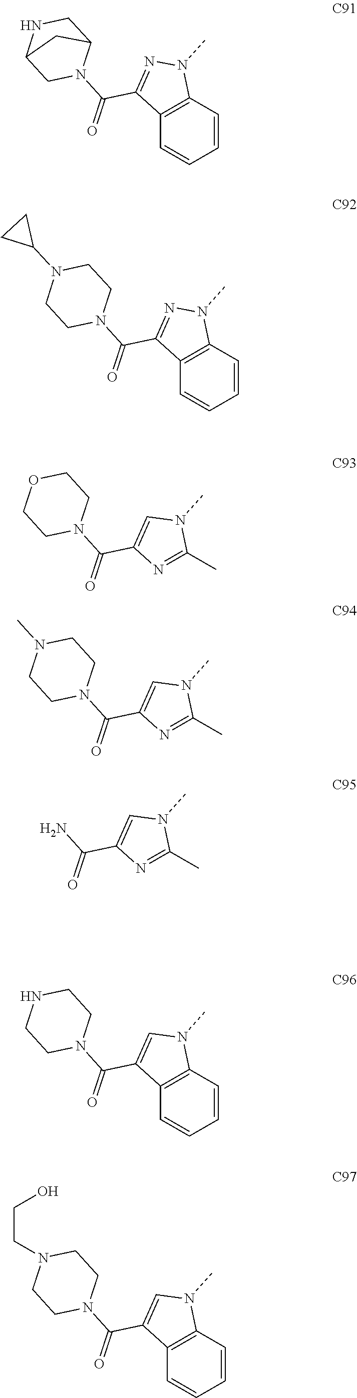

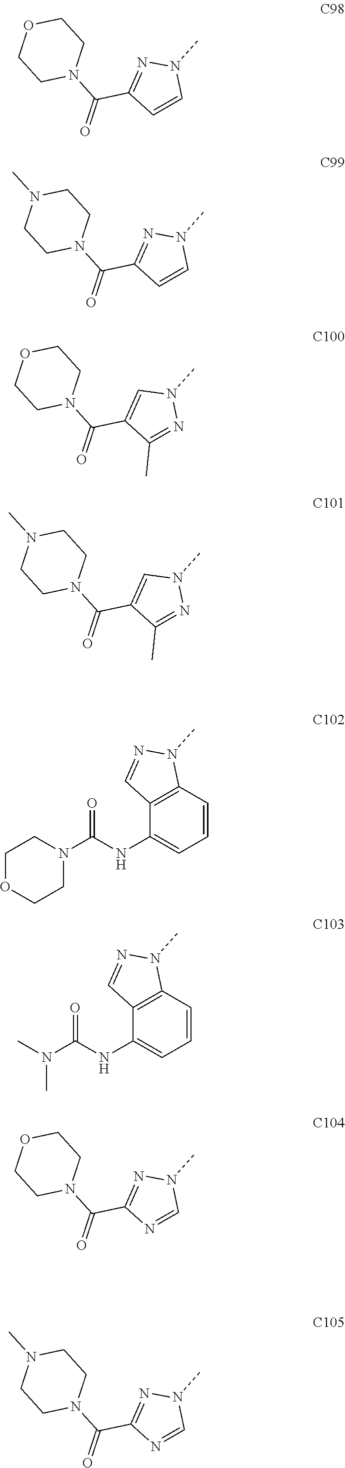

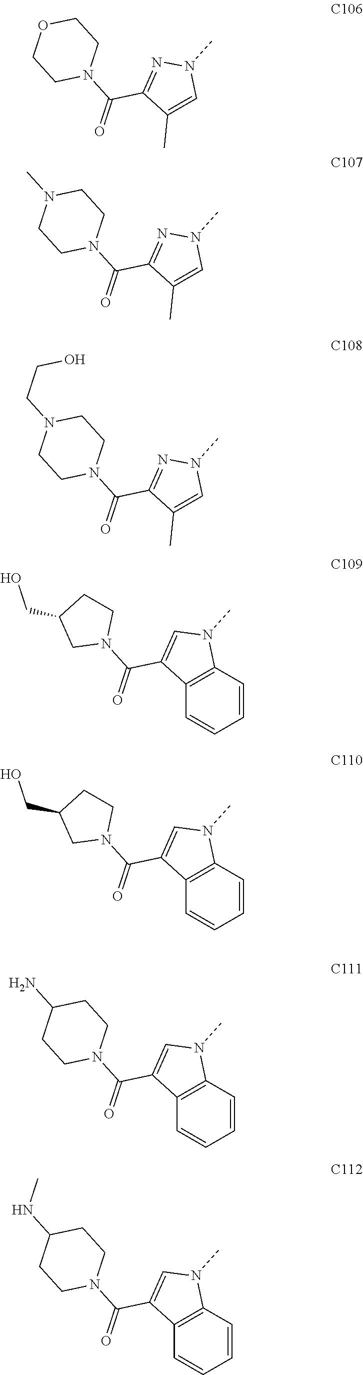

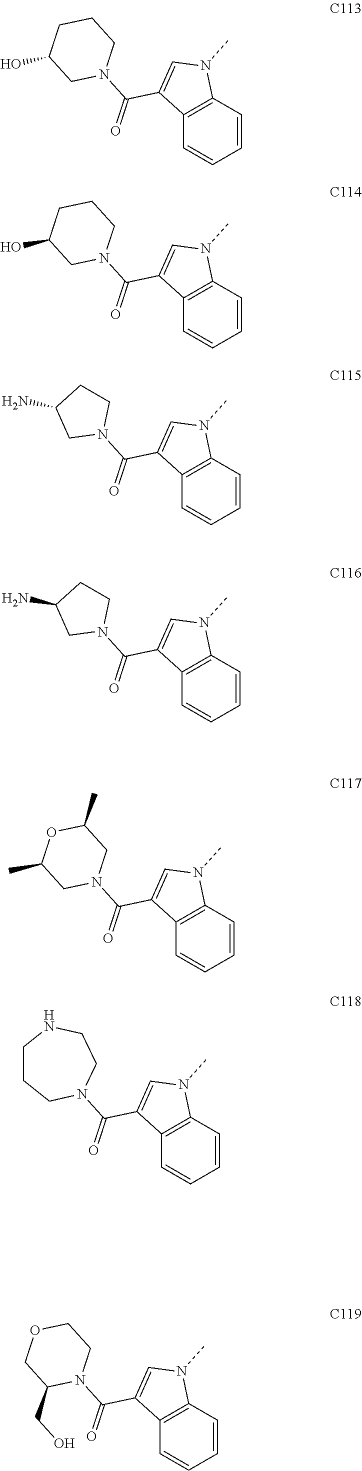

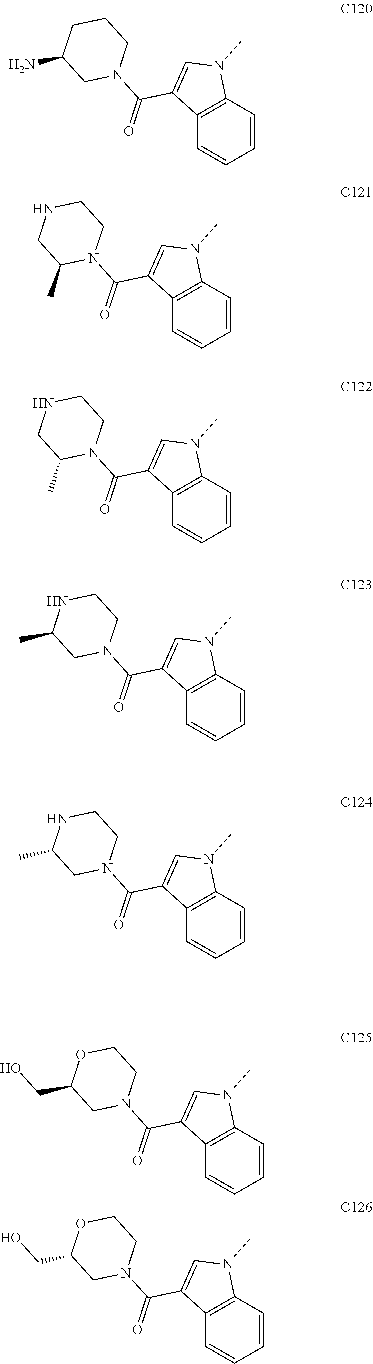

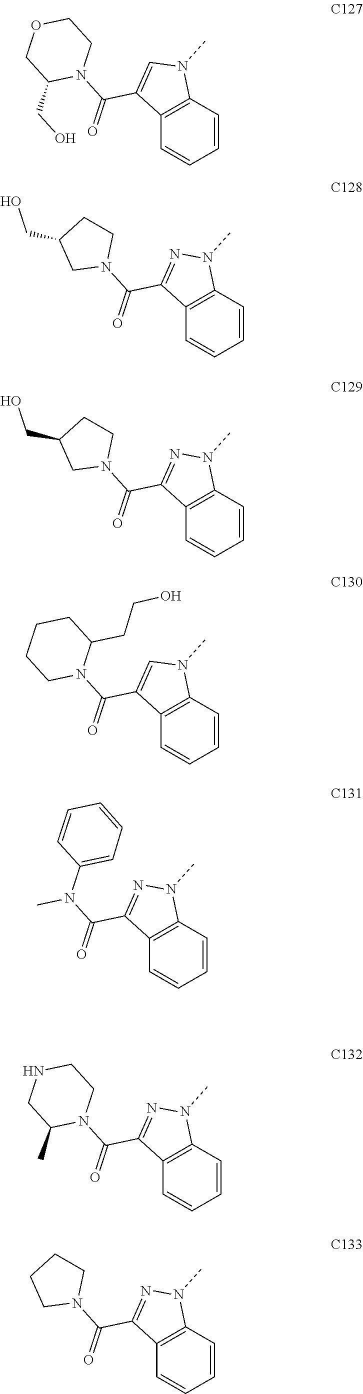

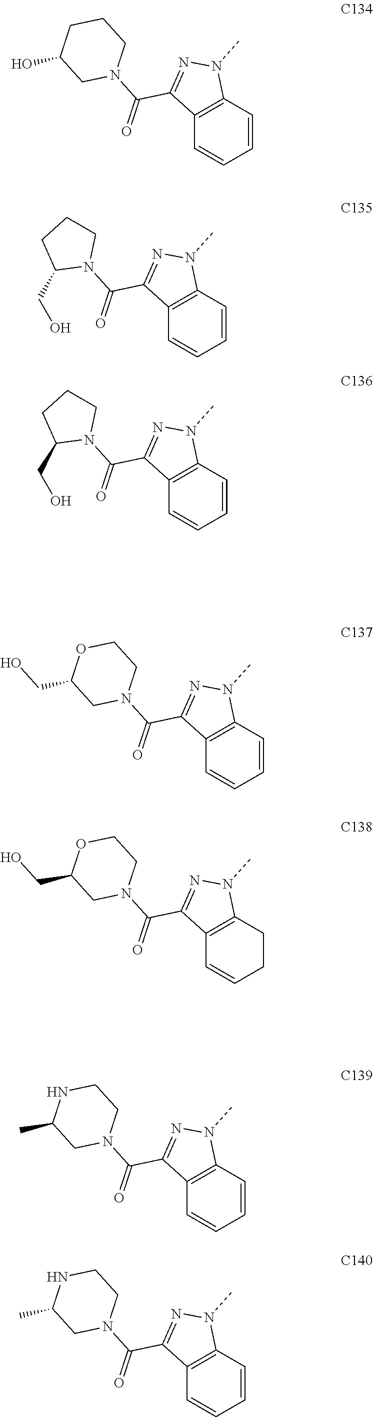

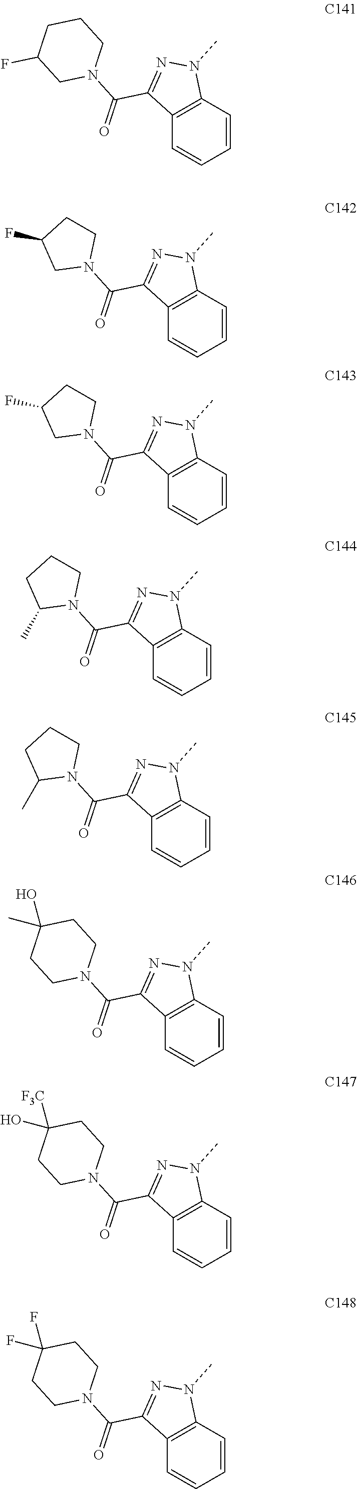

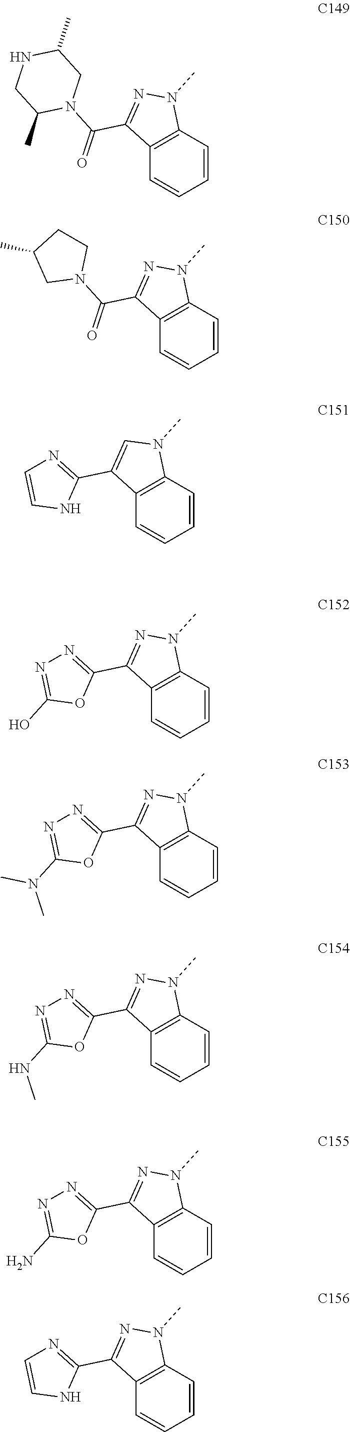

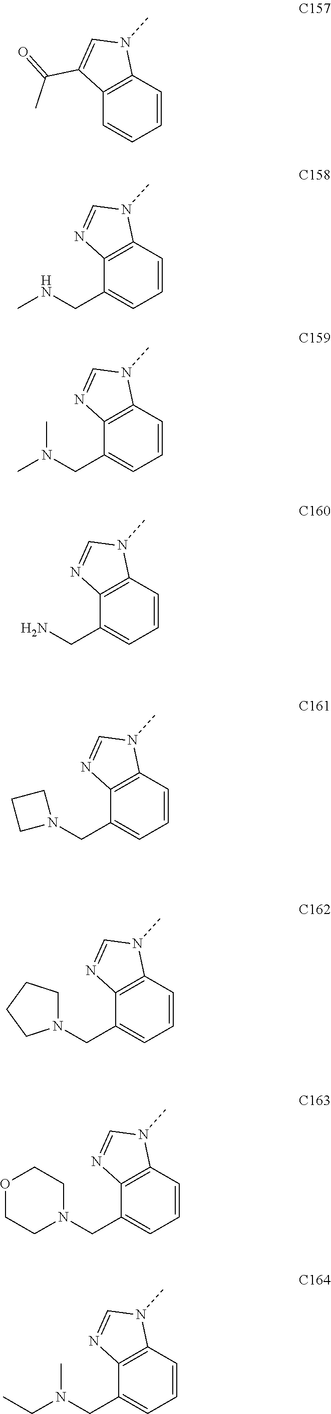

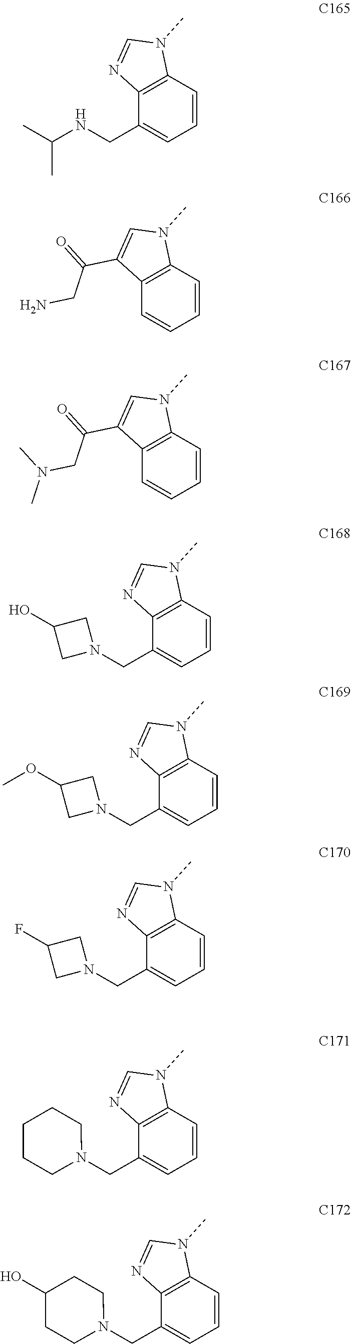

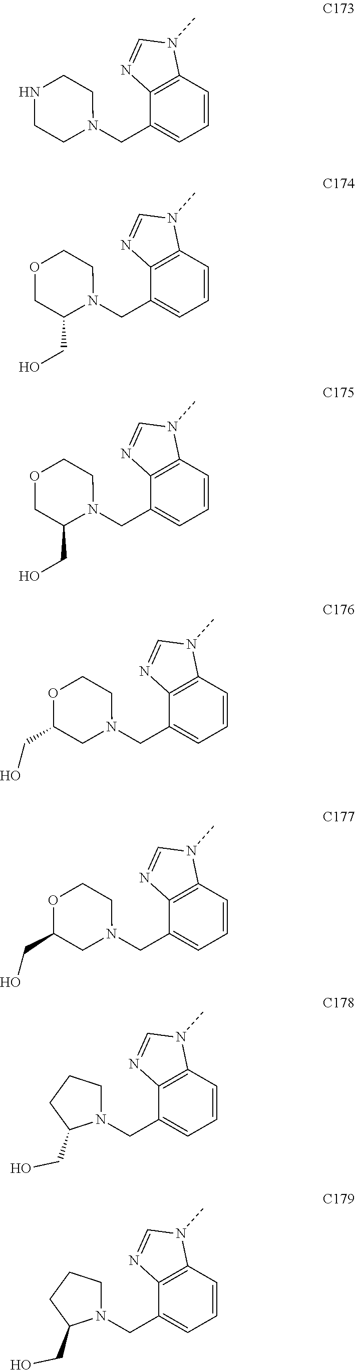

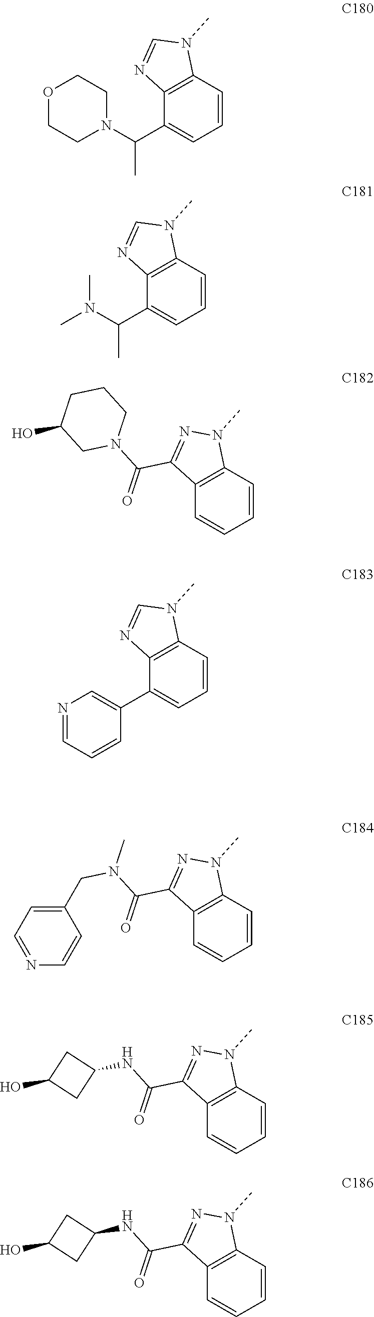

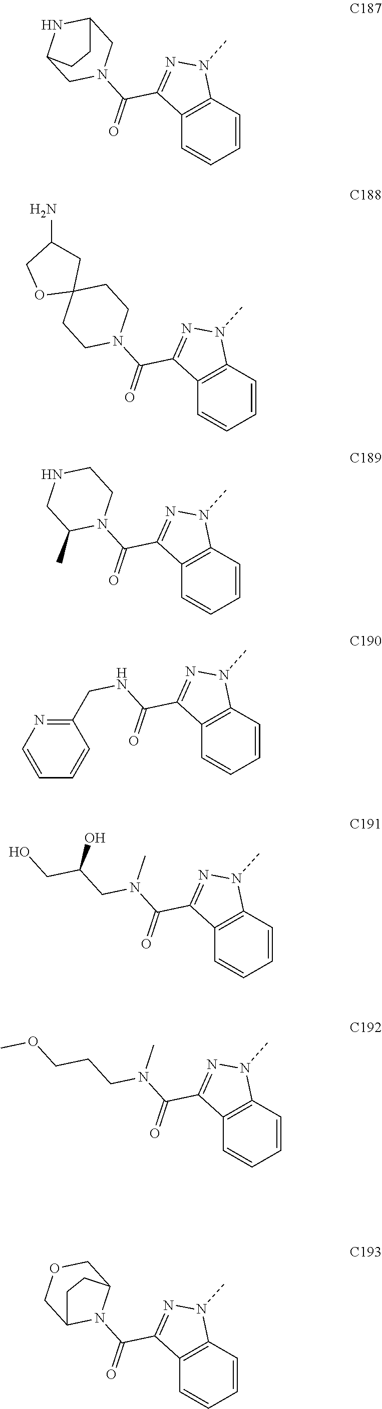

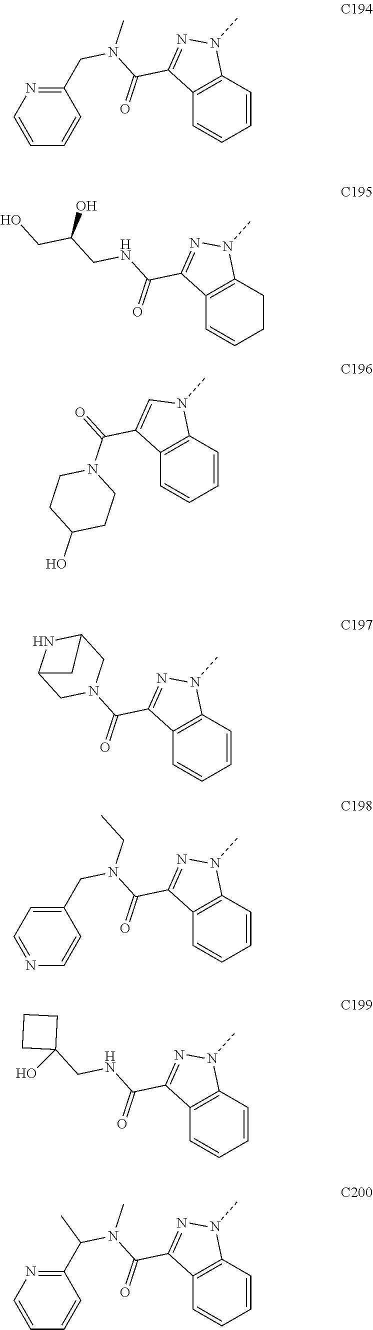

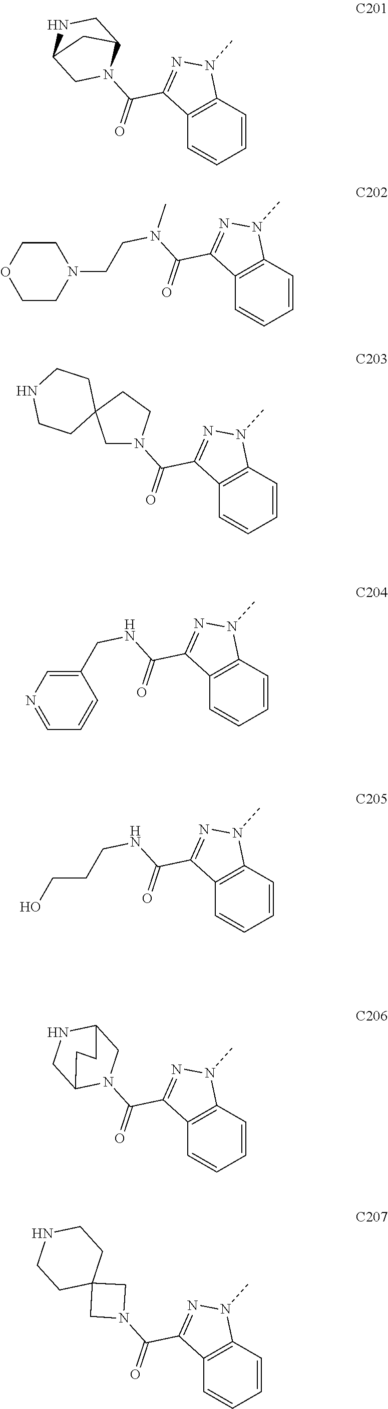

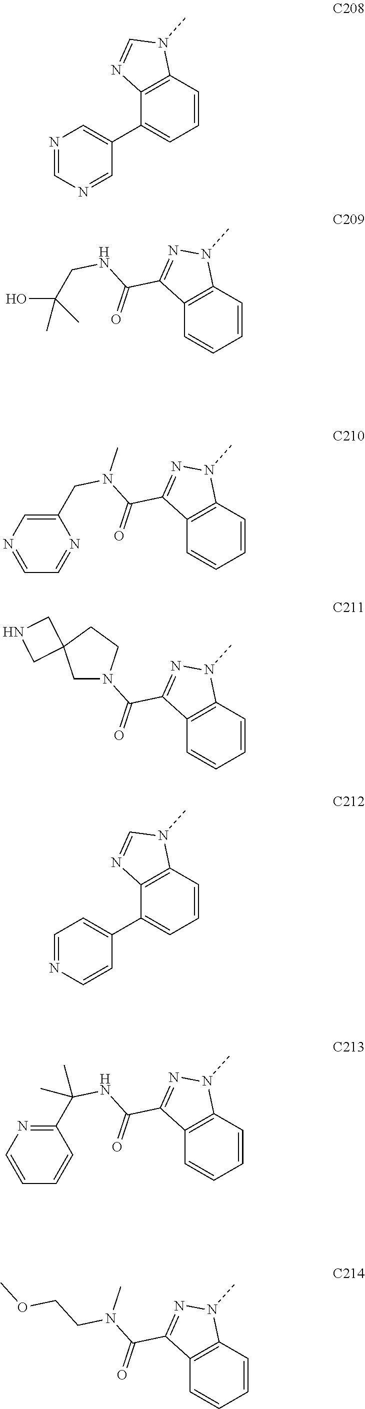

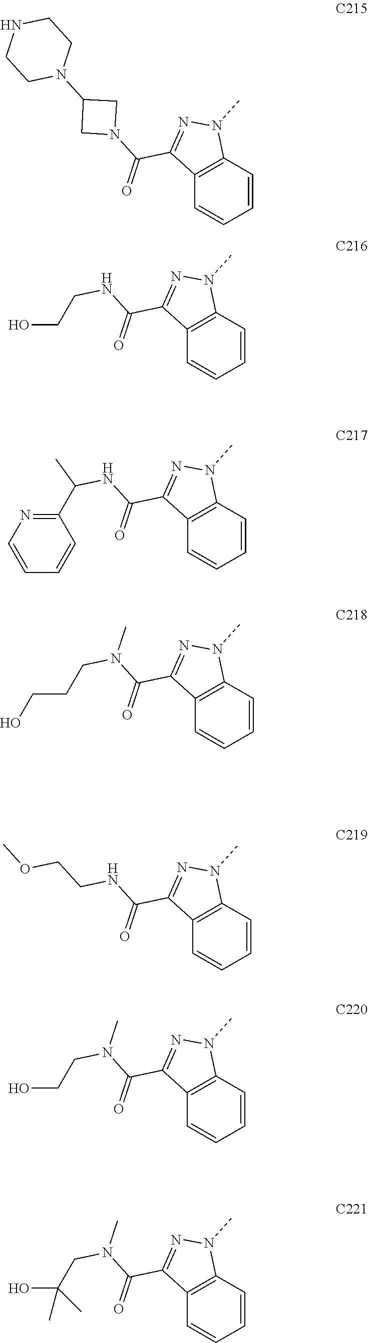

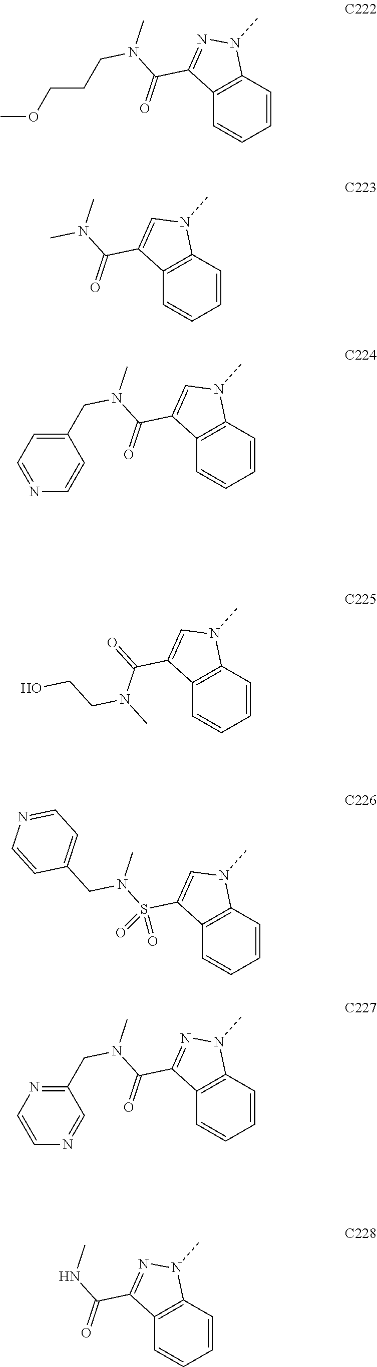

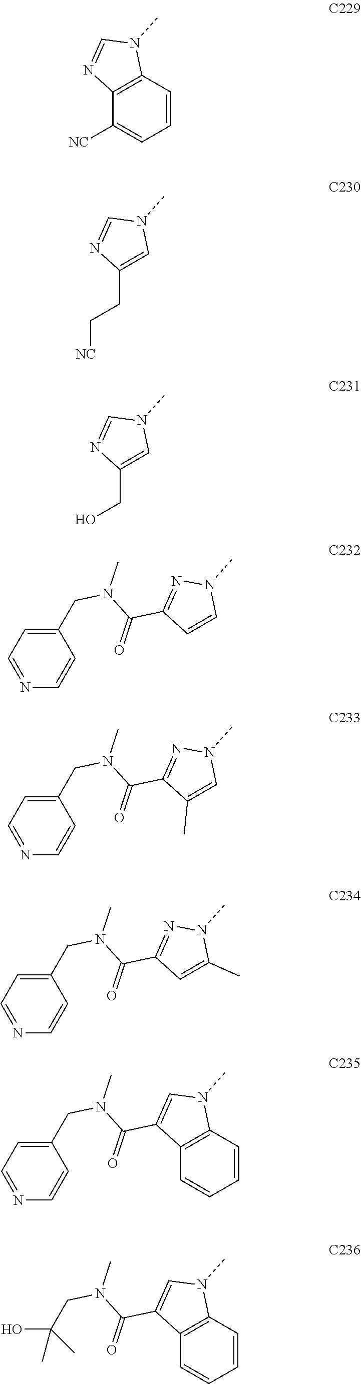

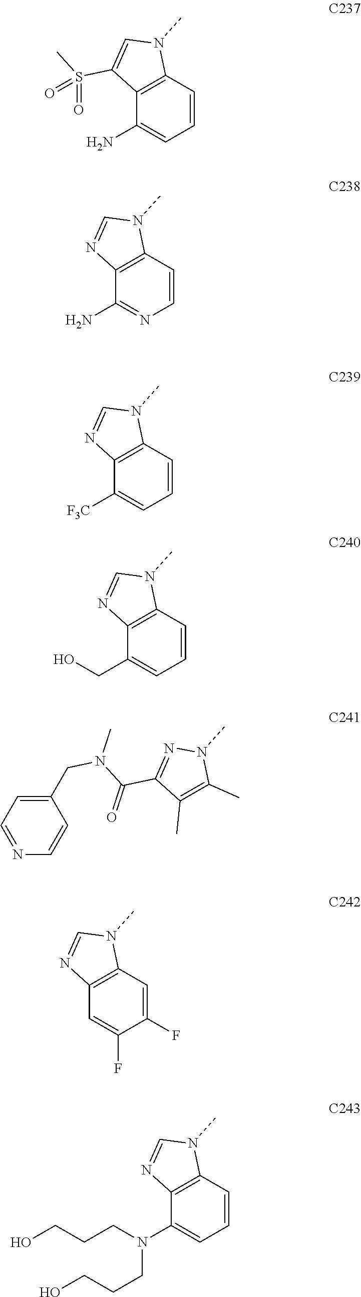

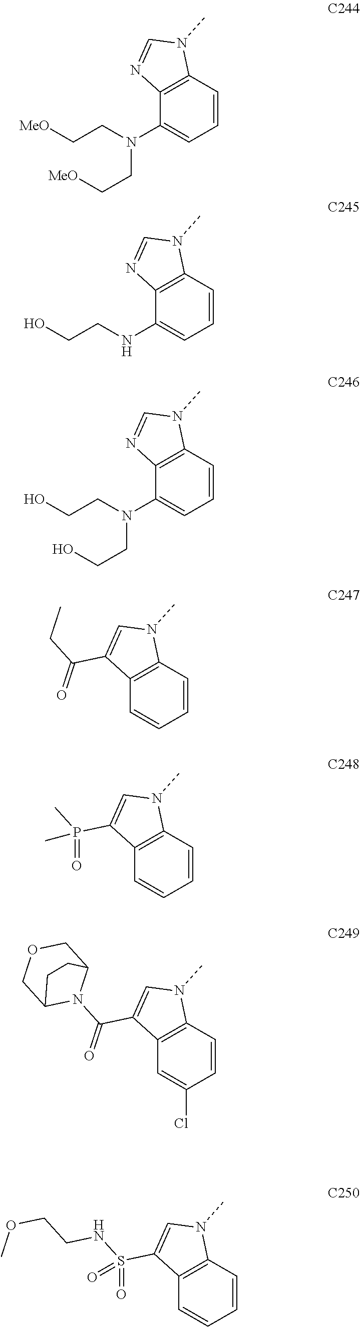

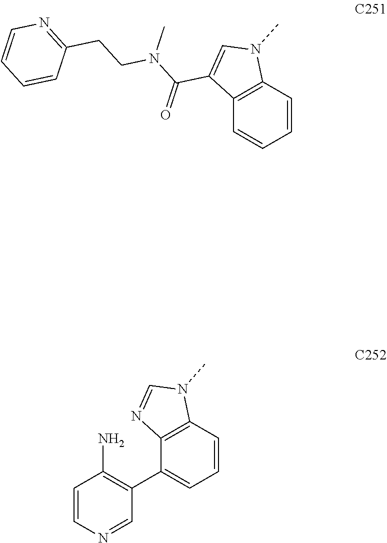

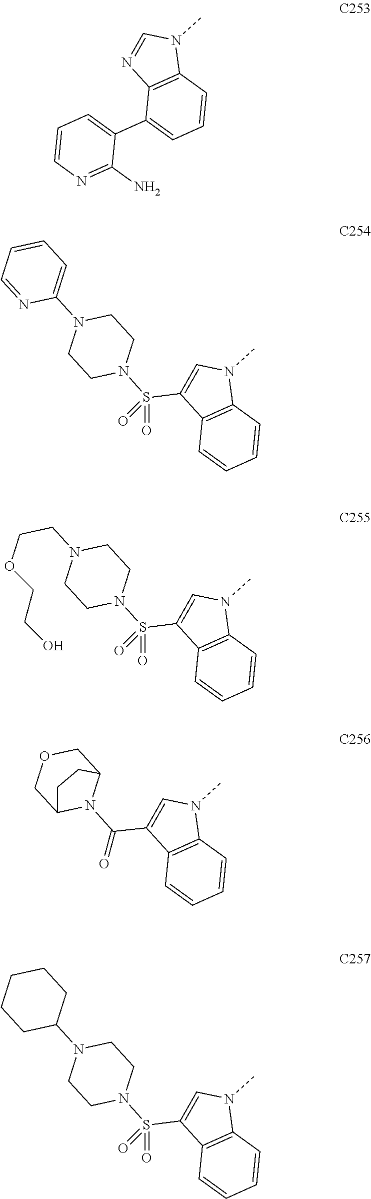

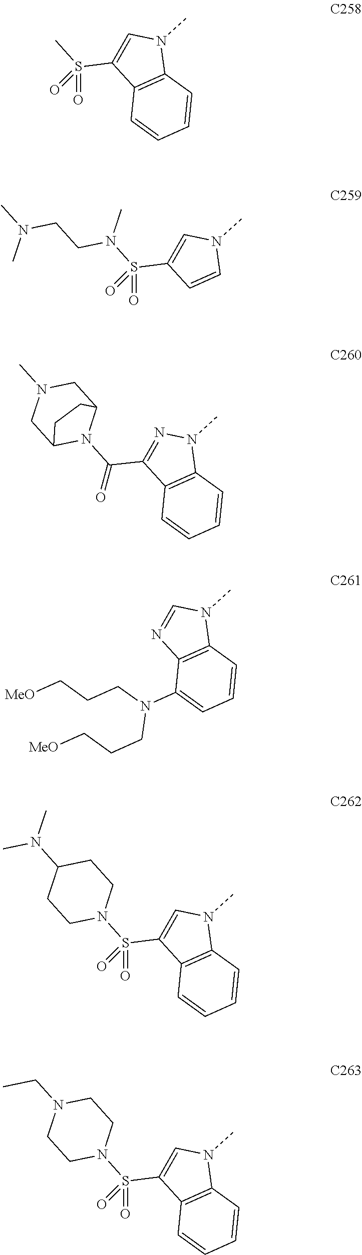

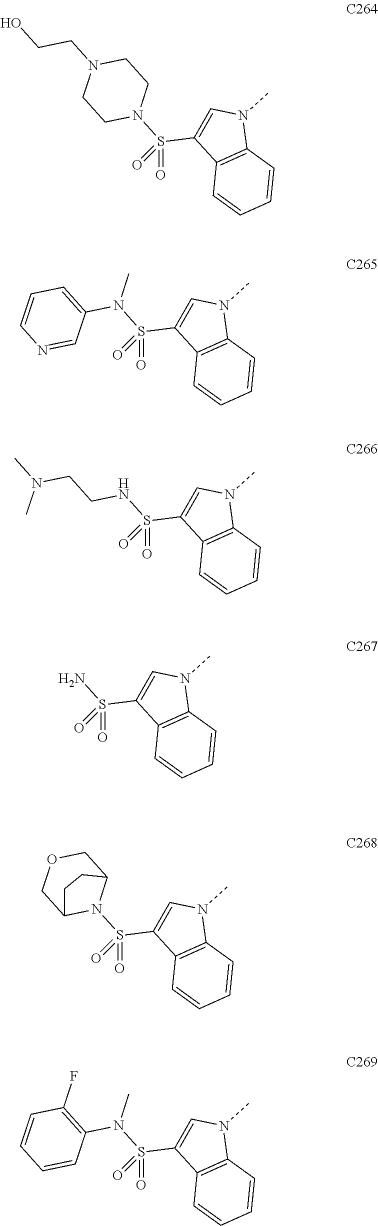

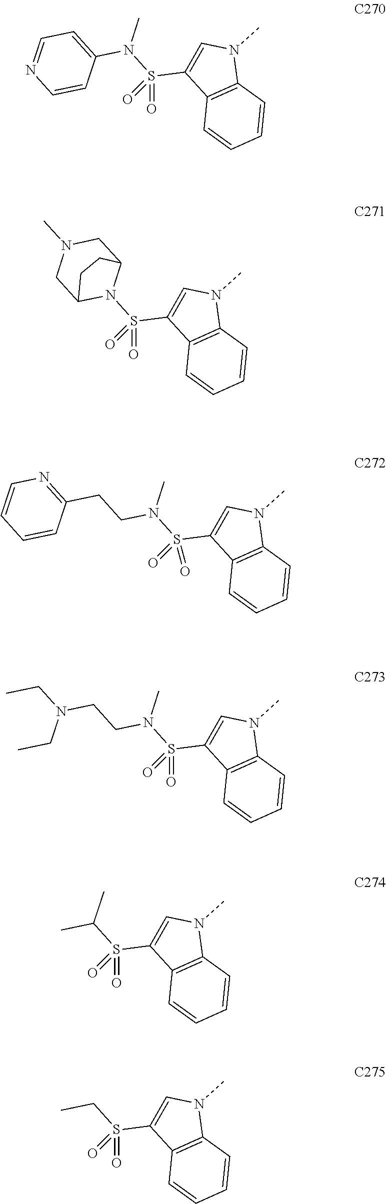

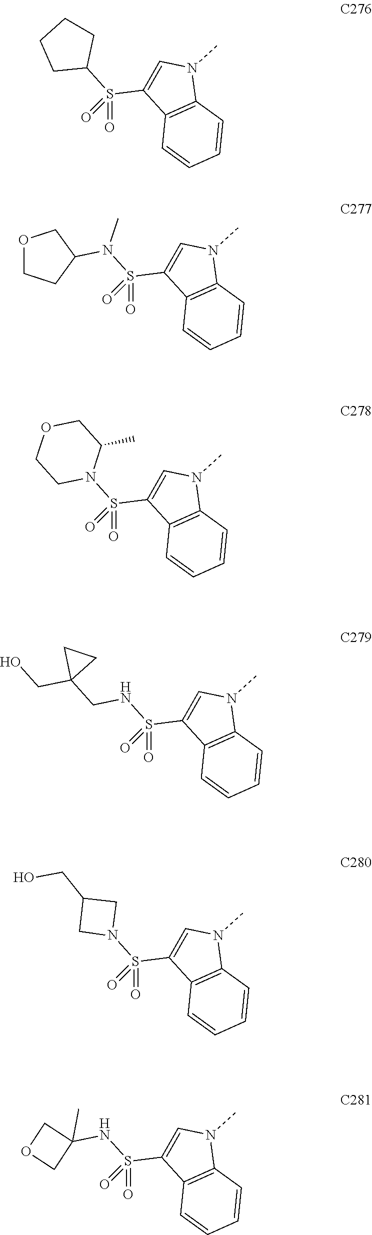

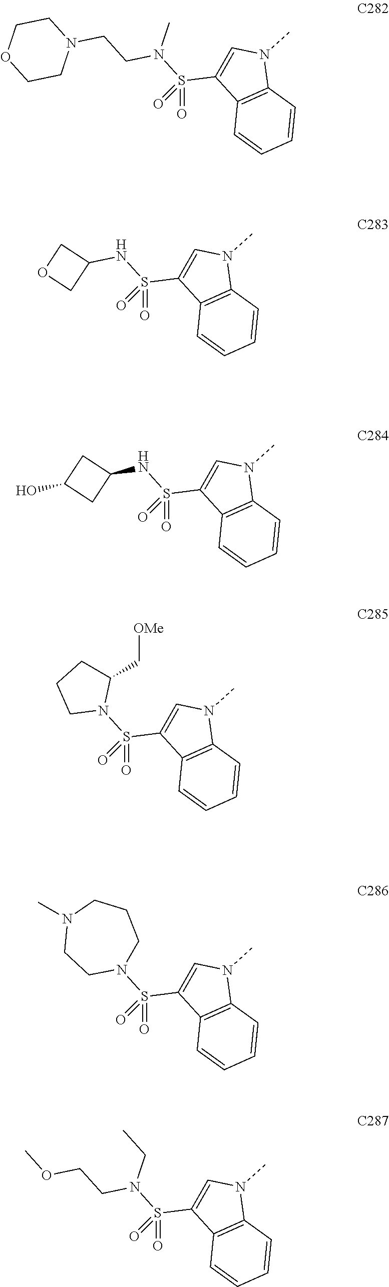

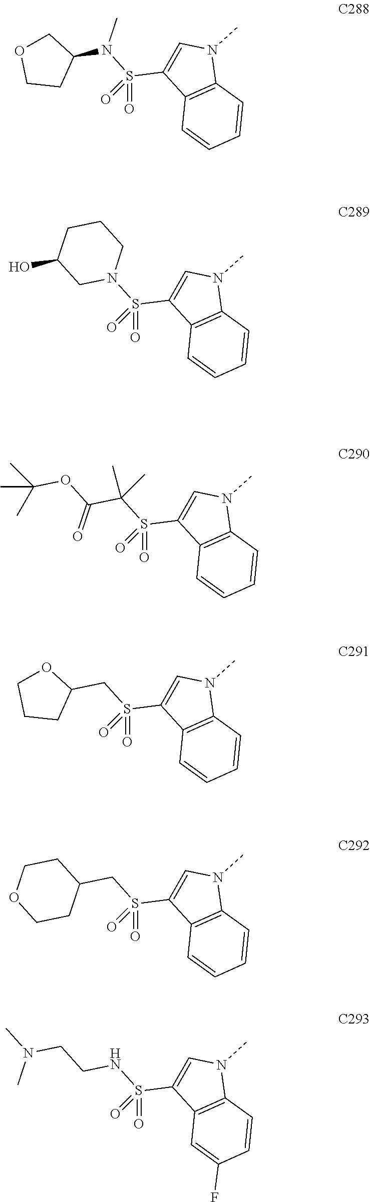

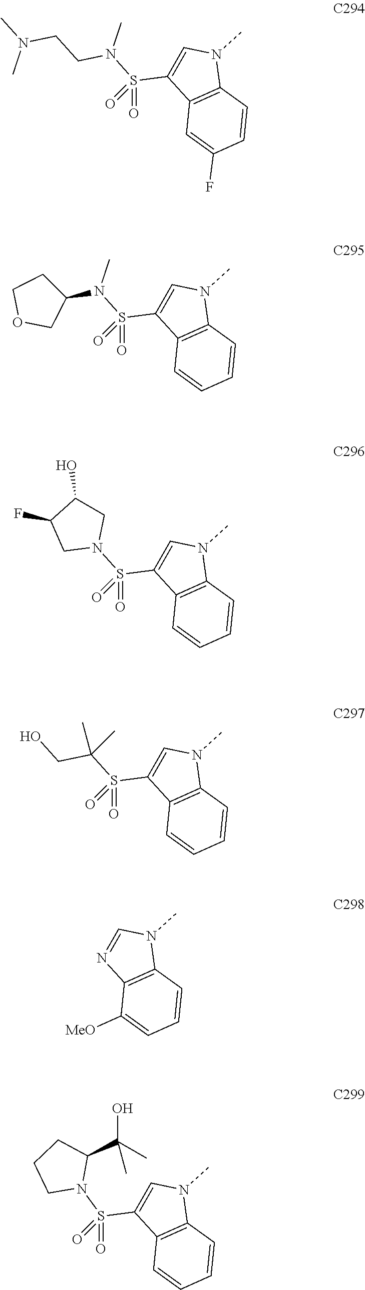

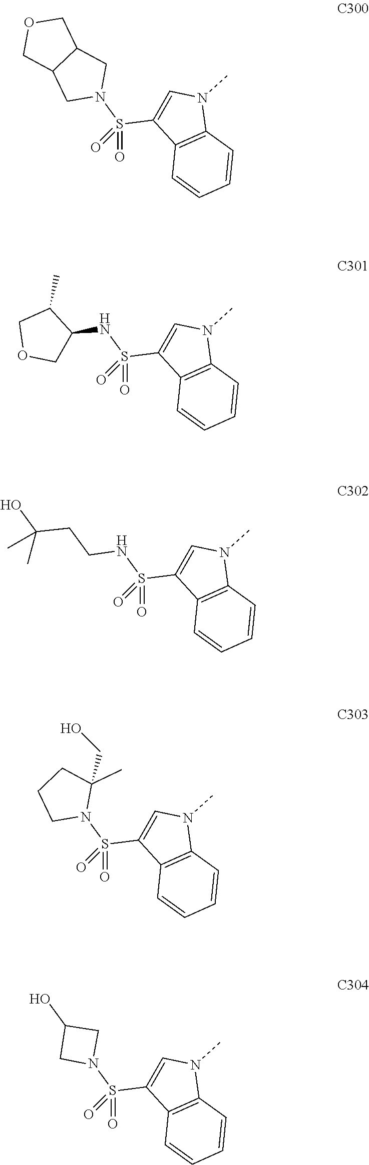

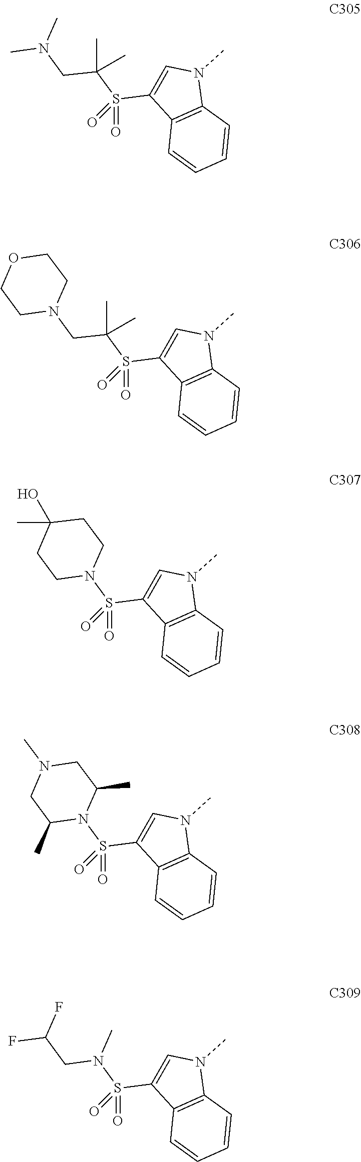

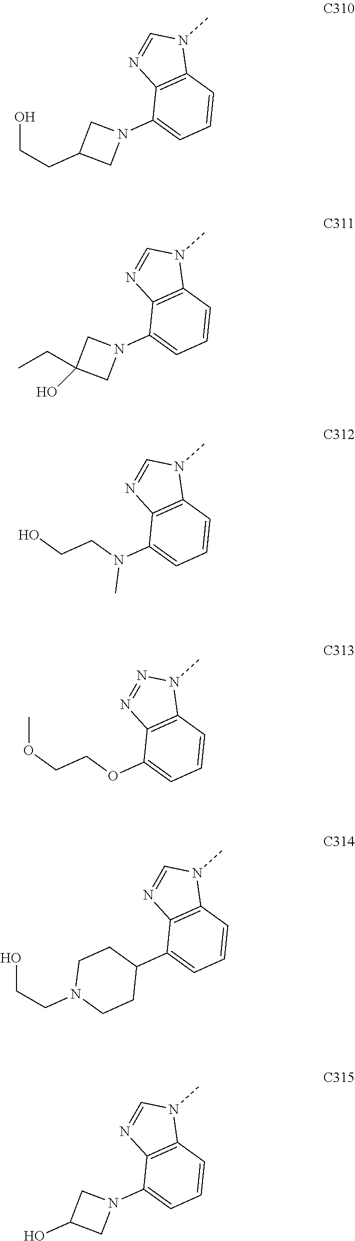

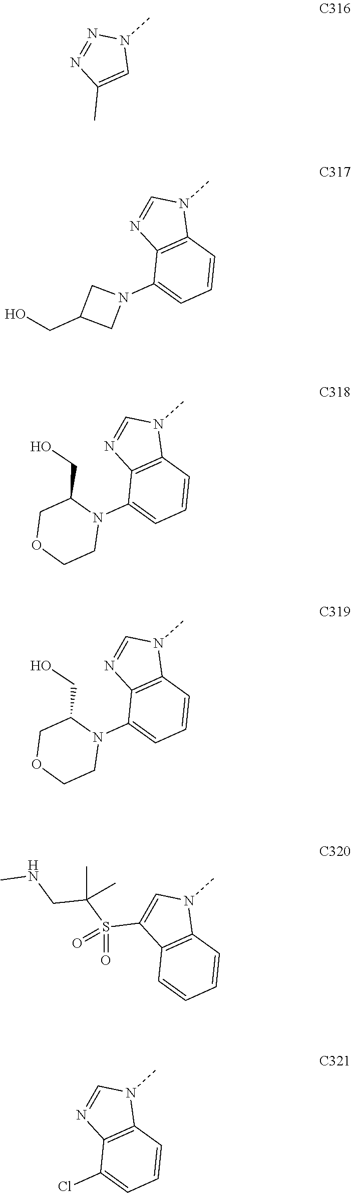

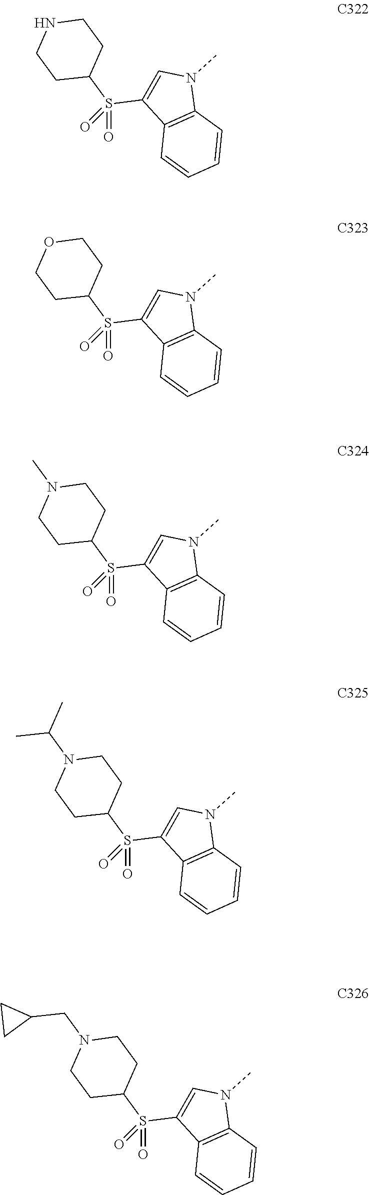

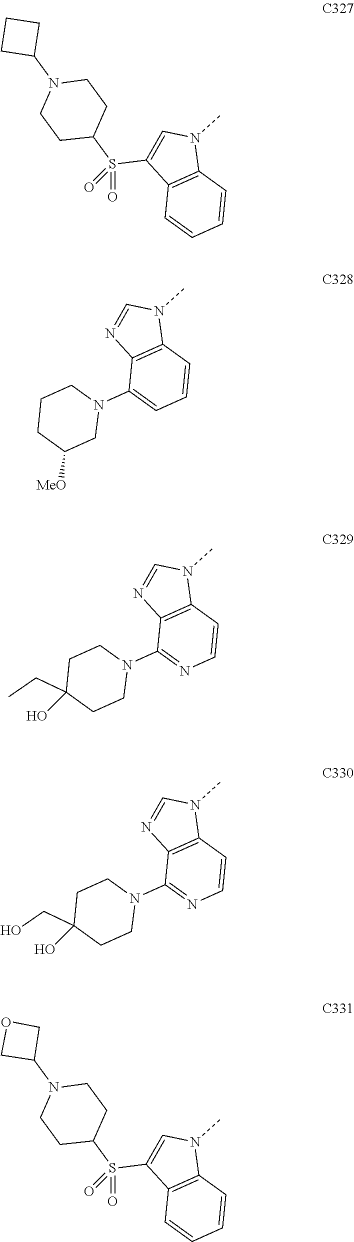

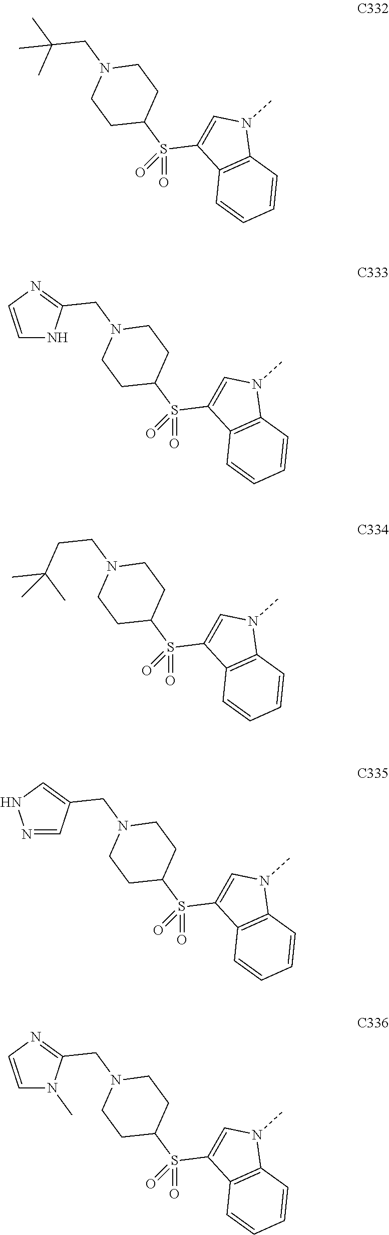

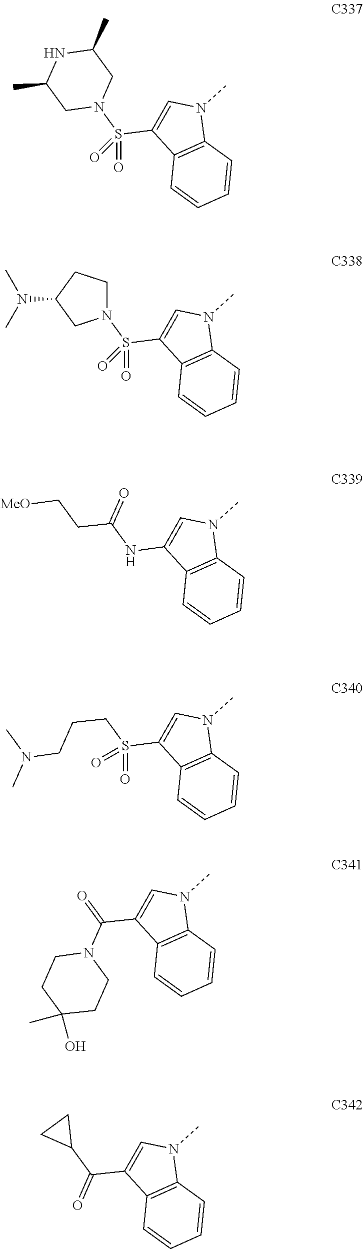

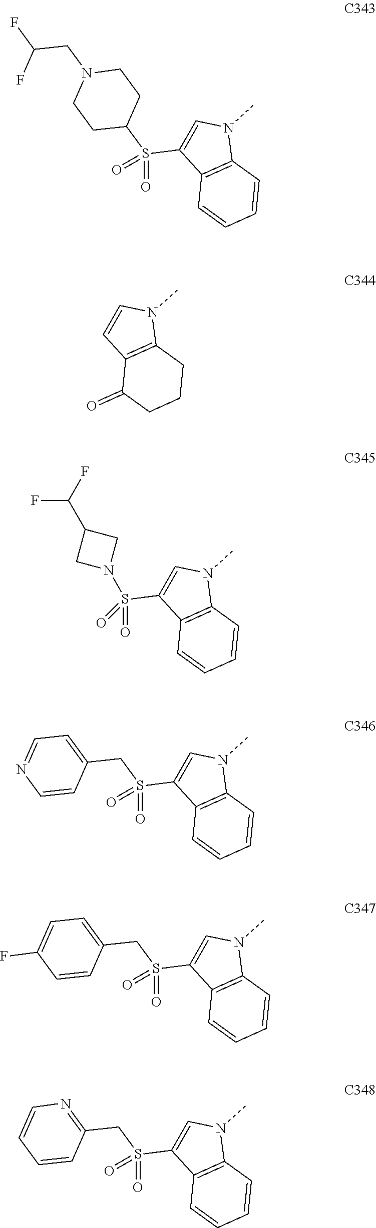

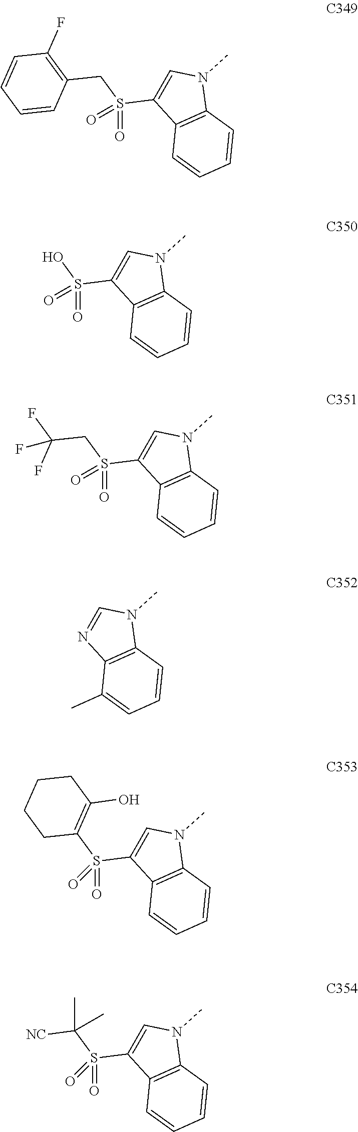

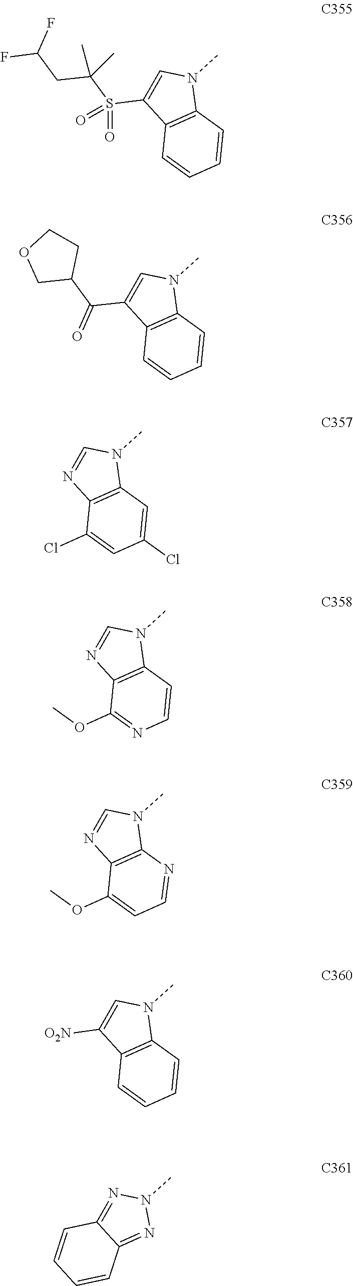

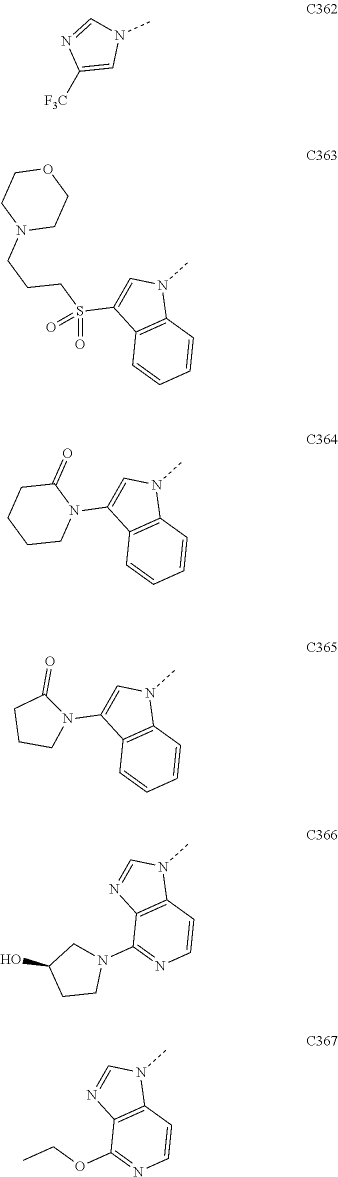

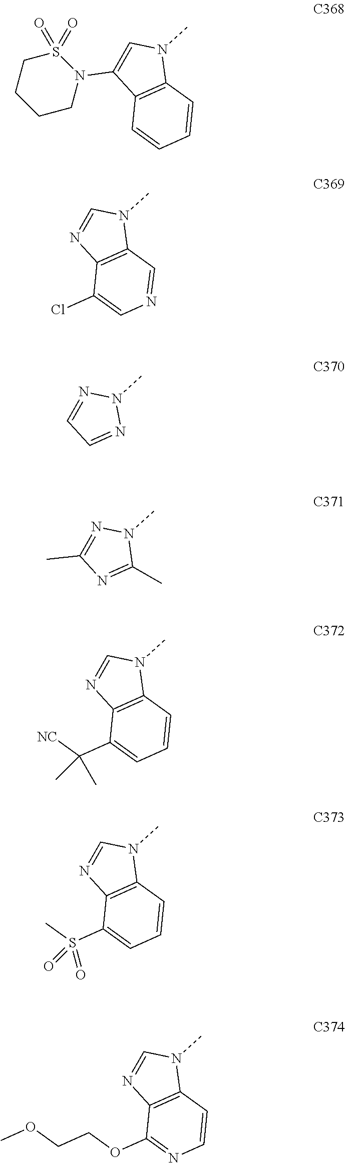

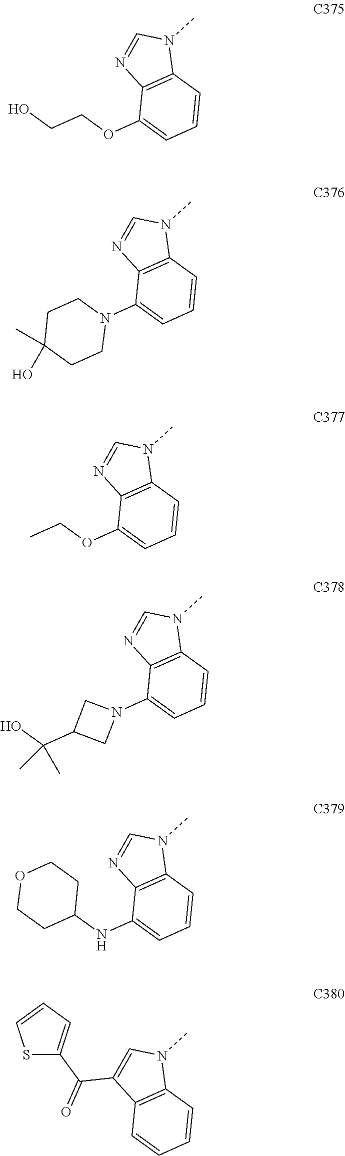

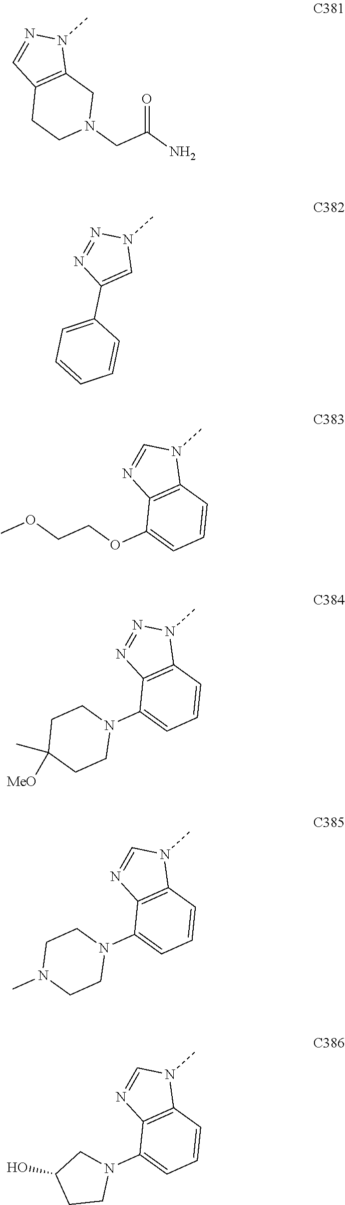

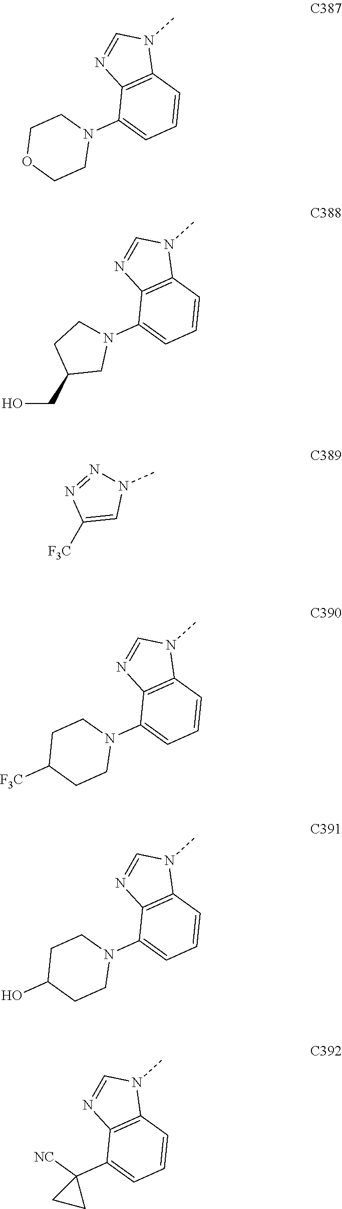

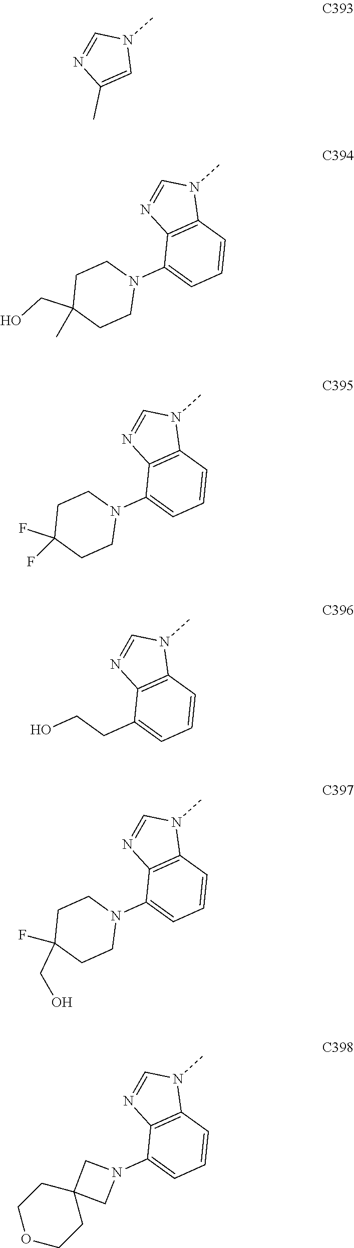

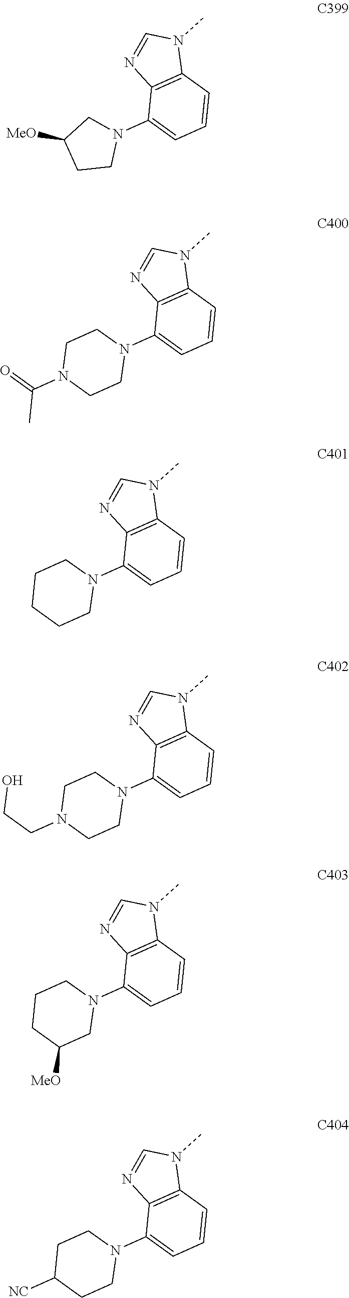

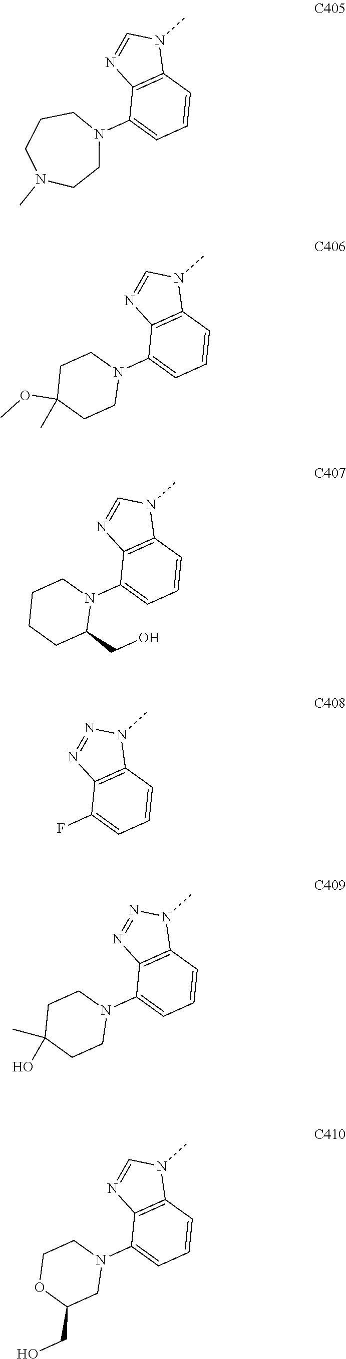

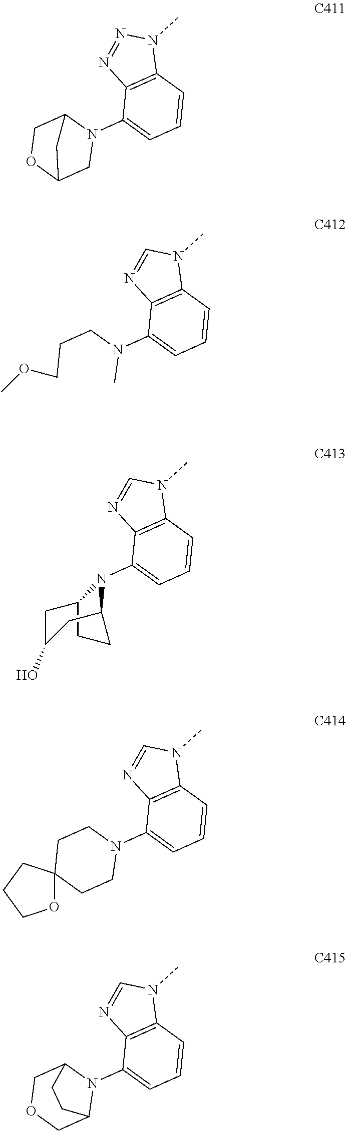

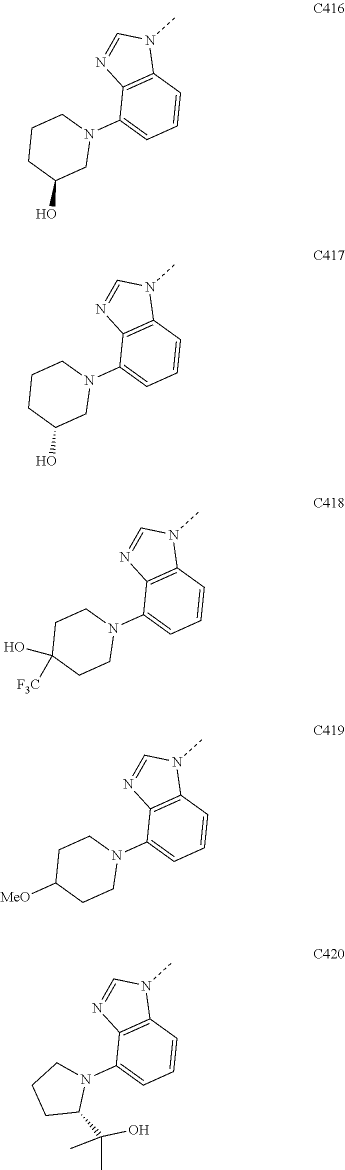

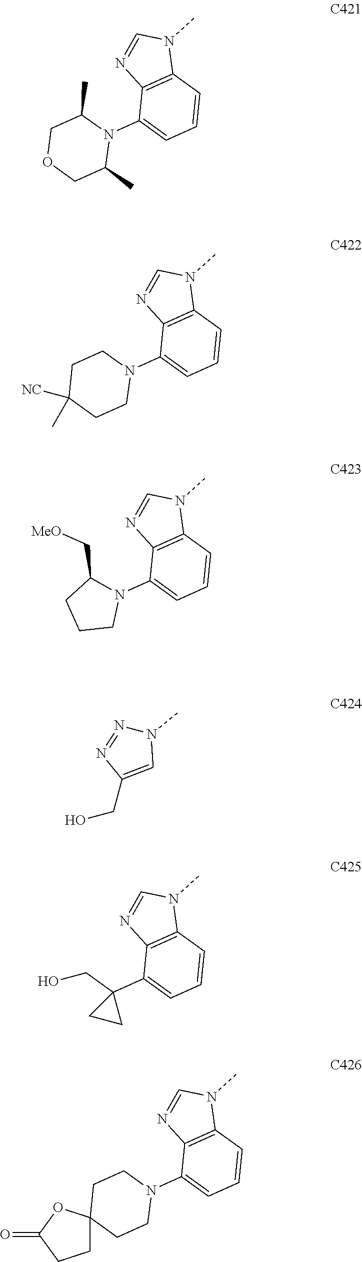

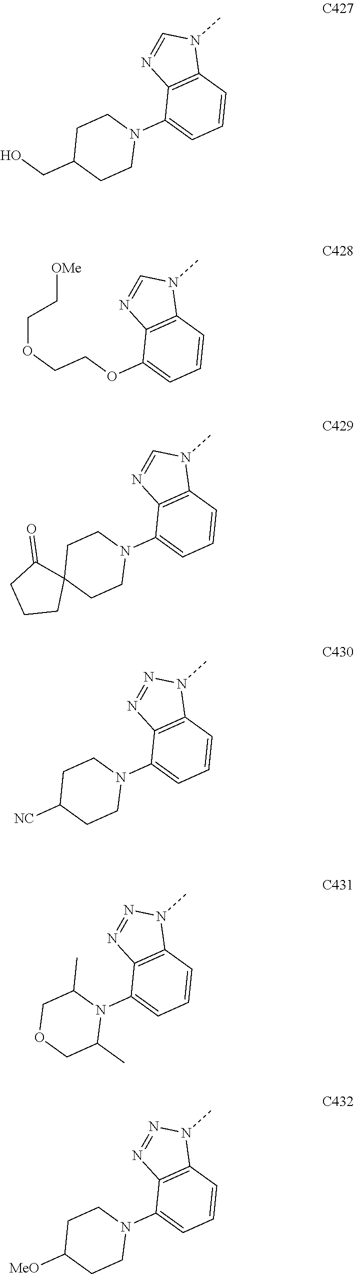

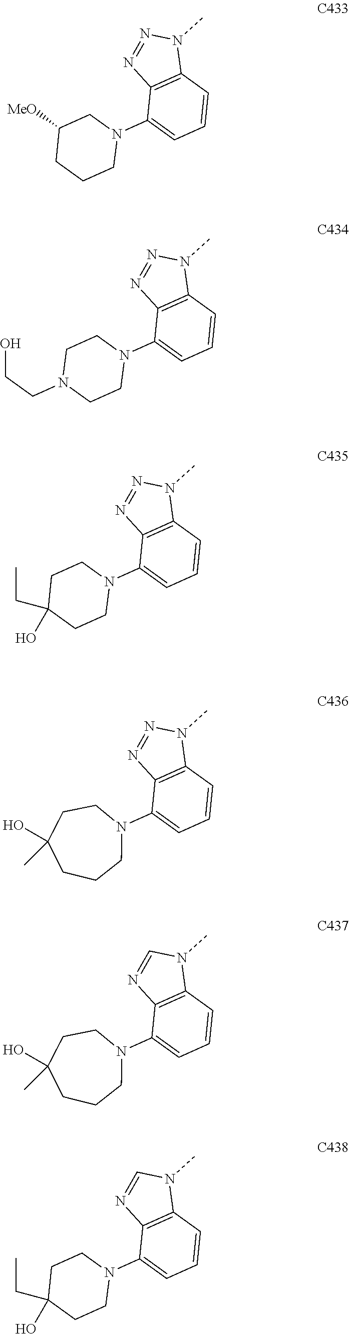

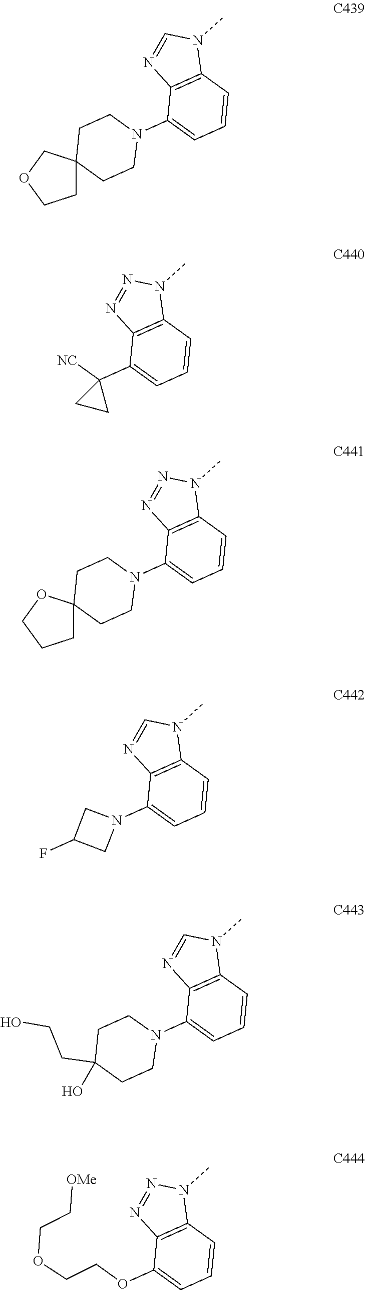

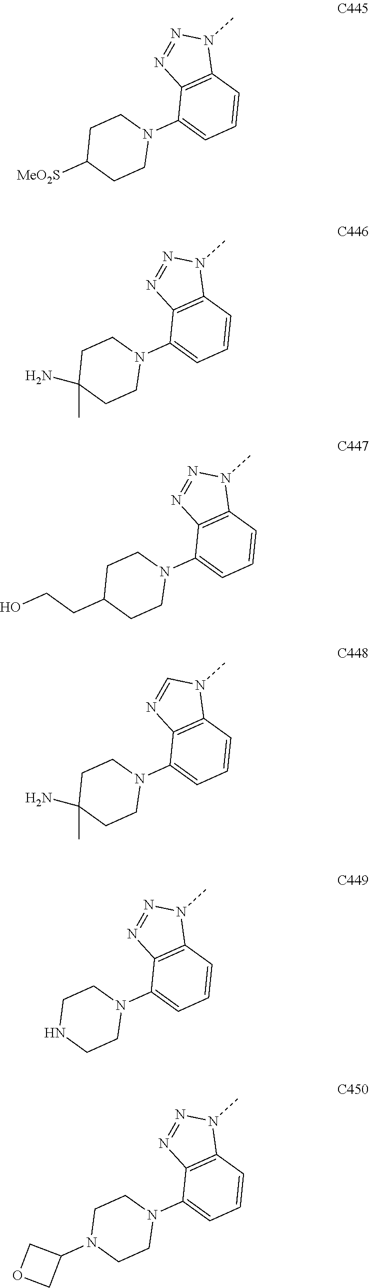

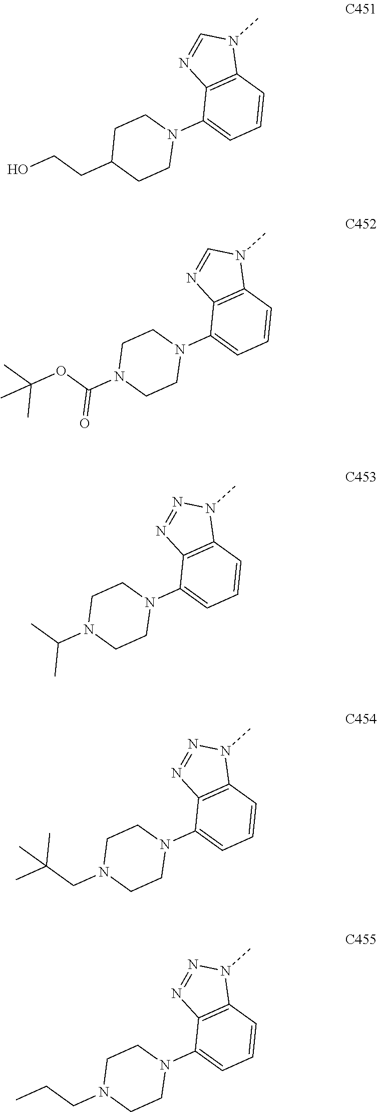

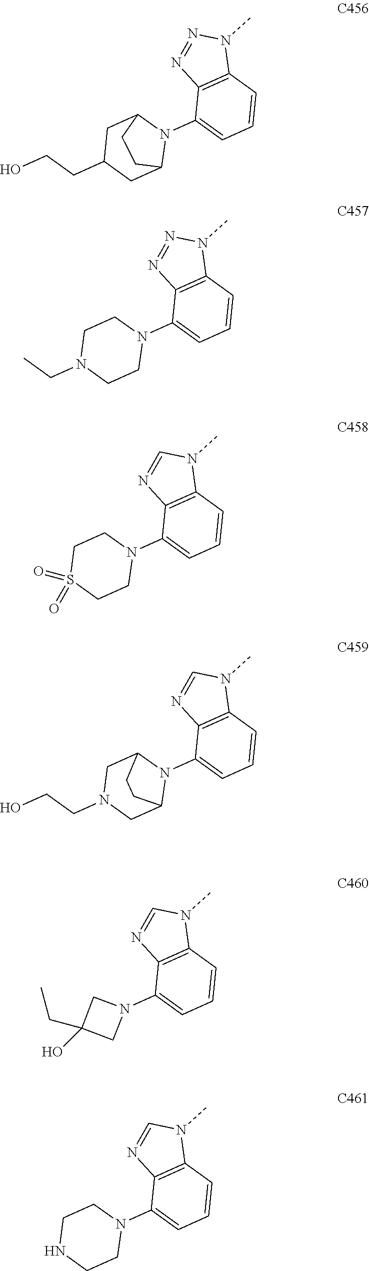

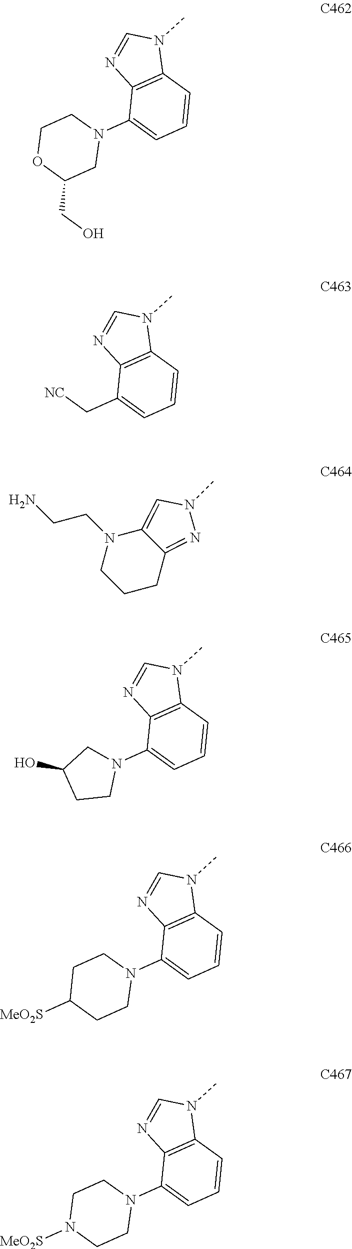

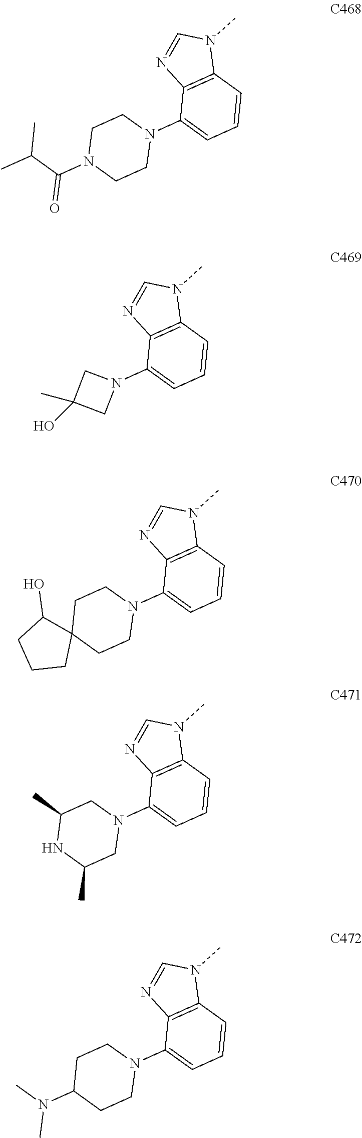

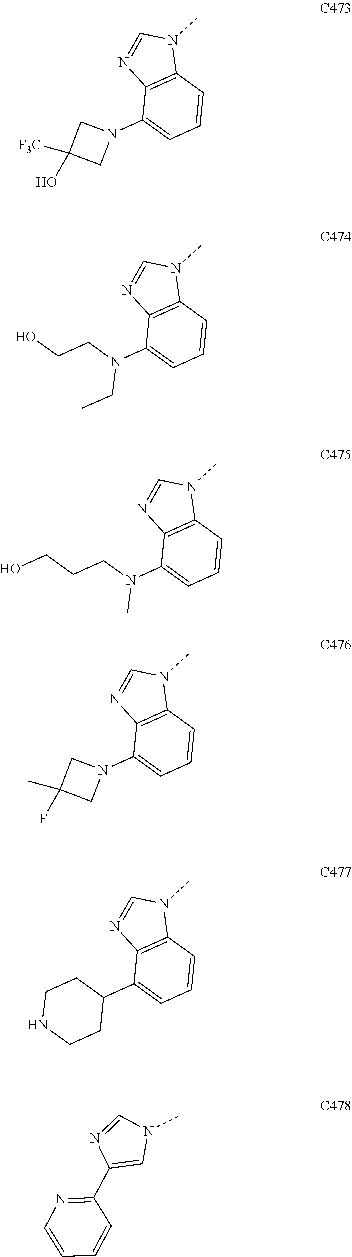

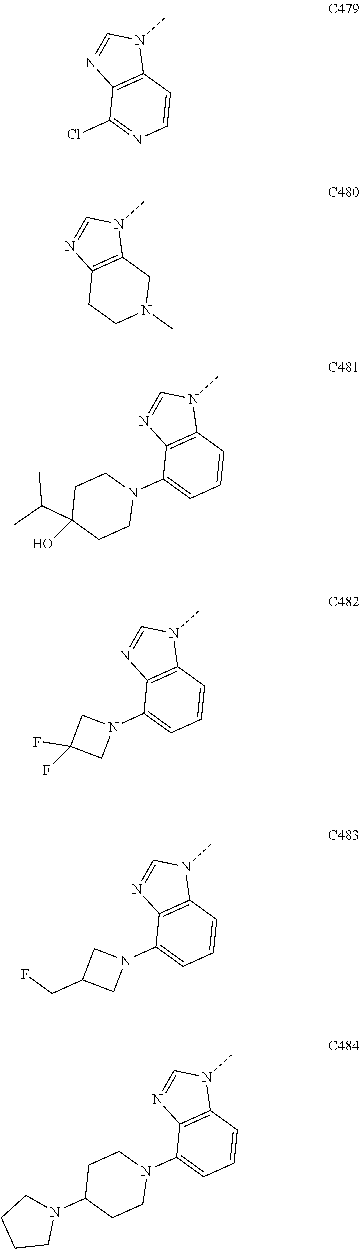

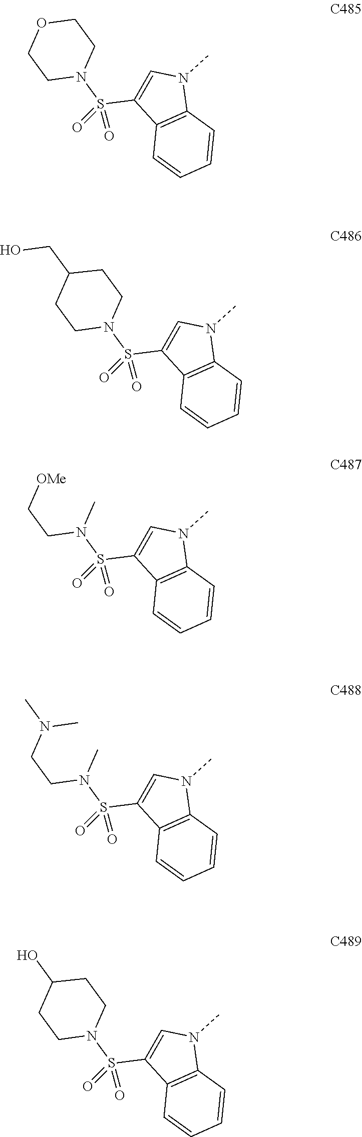

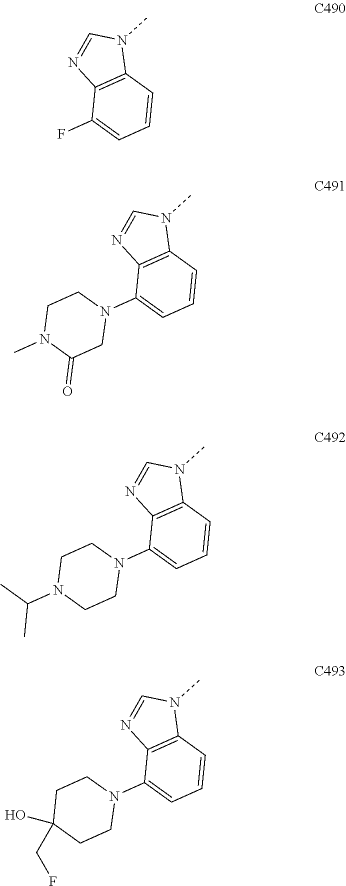

wherein (---) represents a bond.

In one embodiment, R¹ is selected from groups C1 to C493 or R¹ is selected from groups C1 to C23, C27, C60, C69, C71 to C73, C81 to C83, C88, C114, C182 to C184, C196, C220, C223 to C226, C275, C292, C310, C312, C313, C323, C346, C376, C402, C404, C414, C418, C419, C434, C435, C438, C440, C441, C472, C483, C488 and C490, for instance, R¹ is selected from groups C1, C3, C5, C7, C22, C23, C27, C60, C69, C73, C81 to C83, C88, C182 to C184, C196, C224-C226, C313, C323, C376, C402, C404, C414, C418, C419, C438 and C488, for instance, selected from C7, C22, C23 and C60 or from C183, C323, C376, C414, C418, C419, C438 and C488.

The following embodiments depict combinations of R¹ (C1 to C493), R² (1 to B77) and L (L1 to L4) groups that can be combined to produce compounds of Formula I where Y is H or NH₂:
C1-L-B1; C1-L-B2; C1-L-B3; C1-L-B4 to 70; C1-L-B71; C1-L-B72; C1-L-B73; C1-L-74; C1-L-B75 to B77;
C2-L-B1; C2-L-B2; C2-L-B3; C2-L-B4 to 70; C2-L-B71; C2-L-B72; C2-L-B73; C2-L-B74; C2-L-B75 to B77;
C3-L-B1; C3-L-B2; C3-L-B3; C3-L-B4 to 70; C3-L-B71; C3-L-B72; C3-L-B73; C3-L-B74; C3-L-B75 to B77;
C4 to C488-L-B1; C4 to C488-L-B2; C4 to C488-L-B3; C4 to C488-L-B4 to 70; C4 to C488-L-B71; C4 to C488-L-B72; C4 to C488-L-B73; C4 to C488-L-B74; C4 to C488-L-B75 to B77;
C489-L-E1; C489-L-E2; C489-L-E3; C489-L-E4 to B70; C489-L-B71; C489-L-B72; C489-L-B73; C489-L-B74; C489-L-E75 to B77;
C490-L-1; C490-L-E2; C490-L-E3; C490-L-E4 to B70; C490-L-B71; C490-L-B72; C490-L-B73; C490-L-B74; C490-L-E75 to B77;
C491 to C493-L-1; C491 to C493-L-E2; C491 to C493-L-E3; C491 to C493-L-E4 to B70; C491 to C493-L-B71; C491 to C493-L-B72; C491 to C493-L-B73; C491 to C493-L-B74; or C491 to C493-L-E75 to B77.

In one embodiment, the compound is as defined in Formula I, wherein:
R¹ is selected from groups C1 to C493 or R¹ is selected from groups C1 to C23, C27, C60, C69, C71 to C73, C81 to C83, C88, C114, C182 to C184, C196, C220, C223 to C226, C275, C292, C310, C312, C313, C323, C346, C376, C402, C404, C414, C418, C419, C434, C435, C438, C440, C441, C472, C483, C488 and C490, for instance, R¹ is selected from groups C1, C3, C5, C7, C22, C23, C27, C60, C69, C73, C81 to C83, C88, C182 to C184, C196, C224-C226, C313, C323, C376, C402, C404, C414, C418, C419, C438 and C488;
R² is selected from 1, B2, B8, B11, B12, B20 to B23 B34 to B37, B41 to B44, B49, B51 to B54, B57, B59, B62 to B67, B71 to B74 and B77; and
L is a group selected from L1 to L4, and Y is H or NH₂, preferably Y is H.

In another embodiment, the compound is as defined in Formula I, wherein R¹ is selected from C7, C22, C23, C60, C73, C81, C83, C183, C376, C404, C414, C418, C419, C438 and C488, R² is selected from B12, B21, B36, B41, B42, B52 to B54, B59, B65 and B73, L is a group selected from L1 to L4, and Y is H or NH₂, preferably Y is H.

Exemplary compounds as defined herein include each single compound covered in Tables 2, 3, 4, and 5 under Examples 1-163.

Examples of preferred compounds are, namely, Examples 31, 36, 40, 51, 55 to 60, 69, 72, 80 to 83, 88, 93, 94, 96 to 122, 124 to 147, 149, 151 to 160, 162 and 163 from Tables 3, 4, and 5. Examples of more preferred compounds include Examples 80 to 83, 93, 94, 96, 98 to 101, 104, 106, 111, 112, 114 to 116, 119, 120, 122, 125, 128 to 134, 139, 142, 144 to 146, 153, 155, 157, 159 and 162 from Tables 3 and 5.

It is understood that any of the above compounds may be in any amorphous, crystalline or polymorphic form, including any salt or solvate form, or a mixture thereof. The compounds of the present description may be further modified by appending various functionalities via any synthetic means delineated herein to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

These compounds may be prepared by conventional chemical synthesis, such as those exemplified in the Schemes and Examples of the present disclosure. As can be appreciated by the skilled artisan, further methods of synthesizing the compounds of the formulae herein will be evident to those of ordinary skill in the art. Additionally, the various synthetic steps may be performed in an alternate sequence or order to give the desired compounds.

### iii. Methods, Uses, Formulations and Administration

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in treatment, healing, prevention, or amelioration of a disease, disorder, or symptom thereof, or a decrease in the rate of advancement of a disease or disorder. The term also includes within its scope amounts effective to enhance normal physiological function.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

In one embodiment, the disease or condition to be treated is a proliferative disease or disorder or a kinase-mediated disease or disorder. More specifically, the disease or disorder to be treated include a proliferative disease or disorder, a developmental anomaly caused by dysregulation of the RAS-ERK signaling cascade (RASopathies), an inflammatory disease or an immune system disorder.

According to some examples, the proliferative disease or disorder to be treated is a neoplasm, an inflammatory disease or condition or a developmental anomaly, involving a constitutively activating mutation in RAS and/or RAF genes (e.g. KRAS and/or ARAF, BRAF or CRAF mutations). The disease or disorder may also be further associated with a receptor tyrosine kinase mutation or amplification (e.g. EGFR, HER2) or a mutation in a regulator of RAS downstream of the receptor (e.g. SOS1 gain of function, NF1 loss of function). For instance, the compounds as defined herein are inhibitors of signal enzymes (ex. B- and CRAF) which are involved in controlling cell proliferation not only in tumors harboring RAF mutations (e.g. BRAF^{V600E}) but importantly also in the context of mutated RAS-driven cancers. Thus, the present compounds may be used for example for the treatment of diseases connected with the activity of these signal enzymes and characterized by excessive or abnormal cell proliferation.

According to one embodiment, the disease or disorder is characterized by uncontrolled cell proliferation, i.e. a “proliferative disorder” or “proliferative disease”. More specifically, these diseases and disorders relate to cells having the capacity for autonomous growth, i.e. an abnormal state of condition characterized by rapidly proliferating cell growth which generally forms a distinct mass that show partial or total lack of structural organization and functional coordination with normal tissue.

For instance, the proliferative disorder or disease is defined as a “neoplasm”, “neoplastic disorder”, “neoplasia” “cancer,” and “tumor” which terms are collectively meant to encompass hematopoietic neoplasms (e.g. lymphomas or leukemias) as well as solid neoplasms (e.g. sarcomas or carcinomas), including all types of pre-cancerous and cancerous growths, or oncogenic processes, metastatic tissues or malignantly transformed cells, tissues, or organs, irrespective of histopathologic type or stage of invasiveness. Hematopoietic neoplasms are malignant tumors affecting hematopoietic structures (structures pertaining to the formation of blood cells) and components of the immune system, including leukemias (related to leukocytes (white blood cells) and their precursors in the blood and bone marrow) arising from myeloid, lymphoid or erythroid lineages, and lymphomas (related to lymphocytes). Solid neoplasms include sarcomas, which are malignant neoplasms that originate from connective tissues such as muscle, cartilage, blood vessels, fibrous tissue, fat or bone. Solid neoplasms also include carcinomas, which are malignant neoplasms arising from epithelial structures, including external epithelia (e.g., skin and linings of the gastrointestinal tract, lungs, and cervix), and internal epithelia that line various glands (e.g., breast, pancreas, thyroid). Examples of neoplasms include leukemia, and hepatocellular cancers, sarcoma, vascular endothelial cancers, breast cancers, central nervous system cancers (e.g. astrocytoma, gliosarcoma, neuroblastoma, oligodendroglioma and glioblastoma), prostate cancers, lung and bronchus cancers, larynx cancers, esophagus cancers, colon cancers, colorectal cancers, gastro-intestinal cancers, melanomas, ovarian and endometrial cancer, renal and bladder cancer, liver cancer, endocrine cancer (e.g. thyroid), and pancreatic cancer. For instance, the disease or disorder is selected from colon cancer, lung cancer, pancreatic cancer, thyroid cancer, breast cancer and skin cancer. Examples of neoplasm include melanoma, papillary thyroid carcinoma, colorectal, ovarian, breast cancer, endometrial cancer, liver cancer, sarcoma, stomach cancer, Barret's adenocarcinoma, glioma (including ependymoma), lung cancer (including non-small cell lung cancer), head and neck cancer, acute lymphoblastic leukemia, acute myelogenous leukemia, non-Hodgkin's lymphoma, and hairy-cell leukemia.

In an embodiment, patients presenting one of the above-mentioned hematopoietic or solid neoplasms have previously received treatment with a RAS-ERK pathway-targeted inhibitor (including RTK, RAF, MEK or ERK inhibitor) but have developed resistance to the said inhibitor. The inhibitor includes standard of care treatments such as vemurafenib, dabrafenib, cobimetinib, trametinib, YERVOY, OPDIVO or any combination of these pharmaceutical agents.

In an embodiment, the disease to be treated is defined by developmental anomalies caused by dysregulation of the RAS-ERK signaling cascade (RASopathies: e.g. Noonan syndrome, Costello syndrome, LEOPARD syndrome, cardiofaciocutaneous syndrome and hypertrophic cardiomyopathy).

In an embodiment, the disease to be treated is defined as an inflammatory disease or immune system disorder. Examples of such inflammatory diseases or immune system disorders including inflammatory bowel disease, Crohn's disease, ulcerative colitis, systemic lupus erythematosis (SLE), rheumatoid arthritis, multiple sclerosis, thyroiditis, type 1 diabetes, sarcoidosis, psoriasis, allergic rhinitis, asthma, COPD (chronic obstructive pulmonary disease).

In one embodiment, the compounds as herein defined are inhibitors of RAS-ERK signaling and cellular proliferation in tumor cells bearing at least one mutated RAS or RAF genotype, without or substantially without inducing the paradoxical pathway.

The term “patient or subject” as used herein refers to an animal such as a mammal. A subject may therefore refer to, for example, mice, rats, dogs, cats, horses, cows, pigs, guinea pigs, primates including humans and the like. Preferably the subject is a human.

The present description therefore further relates to a method of treating a subject, such as a human subject, suffering from a proliferative disease or disorder, e.g. a RAF-mutated and/or mutated RAS-driven cancer. The method comprises administering a therapeutically effective amount of a compound as defined herein, to a subject in need of such treatment.

In certain embodiments, the present description provides a method of treating a disorder (as described herein) in a subject, comprising administering to the subject identified as in need thereof, a compound of the present description. The identification of those patients who are in need of treatment for the disorders described above is well within the ability and knowledge of one skilled in the art. Certain of the methods for identification of patients which are at risk of developing the above disorders which can be treated by the subject method are appreciated in the medical arts, such as family history, and the presence of risk factors associated with the development of that disease state in the subject patient. A clinician skilled in the art can readily identify such candidate patients, by the use of, for example, clinical tests, physical examination, medical/family history, and genetic determination.

A method of assessing the efficacy of a treatment in a subject includes determining the pre-treatment symptoms of a disorder by methods well known in the art and then administering a therapeutically effective amount of a compound of the present description, to the subject. After an appropriate period of time following the administration of the compound (e.g., 1 week, 2 weeks, one month, six months), the symptoms of the disorder are determined again. The modulation (e.g., decrease) of symptoms and/or of a biomarker (e.g. pERK or pMEK) of the disorder indicates efficacy of the treatment. The symptoms and/or biomarker of the disorder may be determined periodically throughout treatment. For example, the symptoms and/or biomarker of the disorder may be checked every few days, weeks or months to assess the further efficacy of the treatment. A decrease in symptoms and/or biomarker of the disorder indicates that the treatment is efficacious.

In some embodiments, the therapeutically effective amount of a compound as defined herein can be administered to a patient alone or in a composition, admixed with a pharmaceutically acceptable carrier, adjuvant, or vehicle.

The expression “pharmaceutically acceptable carrier, adjuvant, or vehicle” and equivalent expressions, refer to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions described herein may be administered orally, parenterally, by inhalation spray, topically, rectally, nasally, buccally, or via an implanted reservoir. The term “parenteral” as used herein includes subcutaneous, intravenous, intramuscular, intraarticular, intrasynovial, intrasternal, intrathecal, intrahepatic, intralesional and intracranial injection or infusion techniques. Other modes of administration also include intradermal or transdermal administration.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, surfactants, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a provided compound, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled.

Examples of other biodegradable polymers include poly(orthoesters) and poly(anhydrides). Depot injectable formulations are also prepared by entrapping the compound in liposomes or microemulsions that are compatible with body tissues.

Compositions for rectal administration are preferably suppositories which can be prepared by mixing the compounds of the present description with suitable non-irritating excipients or carriers such as cocoa butter, polyethylene glycol or a suppository wax which are solid at ambient temperature but liquid at body temperature and therefore melt in the rectum and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone (PVP), sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The composition can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of the present description include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of the present description. Additionally, the description contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Pharmaceutically acceptable compositions provided herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promotors to enhance bioavailability, fluorocarbons, and/or other conventional solubilizing or dispersing agents.

Pharmaceutically acceptable compositions provided herein may be formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compound that may be combined with carrier materials to produce a composition in a single dosage form will vary depending upon the patient to be treated and the particular mode of administration. Provided compositions may be formulated such that a dosage of between 0.01-100 mg/kg body weight/day of the inhibitor can be administered to a patient receiving these compositions.

It should also be understood that a specific dosage and treatment regimen for any particular patient will depend upon a variety of factors, including age, body weight, general health, sex, diet, time of administration, rate of excretion, drug combination, the judgment of the treating physician, and the severity of the symptoms associated with the proliferative disease or disorder. The amount of a provided compound in the composition will also depend upon the particular compound in the composition.

Compounds or compositions described herein may be administered using any amount and any route of administration effective for treating or lessening the severity of the symptoms as contemplated herein. The exact amount required will vary from subject to subject, depending on the species, age, and general condition of the subject, the severity of the infection, the particular agent, its mode of administration, and the like. Provided compounds are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present disclosure will be decided by the attending physician within the scope of sound medical judgment.

Pharmaceutically acceptable compositions of this disclosure can be administered to humans and other animals orally, rectally, parenterally, intracisternally, intraperitoneally, topically (as by powders, ointments, or drops), buccally, as an oral or nasal spray, or the like, depending on the severity of the infection being treated. In certain embodiments, provided compounds may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

It will be understood, that the total daily usage of the compounds and compositions of the present description will be decided by the attending physician within the scope of sound medical judgment. The total daily inhibitory dose of the compound of the present description administered to a subject in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In one embodiment, treatment regimens according to the present description comprise administration to a patient in need of such treatment from about 10 mg to about 1000 mg of the compound(s) of the present description per day in single or multiple doses.

Depending upon the disease or disorder to be treated, additional therapeutic agents may also be present in the compositions of this disclosure or administered separately as part of a dosage regimen, e.g. an additional chemotherapeutic agent. Non-limiting examples of additional therapeutic agents which could be used in combination with the present compounds include antiproliferative compounds such as aromatase inhibitors; anti-estrogens; anti-androgens; gonadorelin agonists; topoisomerase I inhibitors; topoisomerase II inhibitors; microtubule active agents; alkylating agents; retinoids, carotenoids, tocopherol; cyclooxygenase inhibitors; MMP inhibitors; antimetabolites; platin compounds; methionine aminopeptidase inhibitors; bisphosphonates; antiproliferative antibodies; heparanase inhibitors; inhibitor of Ras oncogenic isoforms; telomerase inhibitors; proteasome inhibitors; compounds used in the treatment of hematologic malignancies; kinesin spindle protein inhibitors; Hsp90 inhibitors; mTOR inhibitors; PI3K inhibitors; Flt-3 inhibitors; CDK4/6 inhibitors; HER2 inhbiitors (Herceptin, Trastuzumab); EGFR inhibitors (Iressa, Tarceva, Nerlynx, Tykerb, Erbitux); RAS inhibitors; MEK inhibitors (Trametinib, Binimetinib, Cobimetinib); ERK inhibitors (Ulixertinib); anti-PD-1 antibodies (Opdivo, Keytruda); anti-CTLA4 antibodies (Yervoy); antitumor antibiotics; nitrosoureas; compounds targeting/decreasing protein or lipid kinase activity, compounds targeting/decreasing protein or lipid phosphatase activity, or any further anti-angiogenic compounds.

The treatment may also be complemented with other treatments or interventions such as surgery, radiotherapy (e.g., gamma-radiation, neutron beam radiotherapy, electron beam radiotherapy, proton therapy, brachytherapy, and systemic radioactive isotopes), a biologic response modifier (e.g., an interferon, an interleukin, tumor necrosis factor (TNF), and agents used to attenuate an adverse effect.

The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### EXAMPLES

### List of Abbreviations:

Ac: acetyl
AcOEt: ethyl acetate
AcOH: acetic acid
Ar: aryl
ATCC: American Type Culture Collection
ATP: adenosine triphosphate
BINOL: [1,1′-binaphthalene]-2,2′-diol
Boc: tert-butyloxycarbonyl
BOP: (benzotriazol-1-yloxy)tris(dimethylamino)phosphonium hexafluorophosphate
br: broad
BSA: bovine serum albumin
CCL: cancer cell lines
DCE: 1,2-dichloroethane
DCM: dichloromethane
DIEA (or DIPEA): N,N-diisopropylethylamine (Huenig's base)
DME: 1,2-dimethoxyethane
DMF: N,N-dimethylformamide
DMSO: dimethylsulfoxide
DTT: dithiothreitol
EA: ethyl acetate
EC₅₀: half-maximal effective concentration
ECL: enhanced chemiluminescence
EDTA: ethylenediamine tetraacetic acid
Et₂O: diethyl ether
EtOH: ethanol
Eu: Europium
FBS: fetal bovine serum
GST: glutathion S-transferase
HATU: O-(7-azabenzotriazol-1-yl)-N,N,N′,N′-tetramethyluronium hexafluorophosphate
HEPES: 4-(2-hydroxyethyl)-1-piperazineethanesulfonic acid
Het: heterocycle
Hex: hexanes
HRMS: high resolution mass spectrometry
HPLC: high performance liquid chromatography
HRP: horseradish peroxidase
IC₅₀: half-maximal inhibitory concentration
IPA or iPrOH: isopropanol
LCMS: liquid chromatography mass spectrometry
MeCN: acetonitrile
MS: mass spectrometry
NMP: N-methylpyrrolidone
NMR: nuclear magnetic resonance
ON: overnight
PBS: phosphate buffered saline
pERK: phosphorylated extracellular signal-regulated kinase
PMB: para-methoxybenzyl
PMSF: phenylmethylsulfonyl-fluoride
Rf: retention factor
RPMI-1640: Roswell Park Memorial Institute medium
RT: room temperature
SDS: sodium dodecylsulfate
SDS-PAGE: sodium dodecyl-sulfate-polyacrylamide gel electrophoresis
SEM: trimethylsilylethoxymethyl
SNAr: Nucleophilic aromatic substitution
TBST: Tris buffered saline with 0.2% Tween-20
TBTU: 0-(benzotriazol-1-yl)-N,N,N′,N′-tetramethyluronium tetrafluoroborate
TEV: tobacco etch virus protease
TFA: trifluoroacetic acid
THF: tetrahydrofuran
TLC: silica gel thin layer chromatography
TR-FRET: time-resolved fluorescence resonance energy transfer
Ts: para-toluenesulfonate
Y_{MIN}: minimal data point of a dosage-activity curve

The following non-limiting examples are illustrative embodiments and should not be construed as further limiting the scope of the present invention. These examples will be better understood with reference to the accompanying figures.

The Examples set forth herein below provide syntheses and experimental results obtained for certain exemplary compounds. As it is well known to a person skilled in the art, reactions are performed in an inert atmosphere (nitrogen or argon) where necessary to protect reaction components from air and moisture. Temperatures are given in degrees Celsius (° C.). Solution percentages and ratios express a volume to volume relationship, unless otherwise stated. The reactants used in the examples below may be obtained either as described herein, or if not described herein, are themselves either commercially available or may be prepared from commercially available materials by methods known in the art. Flash chromatography is carried out on silica (SiO₂) using a Teledyne Isco Rf Combiflash instrument at 254 nm using commercial normal phase silica. Mass spectra analyses are recorded using electrospray mass spectrometry. NMR are recorded on a 400 MHz Varian instrument.

Preparative HPLC was performed using an Agilent instrument using a Phenomenex-Kinetex C18, (21×100 mm, 5 μm) column at a flow rate of 20 mL/min (RT) and UV detection at 220 and 254 nm. The mobile phase consisted of Solvent A (5% MeOH, 95% water+0.1% formic acid) and Solvent B (95% MeOH, 5% water+0.1% formic acid) unless stated otherwise. As specified in the text, 0.05% TFA or 0.1% AcOH or other additives were occasionally used as additives instead of 0.1% formic acid in both solvents. MeCN was also used instead of MeOH in both mobile phases for more challenging separations as specified in the text. Specific gradient conditions are provided in the examples but the following is representative: T(0)→T(3 min) isocratic using between 10 to 50% solvent B depending on compound polarity, followed by a 12 minutes gradient to 100% solvent B. Last 5 minutes 100% solvent B.

LCMS analyses were performed on an Agilent instrument. Liquid chromatography was performed on a Phenomenex Kinetex C18 column (2.6 μm; 100 Å; 3×30 mm) at a flow rate of 1.5 mL/min (RT) with UV detection at 220 and 254 nm. The mobile phase consisted of solvent A (95% H₂O/5% MeOH/0.1% AcOH) and solvent B (95% MeOH/5% H₂O/0.1% AcOH) using the following gradient: T(0) 100% A→T(0.5 min) 100% B→isocratic 100% B to T(2 min). MS detection was performed in parallel using APCI detection in both positive and negative modes.

Unless otherwise indicated, all numbers expressing quantities of ingredients, reaction conditions, concentrations, properties, stabilities, and so forth used in the specification and claims are to be understood as being modified in all instances by the term “about.” At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such.

### Synthesis, Biological Activity and Characterization of Examples

All compounds herein described were prepared according to methods as indicated in Tables 2 to 5. Characterization data by mass spectrometry and NMR are provided for each of the Examples. The compounds are tested in the assays described in the biological Experimental section. The convention used for reporting biological data is provided as a footnote in the respective Tables.

### General Synthetic Method A:

Commercially available 2,6-difluoro-3-nitrobenzoic acid A-1 (Scheme A) can be converted to carbamate A-2 via a Curtius reaction following the procedure described in J. Med. Chem. 2003, 46, 1905. Catalytic hydrogenolysis of nitroarene A-2 using hydrogen gas and a catalyst such as palladium metal on carbon or palladium hydroxide on carbon (Pearlman's catalyst) yields aniline A-3. Reaction of aniline A-3 with sulfonylating agents such as sulfonyl chlorides in the presence of an organic base such as pyridine that can be used as solvent, with or without a catalyst such as 4-dimethylaminopyridine, and in the presence or not of an additional solvent such as dichloromethane or tetrahydrofuran yields sulfonamide intermediate A-4 that can be deprotected to aniline salts such as A-5 using strong acid (e.g. solutions of anhydrous hydrochloric acid in dioxane). Alternatively, 2,6-difluoroaniline A-6 can be converted to its acetanilide A-7 using an acetylating agent such as acetic anhydride and converted to mono-protected dianiline A-8 as described in WO 2012/101238A1. Sulfonylation to sulfonamide A-9 is achieved under similar conditions used for the conversion of carbamate A-3 to sulfonamide A-4 using a sulfonylating reagent in the presence of an organic base such as pyridine, with or without a catalyst such as 4-dimethylaminopyridine and solvents such as dichloromethane or tetrahydrofuran. Treatment of acetanilide A-9 with aqueous hydrochloric acid in the presence of a co-solvent such as an alcohol provides aniline salt A-5.

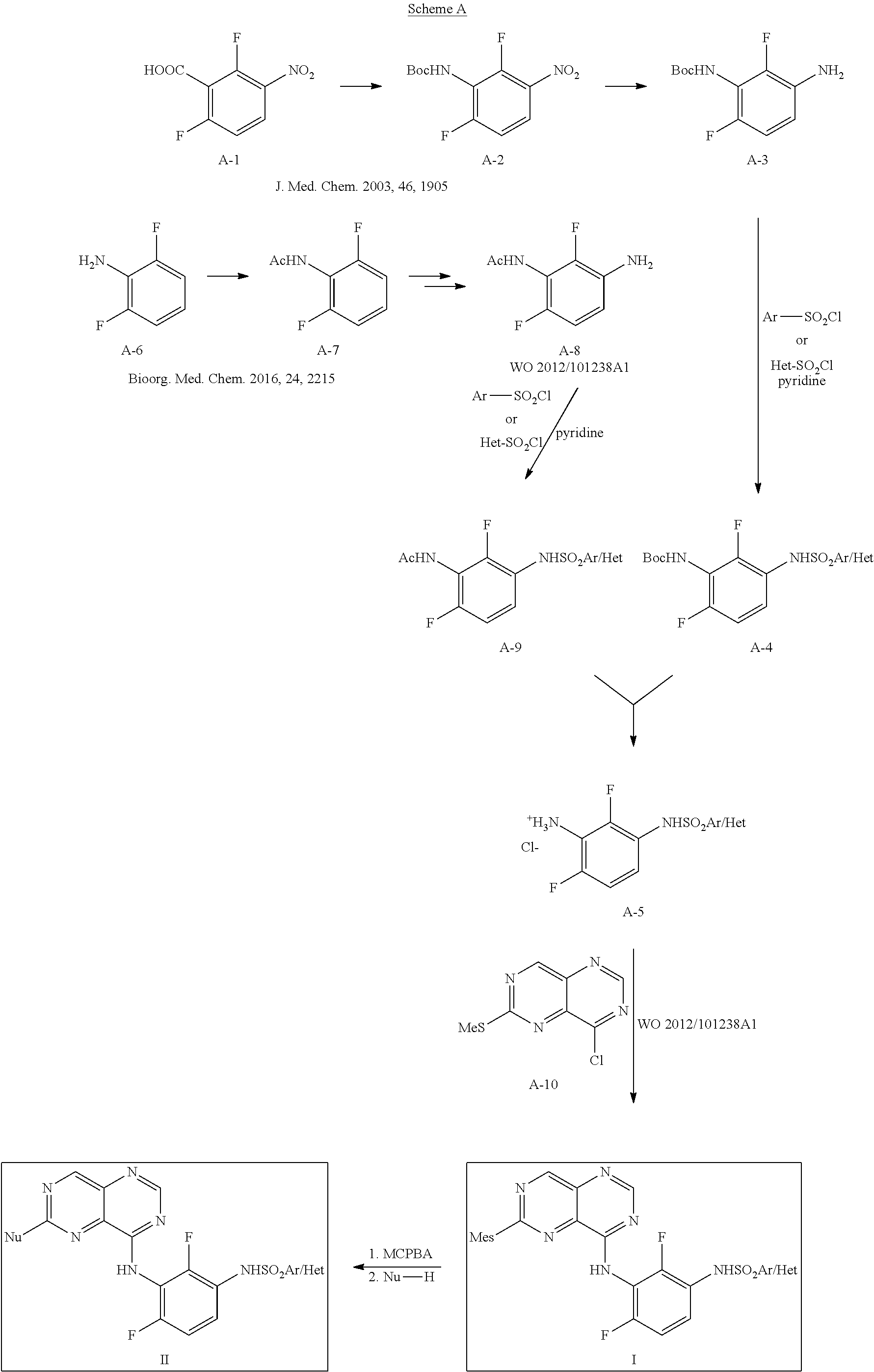

8-Chloro-2-(methylthio)pyrimidopyrimidine A-10 is commercially available or can be prepared as described in WO 2012/101238A1. Inhibitors of general formulae I are prepared by a nucleophilic substitution between A-10 and aniline derivatives A-5 following similar procedures to those described in WO 2012/101238A1. Inhibitors of general formulae II are prepared from inhibitors of general formulae I by a two-step procedure involving first oxidation of the thiomethyl group generally to a mixture of the corresponding methylsulfoxide and methylsulfone which is then reacted with a nucleophile (e.g. a 1° or a 2° amine, alcohol, phenol or a NH-containing heterocycle, etc.) following similar protocols to those described in WO 2012/101238A1. The latter step is usually carried out in the presence of a base (e.g. an organic base such as DIEA, trimethylamine, pyridine and the like) in a solvent such as DMSO or NMP at temperature ranging from 70 to 140° C.

### General Synthetic Method B:

An alternative method of preparing the present inhibitors is described in Scheme B. As shown in Scheme B, intermediate A-2 (described in Scheme A and prepared as described in J. Med. Chem. 2003, 46, 1905 is converted to nitroaniline hydrochloride B-1 by cleavage of the carbamate protecting group under acidic conditions (e.g. HCl in dioxane or TFA). Chloropyrimidopyrimidine A-10 (prepared as described in Scheme A (WO 2012/101238A1) undergoes nucleophilic substitution with aniline salts such as B-1 or preferably with the aniline free base under similar conditions to those described in WO 2012/101238A1 to provide intermediate B-2. The nitro functionality of intermediate B-2 is then reduced to the corresponding aniline B-3 using methods well established and familiar to practitioners of the art such as tin(II)chloride, iron or zin powder under acidic conditions in solvent such as MeOH, EtOH or EtOAc and the likes at temperatures ranging from 50° to 100° C. Aniline B-3 are then sulfonylated as described in Scheme A using sulfonyl chlorides under basic conditions to provide inhibitors of general formulae I. Inhibitors of formulae I are then converted to inhibitors of general formulae II as described in Scheme A.

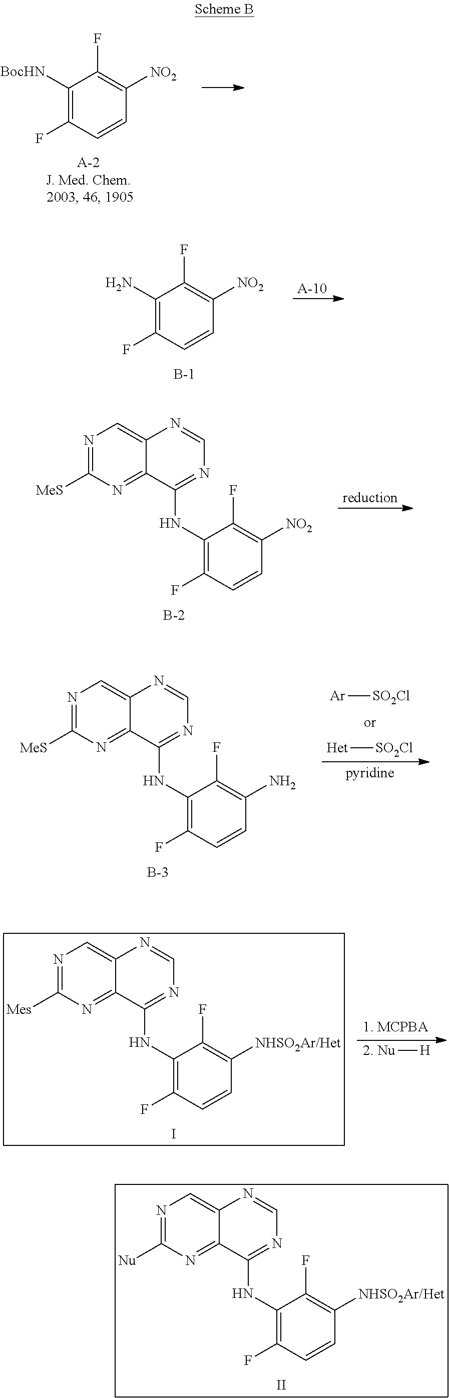

### General Synthetic Method C:

An alternative method of preparing the present inhibitors with general formulae III and IV is described in Scheme C. As shown in Scheme C, intermediate B3 prepared as described in Scheme B can be converted to chlorosulfonylaniline C-1 by treatment with sulfuryl chloride in the presence of an organic base such as a 3° amine (e.g. trimethylamine, DIEA and the likes). Intermediate C-1 reacts with 1° or 2° amines such as pyrrolidine derivatives, to provide inhibitors of general formulae III. Inhibitors of the general formulae IV are then obtained by a two-step procedure involving oxidation of the thiomethylgroup to a mixture of sulfoxide and sulfone followed by reaction with a nucleophile as described in Scheme A.

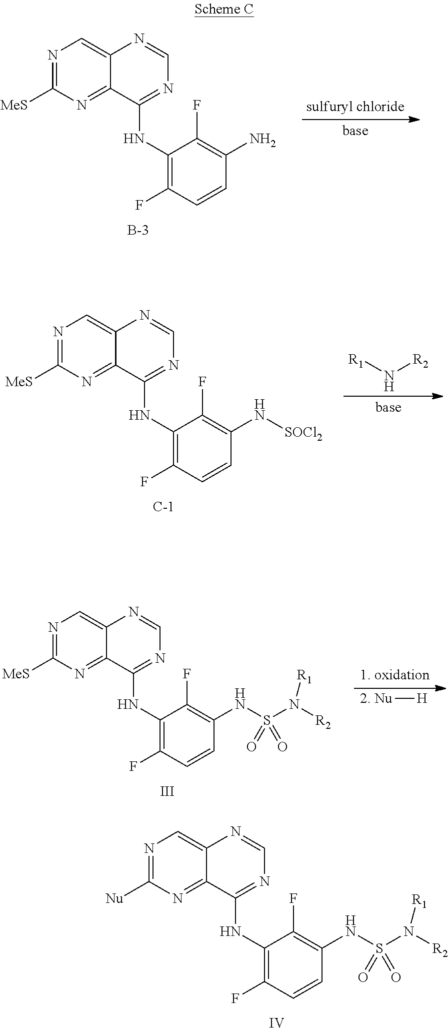

### General Synthetic Method D:

Examples of inhibitors of general formulae V were prepared following the sequence illustrated in Scheme D.

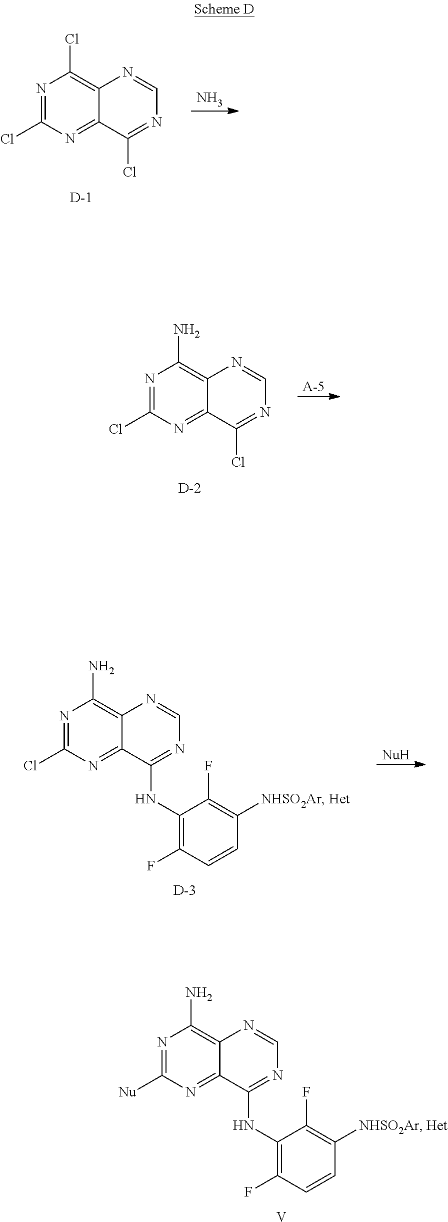

2,4,8-Trichloropyrimidopyrimidine D-1 was prepared by the procedure described in ACS Med. Chem. Lett. 2011, 2, 538 and converted to 4-amino-2,8-dichloropyrimidopyrimidine D-2 by treatment with ammonia as described in WO 2010/026262A1. Dichloropyrimidopyrimidine D-2 underwent regioselective nucleophilic displacement with aniline salts of general formulae A-5 under the general conditions described in Scheme A to provide 8-chloropyrimidopyrimidine intermediates D-3. Chloropyrimidopyrimidine D-3 undergoes a second displacement by nucleophiles such as 2° amines or a NH-containing heterocycle following similar protocols to those described in WO 2012/101238A1 to provide inhibitors of general formulae V. The latter step is usually carried out in the presence of a base (e.g. an organic base such as DIEA, trimethylamine, pyridine and the like) in a solvent such as DMSO or NMP at temperatures ranging from 70° C. to 140° C. Alternatively, intermediate D-3 react with nucleophiles such as 2° amines or NH-containing heteroaryls (e.g. imidazoles, benzimidazoles and the like) under copper-catalyzed cross coupling conditions in the presence of an organic ligand (e.g. racemic-BINOL) and an inorganic base such as cesium carbonate. These reactions are usually conducted in solvents such as DMSO or NMP at temperatures ranging from 80° C. to 140° C. Other methods of coupling chloropyrimidines to such nucleophiles that involve metal-catalyzed processes are well known to those skilled in the art and can be employed to access inhibitors of general formulae V.

### General Synthetic Method E:

Inhibitors of general formulae VI are prepared as described in Scheme E. Intermediates of general formulae I were first prepared following general method A or B and then oxidized to a mixture of methylsulfoxide and methylsulfone, as described in general method A. Intermediates I were then coupled to a 3-indolecarboxylic acid ester (e.g. methyl ester, X═CH) or a 3-indazolecarboxylic acid ester (e.g. methyl ester, X═N) following similar protocols to those described in WO 2012/101238A1. The latter step is usually carried out in the presence of a base (e.g. an organic or inorganic base such as Cs₂CO₃, KOtBu, DIEA, trimethylamine, pyridine and the like) in a solvent such as THF, DMSO or NMP at temperatures ranging from ambient to 140° C. Deprotection of the ester protecting group using an inorganic base (e.g. NaOH or KOH) in mixtures of water and a miscible organic solvent (e.g. methanol, ethanol, THF, dioxane and the likes) at temperatures ranging from ambient to 100° C. followed by acidification with a mineral (e.g. aqueous hydrochloric or sulfuric acid), inorganic salt solution (e.g. aqueous NH₄Cl or KHSO₄) or organic acid (e.g. aqueous citric or acetic acid) provided the corresponding carboxylic acid intermediates E-1. Coupling of intermediates E-1 with amines using standard amide coupling reagents (e.g. TBTU, HATU, DCC, EDC and the likes) provided amide derivatives of general formulae VI.

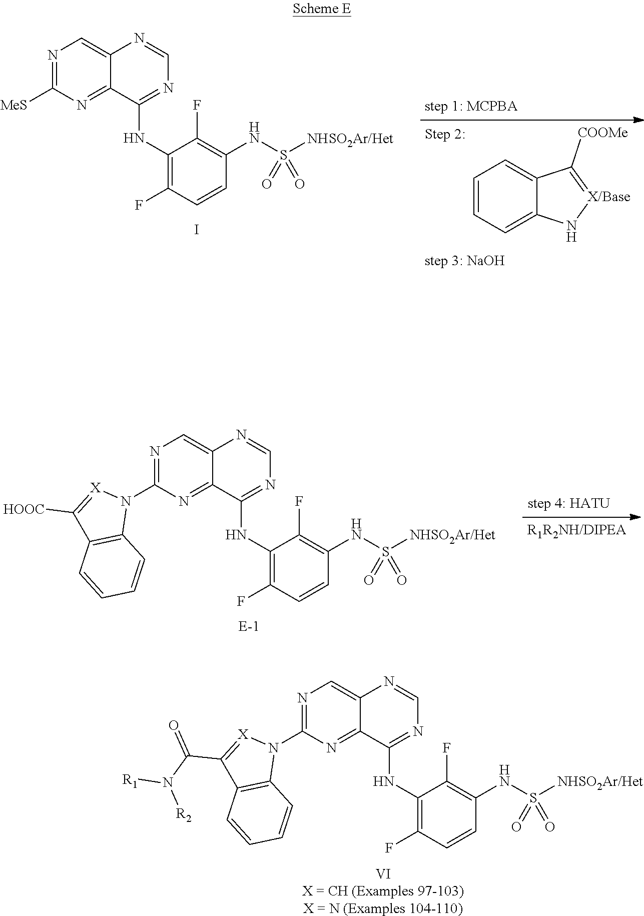

### General Synthetic Method F:

Following the general procedure described in Scheme F, thiomethyl intermediates I, prepared as described following general method A or B were oxidized in step 1 to a mixture of methylsulfoxide and methylsulfone, as described in general method A. Separately in step 2, 3-indolesulfonyl chloride was prepared as described in Org. Lett. 2011, 13, 3588 and condensed with amines in the presence of an organic base (e.g. DIEA, trimethylamine and the likes) in a solvent such as THF to provide intermediates 3-indolesulfonamide intermediates F-1. Alternatively, N-tosyl protected indole-3-sulfonyl chloride (prepared by the procedure described in Chemical and Pharmaceutical Bulletin 2009, 57, 591) is reacted with primary or secondary amines in a solvent such as THF and in the presence of a tertiary base such as DIEA or triethylamine to provide intermediate sulfonamides F-1 after removal of the tosyl protecting group upon treatment with an aqueous inorganic base such as KOH. Intermediates F-1 were then condensed with the oxidized mixture of intermediates I from step 1 following similar protocols to those described in WO 2012/101238A1. The latter step is usually carried out in the presence of a base (e.g. an organic base such as DIEA, trimethylamine, pyridine and the like) in a solvent such as DMSO or NMP at temperatures ranging from 70 to 140° C. to provide inhibitors of general formulae VII.

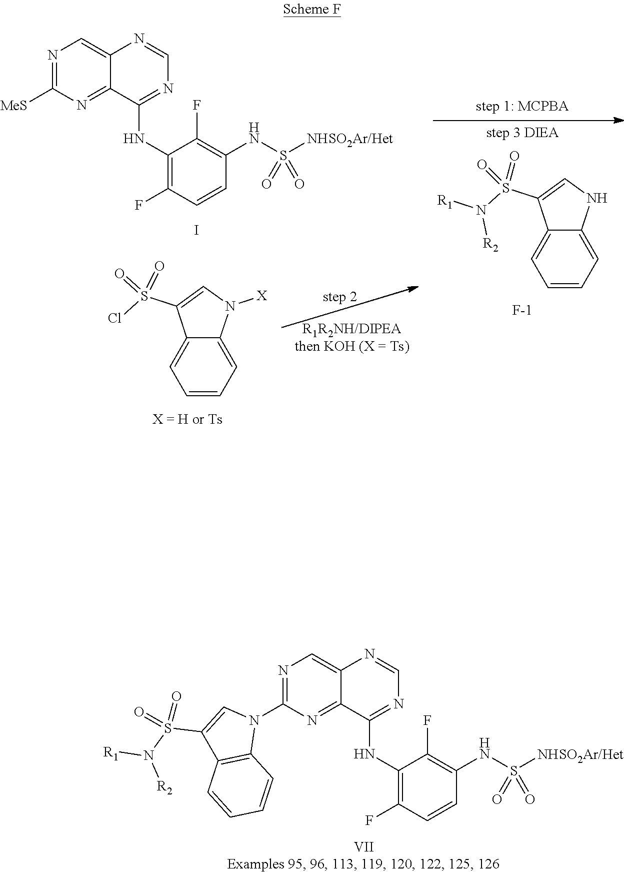

### General Synthetic Method G:

3-Indolethiocyanate G-1 (prepared following the procedure described in Phosphorus, Sulfur and Silicon and the Related Elements 2014, 189, 1378) is reduced to the corresponding sulfide salt using a reducing agent such as sodium sulfide nonahydrate and directly alkylated without isolation with an alkyl halide to provide sulfide intermediates G-2. Sulfide intermediates G-2 are then converted to sulfone intermediates G-3 using an oxidizing agent such as 3-chloroperoxybenzoic acid. Final inhibitors of the general structure VIII are then obtained in the usual fashion as described.

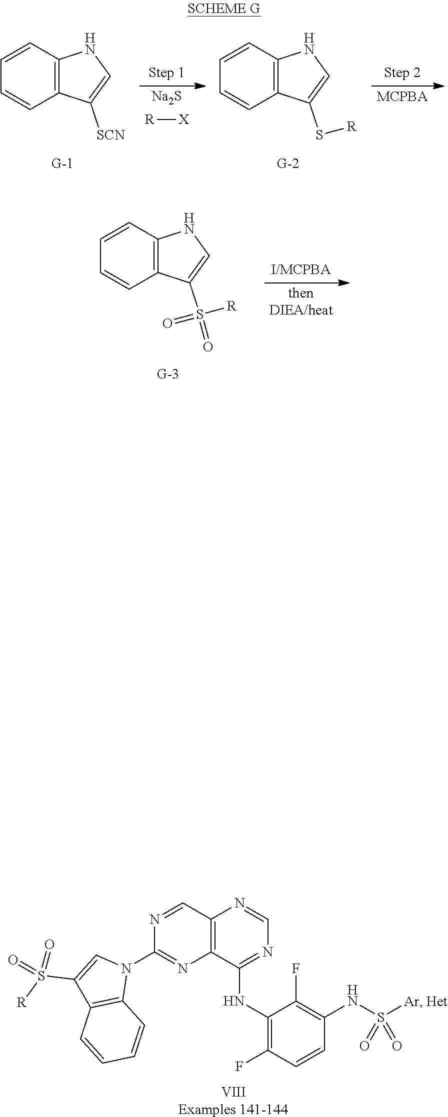

### General Synthetic Method H:

A bright red solution of commercially available 3-fluoro-2-nitroaniline H-1 reacts with primary or secondary amines in solvents such as MeCN, DMSO or NMP and in the presence of an inorganic base such as potassium carbonate or an organic base such as DIEA to provide intermediates H-2 upon heating at temperature ranging from 40° C. to 120° C. under thermal or microwave conditions. Reduction of the nitro group of intermediate H-2 can be achieved using metals such as Fe or Zn in the presence of ammonium chloride at temperatures ranging from 40° C. to 80° C. in an alcoholic solvent such as isopropanol. 1,2-Phenylenediamine intermediates are then converted directly to the desired benzimidazole intermediates H-3 upon heating with formic acid at temperatures ranging from 40° C. to 80° C. Final inhibitors of the general structure IX are then obtained from intermediates benzimidazoles H-3 and I under usual conditions as described previously.

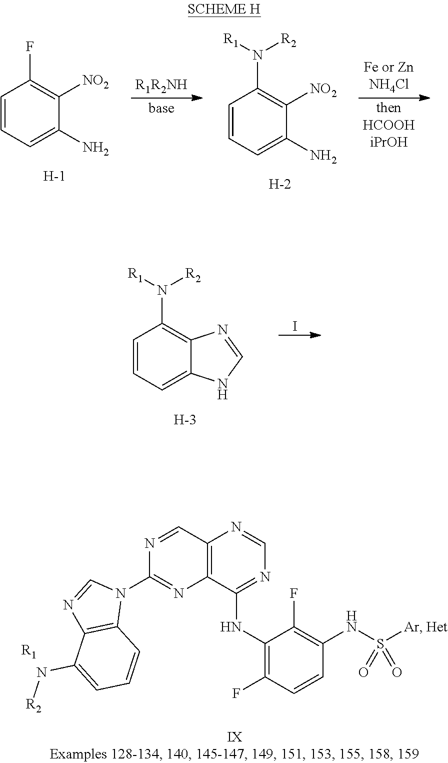

### General Synthetic Method I:

Nitroanilines H-2 obtained as described under general synthetic method H are reduced to 1,2-phenylenediamines I-1 using metals such as Fe or Zn in the presence of ammonium chloride at temperature ranging from 40° C. to 80° C. in an alcoholic solvent such as isopropanol. 1,2-phenylenediamine intermediates I-1 are then converted to the desired benzotriazole intermediates I-2 upon treatment with an inorganic nitrite such as sodium nitrite under acidic conditions (for example AcOH). Final inhibitors of the general structure X are then obtained from intermediates I-2 and I under usual conditions as described previously.

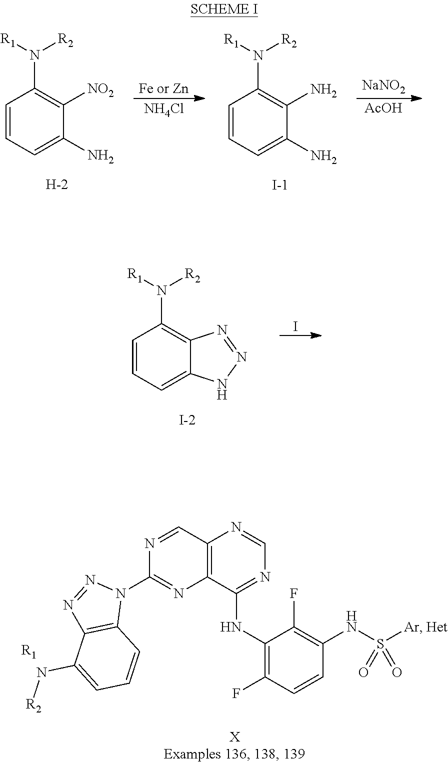

### Sulfonyl Chlorides:

The following sulfonyl chlorides were obtained from commercial sources and used as received: 4-methoxybenzenesulfonyl chloride, 2,4-dichlorobenzenesulfonyl chloride, 2,4-dimethylbenzenesulfonyl chloride, 2-chlorobenzenesulfonyl chloride, 2-methylbenzenesulfonyl chloride, 4-ethylbenzenesulfonyl chloride, 2-cyanobenzenesulfonyl chloride, 2,4-dimethoxybenzenesulfonyl chloride, 2-trifluoromethylbenzenesulfonyl chloride, 3-chlorobenzenesulfonyl chloride, 3-methylbenzenesulfonyl chloride, 2,3-dichlorobenzenesulfonyl chloride, 3-chloro-2-methylbenzenesulfonyl chloride, 2-bromobenzenesulfonyl chloride, 2-chloro-4-fluorobenzenesulfonyl chloride, 2-chloro-6-fluorobenzenesulfonyl chloride, 2,5-dichlorobenzenesulfonyl chloride, 2,5-dimethylbenzenesulfonyl chloride, 2-chloro-6-methylbenzenesulfonyl chloride, 3-fluoro-2-methylbenzenesulfonyl chloride, 2-chloro-4-methylbenzenesulfonyl chloride, 1,3-benzodioxole-5-sulfonyl chloride, 2-chloro-4-(trifluoromethyl)-benzenesulfonyl chloride, 2-methyl-4-nitrobenzenesulfonyl chloride, 2-(difluoromethyl)benzenesulfonyl chloride.

Other sulfonyl chlorides were prepared by using or adapting literature procedures as described below.

### 2-Fluoro-4-methoxybenzenesulfonyl chloride

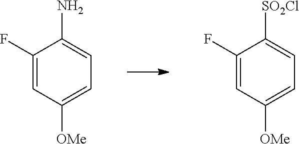 [SMILES: COc1ccc(N)c(F)c1 ; COc1ccc(S(=O)(=O)Cl)c(F)c1]

Following a procedure described in EP2752410A1, 2-fluoro-4-mehoxyaniline (1.00 g, 7.1 mmol) was dissolved in acetonitrile (25 mL) and conc. HCl (10 mL) was added. The mixture was cooled to 0° C. in an ice-salt bath. A solution of NaNO₂ (0.59 g, 8.5 mmol) in water (1 mL) was then added in portions and the mixture stirred for 1.5 h at 0° C. (light brown solution with a small amount of white solids in suspension). To the resulting mixture, AcOH (12 mL) was added and after stirring for 10 minutes at 0° C., NaHSO₃ (7.37 g, 10 equiv., 71 mmol) was added. After stirring for 5 min, Cu(II) chloride (0.96 g, 1 equiv.) and CuCl (70 mg, 0.1 equiv.) were added and the green suspension was stirred in the ice bath, allowing the temperature to rise to RT over 1 h after which it was stirred for an additional 18 h at RT (TLC R_{f}: 0.45 in 2:1 hexane/EtOAc). The reaction mixture was then poured into water (100 mL) and extracted with EtOAc. The extract was washed with water, dried over MgSO₄ and filtered through a pad of silica gel (15 mL) using 1:1 hex/EA as eluent. Removal of volatiles under reduced pressure gave 1.22 g of a clear light brown oil (TLC shows the presence of more polar unidentified impurities following aqueous work-up). ¹H NMR (CDCl₃) δ: 7.88 (t, J=8.6 Hz, 1H), 6.76-6.93 (m, 2H), 3.93 (s, 3H). Homogeneity ˜70% by ¹H NMR.

The following sulfonyl chlorides were prepared using a similar procedure:

2-Cyano-4-fluorobenzenesulfonyl chloride: prepared using 2-cyano-4-fluoroaniline as starting material. The crude material was highly impure but was used successfully in sulfonylation reactions.

4-Methoxy-2-trifluoromethylbenzenesulfonyl chloride: prepared using 4-methoxy-2-trifluoromethylaniline: ¹H NMR (CDCl₃) δ: 8.30 (d, J=9.0 Hz, 1H), 7.42 (d, J=2.3 Hz, 1H), 7.19 (dd, J=9.0, 2.7 Hz, 1H), 3.99 (s, 3H).

### 4-Chloro-2-methylbenzenesulfonyl chloride

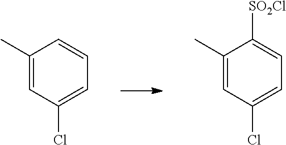

Prepared by chlorosulfonylation of meta-chlorotoluene following the procedure described in Acta Crystallographica Section E 2009, 65 (4), o800. meta-Chlorotoluene (1 mL) was dissolved in CHCl₃ (4 mL) and the solution cooled in an ice bath. Chlorosulfonic acid (2.5 mL) was added dropwise over 15 min as HCl gas was slowly evolved. After completion, the reaction mixture was allowed to warm up to RT. TLC shows no more starting material (Rf=0.8 in 8:2 hex/EA) and a new spot (Rf=0.7 in 8:2 hex/EA) that trails somewhat is formed. The reaction mixture was poured over ice (50 mL), DCM (15 mL) was added and the product organic phase was separated, washed with cold water, dried (MgSO₄) and concentrated to a colorless oil (0.87 g) that was used without further purification: ¹H NMR (CDCl₃) δ: 8.01 (d, J=8.6 Hz, 1H), 7.43 (s, 1H), 7.40 (dd, J=8.6, 2.0 Hz, 1H), 2.78 (s, 3H).

The following sulfonyl chlorides were prepared using a similar procedure with some modifications as described below:

### 4-Methoxy-2-methylbenzenesulfonyl chloride

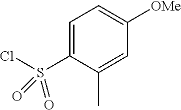

3-Methoxytoluene (6.00 g) was dissolved in CHCl₃ (30 mL) and the solution cooled to −35° C. (bath temperature). Chlorosulfonic acid (15 mL) was added dropwise over 20 min (no HCl/SO₂ gas evolution is noticeable). The clear solution was then stirred for 15 min at −30 to −25° C. (no gas evolution was noticed). The reaction mixture was poured carefully over ice (50 mL), DCM (50 mL) was added and the slightly milky product organic phase was separated, washed with cold water, dried (MgSO₄) and concentrated to a colorless oil that was dried under vacuum (8.28 g, 76% yield). NMR shows the presence of a single isomer: ¹H NMR (CDCl₃) δ: 8.01 (d, J=8.6 Hz, 1H), 6.72-6.95 (m, 2H), 3.90 (s, 3H), 2.75 (s, 3H).

### 2-Chloro-4-methoxybenzenesulfonyl chloride

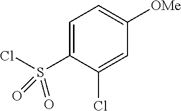 [SMILES: COc1ccc(S(=O)(=O)Cl)c(Cl)c1]

3-Chloroanisole (1.00 g) was dissolved in CHCl₃ (4 mL) and the solution cooled to about −35° C. Chlorosulfonic acid (2.5 mL) in CHCl₃ (2 mL) was added dropwise over 15 min. After completion, there is only baseline material by TLC (SM Rf=0.8 in 8:2 hex/EA). Upon warming the reaction mixture to RT gas evolution was noticed and isomeric products appeared by TLC (Rf=0.30 and 0.25 in 8:2 hex/EA). After stirring at RT for 30 min, a white precipitate began forming. The reaction mixture was poured over ice (50 mL), DCM (15 mL) was added and the organic product phase was separated, washed with cold water, dried (MgSO₄) and concentrated to a colorless oil that crystallized as white needles on standing (0.89 g). ¹H NMR shows a mixture of 2 isomers in a 60:40 ratio that were separated by flash chromatography on silica gel using 8:2 hexane/EtOAc as eluent. Desired isomer (more polar): ¹H NMR (CDCl₃) δ: 7.90 (d, J=8.6 Hz, 1H), 7.04-7.19 (m, 2H), 4.08 (s, 3H).

### 2,3-Dimethylbenzenesulfonyl chloride

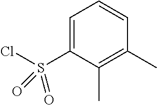

Isolated as the minor isomer after chlorosulfonylation of o-xylene as described in WO2003/055478. ¹H NMR (CDCl₃) δ: 7.96 (d, J=7.8 Hz, 1H), 7.52 (d, J=7.4 Hz, 1H), 7.30 (t, J=7.8 Hz, 1H), 2.71 (s, 3H), 2.41 (s, 3H).

### 3-Fluoro-2-methyl-4-methoxybenzenesulfonyl chloride

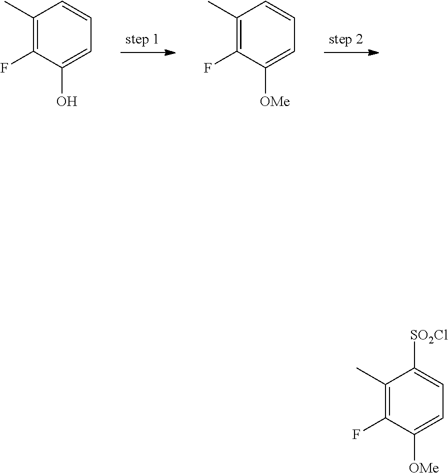

Step 1: To a solution of 2-fluoro-3-methylphenol (4.32 mL, 39.7 mmol) in acetone (50 mL) was added potassium carbonate (6.58 g, 47.6 mmol) followed by iodomethane (2.75 mL, 43.7 mmol). The reaction mixture was then refluxed overnight at 60° C. The reaction mixture was then cooled to RT, filtered (2×10 mL acetone for rinses) and concentrated under reduced pressure. The crude product was extracted from water (30 mL) and EtOAc (2×50 mL). The organic layer was then separated, dried with Na₂SO₄, filtered and concentrated under reduced pressure. The residue was purified by flash chromatography on silica gel using 0 to 5% EtOAc/hexanes to afford 2-fluoro-3-methylanisole as a clear colorless liquid (5.30 g, 95% yield): ¹H NMR (CDCl₃) δ: 6.95 (td, J=8.0, 1.4 Hz, 1H), 6.85-6.71 (m, 2H), 3.87 (s, 3H), 2.28 (d, J=2.3 Hz, 3H).

Step 2: To a solution of 2-fluoro-3-methylanisole from step 1 (1.00 g, 7.13 mmol) in DCM (5.6 mL) was added a solution of chlorosulfonic acid (1.13 mL, 16.5 mmol) in DCM (5.6 mL) over a 5 minute period. The pale brown reaction mixture containing a viscous liquid layer was stirred at RT for 10 min and then quenched by pouring into a mixture of water (10 mL) and ice (5 g). The aqueous phase was extracted with DCM (2×10 mL), dried on Na₂SO₄, filtered and concentrated under reduced pressure to give the desired sulfonyl chloride as a colorless liquid (1.70 g, 100% yield). The material was used without further purification: ¹H NMR (CDCl₃) δ: 7.87 (dd, J=9.0, 1.8 Hz, 1H), 6.97-6.86 (m, 1H), 3.97 (s, 3H), 2.66 (d, J=2.8 Hz, 3H).

### 3-Chloro-2-methyl-4-methoxybenzenesulfonyl chloride

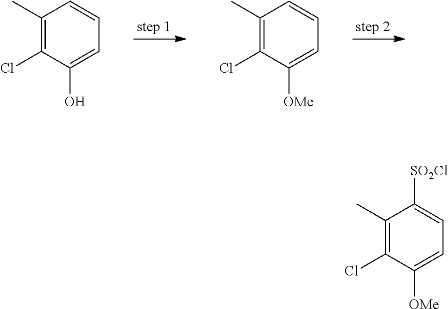

Following a similar procedure (Step 1) as for 3-fluoro-2-methyl-4-methoxybenzenesulfonyl chloride and starting from 2-chloro-3-methylphenol, 2-chloro-3-methylanisole was obtained in quantitative yield as a colorless liquid: ¹H NMR (CDCl₃) δ: 7.12 (t, J=7.9 Hz, 1H), 6.87-6.83 (m, 1H), 6.79 (d, J=8.2 Hz, 1H), 3.89 (s, 3H), 2.38 (s, 3H).

Treatment with chlorosulfonic acid as described for 3-fluoro-2-methyl-4-methoxybenzenesulfonyl chloride (Step 2) provided the desired 3-chloro-2-methyl-4-methoxybenzenesulfonyl chloride as a colorless liquid in 96% yield: ¹H NMR (CDCl₃) δ: 8.02 (d, J=9.1 Hz, 1H), 6.90 (d, J=9.1 Hz, 1H), 4.00 (s, 3H), 2.83 (s, 3H).

### 2-Ethylbenzenesulfonyl chloride

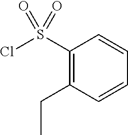

2-Ethylbenzenethiol (1.46 mL, 10.3 mmol) and KCl (776 mg, 10.3 mmol) were dissolved in water (38 mL) and Oxone® (15.8 g, 25.8 mmol) was added in small portions. After stirring for 1 h at RT, the reaction was considered complete by LCMS analysis and the reaction mixture was extracted with EtOAc (4×5 mL). The extract was dried (Na₂SO₄) and concentrated under reduced pressure to give a white crystalline solid (1.43 g, 68% yield) that was used as such: ¹H NMR (CDCl₃) δ: 8.07 (dd, J=8.1, 1.3 Hz, 1H), 7.66 (td, J=7.6, 1.3 Hz, 1H), 7.49 (d, J=7.7 Hz, 1H), 7.45-7.38 (m, 1H), 3.20 (q, J=7.5 Hz), 1.36 (t, J=7.5 Hz).

### 3-Fluoro-2-ethylbenzenesulfonyl chloride

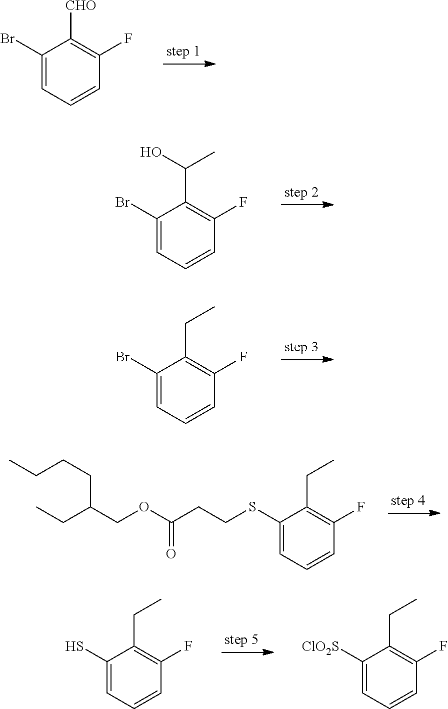

Step 1: 2-Bromo-6-fluorobenzaldehyde (6.00 g, 29.5 mmol) was dissolved in dry THF (60 mL) and the solution was cooled to −78° C. under an argon atmosphere. Methylmagnesium bromide (3.0 M solution in diethylether, 13.4 mL, 40.3 mmol) was added dropwise and the mixture was stirred at −78° C. for 30 min. The reaction was then quenched with 10% hydrochloric acid (50 mL) and the product was extracted into ether (2×50 mL). The extract was dried (MgSO₄) and concentrated and the residue was purified by Combiflash® on silica gel using 10-30% EtOAc in hexanes as eluent to give the desired alcohol derivative as a colorless oil (6.20 g, 96% yield): ¹H NMR (CDCl₃) δ: 7.35 (ddd, J=7.9, 3.1, 2.0 Hz, 1H), 7.16-6.99 (m, 2H), 5.35 (q, J=6.7 Hz, 1H), 1.61 (dd, J=6.8, 1.1 Hz, 3H).

Step 2: Indium (Ill) chloride (412 mg, 1.83 mmol) was suspended in DCM (40 mL) and chlorodiisopropylsilane (8.42 mL, 49.3 mmol) was added. The alcohol from step 1 (4.00 g, 18.3 mmol) in DCM (8 mL) was added and the mixture was stirred for 3 h at RT to give a clear solution. The reaction mixture was quenched with water (50 mL), extracted with diethylether (3×20 mL), washed with brine and dried (MgSO₄). Concentration and purification by flash chromatography using hexanes as eluent provided the silylated ether of the starting alcohol.

The material was dissolved in DCE (41 mL) and chlorodiisopropylsilane (0.78 mL, 4.6 mmol) and indium (Ill) chloride (103 mg, 4.6 mmol) were added. The mixture was stirred for 3 h at 80° C. After cooling to ambient temperature, the reaction mixture was diluted with hexane (100 mL), washed with water (100 mL) and the aqueous phase was back-extracted with hexane (2×50 mL). The combined organic phases were dried (Na₂SO₄) and concentrated to give a colorless oil that was purified by flash chromatography on silica gel using hexanes as eluent to afford the 2-bromo-6-fluoro-ethylbenzene (3.71 g, 100%) as a colorless oil: 1H-NMR (CDCl₃) δ: 7.32 (d, J=7.8 Hz, 1H), 7.11-6.91 (m, 2H), 2.82 (dq, J=7.5, 2.2 Hz, 2H), 1.20-1.15 (m, 3H).

Step 3: the arylbromide from step 2 (3.71 g, 18.3 mmol) was dissolved in toluene (60 mL) and N,N-Diisopropylethylamine (6.40 mL, 36.5 mmol) was added. The solution was then degassed through 3 cycles of evacuation and back-filling with nitrogen. Tris(dibenzylideneacetone) dipalladium(0) (836 mg, 0.9 mmol), 4,4-bis(diphenylphosphino)-9,9-dimethylxanthene (1.08 g, 1.83 mmol) and 2-ethylhexyl-3-mercaptopropionate (4.59 mL, 19.2 mmol) were added and the mixture was degassed twice more before refluxing overnight under a nitrogen atmosphere. The reaction mixture was then cooled to RT, quenched with water (50 mL) and extracted with EtOAc (2×50 mL). The combined organic phases were washed with 10% aqueous HCl (75 mL) and dried (Na₂SO₄). Concentration under reduced pressure and purification of the residue by flash chromatography using 0-15% EtOAc in hexanes as eluent gave the desired sulfide intermediate (4.00 g) as an orange oil contaminated with some unreacted starting thiol. This material was used as such in the next step.

Step 4: the crude sulfide derivative from step 3 (4.00 g, assume 11.7 mmol) was dissolved in THF (41 mL) and potassium tert-butoxide (1.0 M in THF, 14.1 mL, 14.1 mmol) was added dropwise. The resulting solution was stirred at RT for 1 h. The reaction was then quenched by addition of a saturated aqueous NH₄Cl solution (40 mL) and extracted with EtOAc (2×30 mL). The combined organic phases were concentrated and the dark orange residue was passed through a small pad of silica gel using hexanes for washings to afford a mixture of thiol and disulfide (1.50 g) that was used as such in the next step (caution: stench).

Step 5: the crude mixture of thiophenol and disulfide from Step 4 (1.50 g, assume 9.6 mmol) and KCl (723 mg (9.6 mmol) were suspended in water (40 mL) and Oxone® (14.8 g, 24 mmol) was added in portions. After stirring for 1 h at RT, the reaction mixture was extracted with EtOAc (2×20 mL) and the extract was dried (Na₂SO₄) and concentrated under reduced pressure to give the crude sulfonyl chloride that was used as such for preparing the corresponding fragment A-5 and aniline A-8 (see Table 1).

### 3-Chloro-2-ethylbenzenesulfonyl chloride

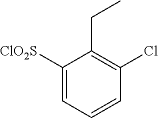

The sulfonyl chloride was prepared following the same procedure as for 3-fluoro-2-ethylbenzenesulfonyl chloride but starting from 2-bromo-6-chlorobenzaldehyde:

Step 1 (98% yield as a white solid): ¹H NMR (CDCl₃) δ: 7.49 (dd, J=8.0, 1.2 Hz, 1H), 7.33 (dd, J=8.0, 1.2 Hz, 1H), 7.05 (t, J=8.0 Hz, 1H), 5.58 (q, J=6.9 Hz, 1H), 1.64 (d, J=6.9 Hz, 3H).

Step 2 (100% yield as a colorless oil): ¹H-NMR (CDCl₃) δ: 7.43 (dd, J=8.0, 1.2 Hz, 1H), 7.29 (dd, J=8.0, 1.2 Hz, 1H), 6.96 (t, J=8.0 Hz, 1H), 2.97 (q, J=7.5 Hz, 2H), 1.17 (t, J=7.5 Hz, 3H).

Step 3 (81% yield as an orange oil): ¹H-NMR (CDCl₃) δ: 7.24-7.18 (m, 2H), 7.08 (t, J=7.9 Hz, 1H), 4.07-3.96 (m, 2H), 3.17 (t, J=7.4 Hz, 2H), 2.96 (q, J=7.5 Hz, 2H), 2.64 (t, J=7.4 Hz, 2H), 1.56 (dd, J=11.9, 5.8 Hz, 2H), 1.40-1.21 (m, 9H), 1.16 (t, J=7.5 Hz, 3H), 0.89 (t, J=7.4 Hz, 6H).

Step 4 (99% yield as a colorless liquid): ¹H-NMR (CDCl₃) δ: 7.15 (d, J=7.9 Hz, 2H), 6.97-6.92 (m, 1H), 3.41 (s, 1H), 2.87 (q, J=7.5 Hz, 2H), 1.18 (t, J=7.5 Hz, 3H).

Step 5 (crude material used without purification): ¹H-NMR (CDCl₃) δ: 8.03 (dd, J=8.2, 1.3 Hz, 1H), 7.73 (dd, J=8.0. 1.3 Hz, 1H), 7.37 (t, J=8.1 Hz, 1H), 3.30 (q, J=7.4 Hz, 2H), 1.33 (t, J=7.4 Hz, 3H).

### 2-Methyl-3-(trifluoromethyl)benzenesulfonyl chloride

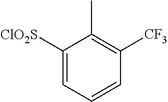

The sulfonyl chloride was prepared following the same procedure as for 3-fluoro-2-ethylbenzenesulfonyl chloride but starting from commercially available 2-methyl-3-(trifluoromethyl)bromobenzene:

Step 3 (100% yield as an orange oil): ¹H-NMR (CDCl₃) δ: 7.49 (d, J=7.9 Hz, 2H), 7.26-7.19 (m, 1H), 4.06-4.00 (m, 2H), 3.18 (dd, J=9.1, 5.6 Hz, 2H), 2.65 (dd, J=9.2, 5.6 Hz, 2H), 2.50 (d, J=1.3 Hz, 3H), 1.33-1.23 (m, 11H), 0.88 (td, J=7.4, 2.3 Hz, 6H).

Step 4 (quantitative yield as a colorless oil): ¹H-NMR (CDCl₃) δ: 7.43 (dd, J=7.9, 2.2 Hz, 2H), 7.12 (t, J=7.8 Hz, 1H), 3.42 (s, 1H), 2.43 (s, 3H).

Step 5 (quantitative yield as a white solid): ¹H-NMR (CDCl₃) δ: 8.31 (d, J=8.1 Hz, 1H), 8.00 (t, J=7.7 Hz, 1H), 7.55 (dd, J=15.6, 7.6 Hz, 1H), 2.93 (s, 3H).

### General Procedure for the Preparation of Sulfonyl Chlorides from Aryl Bromides

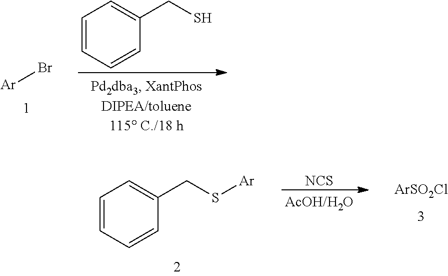

Step 1: Aryl bromide 1 (1.00 mmol) was diluted in toluene (1.70 mL). The mixture was degassed with nitrogen bubbling through the solution for 5 min. Tris(dibenzylideneacetone)-dipalladium(0) (0.02 mmol), 4,4-Bis(diphenylphosphino)-9,9-dimethylxanthene (0.04 mmol) and N,N-diisopropylethylamine (2.0 mmol) were added and then the mixture was degassed again for 5 min. Benzyl mercaptan (1 mmol) was then added and the resulting mixture was heated at reflux overnight (oil bath T=115° C.). After completion, the reaction was cooled down to room temperature, diluted with EtOAc (20 mL) and quenched with H₂O (20 mL). The aqueous layer was extracted with EtOAc (2×20 mL). The combined organic layers were washed with brine (50.0 mL), dried (Na₂SO₄), filtered and concentrated under reduced pressure. The crude was further purified by flash chromatography (0-10% EtOAc in Hexanes, 35 mL/min, product eluted at 100% hexane). Fractions of interest were collected and concentrated under reduced pressure to afford the title compound 2.

Step 2: Compound 2 (1.00 mmol) was dissolved in acetic acid (1.90 mL) and H₂O (0.65 mL) was added to afford a heterogeneous solution. N-Chlorosuccinimide (4.00 mmol) was added portion-wise. The reaction was stirred and monitored by LCMS (samples were quenched with N-methyl piperazine). After completion of the reaction, the mixture was concentrated under reduced pressure. The resulting mixture was slowly poured into a saturated aqueous NaHCO₃ solution which generates a gas release. The mixture was extracted with EtOAc (2×75 mL). The combined organic layers were washed with brine, dried (Na₂SO₄), filtered and concentrated under reduced pressure. The crude compound was further purified by normal phase chromatography (0-40% EtOAc in Hexanes, 60 mL/min, product came out at 100% Hexane). Fractions of interest were collected and concentrated under reduced pressure to afford the title compound 3.

### 2-Chloro-3-methylbenzenesulfonyl chloride

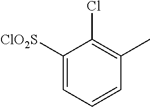

The sulfonyl chloride was prepared following the general procedure starting from commercially available 1-bromo-2-chloro-3-methylbenzene:

Benzyl(2-chloro-3-methylphenyl)sulfide: Yellow solid, 51% yield, 95% purity (at 220 nm). (ES⁻) M−H=247.2; ¹H NMR (400 MHz, CDCl₃) δ 7.39-7.35 (m, 2H), 7.33-7.28 (m, 2H), 7.28-7.23 (m, 1H+CDCl₃), 7.11-7.03 (m, 3H), 4.15 (s, 2H), 2.38 (s, 3H).

2-Chloro-3-methylbenzenesulfonyl chloride: Pale yellow oil, 70% yield, 60% purity (at 254 nm). LCMS: LCMS sample was quenched with N-methylpiperazine (resulting sulfonamide MW=288.8) (ES⁺) M+H=289.2. Used as crude.

### 3-Fluoro-2-(trifluoromethyl)benzenesulfonyl chloride

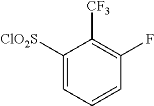 [SMILES: O=S(=O)(Cl)c1cccc(F)c1C(F)(F)F]

The sulfonyl chloride was prepared following the general procedure starting from commercially available 1-bromo-3-fluoro-2-(trifluoromethyl)benzene:

Benzyl(3-fluoro-2-(trifluoromethyl)phenyl)sulfide: yellow oil, 66% yield, 98% purity (at 254 nm). (ES⁻) M−H=285.2.

3-Fluoro-2-(trifluoromethyl)benzenesulfonyl chloride: pale yellow oil, 93% yield, 98% purity (at 254 nm). LCMS sample was quenched with N-methylpiperazine (resulting sulfonamide MW=326.1) (ES⁺) M+H=327.1.

### 3-Chloro-2-(trifluoromethyl)benzenesulfonyl chloride

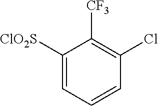 [SMILES: O=S(=O)(Cl)c1cccc(Cl)c1C(F)(F)F]

The sulfonyl chloride was prepared following the general procedure starting from commercially available 1-bromo-3-chloro-2-(trifluoromethyl)benzene:

Benzyl(3-chloro-2-(trifluoromethyl)phenyl)sulfide: white solid, 59% yield, 90% purity (at 220 nm). (ES⁻) M−H=301.2; ¹H NMR (400 MHz, CDCl₃) δ 7.46-7.14 (m, 8H+CDCl₃), 4.16 (s, 2H).

3-Chloro-2-(trifluoromethyl)benzenesulfonyl chloride: colorless oil, 66% yield. LCMS: LCMS sample was quenched with N-methylpiperazine (resulting sulfonamide MW=343.5) (ES⁺) M+H=343.2. Used as crude.

### 3,4-Difluoro-2-methylbenzenesulfonyl chloride

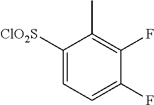

The sulfonyl chloride was prepared following the general procedure starting from commercially available 1-bromo-3-chloro-2-(trifluoromethyl)benzene:

Benzyl(3,4-difluoro-2-methylphenyl)sulfide: orange oil, 96% yield, 96% purity (at 254 nm). (ES⁻) M−H=249.2; ¹H NMR (400 MHz, DMSO-d₆) δ 7.32-7.17 (m, 7H), 4.16 (s, 2H), 2.18 (d, J=2.7 Hz, 3H). 3,4-Difluoro-2-methylbenzenesulfonyl chloride: pale yellow oil, 49% yield. LCMS: LCMS sample was quenched with N-methylpiperazine (resulting sulfonamide MW=290.3) (ES⁺) M+H 5=291.2; ¹H NMR (400 MHz, DMSO-d₆) δ 7.56 (ddd, J=8.4, 5.5, 1.8 Hz, 1H), 7.15 (dd, J=18.4, 8.4 Hz, 1H), 2.46 (d, J=2.8 Hz, 3H).

### 2,4-Dimethyl-3-fluorobenzenesulfonyl chloride

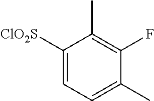

The sulfonyl chloride was prepared following the general procedure starting from commercially available 1-bromo-2,4-dimethyl-3-fluorobenzene:

benzyl(3-fluoro-2,4-dimethylphenyl)sulfide: Orange oil, 95% yield of crude, 80% purity (at 254 nm), used as crude.

3-fluoro-2,4-dimethylbenzene-1-sulfonyl chloride: Orange oil, 89% yield of crude, 74% purity (at 254 nm). LCMS: LCMS sample was quenched with N-methylpiperazine (resulting sulfonamide MW=286.4) (ES⁺) M+H=287.1. Used as crude.

### 2-Methylpyridine-3-sulfonyl chloride

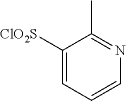

The sulfonyl chloride was prepared following the general procedure starting from commercially available 3-bromo-2-methylpyridine:

3-(Benzylthio)-2-methylpyridine: orange liquid, 88% yield, 94% purity (at 220 nm). (ES⁺) M+H=215.8, (ES⁻) M−H=214.1. ¹H NMR (400 MHz, DMSO-d₆) δ 8.30 (dd, J=4.9, 1.5 Hz, 1H), 7.57-7.45 (m, 1H), 7.31-7.27 (m, 5H), 7.07 (dd, J=7.6, 5.1 Hz, 1H), 4.09 (s, 2H), 2.58 (s, 3H).

2-Methylpyridine-3-sulfonyl chloride: pale yellow oil, 100% yield, 95% purity (at 254 nm). LCMS sample was diluted with H₂O (resulting sulfonic acid MW=173.1) (ES⁻) M−H=171.9; ¹H NMR (400 MHz, CDCl₃) δ 8.80 (dd, J=4.8, 1.6 Hz, 1H), 8.33 (dd, J=8.1, 1.7 Hz, 1H), 7.43-7.36 (m, 1H), 3.02 (s, 3H).

### 6-Methoxy-4-methylpyridine-3-sulfonyl chloride

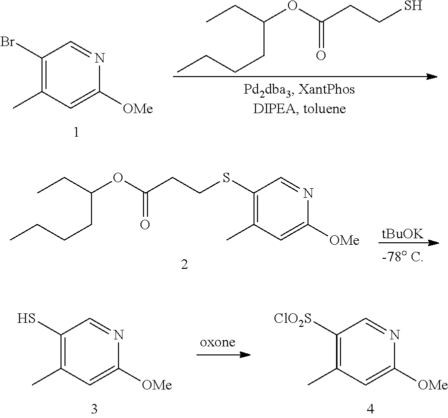

Preparation of 2-ethylhexyl 3-((6-methoxy-4-methylpyridin-3-yl)thio)propanoate (2): 5-bromo-2-methoxy-4-methylpyridine (6.00 g, 29.7 mmol) was dissolved in toluene (100 mL) and N,N-diisopropylethylamine (10.4 mL, 59.4 mmol) was added. The mixture was degassed with nitrogen bubbling through the solution for 5 min. Tris(dibenzylideneacetone)dipalladium(0) (1.36 g, 1.49 mmol), 4,4-bis(diphenylphosphino)-9,9-dimethylxanthene (1.75 g, 2.97 mmol) and 2-ethylhexyl-3-mercaptopropionate (7.47 mL, 31.2 mmol) were added. The mixture was degassed again for 5 min. The mixture was heated at reflux overnight (oil bath T=117° C.). The reaction was cooled down to room temperature, diluted with EtOAc (100 mL) and quenched with H₂O (100 mL). The organic and aqueous layers were separated and the aqueous layer was extracted with EtOAc (2×50.0 mL). The combined organic layers were washed with HCl (10% in H₂O, 50.0 mL), dried (Na₂SO₄), filtered and concentrated under reduced pressure. The crude was purified by flash chromatography (330 g silica gel column, EtOAc-Hexanes, 0-20%) to afford the title compound as an orange oil (10.0 g, 99% yield). (ES⁺) M+H=340.2; ¹H NMR (400 MHz, CDCl₃) δ 8.19 (s, 1H), 6.64 (s, 1H), 3.99 (dd, J=5.9, 2.4 Hz, 2H), 3.93 (s, 3H), 2.96 (t, J=7.3 Hz, 2H), 2.55 (t, J=7.3 Hz, 2H), 2.42 (d, J=0.5 Hz, 3H), 1.56 (dt, J=12.1, 6.0 Hz, 1H), 1.39-1.22 (m, 8H), 0.88 (m, 6H).

Preparation of 6-methoxy-4-methylpyridine-3-thiol (3): to a −78° C. solution of 2 (10.0 g, 29.5 mmol) in THF (105 mL) was added potassium t-butoxide (1.00 M in THF, 35.3 mL, 35.3 mmol) dropwise and a precipitate was formed. The resulting suspension was stirred at −78° C. for 30 minutes. The reaction was quenched by the addition of NH₄Cl (50.0 mL) and the mixture was extracted with CH₂Cl₂ (2×50.0 mL). The combined organic layers were dried (Na₂SO₄), filtered and concentrated under reduced pressure to afford a dark orange liquid. The crude was purified by flash chromatography (100% Hexanes) to afford a mixture of the title compound and its disulfide (4.56 g) which was used in the next step without further purification. LCMS: Thiol 3: (ES⁺) M+H=156.9 and disulfide: (ES⁺) M+H=309.0.

Preparation of 6-methoxy-4-methylpyridine-3-sulfonyl chloride (4): to a mixture of the thiol 3 (4.56 g, 29.4 mmol) in H₂O (123 mL) was added potassium chloride (2.21 g, 29.3 mmol) followed by a portion-wise addition of Oxone® (45.2 g, 73.5 mmol). After completion of the reaction (1 h), the mixture was extracted with EtOAc (2×20.0 mL) and the combined organic layers were dried (Na₂SO₄) and concentrated under reduced pressure. The crude product obtained was used without any further purification.

### 3-Methylpyridine-4-sulfonyl chloride

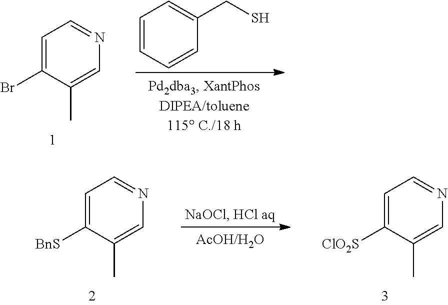

Step 1: 4-Bromopyridine (1.00 mmol) was dissolved in toluene (1.70 mL) and N,N-diisopropylethylamine (2.00 mmol) was added. The mixture was degassed with nitrogen bubbling through the solution for 5 min. Tris(dibenzylideneacetone)-dipalladium(0) (0.02 mmol), 4,4-Bis(diphenylphosphino)-9,9-dimethylxanthene (0.04 mmol) and the phenylmethanethiol/benzyl mercaptan (1.00 mmol) were added. The mixture was degassed again for 5 min. The mixture was heated at reflux for 18 h (oil bath T=115° C.). The reaction was cooled down to room temperature, diluted with EtOAc (10.0 mL) and quenched with H₂O (10.0 mL). The aqueous and organic layers were separated and the aqueous layer was extracted with EtOAc (2×10.0 mL) and the combined organic phases were washed with HCl (10% in H₂O, 10.0 mL), dried (Na₂SO₄), filtered and concentrated under reduced pressure. The crude was purified by flash chromatography (EtOAc-Hexanes, 10% to 35%) to afford sulfide 2: (90% yield). (ES⁺) M+H=216.1; ¹H NMR (400 MHz, CDCl₃) δ 8.28 (d, J=4.6, 1H), 8.24 (s, 1H), 7.43-7.27 (m, 5H), 7.20 (t, J=5.4 Hz, 1H), 4.25 (s, 2H), 2.28 (s, 3H).

Step 2: Compound 2 (1.00 mmol) was dissolved CH₂Cl₂ (11.5 mL) and cooled to −10° C. HCl (1.00 M in H₂O, 5.70 mL) was added and stirred at −10° C. for 5 min. Sodium hypochlorite (10% solution in H₂O, 3.00 mmol) was added over 10 min maintaining temp below 0° C. The mixture was stirred at 0° C. for 10 min. The organic and aqueous layer were separated. Organic layer was dried (Na₂SO₄). The crude sulfonyl chloride was used in the next step without further purification or evaporation: LCMS sample was quenched with N-methylpiperazine (resulting sulfonamide MW=255.3); (ES⁺) M+H+=256.2.

### 2,3-Dimtylpyridine-4-sulfonyl chloride

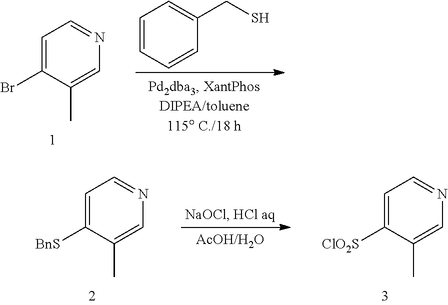

The same procedure as for 3-methylpyridine-4-sulfonyl chloride was used starting from 2,3-dimethyl-4-bromopyridine:

Step 1: 4-(Benzylthio)-2,3-dimethylpyridine (2): (92% yield). LCMS: (ES⁺) M+H=230.2; ¹H NMR (400 MHz, CDCl₃) δ: 8.17 (d, J=5.5 Hz, 1H), 7.42-7.28 (m, 5H), 6.99 (d, J=5.5 Hz, 1H), 4.18 (s, 2H), 2.53 (s, 3H), 2.24 (s, 3H).

Step 2: 2,3-Dimethylpyridine-4-sulfonyl chloride (3): LCMS sample was quenched with N-methylpiperazine (resulting sulfonamide MW=269.3); (ES⁺) M+H⁺=270.2.

### Protected Sulfonyl Chloride for Fragment B69

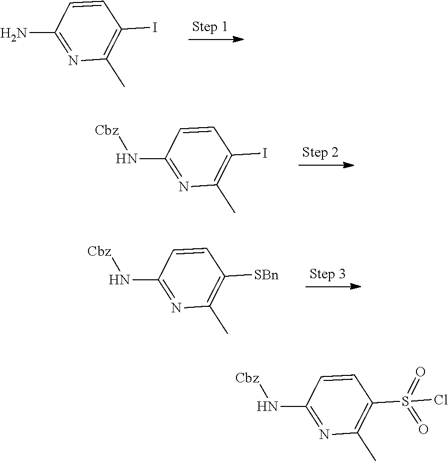

Step 1: to a solution of the commercially available aminopyridine (1.00 g, 4.27 mmol) and N-benzylcarbamoyl chloride (0.9421 g, 5.5546 mmol) in EtOAc (20 mL) was added 20 mL of a saturated solution of aq. NaHCO₃. The solution was stirred 16 h at RT. Upon completion, EtOAc was added to the reaction mixture, and the organic layer was separated, washed with brine, dried over MgSO₄ then filtered and concentrated. The residue was adsorbed on SiO₂ then purified on SiO₂ with EtOAc in hexanes to afford the desired protected aminopyridine (1.00 g, 2.72 mmol, 64%). ¹H NMR (400 MHz, DMSO-d₆) δ: 10.35 (s, 1H), 8.09 (d, J=8.61 Hz, 1H), 7.47 (d, J=9.00 Hz, 1H), 7.23-7.44 (m, 4H), 5.16 (s, 2H), 2.53 (s, 3H) MS m/z 369.2 (MH⁺).

Step 2: A degassed solution of the iodopyridine from step 1 (0.61 g, 1.66 mmol), tris(dibenzylideneacetone)-dipalladium(0) chloroform adduct (86 mg, 0.0828 mmol), 9,9-dimethyl-9h-xanthene-4,5-diyl)bis(diphenylphosphine (96 mg, 0.166 mmol), DIPEA (0.576 mL, 3.31 mmol) and benzyl mercaptan (0.233 mL, 1.99 mmol) in toluene (15 mL) was stirred under N₂ at 115° C. for 3 h. Upon completion, SiO₂ added to the reaction mixture and concentrated under vacuum. The residue was purified on SiO₂ column with EtOAc in hexanes to provide the expected sulfide (560 mg, 93%). ¹H NMR (400 MHz, CDCl₃) δ: 7.71 (d, J=8.61 Hz, 1H), 7.54 (d, J=8.61 Hz, 1H), 7.46 (br s, 1H), 7.31-7.43 (m, 5H), 7.19-7.26 (m, 2H), 7.12-7.19 (m, 2H), 5.22 (s, 2H), 3.96 (s, 2H), 2.41 (s, 3H). MS m/z 365.2 (MH⁺).

Step 3: To a solution of the sulfide from step 2 (300 mg, 0.823 mmol) in 90% AcOH in water (16 mL) was added N-chlorosuccinimide (330 mg, 2.47 mmol). The reaction mixture was stirred at room temperature for 3 hours. The reaction mixture was evaporated to dryness then diluted in EtOAc and washed with water followed by brine. The organic layer was dried over MgSO₄, filtered and concentrated under vacuum to afford the desired sulfonyl chloride of group B49 that was used as is (282 mg, 99%): ¹H NMR (400 MHz, CDCl₃) δ: 8.28 (d, J=9.00 Hz, 1H), 8.02 (d, J=9.00 Hz, 1H), 7.72 (br s, H), 7.34-7.60 (m, 5H), 5.27 (s, 2H), 2.86 (s, 3H). MS m/z 341.2 (MH⁺).

### 2,2-Difluorobenzo[d][1,3]dioxole-4-sulfonyl chloride

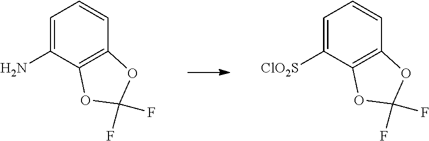 [SMILES: Nc1cccc2c1OC(F)(F)O2 ; O=S(=O)(Cl)c1cccc2c1OC(F)(F)O2]

Thionyl chloride (5.96 mL) was added dropwise over 20 min to water (30 mL) and the mixture stirred for 48 h at RT to produce as sulfur dioxide containing solution. In a separate vessel, 2,2-difluorobenzo[d][1,3]dioxol-4-amine (1.00 g, 5.78 mmol) was added dropwise to ice-cooled conc. HCl (7 mL) to produce a white precipitate. A solution of sodium nitrite (523 mg, 7.5 mmol) in water (2 mL) was added dropwise over 5 min to the aniline hydrochloride to produce an orange reaction mixture. The orange suspension was then added gradually at 5° C. to the sulfur dioxide solution from above to which 10 mg of cuprous chloride had previously been added. The mixture was stirred for an additional 2 h in an ice bath (gas evolution is observed and an orange liquid is deposited at the bottom of the flask). After completion as determined by LCMS analysis, the reaction mixture was extracted with DCM (2×20 mL), dried (Na₂SO₄), filtered and concentrated to give the desired sulfonyl chloride as an orange oil that was used without further purification (100% crude yield): 1H-NMR (400 MHz, CDCl₃) δ 7.65 (dd, J=8.4, 1.1 Hz, 1H), 7.44 (dd, J=8.1, 1.1 Hz, 1H), 7.32 (t, J=8.2 Hz, 1H).

### 4-Chloro-3-fluoro-2-methylbenzenesulfonyl chloride

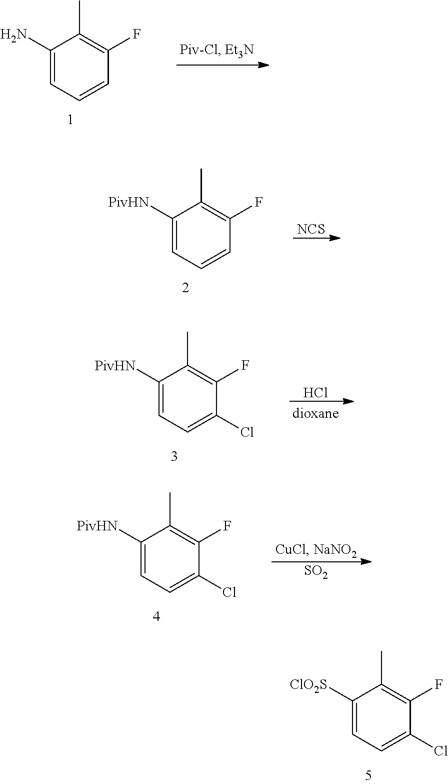

Preparation of N-(3-fluoro-2-methylphenyl)pivalamide (2): to a THF (240 mL) solution of 3-fluoro-2-methylaniline (10.9 mL, 93.0 mmol) at 0° C. was added triethylamine (14.9 mL, 106 mmol) followed by pivaloyl chloride (13.1 mL, 105 mmol) over 10 min. The mixture was warmed up to room temperature and stirred for 2 h. Volatiles were evaporated under reduced pressure and the residue was partitioned between H₂O (250 mL) and EtOAc (150 mL). Organic and aqueous layers were separated. Aqueous layer was extracted with EtOAc (4×60 mL). Combined organic layers were washed with brine (60 mL), dried (Na₂SO₄) and concentrated under reduced pressure to give the title compound 2 (18.5 g, 95% yield) as a solid. (ES⁺) M+H=210.2.

Preparation of N-(4-chloro-3-fluoro-2-methylphenyl)pivalamide (3): to a DMF (50.0 mL) solution of compound 2 (4.20 g, 20.1 mmol) at room temperature was added N-chlorosuccinimide (2.76 g, 20.1 mmol) over 10 min (in 3 portions). The mixture was heated at 80° C. for 90 min. Additional N-chlorosuccinimide (541 mg, 4.01 mmol) was added and this was stirred for 45 min at 80° C. The mixture was cooled to room temperature and was diluted with EtOAc (30 mL) and water (60 mL). Organic and aqueous layers were separated. Aqueous layer was extracted with EtOAc (3×20 mL). Combined organic layers were washed with H₂O (3×30 mL), brine (20.0 mL), dried (Na₂SO₄) and concentrated under reduced pressure to give crude compound (5.20 g). The crude was dissolved in cyclohexane (30 mL) and heated at 45-50° C. until all solid dissolved. The solution was cooled to room temperature. White solid precipitated was filtered and washed with cyclohexane (3×5 mL) to give the title compound 3 as a solid (1.95 g, 40% yield). (ES⁺) M+H=244.1.

Preparation of 4-chloro-3-fluoro-2-methylaniline (4): to a dioxane (18 mL) solution of compound 3 (1.60 g, 6.57 mmol) at room temperature was added HCl (6.00 M in water, 23 mL, 138 mmol) over 5 min. The mixture was heated at 100° C. for 20 h. The mixture was cooled to room temperature. Solid K₂CO₃ (exothermic) was added portion-wise until pH=8-9 was obtained. The mixture was extracted with EtOAc (4×20 mL). Combined organic layers were washed with brine (20 mL), dried (Na₂SO₄) and concentrated under reduced pressure to give 1.6 g of crude which was dried under vacuum for 24 h to give the title compound 4 as an oil (850 mg, 81% yield). This was used in the next reaction without further purification. ¹H NMR (400 MHz, CDCl₃) δ 6.99 (t, J=8.3 Hz, 1H), 6.40 (d, J=8.6 Hz, 1H), 2.22-2.06 (m, 3H).

Preparation of 4-chloro-3-fluoro-2-methylbenzene-1-sulfonyl chloride (5): under ice-cooling, thionyl chloride (29.1 mL, 395 mmol) was added dropwise to H₂O (92.1 mL) over 20 min. This solution containing sulfur dioxide was stirred at 0° C. for 2 h and at room temperature for 18 h. Separately, HCl (conc.) (23 mL) was added portion-wise to compound 4 (3.00 g, 18.8 mmol) at 0° C. to afford a beige precipitate. This was stirred at 0° C. for 5 min. A solution of sodium nitrite (1.70 g, 24.4 mmol) in H₂O (2 mL) was added dropwise over approximately 10 min. The above-mentioned sulfur dioxide solution containing copper (1) chloride (38.4 mg, 376 umol) was added to the reaction mixture gradually at 5° C., over 40 min. Under ice-cooling, the mixture was further stirred for 2 h and then at room temperature for 4 days. The mixture was diluted with CH₂Cl₂ (20 mL). Aqueous and organic layers were separated. Aqueous layer was extracted with CH₂Cl₂ (3×20 mL). Combined organic layers were dried (Na₂SO₄), filtered and concentrated to afford crude compound 5 (1.95 g, 30% yield, 70% purity) as an oil. Crude 5 was used as such without further purification. LCMS: LCMS sample was quenched with N-methylpiperazine (resulting sulfonamide MW=306.784); (ES⁺) M+H=307.1; ¹H NMR (400 MHz, CDCl₃) δ 7.81 (d, J=8.4 Hz, 1H), 7.46 (t, J=7.5 Hz, 1H), 2.69 (s, 3H).

### 3-Methyl-2-thiophenylsulfonyl chloride

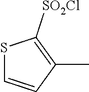

Prepared by chlorosulfonylation of 3-methylthiophene as described in U.S. Pat. No. 3,991,081. ¹H NMR (CDCl₃) δ: 7.67 (d, J=5.1 Hz, 1H), 7.03 (d, J=5.1 Hz, 1H), 2.63 (s, 3H).

### 3-Chloro-2-thiophenylsulfonyl chloride

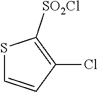

3-Chlorothiophene (1.00 g) was dissolved in CHCl₃ (10 mL) and the solution cooled to −30° C. Chlorosulfonic acid (2.4 mL) was added dropwise over 5 min (no gas evolution is noticeable). The orange-brown solution was then stirred for 30 min allowing temperature to rise to −10° C. then to RT over another 30 min. The reaction mixture was then stirred at RT for 2 h (no gas evolution was noticed, and TLC showed formation of the product (R_{f}=0.4 in 8:2 hex/EA)). The reaction mixture was poured over ice (50 mL), DCM (25 mL) was added and the milky product organic phase was separated, washed with cold water, dried (MgSO₄) and concentrated to a yellow oil that was dried under vacuum and used without further purification (0.55 g). ¹H NMR (CDCl₃) δ: 7.76 (d, J=5.7 Hz, 1H), 7.16 (d, J=5.7 Hz, 1H).

### 4-Chloro-3-methylthiophene-2-sulfonyl chloride

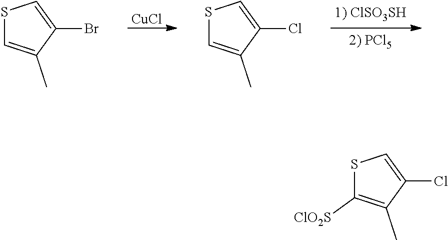

Preparation of 3-chloro-4-methylthiophene: In a 100 mL flask, copper (1) chloride (5.76 g, 56.5 mmol) was added to 3-bromo-4-methylthiophene (3.16 mL, 28.2 mmol) in DMF (20.1 mL) at room temperature. This was heated at 160° C. in an oil bath for 24 h. Crude was poured onto H₂O (50 mL). The resulting mixture was stirred for 10 min at room temperature. Brown-green solid formed was filtered, washed with water (3×10.0 mL) and Et₂O (4×10.0 mL). Filtrate was extracted with Et₂O (3×25.0 mL). Combined organic layers were washed with H₂O (2×20.0 mL), brine (20.0 mL), dried (Na₂SO₄) and concentrated under reduced pressure to give orange oil (0.890 g, 59% yield of crude). ¹H NMR (400 MHz, CDCl₃) δ 7.09 (d, J=3.5 Hz, 1H), 6.99-6.95 (m, 1H), 2.22-2.21 (m, 3H).

Preparation of 4-chloro-3-methylthiophene-2-sulfonyl chloride: 3-Chloro-4-methylthiophene (1.00 g, 7.54 mmol) was dissolved in CHCl₃ (4.43 mL) and a solution of chlorosulfonic acid (1.19 mL, 17.3 mmol) in CHCl₃ (1.48 mL) was added over 5 min at room temperature. The mixture was stirred for 10 min. Phosphorus pentachloride (4.13 g, 18.9 mmol) was added to the reaction mixture followed by CHCl₃ (7.50 mL). This was heated at 50° C. for 1 h. Reaction mixture was slowly added to the aqueous NaHCO₃ solution+ice (30 mL). This was stirred for 10 min. Extracted with CH₂Cl₂ (4×10 mL). Combined organic layers were dried (Na₂SO₄), concentrated under reduced pressure to give the title compound as an oil (1.30 g, 30% yield, 40% purity). This was used in the next reaction without further purification. LCMS: LCMS sample was quenched with N-methyl piperazine; (ES⁺) M+H=295.1; ¹H NMR (400 MHz, CDCl₃) δ 7.57 (s, 1H), 2.57 (s, 3H).

### 3,4-Dichlorothiophene-2-sulfonyl chloride

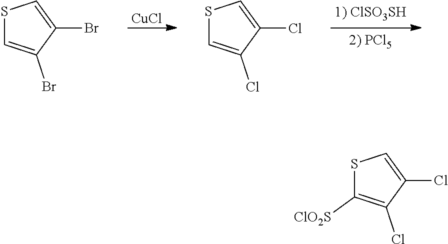

Preparation of 3,4-dichlorothiophene: in a 100 mL flask, copper (1) chloride (13.9 g, 137 mmol) was added to 3,4-dibromothiophene (5.03 mL, 45.5 mmol) in DMF (32 mL) at room temperature. This was heated at 160° C. in an oil bath for 24 h. Crude was poured onto H₂O (100 mL) and diluted with Et₂O (60 mL). The mixture was stirred for 10 min at room temperature. The brown-green solid formed was filtered, washed with H₂O (3×20 mL) then Et₂O (4×20 mL). Filtrate was extracted with Et₂O (4×30 mL). Combined organic layers were washed with H₂O (2×30 mL), brine (30 mL), dried (MgSO₄) and concentrated under reduced pressure to give the title compound 2 (5.50 g, 79% yield) as a red oil. ¹H NMR (400 MHz, CDCl₃) δ 7.21 (s, 2H).

Preparation of 3,4-dichlorothiophene-2-sulfonyl chloride: to a CHCl₃ (5.98 mL) solution of compound 2 (1.61 g, 10.5 mmol) was added a solution of chlorosulfonic acid (757 μL, 11.0 mmol) in CHCl₃ (2 mL) over 5 min. The mixture was stirred for 20 min at room temperature. Phosphorus pentachloride (5.77 g, 26.3 mmol) was added to the mixture in 4 portions. The mixture was heated at 50° C. for 18 h. Volatiles were removed under reduced pressure and the residue was dissolved in CH₂Cl₂ (25 mL), washed with aqueous saturated NaHCO₃ (3×15 mL), H₂O (3×10 mL) and brine (10 mL). Organic layer was dried (Na₂SO₄) and concentrated under reduced pressure to give the title compound 3 (2.32 g, 88% yield). LCMS: LCMS sample was quenched with N-methylpiperazine (resulting sulfonamide MW=315.240); (ES⁺) M+H=315.1.

### (3R)-3-Methoxy 1-pyrrolidinesulfonyl chloride

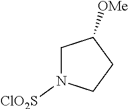 [SMILES: CO[C@@H]1CCN(S(=O)(=O)Cl)C1]

Following a procedure described in US Patent application US 2011/0311474A1, (R)-3-methoxypyrrolidine hydrochloride salt (0.30 g, 2.1 mmol) was suspended in a mixture of 4 mL of toluene and 2 mL of DCM. Triethylamine (0.64 mL, 4.6 mmol) was added and the mixture was sonicated for 4-5 min. affording a fine white suspension. In a separate flask, 4 mL of toluene was cooled to −40° C. in an acetonitrile/dry ice bath. Sulfuryl chloride (0.71 mL, 8.7 mmol) was added and the solution was stirred for 5 min. The pyrrolidine suspension was then added dropwise to the cold (−40° C.) sulfuryl chloride solution over 10 min. The resulting suspension was allowed to stir at the same temperature for 1 hour and was then allowed to warm to room temperature. The solids were filtered out and rinsed with toluene. The filtrate was concentrated to afford 400 mg of the desired product as a pale brown oil that was used without further purification (0.40 g). ¹H NMR (CDCl₃) δ: 4.07 (tt, J=4.6, 2.1 Hz, 1H), 3.48-3.69 (m, 4H), 3.36 (s, 3H), 2.12-2.23 (m, 1H), 1.98-2.12 (m, 1H).

### (3S)-3-Methoxy 1-pyrrolidinesulfonyl chloride

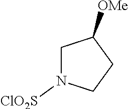 [SMILES: CO[C@H]1CCN(S(=O)(=O)Cl)C1]

Prepared in a similar manner as the (R)-isomer but starting from (S)-3-methoxypyrrolidine hydrochloride salt.

Other sulfamoyl chlorides prepared in a similar fashion using commercially available 2° amines and the procedure described in US 2011/0311474A1 include:

(S)-3-fluoropyrrolidine-1-sulfonyl chloride: obtained as a white solid from (S)-3-fluoropyrrolidine hydrochloride: ¹H NMR (DMSO-d₆) δ: 4.54 (dt, J=52.6, 3.2 Hz, 1H), 2.72-3.03 (m, 5H), 1.34-1.58 (m, 2H)

3,3-Difluoropyrrolidine-1-sulfonyl chloride: obtained as a white solid from 3,3-difluoropyrrolodine hydrochloride: ¹H NMR (CDCl₃) δ: 3.81 (t, J=12.3 Hz, 2H), 3.74 (t, J=7.4 Hz, 2H), 2.53 (tt, J=13.0, 7.5 Hz, 2H).

3-Methoxyazetidine-1-sulfonyl chloride: obtained from 3-methoxyazetidine hydrochloride: ¹H NMR (CDCl₃) δ: 4.22-4.33 (m, 3H), 3.97-4.09 (m, 2H), 3.33 (s, 3H).

### Preparation of (R)-3-(chloromethyl)pyrrolidine-1-sulfonyl chloride

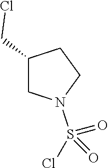 [SMILES: O=S(=O)(Cl)N1CC[C@@H](CCl)C1]

A solution of (R)-3-(hydroxymethyl)pyrrolidine (200 mg, 2 mmol) and triethylamine (0.61 mL, 4.35 mmol) in dry DCM (15 mL) was added dropwise to a stirred solution of sulfuryl chloride (0.48 mL, 5.9 mmol) in DCM (5 mL) at −78° C. The reaction was stirred at −78° C. for 30 minutes, and then allowed to warm to room temperature over 1 h. The reaction mixture was then washed with aqueous 1M hydrochloric acid (5 mL) and brine (5 mL). The organic layer was separated, dried over anhydrous sodium sulfate, filtered and concentrated to afford the title compound as a colorless oil that was used as such for the preparation of Example 28.

### Preparation of difluoroaniline hydrochloride intermediate A-5 (Ar=4-methoxyphenyl) from tert-butylcarbamate A-2

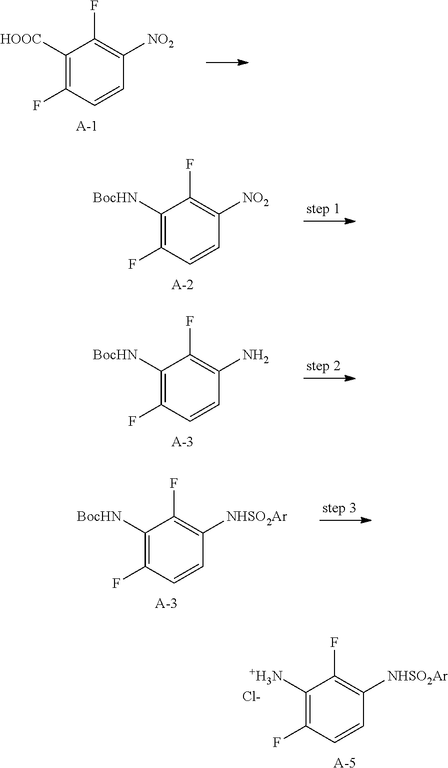

Step 1—Preparation of aniline intermediate A-3: Nitroarene A-2 (5.00 g, 18 mmol, prepared following the procedure described in J. Med. Chem. 2003, 46, 1905) and 20% Pd(OH)₂ on C (130 mg) were suspended in MeOH (50 mL) and the mixture stirred under a hydrogen-containing balloon for 18 h when reduction was shown to be complete by TLC analysis (R_{f}=0.45 in 2:1 hexane/EtOAc). The suspension was filtered through a pad of Celite® to remove the catalyst using MeOH for washings and the solvent evaporated under reduced pressure. Upon exposure to air, the originally colorless solution quickly became a very dark greenish blue. The crude intermediate aniline A-3 was obtained as a dark greenish purple foam that was used immediately in the next step without further purification: ¹H NMR (DMSO-d₆) δ: 8.58 (s, 1H), 6.77 (td, J=9.4, 2.0 Hz, 1H), 6.60 (td, J=9.4, 5.5 Hz, 1H), 4.97 (s, 2H), 1.43 (s, 9H).

Step 2—Preparation of sulfonamide A-4 (Ar=4-methoxyphenyl): Crude aniline A-3 from step 1 (assume 18 mmol) was dissolved in THF (30 mL) and excess 4-methoxyphenylsulfonyl chloride (7.53 g, 36 mmol) was added followed by pyridine (6 mL). The mixture was stirred at 50° C. for 18 h. THF was removed under reduced pressure and the residue partitioned between EtOAc and water. The extract was washed with saturated aqueous NaHCO₃ and brine and dried over MgSO₄. The drying agent slurry was passed through a 75 mL pad of silica gel using EtOAc for washings to remove drying agent and baseline material. Removal of solvent gave a brown oil that was purified by flash chromatography on silica (˜250 mL) using 20-50% EtOAc in hexane as eluent. After drying under vacuum, the product A-4 was obtained as a brownish foam (8.16 g) contaminated with unreacted sulfonyl chloride in a 2:1 ratio by ¹H NMR. The material was used directly as such in the next step: ¹H NMR (CDCl₃) δ: 7.68 (d, J=9.0 Hz, 2H), 7.41 (td, J=8.8, 5.5 Hz, 1H), 6.85-6.98 (m, 3H), 6.57 (br s, 1H), 5.85 (br s, 1H), 3.85 (s, 3H), 1.46 (s, 9H). MS m/z 413.0 (M−H), m/z 313.0 (M−H-Boc),

Step 3—Preparation of aniline hydrochloride A-5 (Ar=4-methoxyphenyl): The crude carbamate A-4 from step 3 (8.16 g) was stirred at RT in 4N HCl in dioxane (25 mL) for 1.5 h during which time a beige solid progressively precipitated After 1.5 h, another 10 mL of 4N HCl in dioxane was added and stirring continued for an additional 1 h. The reaction mixture was then diluted with 50 mL of diethyl ether and the beige precipitate was collected by filtration, washed with ether and dried under vacuum. Aniline salt A-5 (4.38 g) was obtained in pure form in 68% overall yield from nitroarene A-2: ¹H NMR (DMSO-d₆) δ: 9.73 (s, 1H), 7.62 (d, J=8.6 Hz, 2H), 7.06 (d, J=9.0 Hz, 2H), 6.69-6.88 (m, 1H), 6.30 (td, J=8.6, 5.5 Hz, 1H), 3.81 (s, 3H). MS m/z 313.0 (M−H).

### Preparation of difluoroaniline hydrochloride intermediate A-5 (Ar=2,3-dichlorophenyl) from acetanilide A-8

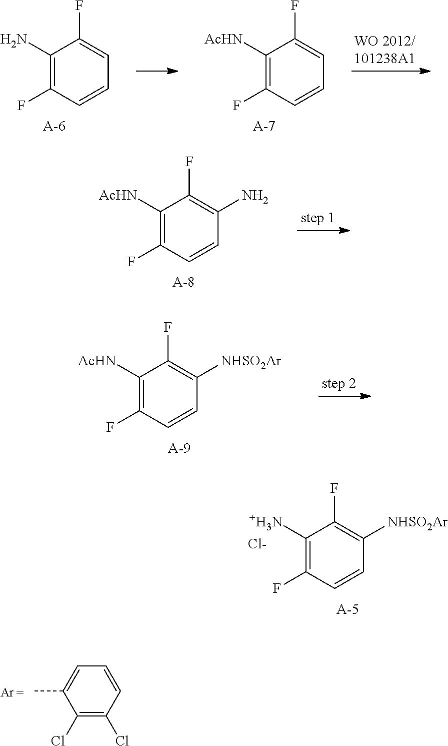

Preparation of acetanilide A-8: Acetanilide A-7 can be prepared by acetylation of 2,6-difluoroaniline A-6 with acetic anhydride following the literature procedure described in Bioorg. Med. Chem. 2016, 24, 2215. Intermediate A-7 was converted to acetanilide A-8 by sequential nitration followed by reduction of the nitro group to the aniline as described in WO 2012/101238A1.

Step 1—Preparation of sulfonamide A-9 (Ar=2,3-dichlorophenyl): Aniline A-8 (8.50 g, 45.5 mmol) was dissolved in THF (145 mL) and pyridine (4 eq., 14.7 mL) was added to the brown solution followed by 2,3-dichlorobenzenesulfonyl chloride (1.2 eq., 13.45 g). The resulting reaction mixture was stirred at 45° C. for 3.5 hours after which conversion was judged to be complete as monitored by LCMS. The reaction mixture was allowed to cool to room temperature then partitioned between EtOAc and 2-Me-THF (1:1) and water. A 1N HCl solution was added until a slightly acid pH was obtained. A significant amount of off-white solids were present in the biphasic mixture and were filtered out (first crop). The layers of the filtrate were separated, and the aqueous layer was extracted two more times with EtOAc. The combined organic extracts were washed once with water then with brine, dried over MgSO4, filtered and concentrated down to ˜20 mL. The resulting suspension was sonicated, and the solids were collected by filtration and washed with EtOH (crop 2). Both crops were combined and dried under reduced pressure. A-9 (15.3 g, 85% yield) was obtained as a beige solid that was used without further purification: ¹H NMR (DMSO-d₆) δ: 10.61 (s, 1H), 9.67 (s, 1H), 7.95 (dd, J=8.0, 1.4 Hz, 1H), 7.85 (dd, J=8.0, 1.4 Hz, 1H), 7.51 (t, J=8.0 Hz, 1H), 7.05-7.18 (m, 2H), 2.00 (s, 3H). MS m/z 395.0 (MH⁺).

Step 2—Preparation of aniline hydrochloride A-5 (Ar=2,3-dichlorophenyl): In a 500 mL round-bottomed flask, acetanilide A-9 (7.00 g, 17.7 mmol) was suspended in ethanol (65 mL) and a 1:1 mixture of concentrated HCl and water (65 mL) was added. The flask was equipped with a stoppered reflux condenser and heated at 80° C. with stirring. After 24 hours, conversion was ˜70% as judged by LCMS monitoring. Additional EtOH (65 mL) and 6N HCl (65 mL) were added to the suspension and stirring at 80° C. resumed for 7 more hours after which LCMS indicated complete conversion to the desired aniline. The reaction mixture was diluted with 50 mL of water while still warm and filtered through a plug of cotton to remove a small amount of insoluble materials. It was then concentrated to dryness under reduced pressure. The residue was azeotropically dried by evaporation of toluene 3× under reduced pressure then dried under vacuum, affording 7.2 g of the desired product A-5 as its HCl salt in the form of a yellow solid: ¹H NMR (DMSO-d₆) δ: 10.30 (s, 1H), 7.93 (dd, J=8.2, 1.2 Hz, 1H), 7.83 (dd, J=8.0, 1.4 Hz, 1H), 7.49 (t, J=8.0 Hz, 1H), 6.68-6.96 (m, 1H), 6.31 (td, J=8.6, 5.5 Hz, 1H). MS m/z 350.9 (M−H).

TABLE 1

| | | MS | |
|---|---|---|---|
| | | m/z | |
| A-5 | SM | (M − H) | ¹H NMR (400 MHz) |
| 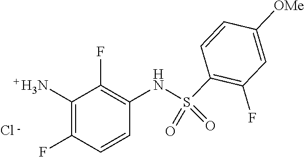 [SMILES: Cl ; COc1ccc(S(=O)(=O)Nc2ccc(F)c(N)c2F)c(F)c1] | A-2 | 331.0 | Not determined |
| 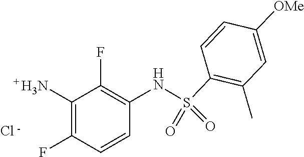 [SMILES: Cl ; COc1ccc(S(=O)(=O)Nc2ccc(F)c(N)c2F)c(C)c1] | A-2 | 327.0 | ¹H NMR (DMSO-d₆) d: 9.77 (s, 1H), 7.60 (d, J = 9.0 Hz, 1H), 6.93 (d, J = 2.3 Hz, 1H), 6.81 (dd, J = 8.8, 2.5 Hz, 1H), 6.72- 6.79 (m, 1H), 6.32 (td, J = 8.6, 5.5 Hz, 1H), 3.78 (s, 3H), 2.56 (s, 3H). |
| 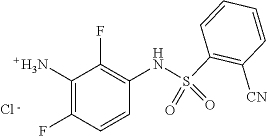 [SMILES: Cl ; N#Cc1ccccc1S(=O)(=O)Nc1ccc(F)c(N)c1F] | A-2 | 308.0 | ¹H NMR (DMSO-d₆) δ: 10.34 (s, 1H), 8.07 (dd, J = 7.4, 1.2 Hz, 1H), 7.77-7.95 (m, 3H), 6.67-6.90 (m, 1H), 6.30 (td, J = 8.4, 5.5 Hz, 1H). |
| 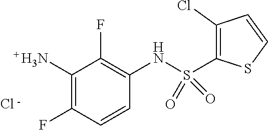 [SMILES: Cl ; Nc1c(F)ccc(NS(=O)(=O)c2sccc2Cl)c1F] | A-2 | 322.9 | Not determined |
| 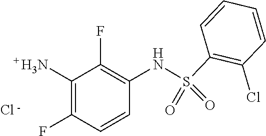 [SMILES: Cl ; Nc1c(F)ccc(NS(=O)(=O)c2ccccc2Cl)c1F] | A-8 | 317.0 | ¹H NMR (DMSO-d₆) δ: 10.12 (s, 1H), 7.85 (dd, J = 7.8, 1.6 Hz, 1H), 7.59-7.71 (m, 2H), 7.46 (td, J = 7.5, 1.4 Hz, 1H), 6.74-6.82 (m, 1H), 6.31 (td, J = 8.6, 5.5 Hz, 1H) |
| 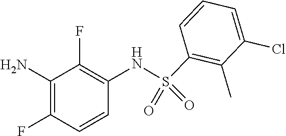 [SMILES: Cc1c(Cl)cccc1S(=O)(=O)Nc1ccc(F)c(N)c1F] | A-8 | 331.0 | ¹H NMR (CDCl₃) δ: 7.84 (d, J = 7.8 Hz, 1H), 7.58 (d, J = 7.8 Hz, 1H), 7.21 (t, J = 8.0 Hz, 1H), 6.68 - 6.74 (m, 2H), 6.63 (br s, 1H), 2.75 (s, 3H) |
| 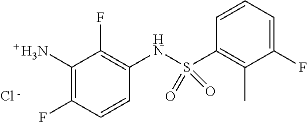 [SMILES: Cl ; Cc1c(F)cccc1S(=O)(=O)Nc1ccc(F)c(N)c1F] | A-8 | 315.0 | ¹H NMR (DMSO-d₆) δ: 10.10 (s, 1H), 7.54 (d, J = 7.8 Hz, 1H), 7.46 (td, J = 9.0, 1.2 Hz, 1H), 7.35 (td, J = 8.2, 5.5 Hz, 1H), 6.79 (ddd, J = 10.9, 9.1, 2.0 Hz, 1H), 6.32 (td, J = 8.6, 5.5 Hz, 1H), 2.49 (d, J = 2.0 Hz, 3H |
| 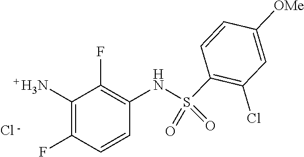 [SMILES: Cl ; COc1ccc(S(=O)(=O)Nc2ccc(F)c(N)c2F)c(Cl)c1] | A-2 | 346.9 | ¹H NMR (CDCl₃) δ: 7.84 (d, J = 8.2 Hz, 1H), 7.74 (br s, 1H), 6.95 (s, 1H), 6.89 (br s, 1H), 6.76 (br. d, J = 7.8 Hz, 1H), 6.53-6.72 (m, 1H), 5.52 (br s, 3H), 3.78 (s, 3H) |
| 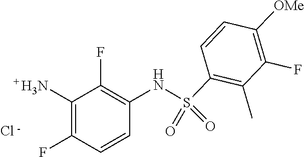 [SMILES: Cl ; COc1ccc(S(=O)(=O)Nc2ccc(F)c(N)c2F)c(C)c1F] | A-8 | 347.1 | ¹H NMR (DMSO-d₆) δ: 9.94 (s, 1H), 7.48 (dd, J = 8.9, 1.5 Hz, 1H), 7.07 (t, J = 8.6 Hz, 1H), 6.78 (ddd, J = 10.8, 9.0, 1.9 Hz, 1H), 6.33 (td, J = 8.6, 5,5 Hz, 1H), 3.86 (s, 3H), 2.48 (d, J = 2.7 Hz, 3H) |
| 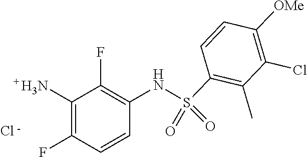 [SMILES: Cl ; COc1ccc(S(=O)(=O)Nc2ccc(F)c(N)c2F)c(C)c1Cl] | A-8 | 363.2 | ¹H NMR (DMSO-d₆) δ: 10.00 (s, 1H), 7.67 (d, J = 8.9 Hz, 1H), 7.07 (d, J = 9.0 Hz, 1H), 6.78 (ddd, J = 9.0, 6.5, 1.9 Hz, 1H), 6.33 (td (J = 8.7, 5.5 Hz, 1H), 3.89 (s, 3H), 2.65 (s, 3H) |
| 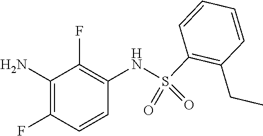 [SMILES: CCc1ccccc1S(=O)(=O)Nc1ccc(F)c(N)c1F] | A-8 | 313.0 | ¹H NMR (CDCl₃) δ: 7.86 (dd, J = 8.0, 1.3 Hz, 1H), 7.49 (td, J = 7.6, 1.3 Hz, 1H), 7.38-7.29 (m, J = 7.7 Hz, 1H), 7.24- 7.19 (m, 1H), 6.72-6.62 (m, 2H), 6.54 (brs, 1H), 3.04 (q, J = 7.5 Hz, 2H), 1.30 (t, J = 7.5 Hz, 3H) |
| 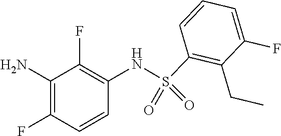 [SMILES: CCc1c(F)cccc1S(=O)(=O)Nc1ccc(F)c(N)c1F] | A-8 | 331.0 | ¹H-NMR (CDCl₃) δ: 7.70-7.64 (m, 1H), 7.24-7.19 (m, 2H), 6.73-6.65 (m, 2H), 6.63 (s, 1H), 3.01 (qd, J = 7.4, 2.1 Hz, 2H), 1.25 (t, J = 7.4 Hz, 3H) |
| 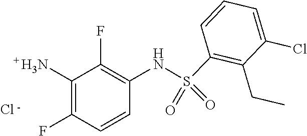 [SMILES: Cl ; CCc1c(Cl)cccc1S(=O)(=O)Nc1ccc(F)c(N)c1F] | A-8 | 346.8 | ¹H-NMR (DMSO-d₆) δ: 10.21 (s, 1H), 7.71 (ddd, J = 8.0, 4.9, 1.2 Hz, 1H), 7.35 (t, J = 8.0 Hz, 1H), 7.02-6.92 (m, 1H), 6.87-6.75 (m, 1H), 6.60 (bs, 1H), 6.28 (td, J = 8.6, 5.6 Hz, 1H), 3.12 (q, J = 7.2 Hz, 2H), 1.18 (t, J = 7.3 Hz, 3H) |
| 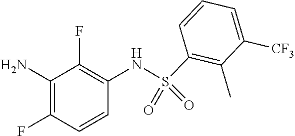 [SMILES: Cc1c(C(F)(F)F)cccc1S(=O)(=O)Nc1ccc(F)c(N)c1F] | A-8 | 366.3 | ¹H-NMR (CDCl₃) δ: 8.12 (d, J = 8.1 Hz, 1H), 7.84 (d, J = 8.0 Hz, 1H), 7.37 (t, J = 8.0 Hz, 1H), 6.74-6.64 (m, 2H), 6.61 (s, 1H), 3.88-3.62 (bs), 2.83 (s, 3H) |
| 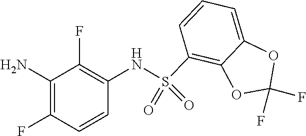 [SMILES: Nc1c(F)ccc(NS(=O)(=O)c2cccc3c2OC(F)(F)O3)c1F] | A-8 | 364.3 | ¹H-NMR (DMSO-d₆) δ 10.38 (s, 1H), 7.70 (dd, J = 7.6, 1.6 Hz, 1H), 7.44-7.26 (m, 2H), 6.82 (ddd, J = 10.7, 9.0, 1.9 Hz, 1H), 6.32 (td, J = 8.6, 5.5 Hz, 1H), 5.27 (s, 2H). |
| 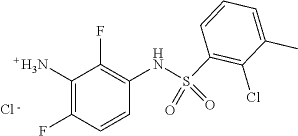 [SMILES: Cl ; Cc1cccc(S(=O)(=O)Nc2ccc(F)c(N)c2F)c1Cl] | A-8 | 333.1 | ¹H NMR (400 MHz, DMSO-d₆) δ 10.06 (s, 1H), 7.72 (dd, J = 7.9, 1.1 Hz, 1H), 7.65-7.61 (m, 1H), 7.36 (t, J = 7.7 Hz, 1H), 6.76 (ddd, J = 10.7, 9.0, 1.9 Hz, 1H), 6.28 (td, J = 8.6, 5.4 Hz, 1H), 2.42 (s, 3H) |
| 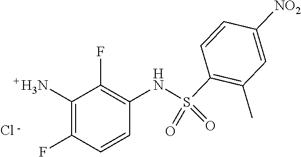 [SMILES: Cl ; Cc1cc([N+](=O)[O-])ccc1S(=O)(=O)Nc1ccc(F)c(N)c1F] | A-8 | 344.2 | ¹H NMR (DMSO-d₆) δ: 10.33 (s, 1 H), 8.29 (d, J = 2.35 Hz, 1 H), 8.13 (dd, J = 8.61, 2.35 Hz, 1 H), 7.91 (d, J = 8.61 Hz, 1 H), 2.72 (s, 2 H), 6.88-7.01 (m, 1 H), 6.70-6.86 (m, 1 H), 6.60 (td, J = 8.41, 5.09 Hz, 1 H), 6.32 (td, J = 8.51, 5.67 Hz, 1H) |
| 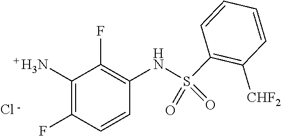 [SMILES: Cl ; Nc1c(F)ccc(NS(=O)(=O)c2ccccc2C(F)F)c1F] | A-8 | 338.2 | ¹H NMR (DMSO-d₆) δ: 10.38 (s, 1H), 8.59 (s, 1H), 7.92- 8.39 (m, 4H), 7.45-7.66 (m, 1H) |
| 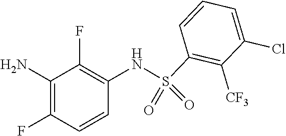 [SMILES: Nc1c(F)ccc(NS(=O)(=O)c2cccc(Cl)c2C(F)(F)F)c1F] | A-8 | 385.2 | ¹H NMR (400 MHz, DMSO-d₆) δ 10.27 (s, 1H), 8.00 (dd, J = 8.0, 4.5 Hz, 2H), 7.81 (t, J = 8.1 Hz, 1H), 6.83 (ddd, J = 10.7, 9.1, 1.8 Hz, 1H), 6.35 (td, J = 8.6, 5.5 Hz, 1H), 5.31 (s, 2H) |
| 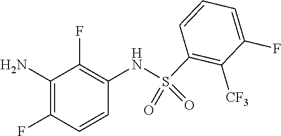 [SMILES: Nc1c(F)ccc(NS(=O)(=O)c2cccc(F)c2C(F)(F)F)c1F] | A-8 | 388.2 | ¹H NMR (400 MHz, DMSO-d₆) δ 10.25 (s, 1H), 7.96-7.70 (m, 3H), 6.83 (m, 1H), 6.37 (m, 1H), 5.30 (s, 2H) |
| 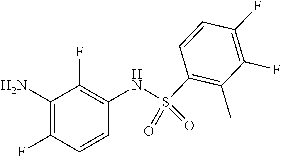 [SMILES: Cc1c(S(=O)(=O)Nc2ccc(F)c(N)c2F)ccc(F)c1F] | A-8 | 335.2 | ¹H NMR (400 MHz, DMSO-d₆) δ 10.15 (s, 1H), 7.54 (ddd, J = 8.8, 5.0, 1.3 Hz, 1H), 7.40 (dd, J = 17.3, 9.1 Hz, 1H), 6.80 (ddd, J = 10.7, 9.1, 1.8 Hz, 1H), 6.32 (td, J = 8.6, 5.5 Hz, 1H), 5.28 (s, 2H), 2.54 (d, J = 2.5 Hz, 3H) |
| 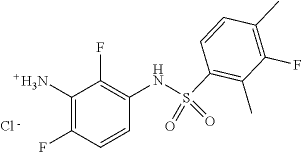 [SMILES: Cl ; Cc1ccc(S(=O)(=O)Nc2ccc(F)c(N)c2F)c(C)c1F] | A-8 | 329.1 | ¹H NMR (400 MHz, DMSO-d₆) δ 10.03 (s, 1H), 7.43 (d, J = 8.1 Hz, 1H), 7.22 (t, J = 7.8 Hz, 1H), 6.78 (ddd, J = 10.8, 9.0, 1.9 Hz, 1H), 6.33 (td, J = 8.6, 5.5 Hz, 1H), 2.48 (d, J = 2.4 Hz, 3H), 2.26 (d, J = 1.9 Hz, 3H) |
| 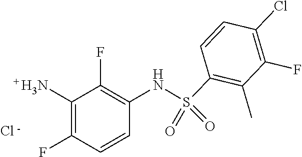 [SMILES: Cl ; Cc1c(S(=O)(=O)Nc2ccc(F)c(N)c2F)ccc(Cl)c1F] | A-8 | 349.1 | ¹H NMR (400 MHz, DMSO-d₆) δ 10.21 (s, 1H), 7.60-7.55 (m, 1H), 7.55-7.50 (m, 1H), 6.81 (ddd, J = 10.7, 9.0, 1.8 Hz, 1H), 6.32 (td, J = 8.6, 5.5 Hz, 1H), 2.53 (d, J = 2.6 Hz, 3H) |
| 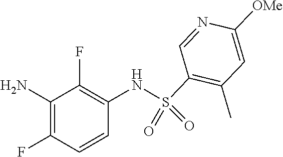 [SMILES: COc1cc(C)c(S(=O)(=O)Nc2ccc(F)c(N)c2F)cn1] | A-8 | 330.0 | Not determined |
| 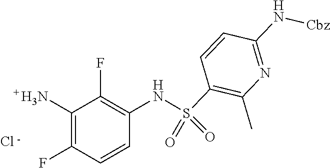 [SMILES: Cl ; Cc1nc(NC(=O)OCc2ccccc2)ccc1S(=O)(=O)Nc1ccc(F)c(N)c1F] | A-8 | 449.2 | ¹H NMR (DMSO-d₆) δ: 10.71 (s, 1H), 10.02 (s, 1H), 7.91- 8.07 (m, 1H), 7.74 (d, J = 8.61 Hz, 1H), 7.28-7.47 (m, 4H), 6.80 (t, J = 8.80 Hz, 1H), 6.33 (dt, J = 5.48, 8.61 Hz, 1H), 5.76 (s, 1H), 5.24 (s, 1H), 5.18 (s, 2H), 2.63-2.66 (m, 3H) |
| 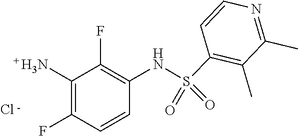 [SMILES: Cl ; Cc1nccc(S(=O)(=O)Nc2ccc(F)c(N)c2F)c1C] | A-8 | 314.2 | ¹H NMR (400 MHz, DMSO-d₆) δ 10.59 (s, 1H), 8.57 (d, J = 5.6 Hz, 1H), 7.72 (d, J = 5.6 Hz, 1H), 6.97 (br s, 3H), 6.80 (ddd, J = 10.7, 9.1, 1.8 Hz, 1H), 6.30 (td, J = 8.6, 5.4 Hz, 1H), 2.69 (s, 3H), 2.58 (s, 3H) |
| 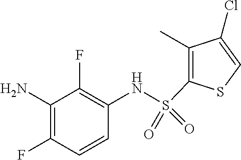 [SMILES: Cc1c(Cl)csc1S(=O)(=O)Nc1ccc(F)c(N)c1F] | A-8 | 337.2 | ¹H NMR (400 MHz, DMSO-d₆) δ 10.27 (s, 1H), 7.92 (s, 1H), 6.93-6.75 (m, 1H), 6.35 (td, J = 8.5, 5.5 Hz, 1H), 5.32 (s, 2H), 2.22 (s, 3H) |
| 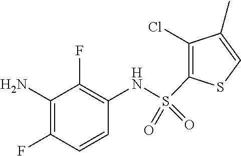 [SMILES: Cc1csc(S(=O)(=O)Nc2ccc(F)c(N)c2F)c1Cl] | A-8 | 337.2 | ¹H NMR (400 MHz, CDCl₃) δ 7.20 (m, 1H), 7.03 (br s, 1H), 6.87 (td, J = 8.8, 5.4 Hz, 1H), 6.73 (td, J = 9.8, 2.0 Hz, 1H), 2.21 (s, 3H) |
| 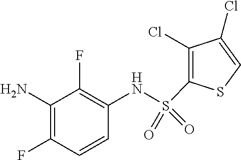 [SMILES: Nc1c(F)ccc(NS(=O)(=O)c2scc(Cl)c2Cl)c1F] | A-8 | 357.2 | ¹H NMR (400 MHz, DMSO-d₆) δ 10.59 (s, 1H), 8.15 (s, 1H), 6.98-6.71 (m, 1H), 6.35 (td, J = 8.5, 5.5 Hz, 1H), 5.34 (s, 2H) |
| 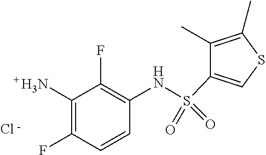 [SMILES: Cl ; Cc1scc(S(=O)(=O)Nc2ccc(F)c(N)c2F)c1C] | A-8 | 319.1 | Not determined |
| 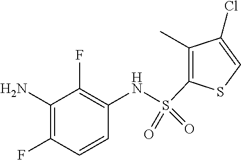 [SMILES: Cc1c(Cl)csc1S(=O)(=O)Nc1ccc(F)c(N)c1F] | A-8 | 337.2 | ¹H NMR (400 MHz, DMSO-d₆) δ 10.27 (s, 1H), 7.92 (s, 1H), 6.93-6.75 (m, 1H), 6.35 (td, J = 8.5, 5.5 Hz, 1H), 5.32 (s, 2H), 2.22 (s, 3H). |

### General Synthetic Method A—Preparation of Inhibitors I and II from Intermediates A-5 (Examples 1 and 36)

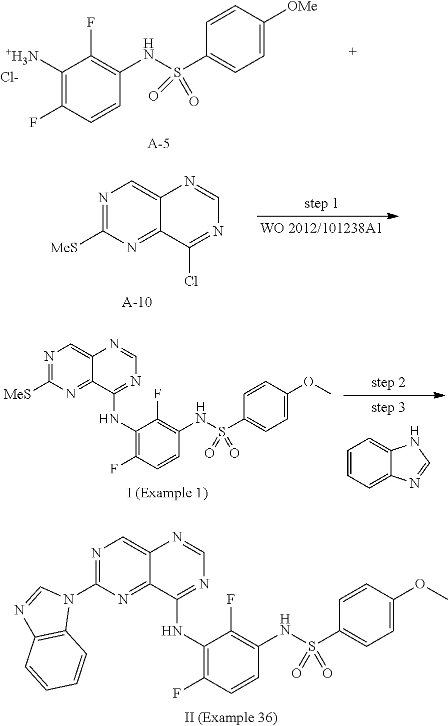

Step 1 (preparation of Example 1): chloropyrimidine A-10 (0.546 g, 1.25 equiv.) and aniline hydrochloride A-5 (Ar=4-methoxyphenyl; 0.750 g, 1 equiv.) were dissolved in AcOH (10 ml-) and the brown solution stirred at 50° C. for 1 h. LCMS shows complete conversion to the desired crude product (Example 1). The reaction mixture was cooled to RT and diluted with water (50 mL), producing a grey precipitate that was collected by filtration, washed with water and dried under vacuum. A 100 mg sample of the crude material was purified by passage through a small pad of silica gel (3 mL) using 1:1 hex/EA as eluent to remove colored baseline material. After removal of volatiles from the light burgundy solution, the material was lyophilized from MeCN-water to provide inhibitor of Example 1 (73 mg).

Step 2 and 3 (Preparation of Example 36): To a solution of the crude thiomethyl derivative (Example 1; 355 mg, 0.72 mmol, 1 equiv.) in 7 mL of DCM was added m-CPBA (185 mg, 1.07 mmol, 1.5 equiv.) at room temperature. The mixture was allowed to stir at that temperature for 1 h. LCMS shows complete conversion to an 80:20 mixture of sulfoxide and sulfone. The mixture was concentrated to remove most of the DCM then taken into EtOAc and washed 3× with a solution of NaHCO₃. The combined aqueous layers were back-extracted with EtOAc and the combined organic layers were washed once with water then brine. The organic layer was then dried over MgSO4, filtered and concentrated to afford 356 mg of a yellow foam that was used as such without further purification: MS m/z 507 and 523 (MH⁺).

The crude mixture of sulfoxide and sulfone from above (25 mg, 0.05 mmol) and benzimidazole (12 mg, 0.1 mmol, 2 equiv.) were charged in a 4 mL vial and dissolved in NMP (1 mL). DIEA (43 μL, 0.25 mmol, 5 equiv.) was then added and the resulting mixture was stirred at 60° C. for 19 h (LCMS shows complete reaction). The reaction was quenched by the addition of 0.2 mL of AcOH then diluted to 2 mL with methanol. The solution was filtered then purified by prep-HPLC (MeOH/H2O/0.1% HCO₂H conditions, 10%-100% methanol gradient). The fractions containing the major peak were pooled and partially concentrated to remove methanol. The resulting suspension was dissolved by the addition of a few milliliters of acetonitrile then the solution was frozen and lyophilized. Obtained 8.1 mg of the desired product (Example 36) as a pinkish solid.

Other Examples of inhibitors of general formulae I and II that were prepared in an analogous fashion are listed in Tables 1 and 2 along with characterization data.

### Synthesis of Inhibitors I and II Using General Synthetic Method B (Examples 12 and 56)

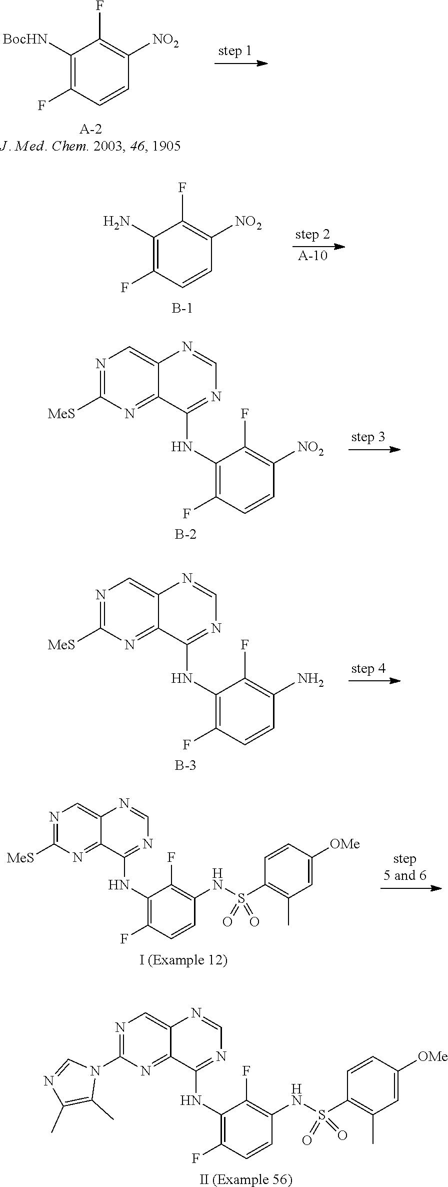

Step 1: Carbamate A-2 (1.50 g) was dissolved in DCM (5 mL) and TFA (2 mL) was added. After stirring for 2 h at room temperature deprotection was complete (LCMS) and the reaction mixture was concentrated and dried under reduced pressure.

Step 2: while the crude TFA salt from step 1 (above) can be used directly in step 2, the desired intermediate B-2 was contaminated with varying amounts of 8-hydroxy-2-thiomethylpyrimidopyrimidine resulting from solvolysis of A-10. This side reaction could be minimized and a cleaner intermediate B-2 was obtained if the aniline TFA salt was neutralized to the free aniline prior to reaction with A-10 as follows. The crude TFA salt from step 1 was dissolved in DCM and the solution washed with NaHCO₃. After drying (MgSO₄), removal of volatiles gave the free aniline B-1 as a brown sticky solid (0.85 g): ¹H NMR (DMSO-d₆) δ: 7.22-7.38 (m, 1H), 7.06-7.19 (m, 1H), 5.92 (br s, 2H).

Chloropyrimidopyrimidine A-10 (550 mg, 2.6 mmol) and the B-1 aniline free-base (0.39 g, 2.25 mmol) were dissolved in acetic acid (7 mL) and the mixture stirred at 55° C. for 1 h. LCMS shows complete conversion to desired product. The reaction mixture was cooled to RT and diluted with trice the volume of water causing precipitation of the product as a cream-colored solid. The material was collected by filtration, washed with water and dried under vacuum (0.58 g): ¹H NMR (DMSO-d₆) δ: 10.28 (s, 1H), 9.35 (s, 1H), 8.60 (s, 1H), 8.23-8.49 (m, 1H), 7.58 (t, J=8.8 Hz, 1H), 2.71 (s, 3H). MS m/z 350.1 (MH⁺).

Step 3: nitro arene B-2 (0.86 g, 2.45 mmol) and tin(II)chloride dihydrate (2.7 equiv., 6.6 mmol, 1.49 g) were suspended in absolute ethanol (10 mL) and the mixture was stirred at 65° C. for 3 h. The reaction mixture was partitioned between 1N NaOH and EtOAc. The organic extract was washed with NaHCO₃, brine and dried (MgSO₄). The drying agent was then separated from the extract by filtration through a pad of silica gel (40 mL) using EtOAc as eluent to remove baseline material. The filtrate was concentrated and the residue triturated with EtOAc/hexane to give aniline B-3 as an orange solid that was collected by filtration, washed with ether and dried (0.438 g): ¹H NMR (DMSO-d₆) δ: 9.98 (s, 1H), 9.28 (s, 1H), 8.53 (s, 1H), 6.93 (td, J=9.4, 1.6 Hz, 1H), 6.77 (td, J=9.4, 5.5 Hz, 1H), 5.12 (br s, 2H), 2.73 (s, 3H). MS m/z 321.1 (MH⁺).

The mother liquors were purified by flash chromatography (30 mL) using 10%-70% EtOAc in hexane (Rf=0.3 in 1:2 hex-EA) to provide an additional 96 mg of aniline B-3.

Step 4 (Example 12): aniline B-3 (25 mg, 0.078 mmol) and 4-methoxy-2-methylbenzenesulfonyl chloride (100 mg, 0.23 mmol, 3 equiv.) were dissolved in THF (1 mL) and pyridine (40 μL) was added. The mixture was stirred at 45° C. for 1 h (50% conversion by LCMS). Another portion of sulfonyl chloride (100 mg) was added and stirring at 45° C. resumed for 18 h (LCMS shows complete conversion). The reaction mixture was acidified with TFA (100 μL) and diluted to 1.8 mL with DMSO. The product (Example 12) was isolated by prep HPLC using 30%-100% MeOH+0.1% HCOOH gradient (13 mg).

Step 5 and 6 (Example 56): To a suspension of the thiomethylpyrimidine (Example 12, 300 mg, 0.59 mmol) in DCM (5 mL) at room temperature was added 1.2 equiv. of m-CPBA (160 mg, 0.71 mmol). The mixture became a yellow solution over 10 minutes. It was allowed to stir at room temperature for a total of 45 minutes at which point LCMS revealed the reaction was complete. The mixture was concentrated to remove most of the DCM then partitioned between EtOAc and aqueous NaHCO₃. The layers were separated, and the organic layer was washed twice more with the NaHCO₃ solution. The combined aqueous layers were back extracted twice with EtOAc and the combined organic layers were washed with brine then dried over MgSO₄ and filtered. The filtrate was concentrated to dryness then dried under vacuum to afford 295 mg of a mixture of sulfoxide and sulfone (˜80:20 ratio by LCMS). The material was used as such in the next step without further purification. 4,5-Dimethyl-1H-imidazole hydrochloride (19 mg, 0.14 mmol, 3 equiv.) and the sulfoxide-sulfone mixture from above (25 mg, 0.048 mmol, 1 equiv.) were charged in a 4 mL vial followed by NMP (o, 5 mL) and DIEA (42 μL, 0.24 mmol, 5 equiv.). The resulting mixture was stirred at 60° C. for 4 h (LCMS shows complete conversion). The reaction mixture was acidified with AcOH (0.2 mL) and the product (Example 56) was isolated by prep-HPLC (MeOH/H₂O/0.1% formic acid, 30%-100% methanol gradient). The fractions containing the major peak were pooled and partially concentrated to remove methanol. The resulting suspension was dissolved by the addition of a few milliliters of acetonitrile then the solution was frozen and lyophilized. Obtained 14 mg of a yellow powder found to be only 88% pure by HPLC. This material was repurified by flash chromatography on a 3 g silica gel cartridge using a 100% DCM to 7% isopropanol in DCM gradient. The appropriate fractions were pooled, concentrated, co-evaporated with acetonitrile once then taken into a 1:1 MeCN/water mixture. After lyophilization 7.5 mg of the desired product (Example 56) were obtained as a yellow solid.

Other Examples of inhibitors of general formulae I and II that were prepared in an analogous fashion are listed in Tables 2 and 3 along with characterization data.

### Synthesis of Inhibitors III and IV Using General Synthetic Method C (Examples 29 and 73)

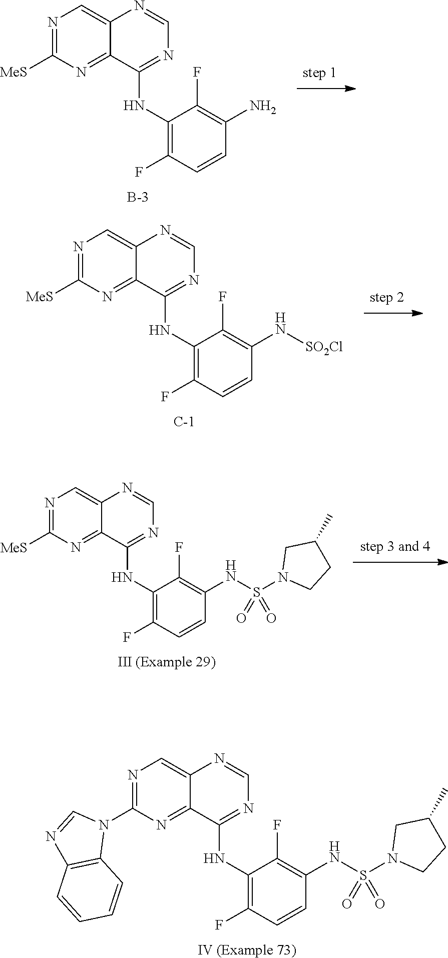

Step 1: sulfuryl chloride (0.051 ml, 0.624 mmol) was dissolved in DCM (2 ml) and the solution cooled to −78° C. A solution of aniline B-3 (50 mg, 0.156 mmol) and triethylamine (0.11 ml, 0.78 mmol) in DCM (5 mL) was added dropwise over 5 min. The reaction mixture was stirred at −78° C. for 90 min to give a solution of intermediate C-1.

Step 2 (Example 29): (R)-3-methylpyrrolidine hydrochloride (76 mg, 0.62 mmol) in DCM (3 mL) was added to the cold solution on intermediate C-1, followed by pyridine (0.5 mL). The reaction mixture was allowed to warm up to RT then stirred for 2 h (LCMS showed the mass of the product and completion of the reaction). The reaction mixture was evaporated to dryness and azeotroped with toluene to remove the pyridine. The residue was purified on ISCO using a RediSep 24 g column (DCM/EtOAc) to provide inhibitor of Example 29 (25 mg) as a brown solid.

Step 3 and 4 (Example 73): the thiomethylpyrimidine from Step 1 (Example 29, 20 mg, 0.043 mmol) was dissolved in DCM (5 mL) and m-CPBA (11.5 mg, 0.051 mmol) was added. The mixture was stirred at RT for 30 min (LCMS shows no more starting material). The reaction mixture was diluted with dichloromethane 25 mL and washed with a saturated NaHCO₃ solution. The organic layer was separated and dried over anhydrous Na₂SO₄ and filtered. The filtrate was evaporated to dryness to provide a mixture of sulfoxide and sulfone as a brown foam solid (18 mg), which was used for the next step without any further purification.

The crude material from above (18 mg, 0.037 mmol) was dissolved in NMP (2 mL) and DIEA (0.033 ml, 0.186 mmol) and benzimidazole (13.2 mg, 0.112 mmol) were added. The reaction mixture was heated at 60° C. for 3 h (LCMS shows complete conversion). The reaction mixture was diluted with MeOH (1 mL), acidified with a few drops of acetic acid and the product isolated by Prep-HPLC using a 30-100% MeOH-Water-0.1% TFA gradient. Product fractions were evaporated partially, dissolved in ACN and water and lyophilized to provide Example 73 (12 mg).

Other Examples of inhibitors of general formula I and II that were prepared in an analogous fashion are listed in Tables 2 and 3 along with characterization data.

### Synthesis of Inhibitors V Using General Synthetic Method D (Example 88)

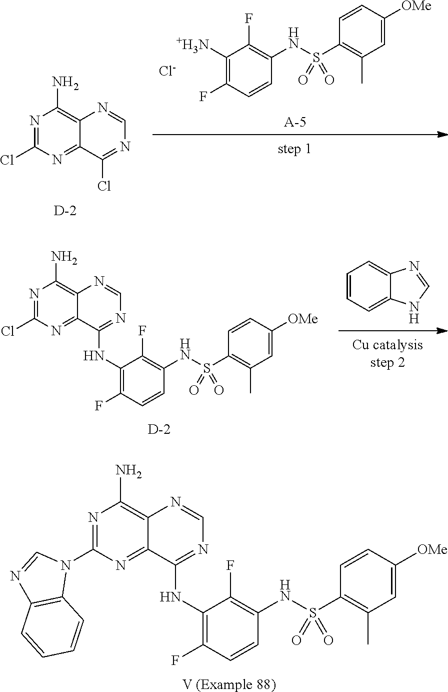

Step 1: amino-dichloropyrimidopyrimidine 0-2 (50 mg, 0.23 mmol, 1 equiv.) and the aniline hydrochloride A-5 (85 mg, 0.23 mmol, 1 equiv.) were dissolved in AcOH (1.5 mL) and the mixture stirred at 55° C. for 1 h (LCMS shows conversion to product, but a small amount of aniline remains. Add another 10 mg of the dichloro derivative D-2 and continue stirring at 55° C. for 30 min. Cool to RT, dilute with 3-fold volume of water, collect cream-colored precipitate, wash with water and dry under vacuum (120 mg): ¹H NMR (DMSO-d₆) δ: 10.10 (s, 1H), 9.78 (s, 1H), 8.61 (br s, 1H), 8.39 (br s, 1H), 8.37 (s, 1H), 7.66 (d, J=9.0 Hz, 1H), 7.17-7.26 (m, 1H), 7.13 (t, J=9.4 Hz, 1H), 6.94 (d, J=2.0 Hz, 1H), 6.86 (dd, J=9.0, 2.0 Hz, 1H), 3.80 (s, 3H), 2.56 (s, 3H). MS m/z 508.0 (MH⁺).

Step 2: The amino-chloropyrimidine (25 mg, 0.05 mmol, 1 equiv.) and benzimidazole (10.5 mg, 0.09 mmol, 1.8 equiv.) were suspended in DMSO (0.7 mL) and cesium carbonate (37 mg, 0.11 mmol, 2.3 equiv.) was added followed by Cu powder (0.3 mg, 0.1 equiv.) and racemic-BINOL (1.5 mg, 0.1 equiv.). The mixture was stirred at 110° C. for 2 h (LCMS shows complete conversion to desired mass). The brown reaction mixture was acidified with TFA (100 μL) and the product isolated by prep-HPLC using a 30-100% MeOH/0.1% HCOOH gradient. Inhibitor of Example 88 (10 mg) was isolated as a beige solid after lyophilization. Examples 85, 86 and 87 were prepared in a similar fashion.

In the case of Example 84, the product was formed using a nucleophilic displacement under basic conditions (intermediate D-3, 4 equiv. of DIEA and 2 equiv. of 3-fluoropyrrolidine hydrochloride were heated in NMP at 100° C. for 1.5 h) as described in the last step of Schemes A and B.

Other Examples of inhibitors of general formula V that were prepared in an analogous fashion are listed in Table 4 along with characterization data.

### Synthesis of Inhibitors VI Using General Synthetic Method E

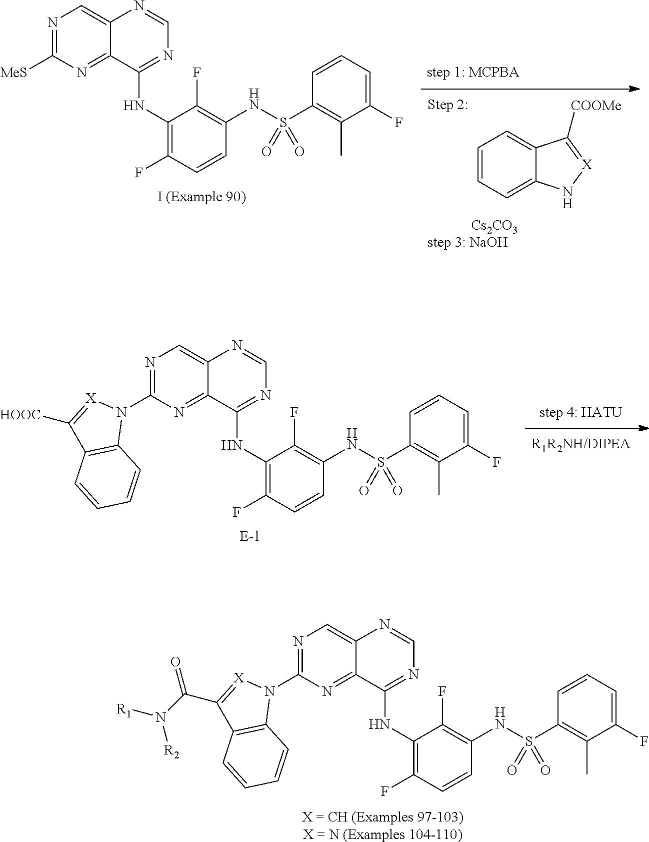

Step 1: Intermediate I (Ar=3-fluoro-2-methylphenyl, Example 90) prepared from the appropriate intermediate A-5, was oxidized to a mixture of sulfoxide and sulfone as described in steps 1 and 2 of general synthetic method A.

Steps 2 and 3 (X═CH): Cesium carbonate (1.16 g, 3.51 mmol) and methylindole-3-carboxylate (X═CH; 0.554 g, 3.1 mmol) were suspended in DMSO (62 mL) and the mixture stirred at RT for 10 min. The mixture of sulfoxide and sulfone from step 1 (1.55 g, 2.95 mmol) was added and the mixture stirred at 80° C. for 18 h, at which point conversion was judged to be complete by LCMS analysis.

NaOH (236 mg, 5.9 mmol) was added to the reaction mixture from step 2 and the mixture was stirred at RT until complete conversion to the desired carboxylic acid intermediate E-1 (X═CH) as determined by LCMS analysis. Citric acid solution (1M) was then added to precipitate the product that was filtered, washed with water and dried to give E-1 (X═CH) as a brown solid (1.67 g, 93% yield): ¹H NMR (DMSO-d₆) δ 12.70 (br, 1H), 10.56 (s, 1H), 10.50 (s, 1H), 9.64 (s, 1H), 9.43 (s, 1H), 8.89 (d, J=8.3 Hz, 1H), 8.55 (s, 1H), 8.20-8.12 (m, 1H), 7.61 (d, J=7.8 Hz, 1H), 7.53-7.45 (m, 1H), 7.45-7.33 (m, 3H), 7.27 (t, J=9.2 Hz, 1H), 2.49-2.48 (m, 3H). MS m/z 620.3 (MH⁺).

Steps 2 and 3 (X═N): Sodium tert-butoxyde (341 mg, 3.54 mmol) was added to methyl 1H-indazole-3-carboxylate (573 mg, 3.19 mmol) in dry THF (6 2 mL) and the mixture stirred at RT for 10 min. The sulfoxide/sulfone mixture from step 1 (1.55 g, 2.95 mmol) was added and the mixture stirred at RT for 18 h when conversion was judged to be complete by LCMS analysis. The reaction mixture was concentrated under reduced pressure to a volume of 20 mL and a solution of NaOH (236 mg, 5.9 mmol) in water (20 mL) was added. The mixture was stirred for 4 h at RT at when conversion to the desired carboxylic acid was complete by LCMS analysis. The reaction mixture was tend concentrated under reduced pressure to remove THF and the aqueous residue was acidified with 1M citric acid to precipitate the product which was collected by filtration, washed with water and dried to give E-1 (X═N) as a brown solid in quantitative yield: ¹H NMR (DMSO-d₆) δ 10.53 (br, 1H), 10.03 (s, 1H), 9.69 (s, 1H), 8.89 (d, J=8.6 Hz, 1H), 8.61 (s, 1H), 8.26 (d, J=8.1 Hz, 1H), 7.72-7.67 (m, 1H), 7.62 (d, J=7.8 Hz, 1H), 7.55 (dt, J=8.0, 1.5 Hz, 1H), 7.48 (d, J=8.5 Hz, 1H), 7.41 (td, J=8.0, 5.6 Hz, 1H), 7.33 (td, J=8.6, 5.9 Hz, 1H), 7.25 (t, J=9.3 Hz, 1H), 2.50-2.49 (m, 3H). MS m/z 607.2 (MH⁺).

Step 4 (preparation of example 103, X═CH): carboxylic acid E-1 (X═CH; 61 mg, 0.1 mmol) and (S)-3-hydroxypiperidine hydrochloride (17 mg, 0.12 mmol) were dissolved in DMF (0.5 mL) and DIPEA (61 μL, 0.35 mmol) was added followed by HATU (58 mg, 0.15 mmol). The mixture was stirred for 18 h at ambient temperature. The reaction mixture was then purified directly by preparative reversed-phase HPLC to provide the compound of example 103 as a yellow powder after lyophilization.

Other examples listed in Table 3 (X═CH or N) and using Method E were prepared in a similar fashion.

### Synthesis of Inhibitors VII Using General Synthetic Method F (Example 95)

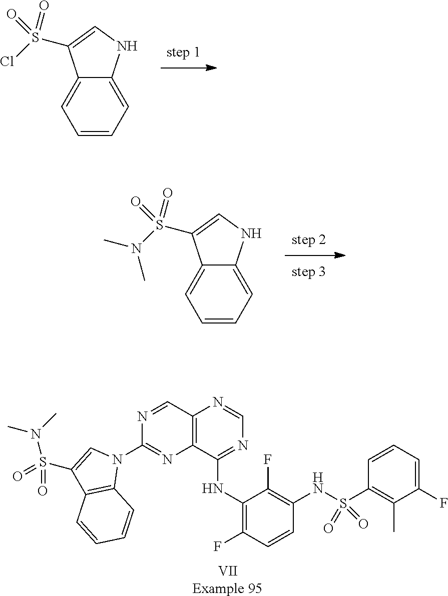

Step 1: 3-indolesulfonyl chloride was prepared as described in Org. Lett. 2011, 13, 3588. The sulfonyl chloride (200 mg, 0.9 mmol) was charged into a 25 mL flask to which was added THF (4 mL) followed dimethylamine hydrochloride (2 equiv., 150 mg, 1.9 mmol) and DIEA (4 equiv., 0.65 mL, 3.7 mmol). The solution quickly became pale yellow and a yellow gummy oil deposited in the bottom. After 20 minutes of stirring at RT (LCMS showed complete consumption of the sulfonyl chloride). The mixture was partitioned between EtOAc and a saturated solution of NH₄Cl. The aqueous layer was extracted with EtOAc and the combined organic layers were washed once more with the saturated NH₄Cl solution then with brine. It was then dried over MgSO₄, filtered and concentrated to dryness affording 65 mg of a beige crystallizing solid that was used as such without further purification: ¹H NMR (DMSO-d₆) δ: 12.17 (br s, 1H), 7.96 (d, J=3.1 Hz, 1H), 7.81 (d, J=7.8 Hz, 1H), 7.49-7.57 (m, 1H), 7.22-7.28 (m, 1H), 7.16-7.22 (m, 1H), 2.58 (s, 6H). MS m/z 225.1 (M H⁺).

Step 2: thiomethyl derivative I (Ar=3-fluoro-2-methylphenyl; Example 90) was oxidized to a mixture of sulfoxide and sulfone as described in general method A.

Step 3 (Example 95): prepared from indole sulfonamide from Step 1 and sulfoxide/sulfone mixture from step 2 using general method A.

Other examples listed in Table 3 were prepared in a similar fashion using general Method F.

### Synthesis of Inhibitors VIII Using General Synthetic Method G (Example 142)

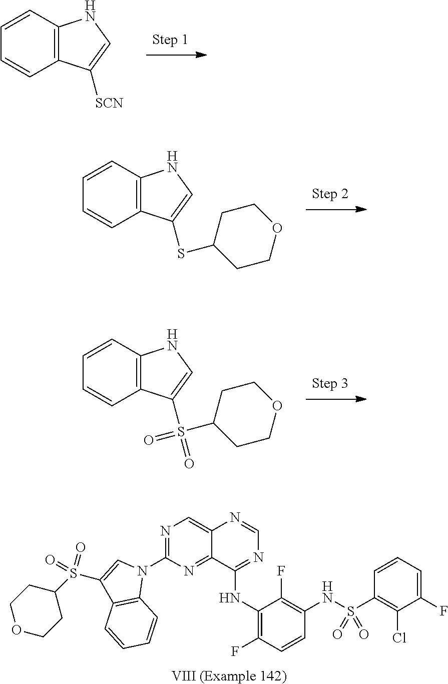

Step 1: To a solution of 1H-indol-3-yl-thiocyanate (Phosphorus, Sulfur and Silicon and the Related Elements 2014, 189, 1378) (100 mg, 0.57 mmol) in iPrOH (5 mL) was added sodium sulfide nonahydrate (414 mg, 1.72 mmol) dissolved in water 0.5 mL, then the resulting mixture was stirred at 50° C. for 2 h. After this time, 4-chlorotetrahydropyran (0.19 mL, 1.72 mmol) was added and stirred at 50° C. overnight. The reaction mixture was diluted with EtOAc (30 mL) and separated. The organic layer was washed with water (15 mL) followed by brine (15 mL), dried over MgSO₄ and then concentrated under vacuum to give the crude sulfide, which was used directly in the next step without further purification.

Step 2: The sulfide from step 1 was dissolved in DCM and 3-chloroperoxybenzoic acid (297 mg, 1.72 mmol) was added and stirred at room temperature for 2 h. After completion, the reaction was quenched by adding 10 mL of a 1:1 solution of saturated aqueous NaHCO₃ and 10% aqueous Na₂SO₃. The resulting suspension was stirred at room temperature for 15 min. EtOAc was added and the organic layer was separated. The organic layer was washed with water (15 mL) then saturated brine solution (15 mL). The organic layer was separated, dried (MgSO₄) and filtered before concentration to dryness to provide the expected sulfone (154 mg, 98%) which was dissolved in DMSO and use directly in the next step without further purification. MS m/z 266.2 (MH⁺).

Step 3 (Example 142): indole from Step 2 was coupled to intermediate I (Ar=2,3-dichlorophenyl) using the procedure described in general method A.

Other examples of inhibitors prepared in a similar fashion using the appropriate alkylating agent in step 1 are listed under method F in Table 3.

### Synthesis of Inhibitors IX Using General Synthetic Method H (Example 130)

Using iron as reducing agent in step 2 (Example 130):

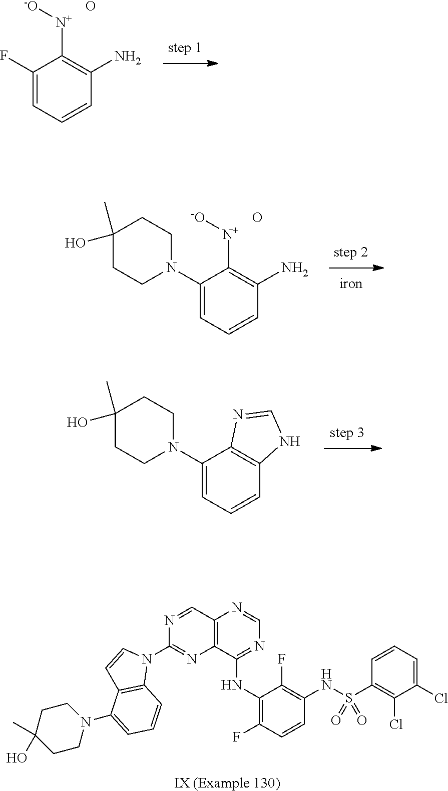

Step 1: 4-Methylpiperidin-4-ol (0.24 g, 1.84 mmol) and potassium carbonate (0.49 g, 3.52 mmol) were added to a solution of 3-fluoro-2-nitro-aniline (0.25 g, 1.60 mmol) in MeCN (2.6 mL). The resulting mixture was stirred at 85° C. for 10 h. MeCN was removed under reduce pressure and EtOAc was added. The suspension was centrifuged and poured into a flask. The solution was concentrated and the crude 1-(3-amino-2-nitro-phenyl)-4-methyl-piperidin-4-ol (0.40 g, 94% yield) was without further purification for the next step. MS m/z 252.2 (MH⁺).

Step 2 (using iron as reducing agent): Iron (0.37 g, 6.70 mmol) and ammonium chloride (0.36 g, 6.70 mmol) were added to a mixture of 1-(3-amino-2-nitro-phenyl)-4-methyl-piperidin-4-ol 0.34 g, 1.34 mmol) in iPrOH (6.5 mL) and formic acid (1.9 mL, 49.6 mmol). The result mixture was heated to 90° C. and stirred for 10 h. The reaction mixture was cooled down to room temperature and filtered through Celite®. The solution was concentrated and the crude was purified by column chromatography (silica gel, 0-15% MeOH in DCM) to afford 1-(1H-benzimidazol-4-yl)-4-methyl-piperidin-4-ol (0.17 g, 55% yield) as a reddish foamy solid. MS m/z 232.2 (MH⁺). ¹H NMR (400 MHz, DMSO-d₆) δ: 12.21 (br s, 1H), 8.02 (s, 1H), 6.85-7.20 (m, 2H), 6.33-6.67 (m, 1H), 4.24 (s, 1H), 3.15-3.26 (m, 2H), 2.48 (td, J=1.66, 3.72 Hz, 2H), 1.41-1.74 (m, 4H), 1.16 (s, 3H).

Step 3 (Example 130): the benzimidazole from Step 2 was coupled to intermediate I (Ar=2,3-dichlorophenyl) using the procedure described in general method A.

Using zinc as reducing agent in step 2 (Preparation of benzimidazole fragment C328):

Step 1: 4-Methylpiperidin-4-ol was replaced by (R)-3-methoxypiperidine hydrochloride. The nitroarene of step 1 was obtained in 88% yield as a red solid and used directly in step 2 without purification: MS m/z 252.1 (MH⁺). ¹H NMR (400 MHz, CDCl₃) δ: 1.2-1.35 (m, 1H), 1.58-1.73 (m, 1H), 1.74-1.85 (m, 1H), 2.06-2.17 (m, 1H), 2.53-2.61 (m, 1H), 2.70 (td, J=11.5, 3.0 Hz, 1H), 3.12 (dt, J=12.0, 3.8 Hz, 1H), 3.35-3.45 (m, 2H), 3.40 (s, 3H), 4.79 (br s, 2H), 6.38 (dd, J=8.25, 1.13 Hz, 1H), 6.41 (dd, J=8.13, 1.13 Hz, 1H), 7.12 (t, J=8.13 Hz, 1H).

Step 2: In a 250 mL round-bottomed flask equipped with a Teflon-coated magnetic stirring bar and under nitrogen, a mixture of the crude product from step 1 (1.37 g, 5.45 mmol) and ammonium chloride (4.08 g, 76.3 mmol) in methanol (18 mL) and 2-methyltetrahydrofuran (36 mL) was treated with zinc dust (2.7 g, 38.16 mmol) added in one portion. An exotherm was observed and after ˜ 10 min reaction mixture became colorless. The reaction mixture was stirred for 1 h and LCMS indicated clean conversion to the 1,2-diaminobenzene. The reaction mixture was filtered through a small celite pad which was washed with DCM:isopropanol (9:1, 30 mL). The filtrate was diluted with DCM-isopropanol (9:1, 100 mL) and washed with 10% aqueous potassium carbonate (30 mL, pH˜10). The organic phase was collected and dried over anhydrous magnesium sulfate. Evaporation of the solvent under reduced pressure gave of a 1.2 g: MS m/z 222.2 (MH⁺). ¹H NMR (400 MHz, CDCl₃) δ: 1.4-1.6 (br s, 1H), 1.61-1.74 (m, 1H), 1.81-1.94 (m, 1H), 2.0 (br s, 1H), 2.65 (br s, 1H), 2.95 (br s, 1H), 3.17 (br s, 1H), 3.37 (br s, 2H), 3.4-3.5 (m, 2H), 3.41 (s, 3H), 3.79 (br s, 2H), 6.53 (dd, J=7.63, 1.45 Hz, 1H), 6.61 (dd, J=7.94, 1.45 Hz, 1H), 6.68 (t, J=7.8 Hz, 1H).

The crude 1,2-dianiline was cyclized to the desired benzimidazole as follows: a 100 mL round-bottomed flask equipped with a Teflon-coated magnetic stirring bar, a reflux condenser and under nitrogen was charged with 2-propanol (20 mL) and the crude product from above (1.20 g, 5.42 mmol). Then, formic acid (5 mL, 132 mmol), was added in one portion and the resulting solution was heated at 60° C. for 16 h. LCMS indicated clean formation of the desired benzimidazole (m/z 232). The cooled reaction mixture was diluted with DCM:2-propanol (9:1, 200 ml) washed with 10% aqueous potassium carbonate (40 mL, pH 10), brine and dried over anhydrous magnesium sulfate. Evaporation of the solvent under reduced pressure gave a brown solid. This solid was chromatographed on silica gel (3×12 cm), elution ethyl acetate-2-propanol (90:5 to 9:1) to give 1.01 g (81% yield). Trituration with ethyl acetate (10 mL) gave 0.88 g of a light orange-brown solid: MS m/z 232.1 (MH⁺). ¹H NMR (400 MHz, CDCl₃) δ: 1.47 (br s, 1H) 1.80 (br s, 1H) 1.94 (br s, 1H), 2.13 (br s, 1H), 2.92 (br s, 2H), 3.47 (s, 3H), 3.53-3.63 (m, 1H), 4.1 (br s, 2H), 6.72 (br s, 1H), 7.07 (br s, 1H), 7.19 (t, J=7.9 Hz, 1H), 7.98 (s, 1H), 9.43 and 9.7 (two br s, ratio 2:1, 1H).

Other examples of inhibitors prepared in a similar fashion using the appropriate amine in step 1 and using either iron or zinc as a reducing agent in step 2, are listed under method H in Table 3.

### Synthesis of Inhibitors X Using General Synthetic Method I (Example 138)

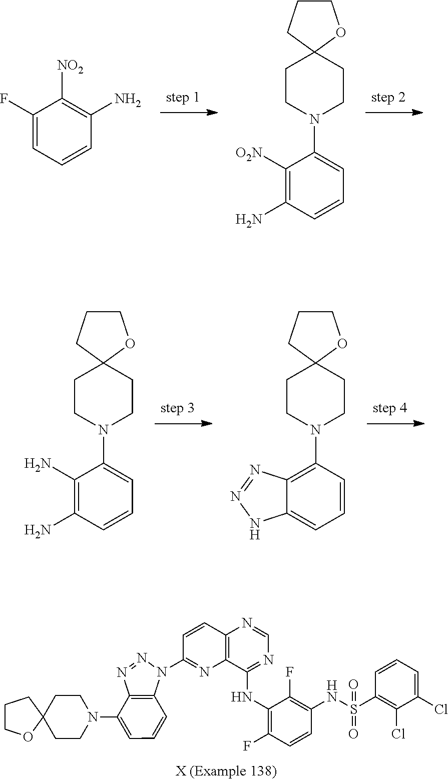

Step 1: 1-oxa-8-azaspiro[4.5]decane hydrochloride (188 mg, 1.06 mmol) and potassium carbonate (2.20 eq, 292 mg, 2.11 mmol) were added to a bright red solution of 3-fluoro-2-nitro-aniline (150 mg, 0.961 mmol) in ACN (4.5 mL). The resulting mixture was stirred at 90° C. for 16 h. Upon completion, the reaction mixture was diluted with ACN and centrifuged. The supernatant was separated and used as is in the next step. MS m/z 278.3 (MH⁺).

Step 2: To 2-nitro-3-(1-oxa-8-azaspiro[4.5]decan-8-yl)aniline (250 mg, 0.901 mmol) in ACN was added ammonium chloride (1028 mg, 19.2 mmol) and zinc (628 mg, 9.61 mmol). The resulting suspension was stirred at 40° C. for 1 h. Upon completion, the reaction mixture was diluted with EtOAc then centrifuged. The supernatant was separated and evaporated under vacuum. The residue 3-(1-oxa-8-azaspiro[4.5]decan-8-yl)benzene-1,2-diamine (194 mg, 0.784 mmol, 82% yield) was used as is in the next step. MS m/z 248.2 (MH⁺).

Step 3: 3-(1-oxa-8-azaspiro[4.5]decan-8-yl)benzene-1,2-diamine (194 mg, 0.78 mmol) was dissolved in AcOH (6 mL) then sodium nitrite (54 mg, 0.78 mmol) was added and stirred 1 h at room temperature. Upon completion, EtOAc and aqueous NaHCO₃ were added and the organic layer was separated. The organic layer was washed with aq. NaHCO₃ then brine, dried over MgSO₄, filtered and concentrated under vacuum to provide 8-(1H-benzotriazol-4-yl)-1-oxa-8-azaspiro[4.5]decane (165 mg, 0.64 mmol, 67% yield) used without purification. MS m/z 259.2 (MH⁺).

Step 4 (Example 138): the benzotriazole from Step 3 was coupled to intermediate I (Ar=2,3-dichlorophenyl) using the procedure described in general method A.

Other examples of inhibitors prepared in a similar fashion using the appropriate amine in step 1 are listed under method I in Table 3.

### Preparation of Example 114

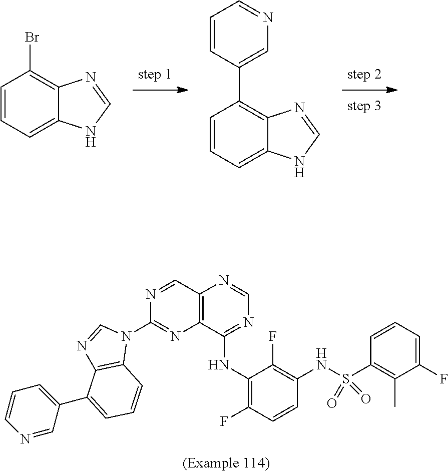 [SMILES: Brc1cccc2[nH]cnc12 ; c1cncc(-c2cccc3[nH]cnc23)c1 ; Cc1c(F)cccc1S(=O)(=O)Nc1ccc(F)c(Nc2ncnc3cnc(-n4cnc5c(-c6cccnc6)cccc54)nc23)c1F ; "(Example 114)"]

Step 1—Preparation of 4-(pyridine-3-yl)-1H-benzo[d]imidazole: Bromobenzimidazole (70 mg, 0.355 mmol), potassium carbonate (196 mg, 1.42 mmol) and 3-pyridylboronic acid (57 mg, 0.46 mmol) were charged in a 4 mL vial and dioxane (2 mL) and water (0.7 mL) were added. Argon gas was bubbled through the mixture for 1 minute and then tetrakis(triphenylphosphine)palladium (0) (16.4 mg, 0.014 mmol) was added. Argon gas was again bubbled through the solution for 3 min, the vial was sealed and heated at 100° C. for 2 hours (conversion to desired product is complete as judged by LCMS analysis). The reaction mixture was allowed to cool to RT, diluted with EtOAc and washed with brine. After drying on MgSO₄, the extract was concentrated under reduced pressure and the residue purified by flash chromatography using Et₃N pre-treated silica and a DCM-20% iPrOH/DCM gradient to provide the desired benzimidazole intermediate (58 mg, 84% yield): ¹H NMR (DMSO-d₆) δ: 12.71 (broad s, 1H), 9.24 (s. 1H), 8.57 (dd, J=5.1, 1.6 Hz, 1H), 8.43 (broad d, J=5.5 Hz, 1H), 8.31 (s, 1H), 7.61 (d, J=7.8 Hz, 1H), 7.52 (ddd, J=7.8, 4.7, 0.8 Hz, 1H), 7.47 (d, J=7.4 Hz, 1H), 7.34 (t, J=7.8 Hz, 1H). MS m/z 196.1 (MH⁺).

Step 2: thiomethyl derivative I (Ar=3-fluoro-2-methylphenyl; Example 90) was oxidized to a mixture of sulfoxide and sulfone as described in general method A.

Step 3 (Example 114): prepared from the benzimidazole derivative described in step 1 and sulfoxide/sulfone mixture from step 2 using general method A.

### Preparation of (R)-3-(difluoromethoxy)pyrrolidine hydrochloride

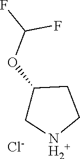

(R)—N-Boc-3-hydroxypyrrolidine was difluoromethylated using 2-fluorosulfonyl-2,2-difluoroacetic acid and copper(I)iodide catalysis as described in J. Org. Chem. 2016, 81, 5803 followed by removal of the N-Boc protecting group with 4N HCl in dioxane.

### Preparation of (S)-3-ethylpyrrolidine hydrochloride

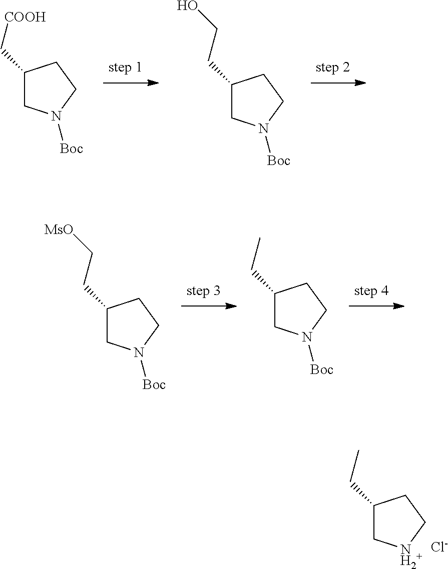

Step 1: commercially available (R)-2-(1-(tert-butoxycarbonyl)pyrrolidin-3-yl)acetic acid (2.0 g, 8.72 mmol) was dissolved in dry THF (25 mL) and 1M BH₃·THF (17.45 mL, 17.45 mmol) was added. The reaction mixture was stirred at RT for 3 h. It was then cooled in an ice-water bath and quenched by slow addition of 1N HCl. The product was extracted into EtOA and washed with saturated aqueous NaHCO₃ and brine. The organic layer was dried over anhydrous Na₂SO₄ and evaporated to dryness to provide (R)-tert-butyl 3-(2-hydroxyethyl)pyrrolidine-1-carboxylate (1.50 g, 6.98 mmol, 80% yield) as an oil: ¹H NMR (CDCl₃) δ: 3.62-3.70 (m, 2H), 3.37-3.60 (m, 2H), 3.15-3.33 (m, 1H), 2.88 (dt, J=18.4, 9.6 Hz, 1H), 2.13-2.35 (m, 1H), 1.94-2.08 (m, 1H), 1.47-1.69 (m, 3H), 1.45 (s, 9H), 1.30-1.43 (m, 1H), 0.93 (t, J=7.4 Hz, 1H).

Step 2: (R)-tert-butyl 3-(2-hydroxyethyl)pyrrolidine-1-carboxylate from step 1 (1.0 g, 4.64 mmol) was dissolved in DCM (15 mL) and triethylamine (1.55 mL, 11 mmol) was added. The mixture was cooled to 0° C. and a solution of methanesulfonyl chloride (0.58 mL, 7.4 mmol) in DCM (2 mL) was added dropwise. The reaction mixture was stirred at 0° C. for about 1 h. The reaction mixture was then diluted with DCM (15 mL) and washed with sat. NaHCO₃. The organic layer was separated, dried over anhydrous Na₂SO₄ and filtered. The filtrate was evaporated to dryness to give the crude mesylate (1.36 g) as an oil that was used in the next step without further purification: ¹H NMR (CDCl₃) δ: 4.26 (dt, J=11.3, 6.3 Hz, 2H), 3.39-3.66 (m, 2H), 3.21-3.35 (m, J=8.2, 8.2 Hz, 1H), 3.03 (s, 3H), 2.85-2.99 (m, 1H), 2.21-2.38 (m, 1H), 1.98-2.15 (m, 1H), 1.80-1.92 (m, 1H), 1.70-1.80 (m, 1H), 1.48-1.70 (m, 2H), 1.46 (s, 9H), 0.97 (t, J=7.2 Hz, 1H).

Step 3: The crude mesylate from Step 2 (0.60 g, 2.0 mmol) was dissolved in THF (10 mL) and the solution cooled in an ice-water bath. 1M Lithium triethylborohydride in THF (4.70 mL, 4.70 mmol) was added slowly after which the ice bath was removed, and the mixture was stirred for 2 h at RT. TLC (2:1, ethyl acetate/hexanes) showed no starting material. Methanol (5 mL) was added slowly to quench the reaction and the organic solvent was removed under reduced pressure. The crude reaction mixture was partitioned in EtOAc (30 mL) and water (15 mL) and the organic layer was washed with brine (10 mL). The organic layer was separated, dried over anhydrous Na₂SO₄, filtered and the filtrate was evaporated to dryness to provide the crude product. The material was purified on ISCO using a RediSep 12 g column (Hex/EtOAc) to provide (S)-tert-butyl 3-ethylpyrrolidine-1-carboxylate (300 mg, 73% yield) as an oil: ¹H NMR (CDCl₃) δ: 3.48 (tt, J=29.7, 9.4 Hz, 2H), 3.16-3.34 (m, 1H), 2.85 (dt, J=19.8, 10.1 Hz, 1H), 1.90-2.11 (m, 2H), 1.45 (s, 9H), 1.36-1.43 (m, 3H), 0.93 (t, J=7.4 Hz, 3H).

Step 4: the carboxylate from Step 3 (250 mg, 1.25 mmol) was dissolved in MeOH (2 mL) and 4M HCl in dioxane (2 mL, 8 mmol) was added. The reaction was stirred at RT for 2 h. Volatiles were then removed under reduced pressure, the residue azeotroped with ethylacetate to dryness and the residue dried under vacuum to provide (S)-3-ethylpyrrolidine hydrochloride (162 mg, 95% yield) as a thick oil.

### Preparation of (R)-3-(methoxymethyl)pyrrolidine hydrochloride

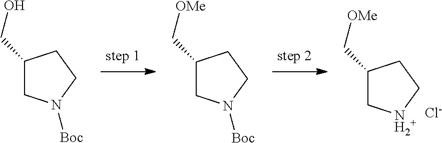 [SMILES: CC(C)(C)OC(=O)N1CC[C@@H](CO)C1 ; COC[C@@H]1CCN(C(=O)OC(C)(C)C)C1 ; COC[C@@H]1CC[NH2+]C1 ; [Cl-]]

Step 1: (R)-tert-butyl 3-(hydroxymethyl)pyrrolidine-1-carboxylate (1.00 g, 4.97 mmol) was dissolved in THF (20 mL) and the solution cooled in an ice-water bath. NaH 60% oil dispersion (0.60 g, 14.91 mmol) was added portion wise and the reaction mixture was stirred at 0° C. for 15 min. Iodomethane (1.55 mL, 24 mmol) was slowly added and the reaction mixture was stirred overnight at RT. The reaction mixture was then quenched with sat. NH₄Cl solution and extracted with EtOAc (2×50 mL). The organic layer was separated, dried over anhydrous Na₂SO₄ and filtered. The filtrate was evaporated to dryness and purified by silica gel column chromatography (80 g) using EtOAc-hex 0 to 100%. (R)-tert-butyl 3-(methoxymethyl)pyrrolidine-1-carboxylate (700 mg, 3.25 mmol, 65.4% yield) was obtained as an colorless oil: ¹H NMR (CDCl₃) δ: 3.49 (dd, J=11.0, 7.6 Hz, 1H), 3.43 (br s, 1H), 3.35 (s, 3H), 3.26-3.35 (m, 3H), 3.06 (dd, J=10.7, 7.3 Hz, 1H), 2.46 (spt, J=7.3 Hz, 1H), 1.91-2.02 (m, 1H), 1.58-1.70 (m, 1H), 1.46 (s, 9H).

Step 2: (R)-tert-butyl 3-(methoxymethyl)pyrrolidine-1-carboxylate from Step 1 (100 mg, 0.46 mmol) in DCM (2 mL) was mixed with 4M HCl in dioxane (2 mL, 8.0 mmol) and the mixture stirred at RT for 2 h. Volatiles were evaporated under reduced pressure, the residue co-evaporated to dryness with EtOAc and the product dried under vacuum to provide (R)-3-(methoxymethyl)pyrrolidine hydrochloride (70 mg) as an oil, that was used without further purification.

### Preparation of Example 89

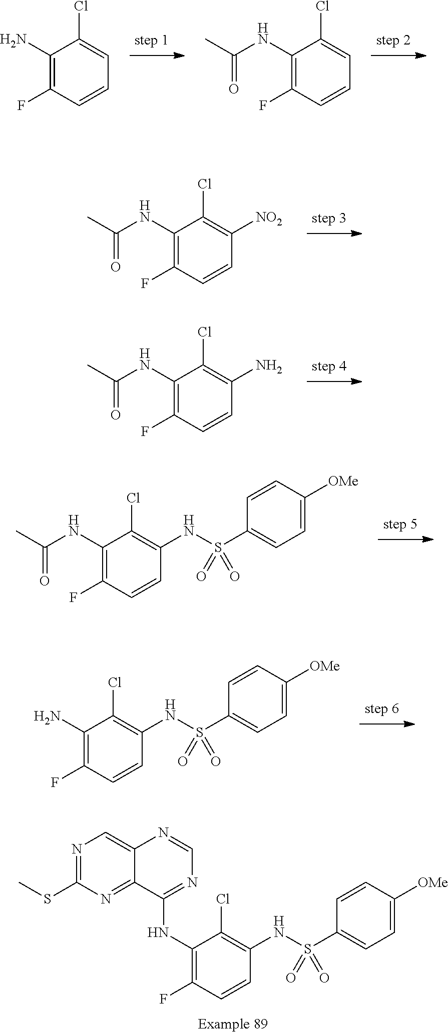

Step 1: 2-chloro-6-fluoroaniline (10 g) were charged in a 250 mL flask and dissolved in 40 mL of glacial acetic acid. Acetic anhydride (7.47 mL) were added at room temperature and the resulting mixture was stirred at 90° C. for 1 h at which point LCMS analysis revealed the reaction was complete. Volatiles were removed under reduced pressure, the residue was dissolved in DCM and slowly neutralized with a saturated solution of NaHCO₃. The layers were separated, and the aqueous layer was extracted 3× with DCM. The combined organic layers were washed once with water then dried over MgSO4, filtered and concentrated. After vacuum drying, the desired product was obtained as white to pale pink crystals (12.79 g): ¹H NMR (CDCl₃) δ: 7.16-7.26 (m, 2H), 7.03-7.13 (m, 1H), 6.93 (br s, 1H), 2.23 (br s, 3H). MS m/z 188.1 (M H⁺).

Step 2: The acetanilide from step 1 (12.75 g) was taken up in 25 mL of conc. sulfuric acid and cooled to 0° C. in an ice bath. Nitric acid (90%, 3.31 mL) was slowly added. After 5-10 minutes the mixture became a solid mass. It was allowed to warm to room temperature which produced a thick burgundy sludge. After a total of 4 h, the reaction was monitored by LCMS which revealed some remaining starting material. Another 5 mL of sulfuric acid was added to improve fluidity followed by 0.3 mL of 90% nitric acid. The mixture was allowed to stir at room temperature for another 18 hours. The mixture was then cooled to 0° C. and poured on crushed ice (˜ 150 mL). Once the ice had melted, the suspension was sonicated, and the yellow solids were collected by filtration, washed with water and dried (15.1 g of crude product). The crude solid was taken into 50 mL of acetonitrile and brought to reflux affording a clear dark red solution. The heating was stopped, and the mixture was allowed to cool to room temperature over 1 hour then stirred at room temperature for 2 h. By that time the mixture had become a solid mass which was broken up with a spatula and sonicated. The solids were then collected by filtration and washed with a small amount of cold acetonitrile. The desired off-white nitro compound (6.63 g) was obtained as a single regioisomer as shown by NMR (mother liquors yielded a second crop of 2.16 g that contained 7% of 6-chloro-2-fluoro-3-nitroacetanilide): ¹H NMR (CDCl₃) δ: 7.90 (dd, J=9.2, 4.9 Hz, 1H), 7.24 (dd, J=9.2, 8.4 Hz, 1H), 6.98 (br s, 1H), 2.28 (s, 3H). MS m/z 233.0 (MH⁺).

Step 3: To a solution of the nitroacetanilide from Step 2 (500 mg, 2.15 mmol) in 15 mL of ethanol was added a solution NH₄Cl (60 mg, 1.12 mmol) in 1.35 mL of water. The mixture was warmed to 70° C. then iron powder (600 mg, 10.75 mmol) was added in three portions, 10 minutes apart. The resulting dark red to burgundy mixture was stirred at 70° C. for 20 h. LCMS of a filtered aliquot of the reaction mixture at that point revealed the reaction was complete. The mixture was filtered through a pad of Celite®. The dark brown filtrate was concentrated to dryness then taken into EtOAc to which was added MgSO₄. The suspension was stirred then filtered affording a clear pale yellow solution. The solution was concentrated to dryness to afford the desired product (440 mg) as a pale yellow solid that was used as such without further purification: ¹H NMR (CDCl₃) δ: 6.92 (t, J=9.0 Hz, 1H), 6.76 (br s, 1H), 6.68 (dd, J=8.6, 4.7 Hz, 1H), 3.98 (br s, 2H), 2.24 (br s, 3H). MS m/z 203.1 (MH⁺).

Step 4: The aniline from Step 3 was sulfonylated using 4-methoxybenzenesulfonyl chloride in the usual manner as described for A-9 in Scheme A: ¹H NMR (DMSO-d₆) δ: 9.89 (s, 1H), 9.67 (s, 1H), 7.57-7.74 (m, 2H), 7.23 (t, J=9.2 Hz, 1H), 7.13 (dd, J=8.8, 5.3 Hz, 1H), 7.02-7.10 (m, 2H), 3.82 (s, 3H), 2.00 (s, 3H). MS m/z 373.0 (MH⁺).

Step 5: the acetanilide from Step 4 (200 mg, 0.54 mmol) was taken into 1.5 mL of ethanol then a 1:1 mixture of concentrated HCl and water (2 mL) was added slowly. The yellow slurry was then brought to 80° C. and stirred for 1 h. At that point, 1 mL of ethanol was added to improve the solubility. The mixture was allowed to stir at the same temperature for another 5 h (the mixture had become a clear yellow solution by then). LCMS analysis at that point showed <3% of remaining starting material. The mixture was concentrated to remove the majority of the ethanol then cooled on ice. It was basified with 4N NaOH to pH 5-6. The resulting suspension was sonicated, and the solids were collected by filtration and washed with water. After drying under reduced pressure, 156 mg of the desired product were obtained as a beige solid: ¹H NMR (DMSO-d₆) δ: 9.53 (s, 1H), 7.53-7.72 (m, 2H), 7.00-7.14 (m, 2H), 6.95 (dd, J=10.8, 8.8 Hz, 1H), 6.36 (dd, J=8.6, 5.1 Hz, 1H), 5.39 (s, 2H), 3.81 (s, 3H). MS m/z 329.0 (M−H).

Step 6: using the protocol described in Scheme A for the preparation of inhibitors of formulae I from synthetic intermediate A-5 (Step 1, Example 1), the aniline from Step 5 was reacted with intermediate A-10 to provide Example 89.

### Preparation of Examples 135

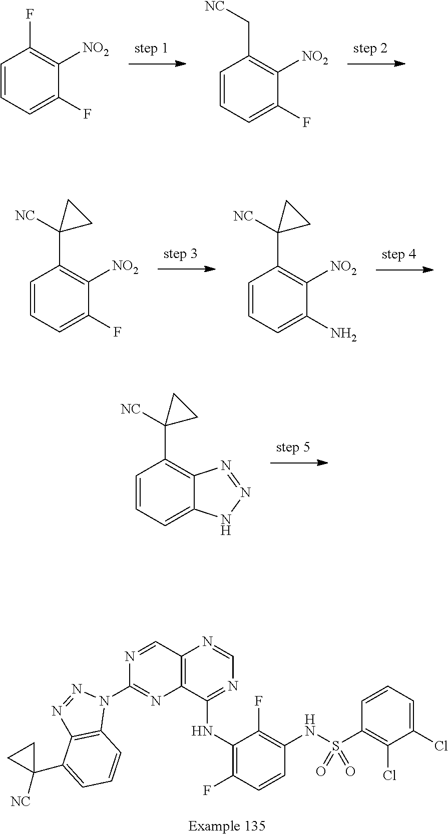

Step 1: To a 100 mL round-bottom flask, was added 2,6-difluoronitrobenzene (1.3 mL, 12.6 mmol) and ethyl cyanoacetate (1.6 mL, 15.1 mmol) in DMF (15 mL). Next, sodium hydride (754 mg, 18.9 mmol) was added slowly at room temperature. The reaction was allowed to stir at room temperature for 15 min. The reaction was quenched with 1M HCl until the deep-red solution turned yellow and then diluted in EtOAc. The organic layer was separated then washed with aq. NH₄Cl, followed by brine. The organic layer was dried by MgSO4, filtered and then concentrated under reduced pressure. The crude material was dissolved in DMSO (9 mL) and water (1 mL) and transferred to a 20 mL microwave vial. The reaction was heated to 120° C. and left to stir for 16 h. The reaction mixture was cooled to room temperature and diluted in EtOAc then washed with aq. NH₄Cl followed by brine. The organic layer was dried by MgSO4, filtered and then concentrated under reduced pressure. The crude material was purified by normal phase, flash column chromatography using hexanes:EtOAc to afford the desired benzylic nitrile (2.12 g, 93%) as a orange solid. ¹H NMR (400 MHz, DMSO-d₆) δ: 7.80 (td, J=7.8, 5.5 Hz, 1H), 7.65 (t, J=9.6 Hz, 1H), 7.54 (d, J=7.4 Hz, 1H), 4.28 (s, 2H). MS m/z 725.4 (MH⁺). MS m/z 181.2 (MH⁺).

Step 2: the phenylacetonitrile derivative from step 1 (200 mg, 1.11 mmol) and DMSO (4 mL) was charged in a 25 mL flask. Then diphenyl(vinyl)sulfonium-trifluoromethanesulfonate (479 mg, 1.32 mmol) was added followed by 1,8-diazabicyclo[5.4.0]undec-7-ene (0.37 ml, 2.48 mmol) at room temperature. The mixture was stirred at that temperature for 16 h. Upon completion, an aqueous solution of NH₄Cl was added and the aqueous layer was extracted with EtOAc. The organic layer was washed with water and once with brine. The organic layer was dried over MgSO₄, filtered and concentrated under vacuum. The resulting residue was purified by flash chromatography through a column of silica gel using EtOAc in hexanes to yield the desired cyclopropane derivative (138 mg, 60% yield). ¹H NMR (400 MHz, CDCl₃) δ: 7.47-7.59 (m, 1H), 7.29-7.41 (m, 2H), 1.69-1.85 (m, 2H), 1.29-1.39 (m, 2H). MS m/z 207.2 (MH⁺).

Step 3: To a 10 mL microwave vial, was added the cyclopropane derivative of step 2 (138 mg, 0.67 mmol). Next, 3 mL of concentrated ammonium hydroxide was added to the reaction. The reaction was heated in the microwave at 130° C. for 1 h. Upon completion, the reaction was diluted with water then extracted with EtOAc. The organic layer was washed with brine, dried over MgSO₄, filtered and then concentrated under reduced pressure to afford 1-(3-amino-2-nitro-phenyl)cyclopropanecarbonitrile (120 mg, 88% yield) as an orange solid. ¹H NMR (400 MHz, CDCl₃) δ: 7.27 (d, J=15.26 Hz, 1H), 6.83 (d, J=8.38 Hz, 1H), 6.86 (d, J=7.50 Hz, 1H), 5.36 (br s, 2H), 1.71 (br s, 2H), 1.24 (br s, 2H).

Step 4: reduction and ring closure of the intermediate 1,2-phenylenediamine to the benzotriazole ring was performed using the procedure described in Steps 2 and 3 of general method I for example 138.

Step 5 (Example 135): the benzotriazole from Step 4 was coupled to intermediate I (Ar=2,3-dichlorophenyl) using the procedure described in general method A.

### Preparation of Examples 137

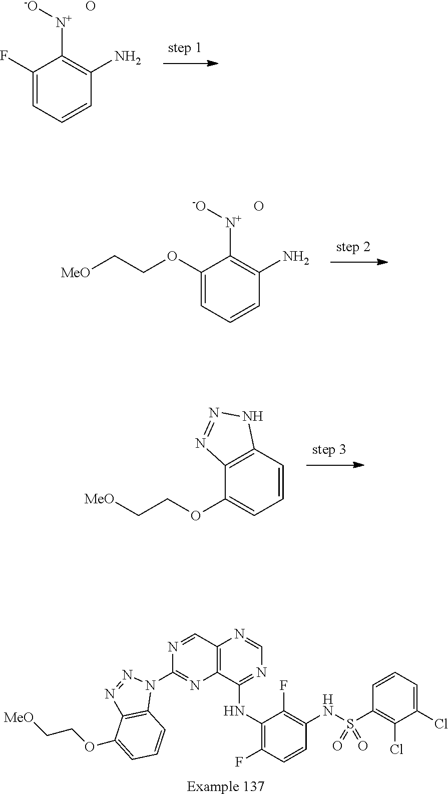

Step 1: Potassium carbonate (0.35 g, 2.56 mmol) and 2-methoxyethanol (0.40 mL, 5.1 mmol) were added to a solution of 3-fluoro-2-nitro-aniline (0.10 g, 0.641 mmol) in DMF (3.2 mL). The resulting mixture was stirred at 80° C. for 10 h. Water was added and the aqueous mixture was extracted with EtOAc. The org layers were combined, washed with brine, dried with NA₂SO₄, filtered and concentrated. The crude was purified by column chromatography (silica gel, 0-100% EtOAc in hexanes) to afford 3-(2-methoxyethoxy)-2-nitro-aniline (52 mg, 38% yield). MS m/z 213.1 (MH⁺).

Step 2: reduction and ring closure of the intermediate 1,2-phenylenediamine to the benzotriazole ring was performed using the procedure described in Steps 2 and 3 of general method I for example 138.

Step 3 (Example 137): the benzentriazole from Step 2 was coupled to intermediate I (Ar=2,3-dichlorophenyl) using the procedure described in general method A.

### Preparation of Examples 160 to 163 (Table 5)

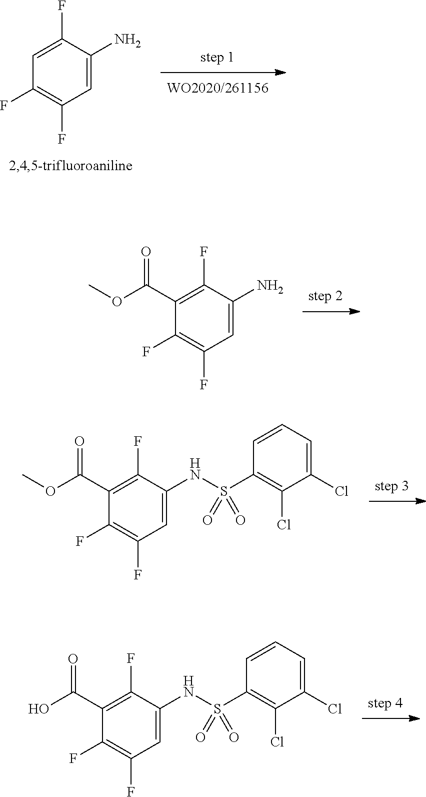 [SMILES: Nc1cc(F)c(F)cc1F ; COC(=O)c1c(F)c(N)cc(F)c1F ; COC(=O)c1c(F)c(F)cc(NS(=O)(=O)c2cccc(Cl)c2Cl)c1F ; O=C(O)c1c(F)c(F)cc(NS(=O)(=O)c2cccc(Cl)c2Cl)c1F]

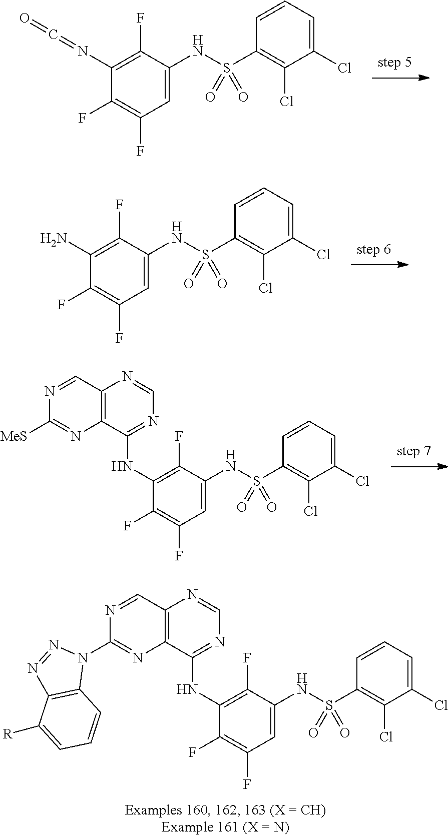

Step 1: carbomethoxylation of 2,4,5-trifluoroaniline was performed as described in patent WO2020/261156.

Step 2: To a solution of methyl 3-amino-2,5,6-trifluorobenzoate (2.72 g, 13.26 mmol) in DCE/pyridine (1:1, 16 mL) was added 2,3-dichlorobenzenesulfonyl chloride (3.91 g, 15.9 mmol) portion wise at rt. The reaction was heated to 70° C. for 16 h. The reaction was monitored by LCMS. When the reaction was completed, it was quenched with HCl 1 M. The aqueous layer was extracted with EtOAc (15 mL) three times. The combined organic layers were washed with brine, dried over MgSO₄, filtered and evaporated to dryness. The residue was purified by chromatography on a silica gel column using 0-30% EtOAc in hexanes. The pure fractions were collected and evaporated to give methyl 3-((2,3-dichlorophenyl)sulfonamido)-2,5,6-trifluorobenzoate (5.14 g, 91% yield) as a light brown solid: m/z=412.0.

Step 3: To a solution of methyl 3-((2,3-dichlorophenyl)sulfonamido)-2,5,6-trifluorobenzoate from step 2 (5.14 g, 12.4 mmol) in 30 mL of THF:MeOH (5:1) was added 2M KOH (37 mL, 74.5 mmol) at rt. The reaction was stirred at rt overnight. When the reaction was completed, it was evaporated to dryness and residue was added water (30 mL) and diethyl ether (30 mL). The aqueous layer was washed with ether (20 mL) twice. The aqueous layer was acidified with HCl 1M to pH=2. The aqueous layer was extracted with EtOAc (30 mL) thrice. The combined organic layers were washed brine, dried over MgSO₄, filtered and evaporated to give 3-((2,3-dichlorophenyl)sulfonamido)-2,5,6-trifluorobenzoic acid (4.5 g, 91% yield) as a light orange oil. The compound was used as such in the next step: m/z=398.0.

Step 4: To a solution of 3-((2,3-dichlorophenyl)sulfonamido)-2,5,6-trifluorobenzoic acid from step 3 (4.50 g, 11.2 mmol) in acetonitrile (30 mL) was added triethylamine (1.71 mL, 12.37 mmol) and diphenyl phosphoryl azide (2.91 mL, 13.50 mmol) at rt. The reaction was heated to 80° C. overnight. The reaction was cooled down to rt and water (30 mL) was added. The aqueous layer was extracted with EtOAc (30 mL) three times. The combined organic layers were washed with brine, dried over MgSO₄, filtered and evaporated into a dark yellow residue. The crude material was purified by chromatography on a silica gel column using 0-50% EtOAc in hexanes. The pure fractions were collected and evaporated to give 2,3-dichloro-N-(2,4,5-trifluoro-3-isocyanatophenyl)benzenesulfonamide (2.29 g, 51% yield) as a brown solid: m/z=391.0.

Step 5: To a solution of 2,3-dichloro-N-(2,4,5-trifluoro-3-isocyanatophenyl)benzenesulfonamide from step 4 (1.35 g, 3.39 mmol) in THF (17 mL) was added aqueous LiOH 4M (17 mL). The pressure vessel was screwed and heated to 100° C. for 1 h in an oil bath. When the reaction was completed, it was quenched with NH₄Cl sat. EtOAc was added and the layers were allowed to separate. The aqueous layer was extracted with EtOAc (20 mL) two more times. The combined organic layers were washed with brine, dried over MgSO₄, filtered and evaporated to give N-(3-amino-2,4,5-trifluorophenyl)-2,3-dichlorobenzenesulfonamide (1.09 g, 86% yield) as a brown solid. The compound was carried to the next step without further purification: ¹H NMR (400 MHz, DMSO-d₆) δ: 10.59 (br s, 1H), 7.94 (dd, J=8.2, 1.6 Hz, 1H), 7.89 (dd, J=8.2, 1.6 Hz, 1H), 7.51 (dd, J=8.0 Hz, 1H), 6.34-6.43 (m, 1H), 5.72 (s, 2H). m/z=369.0.

Step 6: the aniline of step 5 was converted to the pyrimidopyrimidine as described under general Method A (Example 36) where Ar=2,6-dichlorophenyl.

Step 7 (Examples 160-163, Table 5): the thiomethyl intermediate from step 6 was converted to inhibitors following the protocol in general method A, using the appropriately substituted benzimidazoles (Example 160, 162, 163) or benzotriazole (Example 161).

### Biological Activity

### In Vitro Biological Activity

### (a) Kinase Activity Assays for BRAF, CRAF and ARAF

Compound preparation: solid samples of each substances in 1dram vials were suspended in DMSO (Fisher Scientific) at a stock concentration of 20 mM. Stocks were kept at −20° C. and protected from light. If solubility of the compound at 20 mM appeared to be an issue, the initial concentration of the DMSO stock was changed to 10 mM or 5 mM.

In vitro enzymatic reactions were used for evaluating compounds intrinsic activity against BRAF, CRAF and ARAF. For BRAF and CRAF, 0.375 nM of purified GST-tagged kinases (cat #B4062-10UG and cat #R1656-10UG respectively from Millipore Sigma) were incubated with 75 nM of kinase-dead MEK1 substrate (cat #40075; BPS Bioscience) in the presence of 10 μM of Ultrapure ATP (cat #V9102; Promega; part V915A) with and without the test compound in a buffer containing 50 mM HEPES pH 7.5, 10 mM MgCl₂, 1 mM EDTA, 0.01% Brij-35 and 2 mM DTT. Separate reactions were performed with the MEK1 substrate and ATP as a blank control. ARAF, kinase reactions were rigorously the same with the exception that kinase concentration was raised to 3.75 nM (cat #1768-0000-1; Reaction Biology).

For compound treatment, 5 μL/well of test substance solution are placed in a 384-well proxyplate (Perkin Elmer) and mixed with 2× concentrated kinase reactions. The dilution series is selected so that nine concentrations cover a range from 100 nM to 0.01 nM. If necessary (if the compound exhibits low intrinsic potency) the initial concentration of 100 nM is changed to 1 μM, or 0.5 μM and further dilutions are carried out accordingly. The final concentration of DMSO in the assay is set at 0.05%.

BRAF and CRAF kinase reactions were carried out for a total of 2 hours at 30° C. and then stopped by ½ dilution in ADP-Glo Reagent (cat #V9102; Promega; part V912C). Reactions were then incubated for 1 h at room temperature before addition of one volume of Kinase Detection Reagent (cat #V9102; Promega; part V917A). Plates were then equilibrated at room temperature for 30 minutes before detection of luminescence on a Synergy Neo2 plate reader (Biotek). The effect of each compound dilution on BRAF and CRAF kinase activity was expressed as % inhibition and calculated as follows. First, the internal 100% inhibition control (average of luminescence in kinase reactions comprising kinase dead MEK1 substrate alone) was subtracted from each data point. The average of DMSO (vehicle) controls (set as 0% inhibition) was established and used to calculate % inhibition:

$$\% inhibition = 100 * ( 1 - ( ( Luminescence {signal}_{compound} ) / ( Luminescence {signal}_{DMSO} ) ) )$$

ARAF kinase reactions were carried out for a total of 2 hours at 30° C. and then stopped by addition of EDTA at a final concentration of 40 mM. Reactions were then detected using the AlphaLISA® SureFire® Ultra™ p-MEK ½ (Ser218/222) (PerkinElmer) kit. Reactions were performed with 5 μL of kinase reaction according to the manufacturer's specifications in 384-well proxyplates (Perkin Elmer) followed by overnight incubation of the reactions at room temperature in a humidified chamber. After completion of the detection reactions, the signals were recorded on a Synergy Neo2 plate reader (Biotek) equipped with AlphaLISA® filters. The effect of each compound dilution on the pMEK signal generated by ARAF reactions was expressed in % inhibition and calculated as follows. An internal 100% inhibition control (average of luminescence in kinase reactions comprising kinase dead MEK1 substrate alone) was included in each plate to measure of pMEK background and was subtracted from each data point. The average of DMSO (vehicle) controls (set as 0% inhibition) was also established and used to calculate % inhibition:

$$\% inhibition = 100 * ( 1 - ( ( pMEK {signal}_{compound} ) / ( pMEK {signal}_{DMSO} ) ) )$$

IC₅₀ values were obtained by plotting the kinase inhibition values and fitting the dose-activity curves using a log(agonist) versus response−variable slope (four parameters) function using either GraphPadPrism (V7.0) or Dotmatics Screening Ultra platform. Standards included in the ARAF kinase assay were Belvarafenib (MedChem Express cat #HY-109080; CAS #1446113-23-0), LXH254 (MedChem Express cat #HY-112089; CAS #1800398-38-2) and BGB283 (cat #HY-18957; CAS #1446090-79-4).

All substances reported here are thus BRAF, CRAF and ARAF ATP-competitive kinase inhibitors as demonstrated by direct inhibition of enzymatic activity in vitro. BRAF and CRAF inhibition potencies of compounds are listed in Tables 2-5 while ARAF kinase inhibition potencies of representative analogs are listed in Table A. Preferred Examples as defined in the embodiments show BRAF IC₅₀ values<10 nM and even more preferred Examples have BRAF IC₅₀ values<1 nM. Preferred Examples as defined in the embodiments show CRAF IC₅₀ values<50 nM and even more preferred Examples have CRAF IC₅₀ values<10 nM.

TABLE A
ARAF kinase inhibition results

| | Kinase |
|---|---|
| | IC₅₀ (nM) |
| Example | ARAF |
| 98 | ** |
| 112 | * |
| 114 | *** |
| 119 | ** |
| 128 | * |
| 129 | ** |
| Belvarafenib | * |
| BGB283 | * |
| LXH254 | * |

For the ARAF biochemical kinase assay,
* denotes an IC50 > 20 nM,
** denotes a 10-20 nM IC50 range and
*** denotes an IC50 < 10 nM.

### (b) General Cell Culture Methods

All cancer cell lines (A375, A101D, A2058, RKO, HT29 SK-MEL 30, IPC298, HepG2, HCT-116, Lovo, SW620, SW480, NCI-H358, NCI-H2122, Calu-6, NCIH2087, NCIH1755, NCIH1666 and Mewo) were obtained from ATCC and cultured in RPMI-1640 medium (Gibco) supplemented with 5% heat inactivated fetal bovine serum (FBS, Wisent) at 37° C. under 5% CO₂. Cells were maintained in T175 flasks (Greiner). They were passaged by removing the culture medium, washing once in 10 mL of room temperature Phosphate Buffered Saline (PBS; Wisent) and incubating at 37° C. with 2 mL of 0.05% Trypsin (Thermo-Fisher). Trypsin was then inactivated by adding complete growth medium and the cells were then replated in a T175 culture dish at the appropriate dilution. All cell lines were routinely tested for mycoplasma contamination. Tissue type and mutational status of each cell line can be found in Table B.

TABLE B
Tumor type and RAS-ERK pathway mutational status of cancer
cell lines (CCLs) used for pERK and antiproliferative
profiling of substances described in this application.

| Cell line | Tissue type | Mutational status |
|---|---|---|
| A375 | Skin | BRAF V600E |
| A101D | Skin | BRAF V600E |
| A2058 | Skin | BRAF V600E |
| RKO | Colon | BRAF V600E |
| HT29 | Colon | BRAF V600E |
| NCIH2087 | Lung | BRAF L597V; KRAS Q61K |
| NCIH1755 | Lung | BRAF G469A |
| NCIH1666 | Lung | BRAF G466V |
| SK-MEL30 | Skin | NRAS-Q61K |
| IPC298 | Skin | NRAS-Q61L |
| HepG2 | Liver | NRAS-Q61L |
| HCT-116 | Colon | KRAS G13D |
| Lovo | Colon | KRAS-G13D |
| SW620 | Colon | KRAS-G12V |
| SW480 | Colon | KRAS G12D |
| NCI-H358 | Lung | KRAS-G12C |
| NCI-H2122 | Lung | KRAS-G12C |
| Calu-6 | Lung | KRAS Q61K |
| Mewo | Skin | NF1 LOF |

(c) Measurement of Phospho-ERK Inhibition in Cultivated Human Cancer Cell Lines by the AlphaLISA® SureFire® Ultra™ p-ERK ½ (Thr202/Tyr204)

AlphaLISA® SureFire® Ultra™ p-ERK ½ (Thr202/Tyr204) analysis was conducted on cells plated in 100 μL of complete RPMI-1640 growth medium in 96-well flat-bottomed transparent dishes (Costar) at a density indicated in Table C. Cells were maintained overnight at 37° C. under 5% CO₂ before treatment with compounds' dilution series for one hour. The cell density in cells/cm² corresponds to cell number divided by the area of one well of a 96-well plate (0.143 cm²).

TABLE C
For each cancer cell line, number of cells plated per well.

| Cell line | Number of cells/well |
|---|---|
| A375 | 15,000 |
| NCIH2087 | 30,000 |
| NCIH1755 | 30,000 |
| NCIH1666 | 30,000 |
| SK-MEL30 | 24,000 |
| IPC298 | 20,000 |
| HepG2 | 20,000 |
| HCT-116 | 22,000 |
| Lovo | 20,000 |
| SW620 | 30,000 |
| SW480 | 15,000 |
| NCI-H358 | 20,000 |
| NCI-H2122 | 24,000 |
| Calu-6 | 15,000 |
| Mewo | 20,000 |

In a dilution series 100 μL/well of test substance dilution prepared in complete RPMI-1640 growth media was added to the cells. The dilution series is selected so that ten concentrations cover a range from 30 μM or 10 μM to 0.33 nM. If necessary, the initial concentration of 10 μM is increased to 100 μM or lowered to 1 μM (as in the case of A375 and NCIH1666 cells, which are generally more sensitive to the compounds) and further dilution is carried out accordingly. The final concentration of DMSO in the assay is set at 0.5%.

After treatment, media was removed, and cells were lysed in 50 μL of 1× AlphaScreen Ultra Lysis Buffer (Perkin Elmer). The AlphaLISA® SureFire® Ultra™ p-ERK ½ (Thr202/Tyr2O4) (PerkinElmer) reactions were performed with 5 μL of cell lysate according to the manufacturer's specifications in 384-well proxyplates (Perkin Elmer) followed by overnight incubation of the reactions at room temperature in a humidified chamber. After completion of the reactions, the signals were recorded on an EnVision plate reader (Perkin Elmer) using built in AlphaLISA® settings.

The effect of each compound dilution on the pERK signal was expressed in % inhibition and calculated as follows. An internal 100% inhibition control (1 μM trametinib, cat. No. HY-10999; MedChem Express; CAS No. 871700-17-3) was included in each plate and used as a measure of pERK background. First, the value obtained for trametinib was subtracted from each data point. The average of DMSO (vehicle) controls (set as 0% inhibition) was established and used to calculate % inhibition:

$$\% inhibition = 100 * ( 1 - ( ( pERK {signal}_{compound} ) / ( pERK {signal}_{DMSO} ) ) )$$

The ability of each compound to inhibit pERK signal was expressed as an IC₅₀ value obtained by plotting the inhibition values for each data point of a dilution series and fitting the obtained curves using a log(agonist) versus response−variable slope (four parameters) function using GraphPadPrism (V7.0).

When present, paradoxical pERK induction is inferred from the negative % inhibition values observed in the pERK IC₅₀ curves of a compound. To classify a compound as a pERK paradoxical inducer, the % inhibition of the minimal data point of the dosage-activity curve (% Y_{MIN}) was set to be lower than −20% which is considered to be within expected assay variation (e.g. a compound with % Y_{MIN}=−30% or −50% or −150% is considered to produce a paradoxical induction of the pathway. A compound that displays an IC₅₀ curve with a Y_{MIN}=−10% is considered not to produce a paradoxical activation of the pathway). Therefore, a compound was said to inhibit the pathway without paradoxical induction in a given cell line when the following criteria were met:
1. % inhibition at the highest tested dose (30 μM, 10 μM or 1 μM) exceeded 50%.
2. % Y_{MIN} of IC₅₀ curves was greater than −20%; where Y_{MIN} corresponded to the data point having the lowest value in the IC₅₀ curve of the said compound.

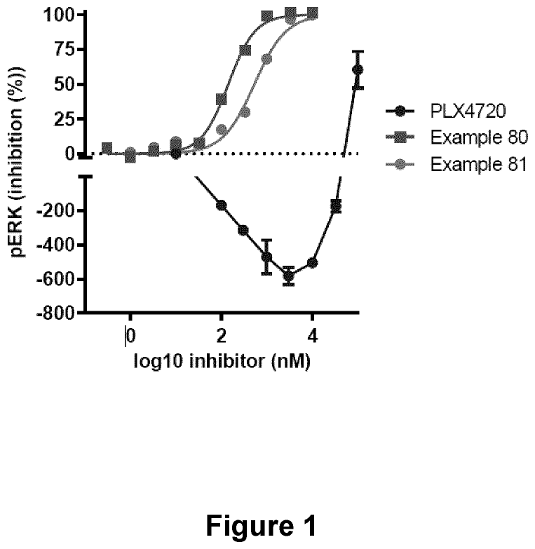

It is well known to someone skilled in the art that some variation of inhibition values is expected in such experiments. Y_{MIN} values of ±20% are considered within experimental error and are not significant. Therefore, only compounds with negative values in excess of assay variation (ca.>20%) are considered to induce a paradoxical activation of the signaling cascade and are not included within the scope of the present disclosure. FIG. 1 provides a visualization of the IC₅₀ curves for a compound that induces paradoxical pathway activation (PLX4720, commercially available from Selleck Chemicals; CAS #918505-84-7) and representative compounds as herein described exhibiting the unexpected and distinct induction-free profile.

FIG. 1 shows representative IC₅₀ inhibition dose response curves for compounds as described herein that do not induce paradoxical induction of pERK signaling (Y_{MIN}>−20%) in RAS-mutant HCT116 cells (Examples 80 and 81) and a compound (PLX4720) that causes strong induction of the pathway in the same cell line (Y_{MIN}˜−600%).

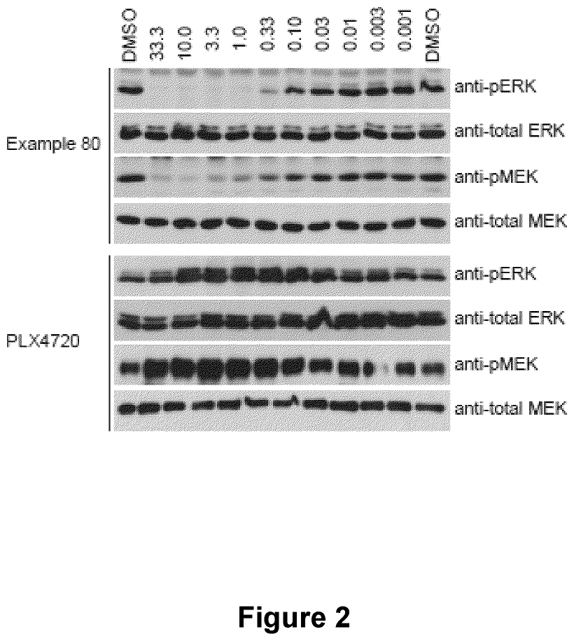

Significantly, compounds as herein defined do not induce paradoxical activation of the pathway according to the criteria described above. Further illustration of this highly desirable property can be illustrated using immunoblotting analysis as described below and depicted in FIG. 2 for the induction free compound (Example 80) and the inducer PLX4720.

For immunoblotting analysis, 500,000 HCT-116 cells were plated in 1 mL of complete RPMI-1640 growth medium in 24-well flat-bottomed transparent dishes (Costar). Cells were maintained overnight at 37° C. under 5% CO₂ before treatment with compounds' dilution series for one hour. Cells were then washed once in PBS and lized in 250 μL of Igepal Lysis Buffer (50 mM Tris-HCl pH 7.5, 150 mM NaCl, 1% Igepal-CA630, 1 mM EDTA, 10% Glycerol) supplemented with Leupeptin, Aprotinin, PMSF, phosphatase inhibitor cocktail (Sigma) and Na₃VO₄ at 4° C. for 15 minutes with gentle rocking. Cell extracts were then cleared by centrifugation at 20,000 g at 4° C. for 10 minutes. Cleared lysates were then transferred on ice in fresh tubes and then boiled in sample loading buffer (100 mM Tris-HCl pH 6.8, 4% SDS, 0.2% bromophenol blue, 20% glycerol, 200 mM β-mercaptoethanol) for 5 minutes prior to fractionation by SDS-PAGE and transfer to nitrocellulose membranes (PALL). Membranes were blocked for 1 hour in Tris Buffered Saline 0.2% Tween-20 (TBST; 10 mM Tris-HCl pH 8.0, 0.2% Tween-20, 150 mM NaCl) containing 2% BSA (Sigma) and then incubated at 4° C. overnight with a dilution of the following primary antibodies prepared in TBST:anti-pERK (1:2000 dilution; Sigma-Aldrich; cat. number M9692), anti-total ERK (1:1000 dilution; Cell Signaling Technology; cat. number 4695), anti-pMEK (1:1000 dilution; Cell Signaling Technology; cat. number 9121) and anti-total MEK (1:1000 dilution; Cell Signaling Technology; cat. number 9122). Secondary anti-mouse-HRP and anti-rabbit-HRP (Jackson Immunoresearch Labs; cat. number 115-035-146 and 111-035-144, respectively) were prepared in TBST at 1:5000 and 1:10000 dilutions, respectively. Immunoblots were revealed by exposition to X-ray films after one minute of incubation in ECL reagent.

FIG. 2 shows results of immunoblot analysis of RAS-mutant HCT-116 cells treated with a representative compound (Example 80; top panels) that does not induce paradoxical induction of pERK or pMEK signaling and by comparison, a compound (PLX4720; bottom panels) that induces the pathway in the same cell line. Total MEK and total ERK signals were also probed by immunoblotting to ensure equal loading of protein samples across conditions. Compound concentrations in micromolar are indicated above the immunoblot panels. The concentration range used for treatment was the same for Example 80 and PLX4720.

Example compounds 1 to 163 show pERK inhibition activity in the colon G13D Ras-mutated HCT-116 cell line as shown in Tables 2-5. In addition, some Examples were also shown to display paradoxical induction-free inhibition of pERK signaling in the SW480 colon cell line harboring the G12D allele of KRAS (Tables 2-5). Furthermore, some Examples from Tables 2-5 were also tested for inhibition of pERK in A375 cells that comprise the BRAF^{V600E} driver mutation and were found to be active as well (Table D). In Tables 2-5, all compound Examples 1-163 displayed pERK IC₅₀ values in the HCT116 cell line that were <30 μM. Preferred compounds had IC₅₀ values 1-10 μM, more preferred compounds had IC₅₀ values 0.5-1 μM, while even more preferred compounds had IC₅₀ values<0.5 μM.

Representative compounds as herein defined were also tested on additional tumor cells for their pERK inhibition activity and showed good to very good pERK inhibition activity in cancer cell lines bearing various NRAS, KRAS and NF1 alleles and representative of a large diversity of tissue types (i.e., SK-MEL 30, IPC298, HepG2, HCT-116, Lovo, SW620, SW480, NCI-H358, NCI-H2122, Calu-6 and Mewo; Table D and refer to Table B for genotypes). The pERK inhibition activity of compounds is stronger in cancer cell lines bearing BRAF alleles (A375, A101D, A2058, RKO, HT29, NCIH2087, NCIH1755 and NCIH1666) (Table D).

Table D (D-1 and D-2). Induction-free PERK IC₅₀ values and anti-proliferative EC₅₀ values for selected compounds in a panel of RAS-mutant cancer cell lines (see Table B for genotypes) and BRAF^{V600E} mutant A375.

D-1.

| | | | | | | SK- | | | |
|---|---|---|---|---|---|---|---|---|---|
| | A375 | A2058 | HT-29 | RKO | A101D | MEL30 | IPC298 | HepG2 | HCT116 |
| | pERK | pERK | pERK | pERK | pERK | pERK | pERK | pERK | pERK |
| Ex. | Prolif. | Prolif. | Prolif. | Prolif. | Prolif. | Prolif. | Prolif. | Prolif. | Prolif. |
| 98 | *** | *** | *** | *** | *** | +++ | ++ | +++ | ++ |
| 98 | | | | | | (10) | (13) | (27) | (−6.0) |
| | | | | | | *** | *** | *** | *** |
| 112 | *** | *** | *** | *** | *** | +++ | ++ | ++ | + |
| 112 | | | | | | (6) | (1) | (−10) | (−0.8) |
| | | | | | | *** | | *** | ** |
| 114 | *** | *** | *** | *** | *** | ++ | ++ | ++ | + |
| 114 | | | | | | (−9) | (1) | (14) | (−4.9) |
| | | | | | | ** | *** | *** | ** |
| 119 | *** | *** | *** | *** | *** | ++ | ++ | ++ | + |
| 119 | | | | | | (−12) | (−4) | (30) | (−7) |
| | | | | | | *** | | *** | ** |
| 128 | *** | *** | *** | *** | *** | +++ | ++ | +++ | ++ |
| 128 | | | | | | (13) | (8) | (34) | (7) |
| | | | | | | *** | *** | *** | *** |
| 129 | *** | *** | *** | *** | *** | +++ | ++ | ++ | ++ |
| 129 | | | | | | (16) | (17) | (−2) | (−8) |
| | | | | | | *** | *** | *** | ** |
| Belv. | *** | | *** | *** | | +++ | ++ | ++ | ++ |
| Belv. | | | | | | (−45) | (−63) | (−36) | (−46) |
| | | | | | | *** | *** | *** | ** |

In pERK: + denotes a IC₅₀ >300 nM, ++ denotes a 30-300 nM IC₅₀ range, and +++ denotes a IC₅₀ <30 nM.
For proliferation: * denotes a EC₅₀ >3000 nM, ** denotes a 300-3000 nM EC₅₀ range, *** denotes a EC₅₀ <300 nM.
Belv.: Belvarafenib.
Values in parentheses are % inhibition.
Blank means the value was not determined.

D-2.

| | | NCI- | | | NCI- | | | | |
|---|---|---|---|---|---|---|---|---|---|
| | Lovo | H358 | SW620 | Calu6 | H2122 | SW480 | Mewo | NCIH2087 | NCIH1755 |
| | pERK | pERK | pERK | pERK | pERK | pERK | pERK | pERK | pERK |
| Ex. | Prolif. | Prolif. | Prolif. | Prolif. | Prolif. | Prolif. | Prolif. | Prolif. | Prolif. |
| 98 | ++ | ++ | ++ | ++ | ++ | ++ | ++ | +++ | +++ |
| 98 | (27) | (20) | (36) | (5) | (26) | (−1) | (9) | (6) | (9) |
| | *** | *** | ** | *** | ** | ** | *** | *** | *** |
| 112 | ++ | ++ | ++ | ++ | ++ | ++ | +++ | +++ | +++ |
| 112 | (−1) | (−8) | (11) | (1) | (−15) | (−7) | (12) | (−7) | (12) |
| | *** | *** | ** | ** | ** | ** | ** | ** | |
| 114 | +++ | + | + | ++ | ++ | ++ | ++ | ++ | ++ |
| 114 | (4) | (−4) | (−13) | (8) | (0) | (1) | (−12) | (2) | (−5) |
| | *** | *** | * | * | * | * | ** | ** | * |
| 119 | ++ | ++ | ++ | ++ | + | ++ | ++ | +++ | ++ |
| 119 | (0) | (29) | (−1) | (−16) | (−3) | (−5) | (−12) | (26) | (0) |
| | | | | ** | * | * | | | |
| 128 | ++ | ++ | ++ | ++ | + | ++ | ++ | +++ | +++ |
| 128 | (−12) | (14) | (11) | (1) | (−4) | (−14) | (−7) | (−7) | (1) |
| | *** | *** | *** | *** | ** | ** | *** | *** | *** |
| 129 | ++ | ++ | + | ++ | + | ++ | ++ | +++ | +++ |
| 129 | (−2) | (−3) | (−7) | (−1) | (−14) | (3) | (−5) | (3) | (−2) |
| | *** | *** | ** | ** | ** | ** | *** | ** | ** |
| Belv. | +++ | ++ | ++ | ++ | ++ | ++ | ++ | +++ | ++ |
| Belv. | (−12) | (−122) | (−151) | (−39) | (−9) | (−100) | (−116) | (−31) | (−79) |
| | *** | ** | ** | *** | ** | ** | ** | *** | * |

In pERK: + denotes a IC₅₀ >300 nM, ++ denotes a 30-300 nM IC₅₀ range, and +++ denotes a IC₅₀ <30 nM.
For proliferation: * denotes a EC50 >3000 nM, ** denotes a 300-3000 nM EC50 range, *** denotes a EC50 <300 nM.
Belv.: Belvarafenib.
Values in parentheses are % inhibition.
Blank means the value was not determined.

For RAS-mutant cancer cell lines, the % Yₘ in values for pERK IC₅₀ curves were all above −20% and considered to display minimal or no induction and thus compounds do not cause detectable paradoxical activation of the pathway in this panel of cancer cell lines. In contrast, the comparative results for the molecule Belvarafenib (obtained from MedChem Express cat #HY-109080; CAS #1446113-23-0) show mild to strong induction of the pathway in the same cell lines (Y_{MIN}<−30% in 11 of the 13 RAS-mutant cell lines tested).

### (d) Measurement of Proliferation Inhibition of Cultivated Human Cancer Cell Lines (CCLs) Using CellTiter-Glo® Reagent

CellTiter-Glo® viability analysis was conducted on cells plated in 40 μL of complete RPMI-1640 growth medium in 96-well flat-bottomed white opaque plates (Greiner or Corning) at a density indicated in Table E (for each CCL, number of cells plated per well of a 96-well plate to perform the CellTiter-Glo® cell viability assay). The cell density in cells/cm² would correspond to cell number divided by the area of one well of a 96-well plate (0.32 cm²). Cells were maintained overnight at 37° C. under 5% CO₂ before treatment with compounds' dilution series for 3 days.

TABLE E
Number of cells plated per well of a 96-well plate to perform
the CellTiter-Glo ® cell viability assay

| Cell Line | Number of cells/well |
|---|---|
| A375 | 2000 |
| A101D | 2000 |
| A2058 | 2000 |
| RKO | 2000 |
| HT29 | 2000 |
| NCIH2087 | 3600 |
| NCIH1755 | 3600 |
| NCIH1666 | 3600 |
| SK-MEL30 | 2400 |
| IPC298 | 2000 |
| HepG2 | 7200 |
| HCT-116 | 1500 |
| Lovo | 4800 |
| SW620 | 3600 |
| SW480 | 4800 |
| NCI-H358 | 2400 |
| NCI-H2122 | 4800 |
| Calu-6 | 2400 |
| Mewo | 2400 |

In a dilution series 100 μL/well of test substance dilution prepared in complete RPMI-1640 growth media was added to the cells plated initially in 100 μL of growth media. The dilution series is selected so that ten concentrations cover a range from 30 μM or 10 μM to 0.33 μM. If necessary (as in the case of A375 cells, which were more sensitive to the compounds) the initial concentration of 10 μM is lowered to 1 μM and further dilution is carried out accordingly. The final concentration of DMSO in the assay is set to 0.5%.

After 3 days of incubation, the growth media were removed by aspiration and 60 μL of diluted CellTiter-Glo® reagent (10 μL CellTiter-Glo® reagent+50 μL of PBS) was added to each well. Cells were allowed to lyse and to equilibrate in CellTiter-Glo® reagent by 5 min incubation on a plate shaker followed by 10 min incubation at room temperature. Luminescence signals were then acquired on a Synergy Neo2 plate reader (Biotek).

The effect of each compound dilution on the proliferation of cancer cell lines was expressed in % inhibition and calculated as follows. An internal 100% inhibition control (1 μM of trametinib; cat. No. HY-10999; MedChem Express; CAS No. 871700-17-3) was included in each plate and used as a measure of CellTiter-Glo® signal background. The value obtained for trametinib was subtracted from each data point. The average of DMSO (vehicle) controls (set as 0% inhibition) was established and used to calculate % inhibition:

$$\% inhibition = 100 * ( 1 - ( ( CellTiter - Glo ® {signal}_{compound} ) / ( CellTiter - Glo ® {signal}_{DMSO} ) ) )$$

The ability of each compound to inhibit proliferation was expressed as a EC₅₀ value obtained by plotting the effect values for each data point of a dilution series and fitting the obtained curves using a log(agonist) versus response−variable slope (four parameters) function using GraphPadPrism (V7.0) or Dotmatics Screening Ultra platform.

As shown in Table D, the active substances show antiproliferative activity in various NRAS-, KRAS- and NF1-mutant cancer cell lines that are representative of a large diversity of tissue types (i.e., SK-MEL 30, IPC298, HepG2, HCT-116, Lovo, SW620, SW480, NCI-H358, NCI-H2122, Calu-6 and Mewo; Table D and refer to Table B for genotypes). Antiproliferative activity is often even stronger in cell lines carrying the BRAF driver mutation (Table D). Of note, the IC₅₀ values of pERK reduction and the EC₅₀ values of the antiproliferative activity of the substances in KRAS- and BRAF-mutated cell lines correlate reasonably well with each other (Table D). The present compounds are thus effective against several tumor types and may be used in these and other indications. This demonstrates the usefulness of the compounds as herein defined for the treatment of different types of tumors.

### (e) Results

The following Tables 2 to 5 summarize exemplary compounds structures, methods of synthesis, and biological results. Each of these tables are followed by their respective table summarizing chemical characterization of the compounds.

TABLE 2

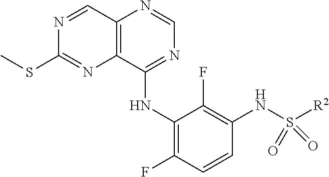

| | | HCT116 | SW480 | Kinase | Kinase |
|---|---|---|---|---|---|
| | | PERK IC₅₀ | PERK IC₅₀ | IC₅₀ | IC₅₀ |
| | | (μM) | (UM) | BRAF | CRAF |
| Example | R² | (Yₘᵢₙ %) | (Yₘᵢₙ %) | (nM) | (nM) |
| 1 | B1 | + (1) | ++ (6) | * | § |
| 2 | B2 | ++ (−14) | ++ (5) | | |
| 3 | B3 | ++ (−7) | ++ (9) | | |
| 4 | B4 | ++ (−5) | ++ (−2) | | |
| 5 | B5 | + (8) | ++ (8) | | |
| 6 | B6 | + (−10) | ++ (1) | | |
| 7 | B7 | + (7) | ++ (17) | | |
| 8 | B8 | ++ (−3) | ++ (15) | | |
| 9 | B9 | + (1) | + (−3) | | |
| 10 | B10 | + (9) | ++ (−9) | | |
| 11 | B11 | ++ (10) | ++ (8) | | |
| 12 | B12 | ++ (−7) | ++ (−10) | | |
| 13 | B13 | + (−2) | + (4) | | |
| 14 | B14 | ++ (−5) | ++ (−6) | | |
| 15 | B15 | + (−3) | + (0) | | |
| 16 | B16 | + (1) | ++ (3) | | |
| 17 | B17 | + (5) | ++ (3) | | |
| 18 | B18 | + (4) | ++ (11) | | |
| 19 | B19 | + (−4) | ++ (4) | | |
| 20 | B20 | ++ (7) | ++ (12) | | |
| 21 | B21 | ++ (2) | ++ (9) | | |
| 22 | B22 | ++ (−1) | ++ (−5) | | |
| 23 | B23 | ++ (0) | ++ (−7) | | |
| 24 | B24 | + (10) | ++ (13) | | |
| 25 | B25 | + (7) | + (−6) | | |
| 26 | B26 | ++ (11) | ++ (9) | | |
| 27 | B27 | + (−6) | | | |
| 28 | B28 | ++ (3) | ++ (−3) | | |
| 29 | B29 | + (−9) | + (3) | | |
| 90 | B36 | ++ (−9.2) | | ** | |
| 91 | B53 | ++ (−8) | | * | §§ |
| 92 | B54 | ++ (−12) | | ** | |

For pERK assays, + denotes a 10-30 μM IC₅₀ range, ++ denotes a 1-10 μM IC₅₀ range, +++ denotes a 0.5-1 μM IC₅₀ range and ++++ denotes an IC₅₀ <0.5 μM. The % Yₘᵢₙ value indicates the lowest value of each IC₅₀ curve. Compounds that exhibit IC₅₀ curves with Yₘᵢₙ values above −20% are considered to display minimal or no induction and do not cause detectable paradoxical activation of the pathway. For the BRAF biochemical kinase assay, * denotes an IC₅₀ >10 nM, ** denotes a 1-10 nM IC₅₀ range and *** denotes an IC₅₀ <1 nM. For the CRAF biochemical kinase assay, § denotes an IC₅₀ >50 nM, §§ denotes a 10-50 nM IC₅₀ range and §§§ denotes an IC50 <10 nM.

### Characterization of Compounds in Table 2

| | | HRMS | |
|---|---|---|---|
| | Synthesis | m/z | |
| Example | Method | (MH⁺) | ¹H NMR (400 MHz) |
| 1 | A | 491.1 | ¹H NMR (DMSO-d₆) δ: 10.14 (s, 1H), 10.03 (s, 1H), 9.27 (s, 1H), |
| | | | 8.49 (s, 1H), 7.56-7.76 (m, 2H), 7.16-7.31 (m, 2H), 7.06- |
| | | | 7.12 (m, 2H), 3.81 (s, 3H), 2.68 (s, 3H) |
| 2 | B | 521.1 | ¹H NMR (DMSO-d₆) δ: 10.12 (s, 1H), 10.04 (s, 1H), 9.29 (s, 1H), |
| | | | 8.48 (s, 1H), 7.17-7.33 (m, 4H), 7.09 (d, J = 8.6 Hz, 1H), 3.83 |
| | | | (s, 3H), 3.72 (s, 3H), 2.71 (s, 3H) |
| 3 | B | 525.3 | ¹H NMR (DMSO-d₆) δ: 10.06 (s, 1H), 9.88 (s, 1H), 9.29 (s, 1H), |
| | | | 8.50 (s, 1H), 7.98 (d, J = 5.0 Hz, 1H), 7.38 (d, J = 5.0 Hz, 1H), |
| | | | 7.34 (td, J = 8.7, 5.8 Hz, 1H), 7.23 (td, J = 9.1, 0.9 Hz, 1H), 3.85 |
| | | | (s, 3H), 2.71 (s, 3H) |
| 4 | B | 503.5 | ¹H NMR (DMSO-d₆) δ: 10.08 (s, 1H), 10.04 (s, 1H), 9.30 (s, 1H), |
| | | | 8.50 (s, 1H), 7.56-7.63 (m, 1H), 7.50 (dd, J = 8.4, 2.0 Hz, 1H), |
| | | | 7.26 (td, J = 9.1, 6.0 Hz, 1H), 7.20 (t, J = 9.0 Hz, 1H), 6.90 (d, J = |
| | | | 8.5 Hz, 1H), 4.62 (t, J = 8.8 Hz, 2H), 3.21 (t, J = 8.8 Hz, 2H), |
| | | | 2.71 (s, 3H) |
| 5 | B | 486.1 | ¹H NMR (DMSO-d₆) δ: 10.78 (s, 1H), 10.04 (s, 1H), 9.29 (s, 1H), |
| | | | 8.49 (s, 1H), 8.10 (dd, J = 7.2, 1.4 Hz, 1H), 7.95 (dd, J = 8.2, 1.0 |
| | | | Hz, 1H), 7.90 (td, J = 7.5, 1.4 Hz, 1H), 7.84 (td, J = 7.4, 1.0 Hz, |
| | | | 1H), 7.34 (td, J = 8.7, 5.7 Hz, 1H), 7.26 (t, J = 9.2 Hz, 1H), 2.69 |
| | | | (s, 3H) |
| 6 | B | 505.1 | ¹H NMR (DMSO-d₆) δ: 10.19 (s, 1H), 10.05 (s, 1H), 9.30 (s, 1H), |
| | | | 8.50 (s, 1H), 7.16-7.32 (m, 4H), 7.06 (d, J = 8.2 Hz, 1H), 6.15 |
| | | | (s, 2H), 2.71 (s, 3H) |
| 7 | B | 516.1 | ¹H NMR (DMSO-d₆) δ: 10.58 (s, 1H), 10.05 (s, 1H), 9.29 (s, 1H), |
| | | | 8.48 (s, 1H), 7.84 (d, J = 9.0 Hz, 1H), 7.67 (d, J = 2.7 Hz, 1H), |
| | | | 7.40 (dd, J = 9.0, 2.7 Hz, 1H), 7.33 (td, J = 8.6, 5.9 Hz, 1H), 7.25 |
| | | | (t, J = 9.0 Hz, 1H), 3.88 (s, 3H), 2.69 (s, 3H) |
| 8 | A | 509.1 | ¹H NMR (DMSO-d₆) δ: 10.40 (s, 1H), 10.07 (s, 1H), 9.29 (s, 1H), |
| | | | 8.47 (s, 1H), 7.61 (t, J = 8.6 Hz, 1H), 7.29 (td, J = 8.2, 5.9 Hz, |
| | | | 1H), 7.22 (t, J = 9.4 Hz, 1H), 7.06 (dd, J = 12.1, 2.0 Hz, 1H), 6.89 |
| | | | (dd, J = 9.0, 2.0 Hz, 1H), 3.83 (s, 3H), 2.71 (s, 3H) |
| 9 | B | 472.4 | ¹H NMR (DMSO-d₆) δ: 10.12 (s, 1H), 9.81 (s, 1H), 9.31 (s, 1H), |
| | | | 8.54 (s, 1H), 7.48 (td, J = 8.6, 5.8 Hz, 1H), 7.26 (td, J = 9.1, 0.9 |
| | | | Hz, 1H), 5.33 (dt, J = 53.3, 3.5 Hz, 1H), 3.36-3.52 (m, 3H), |
| | | | 3.31 (td, J = 9.9, 6.6 Hz, 1H), 2.73 (s, 3H), 1.96-2.17 (m, 2H) |
| 10 | B | 504.1 | ¹H NMR (DMSO-d₆) δ: 10.81 (s, 1H), 10.05 (br s, 1H), 9.29 (s, |
| | | | 1H), 8.49 (s, 1H), 8.19 (dd, J = 8.6, 2.7 Hz, 1H), 7.99 (dd, J = |
| | | | 9.0, 5.5 Hz, 1H), 7.79 (td, J = 8.4, 2.7 Hz, 1H), 7.36 (td, J = 8.6, |
| | | | 5.9 Hz, 1H), 7.27 (t, J = 9.0 Hz, 1H), 2.70 (s, 3H) |
| 11 | B | 484.2 | ¹H NMR (DMSO-d₆) δ: 10.11 (s, 1H), 9.70 (s, 1H), 9.31 (s, 1H), |
| | | | 8.53 (s, 1H), 7.49 (td, J = 8.8, 5.7 Hz, 1H), 7.25 (td, J = 9.1, 1.3 |
| | | | Hz, 1H), 3.91-4.01 (m, 1H), 3.29-3.34 (m, 2H), 3.21-3.29 |
| | | | (m, 2H), 3.19 (s, 3H), 2.73 (s, 3H), 1.84-1.97 (m, 2H) |
| 12 | A, B | 505.1 | ¹H NMR (DMSO-d₆) δ: 10.17 (s, 1H), 10.03 (s, 1H), 9.29 (s, 1H), |
| | | | 8.48 (s, 1H), 7.67 (d, J = 8.6 Hz, 1H), 7.26 (td, J = 9.0, 5.9 Hz, |
| | | | 1H), 7.19 (t, J = 9.0 Hz, 1H), 6.95 (d, J = 2.3 Hz, 1H), 6.86 (dd, |
| | | | J = 8.8, 2.5 Hz, 1H), 3.79 (s, 3H), 2.71 (s, 3H), 2.57 (s, 3H) |
| 13 | B | 472.4 | ¹H NMR (DMSO-d₆) δ: 10.12 (s, 1H), 9.81 (s, 1H), 9.31 (s, 1H), |
| | | | 8.54 (s, 1H), 7.48 (td, J = 8.6, 5.8 Hz, 1H), 7.26 (td, J = 9.1, 0.9 |
| | | | Hz, 1H), 5.33 (dt, J = 53.3, 3.5 Hz, 1H), 3.36-3.52 (m, 3H), |
| | | | 3.31 (td, J = 9.9, 6.6 Hz, 1H), 2.73 (s, 3H), 1.96-2.17 (m, 2H) |
| 14 | B | 559.1 | ¹H NMR (DMSO-d₆) δ: 10.29 (s, 1H), 10.04 (s, 1H), 9.29 (s, 1H), |
| | | | 8.48 (s, 1H), 7.95 (d, J = 9.0 Hz, 1H), 7.41 (d, J = 2.7 Hz, 1H), |
| | | | 7.37 (dd, J = 9.0, 2.7 Hz, 1H), 7.30 (td, J = 8.6, 5.9 Hz, 1H), 7.23 |
| | | | (t, J = 9.4 Hz, 1H), 3.89 (s, 3H), 2.68 (s, 3H) |
| 15 | B | 529.0 | ¹H NMR (DMSO-d₆) δ: 10.63 (s, 1H), 10.04 (s, 1H), 9.29 (s, 1H), |
| | | | 8.48 (s, 1H), 7.91 (d, J = 2.3 Hz, 1H), 7.87 (d, J = 8.6 Hz, 1H), |
| | | | 7.61 (dd, J = 8.4, 2.2 Hz, 1H), 7.30 (td, J = 8.6, 5.9 Hz, 1H), 7.22 |
| | | | (t, J = 9.0 Hz, 1H), 2.69 (s, 3H) |
| 16 | B | 557.0 | ¹H NMR (DMSO-d₆) δ: 10.70 (s, 1H), 10.04 (s, 1H), 9.29 (s, 1H), |
| | | 559.0 | 8.48 (s, 1H), 7.87 (dd, J = 9.8, 1.0 Hz, 1H), 7.56-7.67 (m, 2H), |
| | | | 7.33 (td, J = 8.6, 5.9 Hz, 1H), 7.24 (t, J = 9.2 Hz, 1H), 2.68 (s, |
| | | | 3H) |
| 17 | B | 553.0 | ¹H NMR (DMSO-d₆) δ: 10.43 (s, 1H), 10.03 (s, 1H), 9.29 (s, 1H), |
| | | 555.0 | 8.49 (s, 1H), 7.69 (d, J = 1.2 Hz, 1H), 7.62 (d, J = 8.2 Hz, 1H), |
| | | | 7.56 (dd, J = 8.6, 2.0 Hz, 1H), 7.29 (td, J = 9.8, 5.9 Hz, 1H), 7.22 |
| | | | (t, J = 9.8 Hz, 1H), 2.71 (s, 3H), 2.59 (s, 3H) |
| 18 | B | 475.1 | ¹H NMR (DMSO-d₆) δ: 10.24 (br s, 1H), 10.03 (s, 1H), 9.29 (s, |
| | | | 1H), 8.50 (s, 1H), 7.53-7.77 (m, J = 8.2 Hz, 2H), 7.35-7.42 |
| | | | (m, J = 7.8 Hz, 2H), 7.16-7.30 (m, 2H), 2.71 (s, 3H), 2.36 (s, |
| | | | 3H) |
| 19 | B | 521.1 | ¹H NMR (DMSO-d₆) δ: 10.04 (br s, 1H), 9.29 (s, 1H), 8.48 (s, |
| | | | 1H), 7.58 (d, J = 9.0 Hz, 1H), 7.26 (td, J = 8.7, 5.7 Hz, 1H), 7.15 |
| | | | (td, J = 9.4, 1.0 Hz, 1H), 6.69 (d, J = 2.3 Hz, 1H), 6.58 (dd, J = |
| | | | 8.6, 2.3 Hz, 1H), 3.83 (s, 3H), 3.82 (s, 3H), 2.68 (s, 3H) |
| 20 | B | 475.1 | ¹H NMR (DMSO-d₆) δ: 10.33 (s, 1H), 10.03 (s, 1H), 9.29 (s, 1H), |
| | | | 8.49 (s, 1H), 7.75 (dd, J = 8.0, 1.0 Hz, 1H), 7.52 (td, J = 7.4, 1.2 |
| | | | Hz, 1H), 7.40 (d, J = 7.8 Hz, 1H), 7.34 (t, J = 7.6 Hz, 1H), 7.26 |
| | | | (td, J = 9.0, 5.9 Hz, 1H), 7.19 (t, J = 9.4 Hz, 1H), 2.68 (s, 3H), |
| | | | 2.61 (s, 3H) |
| 21 | B | 495.0 | ¹H NMR (DMSO-d₆) δ: 10.53 (s, 1H), 10.06 (br s, 1H), 9.29 (s, |
| | | | 1H), 8.48 (s, 1H), 7.91 (dd, J = 7.8, 1.6 Hz, 1H), 7.62-7.72 (m, |
| | | | 2H), 7.47-7.54 (m, 1H), 7.17-7.33 (m, 2H), 2.68 (s, 3H) |
| 22 | B | 509.0 | ¹H NMR (DMSO-d₆) δ: 10.43 (s, 1H), 10.03 (s, 1H), 9.29 (s, 1H), |
| | | | 8.48 (s, 1H), 7.70 (d, J = 8.2 Hz, 1H), 7.55 (s, 1H), 7.43 (dd, J = |
| | | | 8.6, 2.0 Hz, 1H), 7.29 (td, J = 9.0, 6.7 Hz, 1H), 7.22 (t, J = 9.0 |
| | | | Hz, 1H), 2.71 (s, 3H), 2.60 (s, 3H) |
| 23 | B | 489.1 | ¹H NMR (DMSO-d₆) δ: 10.26 (br s, 1H), 10.03 (s, 1H), 9.29 (s, |
| | | | 1H), 8.49 (s, 1H), 7.63 (d, J = 8.2 Hz, 1H), 7.09-7.32 (m, 4H), |
| | | | 2.70 (s, 3H), 2.57 (s, 3H), 2.30 (s, 3H) |
| 24 | B | 454.1 | ¹H NMR (DMSO-d₆) δ: 10.11 (br s, 1H), 9.31 (s, 1H), 8.52 (s, |
| | | | 1H), 7.49 (td, J = 8.8, 5.9 Hz, 1H), 7.25 (t, J = 8.6 Hz, 1H), 3.18 |
| | | | (t, J = 6.7 Hz, 4H), 2.73 (s, 3H), 1.74-1.87 (m, 4H) |
| 25 | B | 470.4 | ¹H NMR (CDCl₃) δ: 9.33 (s, 1H), 8.74 (s, 1H), 8.15 (br s, 1H), |
| | | | 7.60 (td, J = 8.8, 5.4 Hz, 1H), 7.22 (br s, 1H), 7.08 (td, J = 9.1, |
| | | | 1.9 Hz, 1H), 4.13-4.20 (m, 1H), 4.04-4.11 (m, 2H), 3.84- |
| | | | 3.96 (m, 2H), 3.26 (s, 3H), 2.71 (s, 3H) |
| 26 | B | 484.4 | ¹H NMR (DMSO-d₆) δ: 10.11 (s, 1H), 9.70 (s, 1H), 9.31 (s, 1H), |
| | | | 8.53 (s, 1H), 7.49 (td, J = 8.8, 5.7 Hz, 1H), 7.25 (td, J = 9.1, 1.3 |
| | | | Hz, 1H), 3.91-4.01 (m, 1H), 3.29-3.34 (m, 2H), 3.21-3.29 |
| | | | (m, 2H), 3.19 (s, 3H), 2.73 (s, 3H), 1.84-1.97 (m, 2H) |
| 27 | B | 490.4 | ¹H NMR (MeOH-d₄) δ: 9.18 (s, 1H), 8.49 (s, 1H), 7.56-7.63 (m, |
| | | | 1H), 7.15 (td, J = 9.1, 1.9 Hz, 1H), 3.63 (t, J = 12.8 Hz, 2H), 3.52 |
| | | | (t, J = 7.3 Hz, 2H), 2.74 (s, 3H), 2.39 (tt, J = 13.9, 7.1 Hz, 2H) |
| 28 | B | 501.9 | ¹H NMR (MeOH-d₄) δ: 9.20 (s, 1H), 8.53 (s, 1H), 7.63 (td, J = |
| | | | 8.8, 5.7 Hz, 1H), 7.14 (td, J = 9.1, 1.9 Hz, 1H), 3.48-3.59 (m, |
| | | | 4H), 3.39-3.44 (m, 1H), 3.12 (dd, J = 9.9, 7.1 Hz, 1H), 2.75 (s, |
| | | | 3H), 2.61 (dt, J = 14.4, 7.1 Hz, 1H), 2.09 (dtd, J = 12.3, 7.3, 4.6 |
| | | | Hz, 1H), 1.76 (dq, J = 12.6, 8.1 Hz, 1H) |
| 29 | C | 468.1 | ¹H NMR (CDCl₃) δ: 9.29 (s, 1H), 8.67 (s, 1H), 8.11 (br s, 1H), |
| | | | 7.61 (td, J = 9.0, 5.5 Hz, 1H), 7.07 (td, J = 9.2, 2.0 Hz, 1H), 6.67 |
| | | | (br s, 1H), 3.44-3.54 (m, 2H), 3.27-3.38 (m, 1H), 2.88 (dd, J = |
| | | | 9.2, 8.0 Hz, 1H), 2.71 (s, 3H), 2.30 (dq, J = 15.2, 7.3 Hz, 1H), |
| | | | 1.98-2.09 (m, 1H), 1.48-1.58 (m, 1H), 1.04 (d, J = 6.7 Hz, |
| | | | 3H) |
| 90 | A | 493.2 | ¹H NMR (DMSO-d₆) δ: 10.50 (s, 1H), 10.04 (s, 1H), 9.29 (s, 1H), |
| | See Ex. 1 | | 8.48 (s, 1H), 7.61 (d, J = 8.2 Hz, 1H), 7.48 (dd, J = 9.8, 8.2 Hz, |
| | | | 1H), 7.36-7.43 (m, 1H), 7.25-7.34 (m, 1H), 7.21 (dd, J = 11.3, |
| | | | 8.6 Hz, 1H), 2.71 (s, 3H), 2.50 (s, 3H) |
| 91 | A | 523.2 | ¹H NMR (DMSO-d₆) δ: 10.33 (br s, 1H), 10.03 (s, 1H), 9.29 (s, |
| | See Ex. 1 | | 1H), 8.46 (s, 1H), 7.57 (d, J = 8.2 Hz, 1H), 7.25-7.34 (m, 1H), |
| | | | 7.16-7.23 (m, 1H), 7.06-7.15 (m, 1H), 3.87 (s, 3H), 2.71 (s, |
| | | | 3H), 2.48 (s, 3H) |
| 92 | A | 539.2 | ¹H NMR (DMSO-d₆) δ: 10.39 (br s, 1H), 10.03 (s, 1H), 9.29 (s, |
| | See Ex. 1 | | 1H), 8.46 (s, 1H), 7.76 (d, J = 9.0 Hz, 1H), 7.25-7.33 (m, 1H), |
| | | | 7.15-7.23 (m, 1H), 7.12 (d, J = 9.0 Hz, 1H), 3.90 (s, 3H), 2.71 |
| | | | (s, 3H), 2.65 (s, 3H) |

TABLE 3

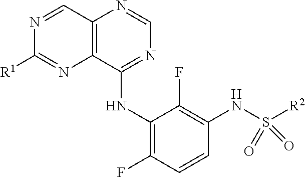

| | | | HCT116 | SW480 | Kinase | Kinase |
|---|---|---|---|---|---|---|
| | | | PERK IC₅₀ | PERK IC₅₀ | IC₅₀ | IC₅₀ |
| | | | (μM) | (μM) | BRAF | CRAF |
| Example | R¹ | R² | (Yₘᵢₙ %) | (Yₘᵢₙ %) | (nM) | (nM) |
| 30 | C2 | B2 | ++ (−6) | + (−5) | | |
| 31 | C3 | B1 | ++ (13) | ++ (11) | | |
| 32 | C4 | B1 | + (1) | ++ (8) | | |
| 33 | C3 | B5 | + (−4) | ++ (13) | | |
| 34 | C5 | B5 | + (−3) | ++ (−2.0) | | |
| 35 | C6 | B1 | + (−4) | ++ (14) | | |
| 36 | C7 | B1 | ++ (10) | ++ (10) | | |
| 37 | C8 | B1 | ++ (−5) | ++ (−6) | | |
| 38 | C9 | B1 | + (−1) | + (−3) | | |
| 39 | C10 | B1 | + (5) | + (−3) | | |
| 40 | C5 | B1 | ++ (12) | ++ (−7) | | |
| 41 | C11 | B1 | + (13) | + (6) | | |
| 42 | C12 | B1 | + (−2) | + (11) | | |
| 43 | C13 | B1 | + (11) | + (0) | | |
| 44 | C3 | B8 | ++ (−2) | ++ (12) | | |
| 45 | C7 | B8 | ++ (11) | ++ (−8) | | |
| 46 | C14 | B1 | + (8) | + (12) | | |
| 47 | C15 | B1 | ++ (6) | ++ (−1) | | |
| 48 | C16 | B1 | ++ (13) | ++ (6) | | |
| 49 | C17 | B1 | + (8) | + (−9) | | |
| 50 | C16 | B8 | ++ (8) | ++ (−9) | | |
| 51 | C7 | B9 | ++ (−8) | ++ (−1) | | |
| 52 | C15 | B8 | ++ (−4) | ++ (−8) | | |
| 53 | C5 | B8 | ++ (15) | ++ (−4) | | |
| 54 | C18 | B1 | + (−7) | + (8) | | |
| 55 | C7 | B12 | ++ (−4) | ++ (−2) | | |
| 56 | C5 | B12 | ++ (−10) | ++ (4) | | |
| 57 | C3 | B12 | ++ (−2) | ++ (−2) | * | § |
| 58 | C5 | B11 | ++(11) | ++ (16) | | |
| 59 | C7 | B11 | +++ (11) | +++ (2) | | |
| 60 | C3 | B11 | ++ (9) | ++ (15) | | |
| 61 | C3 | B15 | ++ (11) | ++ (9) | | |
| 62 | C5 | B15 | ++ (11) | +++ (2) | ** | §§ |
| 63 | C7 | B15 | ++ (−2) | ++ (1) | ** | §§§ |
| 64 | C7 | B4 | ++ (2) | ++ (19) | | |
| 65 | C5 | B4 | ++ (4) | ++ (5) | | |
| 66 | C3 | B4 | + (10) | ++ (0) | | |
| 67 | C19 | B11 | ++ (6) | ++ (18) | | |
| 68 | C20 | B11 | ++ (17) | ++ (0) | | |
| 69 | C27 | B11 | +++ (8) | ++++ (12) | | |
| 70 | C7 | B30 | ++ (0) | ++ (1) | | |
| 71 | C21 | B11 | ++ (8) | ++ (−4) | | |
| 72 | C22 | B11 | ++ (20 | ++ (1) | | |
| 73 | C7 | B29 | ++ (−2) | ++ (7) | | |
| 74 | C7 | B31 | ++ (−3) | ++ (−7) | | |
| 75 | C7 | B28 | ++ (−10) | ++ (−6) | | |
| 76 | C7 | B32 | ++ (−1) | ++ (−9) | | |
| 77 | C7 | B24 | ++ (−4) | ++ (−4) | | |
| 78 | C7 | B33 | ++ (6) | +++ (10) | | |
| 79 | C23 | B8 | +++ (−6) | ++++ (−2) | | |
| 80 | C23 | B41 | ++++ (−7) | ++++ (−2) | *** | §§§ |
| 81 | C7 | B41 | ++++ (−4) | ++++ (3) | *** | §§§ |
| 82 | C23 | B36 | ++++ (−10) | ++++ (3.2) | ** | §§§ |
| 83 | C7 | B36 | ++++ (−10) | ++++ (−2) | ** | §§§ |
| 93 | C60 | B36 | ++++ (1.3) | | ** | §§§ |
| 94 | C60 | B41 | ++++ (7.7) | | ** | §§§ |
| 95 | C72 | B36 | ++ (−5) | | ** | §§ |
| 96 | C81 | B36 | ++++ (−3.8) | | *** | §§§ |
| 97 | C223 | B36 | ++++ (−6.3) | | *** | §§ |
| 98 | C224 | B36 | (−6.0) | ++++ (−1) | *** | §§ |
| 99 | C73 | B36 | (−3.6) | | *** | §§ |
| 100 | C69 | B36 | ++++ (2.2) | | *** | §§ |
| 101 | C225 | B36 | ++++ (3.5) | | *** | §§§ |
| 102 | C196 | B36 | +++ (−9.5) | | *** | §§§ |
| 103 | C114 | B36 | +++ (−9.9) | | *** | §§§ |
| 104 | C83 | B36 | ++++ (−9.8) | | *** | § |
| 105 | C71 | B36 | ++++ (−8.7) | | ** | § |
| 106 | C184 | B36 | ++++ (−2.3) | | *** | §§ |
| 107 | C220 | B36 | +++ (−5.2) | | *** | §§ |
| 108 | C88 | B36 | ++ (−7.0) | | +++ | §§ |
| 109 | C182 | B36 | ++++ (−6.5) | | *** | §§ |
| 110 | C82 | B36 | ++++ (−3.7) | | *** | §§ |
| 111 | C23 | B53 | ++++ (−8.1) | | ** | §§§ |
| 112 | C23 | B54 | +++ (−0.8) | ++++ (−7) | * | §§ |
| 113 | C226 | B36 | +++ (−7.0) | | ** | §§ |
| 114 | C183 | B36 | ++++ (−4.9) | ++++ (2) | ** | §§ |
| 115 | C7 | B54 | ++++ (2) | | ** | §§ |
| 116 | C7 | B53 | ++++ (−1) | | ** | § |
| 117 | C22 | B54 | ++ (−1) | | * | §§ |
| 118 | C22 | B53 | ++ (−14) | | * | §§ |
| 119 | C488 | B54 | ++++ (−7) | ++++ (−5) | *** | §§ |
| 120 | C488 | B53 | ++++ (−4) | | ** | §§ |
| 121 | C22 | B36 | ++ (−4) | | ** | §§§ |
| 122 | C488 | B41 | ++++ (−4) | | *** | §§§ |
| 123 | C490 | B41 | ++ (3) | | ** | §§§ |
| 124 | C22 | B42 | ++ (−9) | | ** | §§§ |
| 125 | C488 | B42 | +++ (−5) | | +++ | §§§ |
| 126 | C488 | B36 | ++++ (−3) | | *** | §§§ |
| 127 | C7 | B42 | ++++ (0) | | ** | §§§ |
| 128 | C418 | B41 | ++++ (7) | ++++ (−14) | * | §§ |
| 129 | C414 | B41 | ++++ (−8) | ++++ (3) | * | §§ |
| 130 | C376 | B41 | ++++ (−13) | | ** | §§§ |
| 131 | C419 | B41 | ++++ (−4) | | ** | §§ |
| 132 | C404 | B41 | ++++ (−4) | | ** | §§§ |
| 133 | C438 | B41 | ++++ (−6) | | ** | §§§ |
| 134 | C312 | B41 | ++++ (8) | | ** | §§§ |
| 135 | C440 | B41 | +++ (−14) | | ** | §§§ |
| 136 | C435 | B41 | ++++ (19) | | ** | §§§ |
| 137 | C313 | B41 | ++++ (11) | | ** | §§§ |
| 138 | C441 | B41 | ++++ (1) | | * | §§ |
| 139 | C434 | B41 | ++++ (2) | | *** | §§§ |
| 140 | C483 | B41 | ++++ (−16) | | * | §§ |
| 141 | C275 | B41 | ++++ (−3) | | ** | §§ |
| 142 | C323 | B41 | ++++ (−15) | | *** | §§§ |
| 143 | C346 | B41 | ++++ (−7) | | ** | §§ |
| 144 | C292 | B41 | ++++ (−9) | | ** | §§ |
| 145 | C472 | B41 | ++++ (−8) | | *** | §§§ |
| 146 | C402 | B41 | ++++ (−9) | | *** | §§§ |
| 147 | C310 | B41 | ++++ (−9) | | ** | §§ |
| 148 | C7 | B55 | ++ (−1) | | * | §§ |
| 149 | C438 | B55 | ++++ (4) | | * | §§ |
| 150 | C7 | B77 | ++ (−8) | | ** | §§ |
| 151 | C438 | B77 | ++++ (10) | | ** | §§ |
| 152 | C7 | B59 | ++ (−15) | | ** | § |
| 153 | C438 | B59 | ++++ (−6) | | ** | §§ |
| 154 | C7 | B65 | +++ (3) | | ** | § |
| 155 | C438 | B65 | ++++ (−8) | | ** | § |
| 156 | C7 | B63 | +++ | | ** | §§§ |
| 157 | C7 | B73 | ++++ (0) | | *** | §§§ |
| 158 | C438 | B63 | ++++ (−1) | | ** | §§§ |
| 159 | C438 | B73 | ++++ (5) | | ** | §§§ |

For pERK assays, + denotes a 10-30 μM IC₅₀ range, ++ denotes a 1-10 μM IC₅₀ range, +++ denotes a 0.5-1 μM IC₅₀ range and ++++ denotes an IC₅₀ <0.5 μM. The % Yₘᵢₙ value indicates the lowest value of each IC₅₀ curve. Compounds that exhibit IC₅₀ curves with Yₘᵢₙ values above −20% are considered to display minimal or no induction and do not cause detectable paradoxical activation of the pathway. For the BRAF biochemical kinase assay, * denotes an IC₅₀ >10 nM, ** denotes a 1-10 nM IC₅₀ range and *** denotes an IC₅₀ <1 nM. For the CRAF biochemical kinase assay, § denotes an IC₅₀ >50 nM, §§ denotes a 10-50 nM IC₅₀ range and §§§ denotes an IC₅₀ <10 nM.

### Characterization of Compounds in Table 3

| | | HRMS | |
|---|---|---|---|
| | Synthesis | m/z | |
| Ex. | Method | (MH⁺) | ¹H NMR (400 MHz) |
| 30 | B | 555.6 | ¹H NMR (DMSO-d₆) δ: 10.31 (s, 1H), 9.53 (s, 1H), 8.77 (s, 1H), 8.55 |
| | | | (s, 1H), 7.99 (s, 1H), 7.30 (dd, J = 8.5, 2.2 Hz, 1H), 7.24-7.28 (m, |
| | | | 1H), 7.23 (d, J = 2.2 Hz, 1H), 7.07 (d, J = 8.5 Hz, 1H), 3.80 (s, 3H), |
| | | | 3.74 (s, 3H), 2.22 (d, J = 0.9 Hz, 3H) |
| 31 | B | 532.1 | ¹H NMR (DMSO-d₆) δ: 10.11 (br s, 1H), 9.56 (s, 1H), 9.13 (s, 1H), |
| | | | 8.23 (s, 1H), 7.67 (d, J = 9.0 Hz, 2H), 7.13-7.31 (m, 2H), 7.08 (d, |
| | | | J = 9.0 Hz, 2H), 5.50 (d, J = 54 Hz, 1H), 3.99 (br s, 1H), 3.81 (s, |
| | | | 3H), 3.54-3.72 (m, 1H), 2.24-2.40 (m, 3H) |
| 32 | B | 525.1 | ¹H NMR (DMSO-d₆) δ: 10.41 (s, 1H), 10.17 (s, 1H), 9.73 (s, 1H), |
| | | | 8.70 (s, 1H), 8.68 (br s, 1H), 7.77 (d, J = 2.0 Hz, 1H), 7.62-7.73 |
| | | | (m, 2H), 7.23-7.31 (m, 2H), 7.05-7.14 (m, 2H), 3.81 (s, 3H), 3.05 |
| | | | (s, 3H) |
| 33 | B | 527.1 | ¹H NMR (DMSO-d₆) δ: 10.76 (s, 1H), 9.69 (br s, 1H), 9.13 (s, 1H), |
| | | | 8.26 (s, 1H), 8.10 (d, J = 7.4 Hz, 1H), 7.93-7.98 (m, 1H), 7.89 (t, |
| | | | J = 7.6 Hz, 1H), 7.84 (td, J = 7.8, 1.0 Hz, 1H), 7.31 (td, J = 8.6, 6.3 |
| | | | Hz, 1H), 7.24 (t, J = 9.0 Hz, 1H), 3.97 (br s, 1H), 3.84 (br s, 1H), |
| | | | 3.73 (br s, 1H), 3.63 (s, 1H), 2.24-2.36 (m, 3H) |
| 34 | A | 534.1 | ¹H NMR (DMSO-d₆) δ: 10.80 (br s, 1H), 10.34 (s, 1H), 9.72 (br s, |
| | | | 1H), 9.70 (s, 1H), 8.68 (s, 1H), 8.11 (dd, J = 7.4, 1.6 Hz, 1H), 7.98 |
| | | | (dd, J = 8.2, 1.2 Hz, 1H), 7.91 (td, J = 7.6, 1.2 Hz, 1H), 7.86 (td, J = |
| | | | 7.8, 1.6 Hz, 1H), 7.25-7.43 (m, 2H), 2.64 (s, 3H), 2.31 (s, 3H) |
| 35 | A | 511.1 | ¹H NMR (DMSO-d₆) δ: 10.37 (s, 1H), 10.18 (br s, 1H), 9.57 (s, 1H), |
| | | | 8.89 (s, 1H), 8.59 (s, 1H), 8.28 (s, 1H), 7.68 (d, J = 9.0 Hz, 2H), |
| | | | 7.18-7.34 (m, 3H), 7.09 (d, J = 9.0 Hz, 2H), 3.81 (s, 3H) |
| 36 | A | 561.1 | ¹H NMR (DMSO-d₆) δ: 10.29 (s, 1H), 9.68 (s, 1H), 9.61 (s, 1H), 8.70 |
| | | | (d, J = 8.2 Hz, 1H), 8.60 (s, 1H), 7.82 (d, J = 7.8 Hz, 1H), 7.65- |
| | | | 7.75 (m, 2H), 7.48 (td, J = 7.8, 0.8 Hz, 1H), 7.41 (td, J = 7.4, 1.0 Hz, |
| | | | 1H), 7.19-7.35 (m, 2H), 7.05-7.16 (m, J = 9.0 Hz, 2H), 3.81 (s, |
| | | | 3H) |
| 37 | A | 539.1 | ¹H NMR (DMSO-d₆) δ: 10.17 (br s, 1H), 10.14 (s, 1H), 9.54 (s, 1H), |
| | | | 8.56 (s, 1H), 8.00 (s, 1H), 7.68 (d, J = 8.6 Hz, 2H), 7.18-7.34 (m, |
| | | | 2H), 7.09 (d, J = 9.0 Hz, 2H), 3.81 (s, 3H), 2.75 (s, 3H), 2.14 (s, 3H) |
| 38 | A | 532.1 | ¹H NMR (DMSO-d₆) δ: 10.12 (br s, 1H), 9.56 (s, 1H), 9.13 (s, 1H), |
| | | | 8.23 (s, 1H), 7.57-7.75 (m, 2H), 7.13-7.27 (m, 2H), 7.08 (d, J = |
| | | | 9.0 Hz, 2H), 5.51 (d, J = 53.2 Hz, 1H), 3.89-4.05 (m, 2H), 3.81 (s, |
| | | | 3H), 3.56-3.77 (m, 2H), 2.12-2.38 (m, 2H) |
| 39 | A | 550.1 | ¹H NMR (DMSO-d₆) δ: 10.11 (br s, 1H), 9.66 (s, 1H), 9.18 (s, 1H), |
| | | | 8.28 (s, 1H), 7.54-7.78 (m, 2H), 7.14-7.28 (m, 2H), 7.04-7.12 |
| | | | (m, 2H), 4.09 (t, J = 13.3 Hz, 2H), 3.90 (t, J = 7.2 Hz, 2H), 3.81 (s, |
| | | | 3H), 2.53-2.68 (m, 2H) |
| 40 | A | 539.1 | ¹H NMR (DMSO-d₆) δ: 10.16 (s, 2H), 9.55 (s, 1H), 8.75 (s, 1H), 8.57 |
| | | | (s, 1H), 7.53-7.75 (m, 2H), 7.18-7.38 (m, 2H), 6.94-7.18 (m, 2H), |
| | | | 3.81 (s, 3H), 2.55 (s, 3H), 2.14 (s, 3H) |
| 41 | A | 530.1 | ¹H NMR (DMSO-d₆) δ: 10.11 (s, 1H), 9.49 (br s, 1H), 9.09 (s, 1H), |
| | | | 8.20 (s, 1H), 7.51-7.76 (m, 2H), 7.12-7.26 (m, 2H), 7.05-7.12 |
| | | | (m, 2H), 5.03 (d, J = 3.5 Hz, 1H), 4.43 (sxt, J = 3.5 Hz, 1H), 3.81 (s, |
| | | | 3H), 3.76 (br s, 1H), 3.61-3.71 (m, 3H), 2.06 (s, 1H), 1.87-2.00 |
| | | | (m, 1H) |
| 42 | A | 530.1 | ¹H NMR (DMSO-d₆) δ: 10.11 (s, 1H), 9.49 (br s, 1H), 9.09 (s, 1H), |
| | | | 8.20 (s, 1H), 7.51-7.76 (m, 2H), 7.12-7.26 (m, 2H), 7.05-7.12 |
| | | | (m, 2H), 5.03 (d, J = 3.5 Hz, 1H), 4.43 (sxt, J = 3.5 Hz, 1H), 3.81 (s, |
| | | | 3H), 3.76 (br s, 1H), 3.61-3.71 (m, 3H), 2.06 (s, 1H), 1.87-2.00 |
| | | | (m, 1H) |
| 43 | A | 500.1 | ¹H NMR (DMSO-d₆) δ: 10.10 (br s, 1H), 9.47 (s, 1H), 9.07 (s, 1H), |
| | | | 8.23 (s, 1H), 7.52-7.77 (m, 2H), 7.21 (td, J = 9.0, 5.9 Hz, 1H), 7.15 |
| | | | (t, J = 9.0 Hz, 1H), 7.03-7.11 (m, J = 9.0 Hz, 2H), 4.20 (t, J = 7.6 |
| | | | Hz, 4H), 3.80 (s, 3H), 2.37 (quin, J = 7.5 Hz, 2H) |
| 44 | A | 550.1 | ¹H NMR (DMSO-d₆) δ: 10.37 (br s, 1H), 9.58 (s, 1H), 9.13 (s, 1H), |
| | | | 8.21 (s, 1H), 7.61 (t, J = 8.8 Hz, 1H), 7.26 (td, J = 9.0, 6.3 Hz, 1H), |
| | | | 7.18 (t, J = 9.4 Hz, 1H), 7.05 (dd, J = 12.3, 2.2 Hz, 1H), 6.88 (dd, J = |
| | | | 8.8, 2.2 Hz, 1H), 3.87-4.17 (m, 2H), 3.82 (s, 3H), 3.52-3.77 (m, |
| | | | 2H), 2.12-2.38 (m, 2H) |
| 45 | A | 579.1 | ¹H NMR (DMSO-d₆) δ: 10.45 (s, 1H), 10.31 (s, 1H), 9.68 (s, 1H), |
| | | | 9.62 (s, 1H), 8.71 (d, J = 8.2 Hz, 1H), 8.58 (s, 1H), 7.82 (d, J = 7.8 |
| | | | Hz, 1H), 7.63 (t, J = 8.8 Hz, 1H), 7.48 (t, J = 7.6 Hz, 1H), 7.41 (t, J = |
| | | | 7.6 Hz, 1H), 7.33 (td, J = 8.6, 6.3 Hz, 1H), 7.27 (t, J = 8.6 Hz, 1H), |
| | | | 7.07 (dd, J = 12.3, 2.2 Hz, 1H), 6.90 (dd, J = 9.0, 2.3 Hz, 1H), 3.83 |
| | | | (s, 3H) |
| 46 | A | 475.1 | ¹H NMR (DMSO-d₆) δ: 10.14 (br s, 1H), 10.00 (s, 1H), 9.36 (s, 1H), |
| | | | 8.46 (s, 1H), 7.61-7.71 (m, 2H), 7.24 (td, J = 8.6, 5.5 Hz, 1H), 7.19 |
| | | | (t, J = 9.8 Hz, 1H), 7.04-7.12 (m, 2H), 4.12 (s, 3H), 3.81 (s, 3H) |
| 47 | A | 518.1 | ¹H NMR (DMSO-d₆) δ: 10.11 (br s, 1H), 9.59 (s, 1H), 9.13 (s, 1H), |
| | | | 8.27 (s, 1H), 7.48-7.75 (m, 2H), 7.22 (td, J = 9.0, 5.9 Hz, 1H), 7.17 |
| | | | (t, J = 9.0 Hz, 1H), 7.04-7.13 (m, J = 9.0 Hz, 2H), 5.55 (dspt, J = |
| | | | 57.5, 2.9 Hz, 0H), 4.54 (ddd, J = 21.5, 11.7, 6.3 Hz, 2H), 4.25 (dd, |
| | | | J = 24.3, 10.2 Hz, 2H), 3.81 (s, 3H) |
| 48 | A | 536.1 | ¹H NMR (DMSO-d₆) δ: 10.12 (br s, 1H), 9.71 (s, 1H), 9.20 (s, 1H), |
| | | | 8.33 (s, 1H), 7.64-7.71 (m, J = 9.0 Hz, 2H), 7.23 (td, J = 9.0, 6.3 |
| | | | Hz, 1H), 7.18 (t, J = 9.0 Hz, 1H), 7.04-7.13 (m, J = 9.0 Hz, 2H), |
| | | | 4.62 (t, J = 12.3 Hz, 4H), 3.81 (s, 3H) |
| 49 | A | 562.1 | ¹H NMR (DMSO-d₆) δ: 10.13 (br s, 1H), 10.11 (s, 1H), 9.41 (s, 1H), |
| | | | 8.52 (s, 1H), 7.90-7.95 (m, 1H), 7.77 (dt, J = 7.0, 1.6 Hz, 1H), 7.62- |
| | | | 7.75 (m, 4H), 7.24 (td, J = 9.0, 6.3 Hz, 1H), 7.18 (t, J = 9.4 Hz, |
| | | | 1H), 7.09 (d, J = 9.0 Hz, 2H), 3.81 (s, 3H) |
| 50 | A | 554.1 | ¹H NMR (DMSO-d₆) δ: 10.38 (s, 1H), 9.73 (s, 1H), 9.21 (s, 1H), 8.30 |
| | | | (s, 1H), 7.60 (t, J = 8.6 Hz, 1H), 7.27 (td, J = 9.0, 6.3 Hz, 1H), 7.19 |
| | | | (t, J = 9.4 Hz, 1H), 7.05 (dd, J = 12.3, 2.2 Hz, 1H), 6.88 (dd, J = 8.8, |
| | | | 2.2 Hz, 1H), 4.62 (t, J = 12.3 Hz, 4H), 3.82 (s, 3H) |
| 51 | B | 542.6 | ¹H NMR (DMSO-d₆) δ: 10.38 (br s, 1H), 9.85 (s, 1H), 9.70 (s, 1H), |
| | | | 9.65 (s, 1H), 8.73 (d, J = 8.5 Hz, 1H), 8.65 (s, 1H), 7.83 (d, J = 7.6 |
| | | | Hz, 1H), 7.47-7.56 (m, 2H), 7.42 (td, J = 7.6, 1.1 Hz, 1H), 7.32 (td, |
| | | | J = 9.1, 1.3 Hz, 1H), 5.34 (dt, J = 53.6, 3.2 Hz, 1H), 3.49-3.52 (m, |
| | | | 1H), 3.39-3.48 (m, 2H), 3.33 (td, J = 9.9, 6.8 Hz, 1H), 1.98-2.18 |
| | | | (m, 2H) |
| 52 | A | 536.1 | ¹H NMR (DMSO-d₆) δ: 10.37 (br s, 1H), 9.60 (s, 1H), 9.13 (s, 1H), |
| | | | 8.25 (s, 1H), 7.61 (t, J = 8.6 Hz, 1H), 7.25 (td, J = 8.6, 6.3 Hz, 1H), |
| | | | 7.17 (t, J = 9.4 Hz, 1H), 7.04 (dd, J = 12.3, 2.2 Hz, 1H), 6.88 (dd, J = |
| | | | 9.0, 2.3 Hz, 1H), 5.55 (dspt, J = 57.5, 3.1 Hz, 0H), 4.54 (ddd, J = |
| | | | 21.5, 11.0, 5.9 Hz, 2H), 4.25 (dd, J = 23.9, 10.6 Hz, 2H), 3.80 (s, |
| | | | 3H) |
| 53 | A | 557.1 | ¹H NMR (DMSO-d₆) δ: 10.42 (br s, 1H), 10.17 (s, 1H), 9.55 (s, 1H), |
| | | | 8.75 (s, 1H), 8.54 (s, 1H), 7.62 (t, J = 8.6 Hz, 1H), 7.31 (td, J = 8.6, |
| | | | 5.9 Hz, 1H), 7.24 (t, J = 9.0 Hz, 1H), 7.06 (dd, J = 12.3, 2.2 Hz, 1H), |
| | | | 6.89 (dd, J = 8.6, 2.3 Hz, 1H), 3.83 (s, 3H), 2.55 (s, 3H), 2.14 (s, |
| | | | 3H |
| 54 | A | 552.1 | ¹H NMR (DMSO-d₆) δ: 10.17 (br s, 1H), 9.35 (s, 1H), 8.48 (s, 1H), |
| | | | 7.67 (d, J = 9.0 Hz, 2H), 7.20-7.29 (m, 1H), 7.16 (t, J = 9.0 Hz, |
| | | | 1H), 7.00-7.13 (m, 4H), 6.45 (d, J = 7.8 Hz, 1H), 6.41 (t, J = 2.0 |
| | | | Hz, 1H), 6.36 (dd, J = 7.8, 2.0 Hz, 1H), 5.28 (br s, 2H), 3.81 (s, 3H) |
| 55 | A | 575.1 | ¹H NMR (DMSO-d₆) δ: 10.29 (s, 1H), 10.22 (br s, 1H), 9.68 (s, 1H), |
| | | | 9.61 (s, 1H), 8.70 (d, J = 8.2 Hz, 1H), 8.59 (s, 1H), 7.82 (d, J = 7.8 |
| | | | Hz, 1H), 7.68 (d, J = 8.6 Hz, 1H), 7.45-7.53 (m, 1H), 7.37-7.45 |
| | | | (m, 1H), 7.18-7.35 (m, 2H), 6.96 (d, J = 2.3 Hz, 1H), 6.87 (dd, J = |
| | | | 8.8, 2.5 Hz, 1H), 3.79 (s, 3H), 2.59 (s, 3H) |
| 56 | A | 553.2 | ¹H NMR (DMSO-d₆) δ: 10.18 (s, 1H), 10.16 (s, 1H), 9.54 (s, 1H), |
| | | | 8.75 (s, 1H), 8.56 (s, 1H), 7.67 (d, J = 8.6 Hz, 1H), 7.28 (td, J = 8.6, |
| | | | 5.9 Hz, 1H), 7.21 (t, J = 9.4 Hz, 1H), 6.95 (d, J = 2.3 Hz, 1H), 6.86 |
| | | | (dd, J = 8.8, 2.5 Hz, 1H), 3.79 (s, 3H), 2.58 (s, 3H), 2.55 (s, 3H), |
| | | | 2.14 (s, 3H) |
| 57 | A | 546.2 | ¹H NMR (DMSO-d₆) δ: 10.14 (br s, 1H), 9.57 (s, 1H), 9.13 (s, 1H), |
| | | | 8.23 (s, 1H), 7.67 (d, J = 9.0 Hz, 1H), 7.22 (td, J = 9.0, 5.9 Hz, 1H), |
| | | | 7.16 (t, J = 9.4 Hz, 1H), 6.94 (d, J = 2.3 Hz, 1H), 6.85 (dd, J = 8.8, |
| | | | 2.5 Hz, 1H), 5.44 (d, J = 53.6 Hz, 1H), 3.90-4.07 (m, 2H), 3.78 (s, |
| | | | 3H), 3.54-3.71 (m, 2H), 2.57 (s, 3H), 2.23-2.39 (m, 2H) |
| 58 | B | 532.2 | ¹H NMR (DMSO-d₆) δ: 10.49 (br s, 1H), 9.74 (s, 2H), 8.74 (s, 1H), |
| | | | 8.47 (s, 1H), 7.54 (td, J = 8.7, 5.8 Hz, 1H), 7.32 (td, J = 9.1, 1.6 Hz, |
| | | | 1H), 3.94-3.99 (m, 1H), 3.22-3.38 (m, 4H), 3.19 (s, 3H), 3.06 (s, |
| | | | 3H), 2.37 (d, J = 1.3 Hz, 3H), 1.93 (dq, J = 6.5, 4.1 Hz, 2H) |
| 59 | B | 554.1 | ¹H NMR (DMSO-d₆) δ: 10.37 (s, 1H), 9.78 (br s, 1H), 9.70 (s, 1H), |
| | | | 9.64 (s, 1H), 8.73 (d, J = 8.2 Hz, 1H), 8.65 (s, 1H), 7.83 (d, J = 7.4 |
| | | | Hz, 1H), 7.45-7.58 (m, 2H), 7.38-7.45 (m, 1H), 7.31 (td, J = 9.0, |
| | | | 1.6 Hz, 1H), 3.90-4.03 (m, 1H), 3.22-3.37 (m, 4H), 3.20 (s, 3H), |
| | | | 1.93 (td, J = 7.6, 4.3 Hz, 2H) |
| 60 | B | 525.2 | ¹H NMR (DMSO-d₆) δ: 9.78 (br s, 1H), 9.69 (s, 1H), 9.16 (s, 1H), |
| | | | 8.32 (s, 1H), 7.47 (td, J = 8.8, 5.7 Hz, 1H), 7.24 (td, J = 9.1, 1.3 Hz, |
| | | | 1H), 5.47 (d, J = 53.6 Hz, 1H), 3.92-3.99 (m, 1H), 3.80 (dd, J = |
| | | | 39.7, 13.6 Hz, 1H), 3.61-3.71 (m, 1H), 3.29-3.34 (m, 1H), 3.20- |
| | | | 3.29 (m, 3H), 3.19 (s, 3H), 2.13-2.39 (m, 2H), 1.88-1.95 (m, 2H) |
| 61 | B | 570.1 | ¹H NMR (DMSO-d₆) δ: 10.61 (br s, 1H), 9.57 (s, 1H), 9.13 (s, 1H), |
| | | | 8.22 (s, 1H), 7.90 (d, J = 2.0 Hz, 1H), 7.87 (d, J = 8.6 Hz, 1H), 7.60 |
| | | | (dd, J = 8.4, 2.2 Hz, 1H), 7.22-7.31 (m, 1H), 7.18 (t, J = 9.0 Hz, |
| | | | 1H), 5.50 (d, J = 53.6 Hz, 1H), 3.88-4.20 (m, 2H), 3.78 (dd, J = |
| | | | 38.0, 11.7 Hz, 1H), 3.63 (q, J = 7.8 Hz, 1H), 2.12-2.41 (m, 2H) |
| 62 | B | 577.1 | ¹H NMR (DMSO-d₆) δ: 10.65 (br s, 1H), 10.14 (s, 1H), 9.53 (s, 1H), |
| | | | 8.73 (s, 1H), 8.53 (s, 1H), 7.89 (d, J = 2.0 Hz, 1H), 7.86 (d, J = 8.6 |
| | | | Hz, 1H), 7.59 (dd, J = 8.6, 2.0 Hz, 1H), 7.29 (td, J = 8.6, 5.9 Hz, |
| | | | 1H), 7.21 (t, J = 9.0 Hz, 1H), 2.53 (s, 3H), 2.12 (s, 3H) |
| 63 | B | 599.0 | ¹H NMR (DMSO-d₆) δ: 10.69 (s, 1H), 10.30 (s, 1H), 9.68 (s, 1H), |
| | | | 9.61 (s, 1H), 8.70 (d, J = 7.8 Hz, 1H), 8.59 (s, 1H), 7.93 (d, J = 2.0 |
| | | | Hz, 1H), 7.89 (d, J = 8.6 Hz, 1H), 7.82 (d, J = 7.8 Hz, 1H), 7.63 (dd, |
| | | | J = 8.6, 2.0 Hz, 1H), 7.48 (t, J = 7.2 Hz, 1H), 7.41 (t, J = 8.2 Hz, |
| | | | 1H), 7.35 (td, J = 9.0, 5.5 Hz, 1H), 7.28 (t, J = 9.0 Hz, 1H) |
| 64 | B | 573.2 | ¹H NMR (DMSO-d₆) δ: 10.31 (br.s., 1H), 10.14 (s, 1H), 9.68 (s, 1H), |
| | | | 9.64 (s, 1H), 8.71 (d, J = 8.2 Hz, 1H), 8.62 (s, 1H), 7.82 (d, J = 7.9 |
| | | | Hz, 1H), 7.62 (d, J = 1.6 Hz, 1H), 7.37-7.57 (m, 3H), 7.17-7.37 |
| | | | (m, 2H), 6.91 (d, J = 8.5 Hz, 1H), 4.62 (t, J = 8.8 Hz, 2H), 3.22 (t, J = |
| | | | 8.8 Hz, 1H) |
| 65 | B | 551.5 | ¹H NMR (DMSO-d₆) δ: 10.42 (br s, 1H), 10.12 (s, 1H), 9.73 (s, 1H), |
| | | | 8.70 (s, 1H), 8.45 (br s, 1H), 7.60-7.63 (m, 1H), 7.50 (dd, J = 8.5, |
| | | | 2.2 Hz, 1H), 7.25-7.28 (m, J = 8.2 Hz, 2H), 6.90 (d, J = 8.5 Hz, |
| | | | 1H), 4.62 (t, J = 8.8 Hz, 2H), 3.22 (t, J = 8.8 Hz, 2H), 3.05 (s, 3H), |
| | | | 2.36 (d, J = 1.3 Hz, 3H) |
| 66 | B | 544.5 | ¹H NMR (DMSO-d₆) δ: 10.07 (s, 1H), 9.73 (br.s., 1H), 9.14 (s, 1H), |
| | | | 8.29 (s, 1H), 7.60 (d, J = 1.6 Hz, 1H), 7.50 (d, J = 8.5 Hz, 1H), 7.16- |
| | | | 7.26 (m, 2H), 6.89 (d, J = 8.5 Hz, 1H), 5.51 (d, J = 52 Hz, 1H), |
| | | | 4.62 (t, J = 8.8 Hz, 2H), 3.60-4.10 (m, 4H), 3.21 (t, J = 9.0 Hz, |
| | | | 2H), 2.11-2.34 (m, 2H) |
| 67 | B | 555.6 | ¹H NMR (CDCl₃) δ: 9.77 (br s, 1H), 9.69 (s, 1H), 9.22 (s, 1H), 8.32 |
| | | | (s, 1H), 7.44-7.51 (m, 2H), 7.32-7.39 (m, 2H), 7.24 (t, J = 9.1 Hz, |
| | | | 1H), 5.02 (br s, 4H), 3.91-4.00 (m, 1H), 3.33 (dd, J = 10.7, 4.7 Hz, |
| | | | 1H), 3.21-3.30 (m, 3H), 3.19 (s, 3H), 1.84-1.97 (m, 2H) |
| 68 | B | 553.2 | ¹H NMR (DMSO-d₆) δ: 10.26 (s, 1H), 9.74 (s, 1H), 9.62 (s, 1H), 8.87 |
| | | | (d, J = 8.5 Hz, 1H), 8.82 (d, J = 3.5 Hz, 1H), 8.58 (s, 1H), 7.69 (dt, |
| | | | J = 7.6, 0.9 Hz, 1H), 7.52 (td, J = 8.9, 5.8 Hz, 1H), 7.38 (ddd, J = |
| | | | 8.4, 7.3, 1.3 Hz, 1H), 7.30 (td, J = 9.1, 1.6 Hz, 1H), 7.27 (td, J = 7.3, |
| | | | 0.9 Hz, 1H), 6.91 (dd, J = 3.8, 0.6 Hz, 1H), 3.95-3.99 (m, 1H), 3.34 |
| | | | (dd, J = 10.7, 4.7 Hz, 1H), 3.22-3.31 (m, 3H), 3.20 (s, 3H), 1.83- |
| | | | 2.03 (m, 2H) |
| 69 | B | 554.5 | ¹H NMR (DMSO-d₆) δ: 10.36 (br s, 1H), 9.86 (s, 1H), 9.74 (s, 1H), |
| | | | 9.70 (s, 1H), 8.72 (s, 1H), 7.53 (td, J = 8.8, 5.7 Hz, 1H), 7.31 (td, J = |
| | | | 9.1, 1.6 Hz, 1H), 3.92-4.00 (m, 1H), 3.33 (dd, J = 10.7, 4.7 Hz, |
| | | | 1H), 3.20-3.31 (m, 5H), 3.19 (s, 3H), 2.67-2.76 (m, 2H), 1.90- |
| | | | 1.97 (m, 2H), 1.80-1.90 (m, 4H) |
| 70 | C | 568.5 | ¹H NMR (DMSO-d₆) δ: 10.38 (s, 1H), 9.77 (s, 1H), 9.70 (s, 1H), 9.65 |
| | | | (s, 1H), 8.73 (d, J = 8.2 Hz, 1H), 8.64 (s, 1H), 7.83 (d, J = 7.9 Hz, |
| | | | 1H), 7.54 (td, J = 8.8, 5.7 Hz, 1H), 7.47-7.51 (m, 1H), 7.40-7.45 |
| | | | (m, 1H), 7.32 (td, J = 9.5, 1.6 Hz, 1H), 3.34 (dd, J = 9.8, 7.6 Hz, |
| | | | 1H), 3.18-3.32 (m, 4H), 3.20 (s, 3H), 2.96 (dd, J = 9.6, 7.1 Hz, 1H), |
| | | | 2.45 (dt, J = 14.3, 7.3 Hz, 1H), 1.94 (dtd, J = 12.2, 7.3, 4.4 Hz, 1H), |
| | | | 1.58 (dq, J = 12.6, 7.9 Hz, 1H) |
| 71 | B | 554.5 | ¹H NMR (DMSO-d₆) δ: 10.39 (s, 1H), 9.74 (br s, 1H), 9.67 (s, 1H), |
| | | | 8.85 (br s, 1H), 8.61 (s, 1H), 8.47 (dd, J = 4.7, 1.6 Hz, 1H), 8.12 |
| | | | (dd, J = 7.7, 1.7 Hz, 1H), 7.52 (td, J = 8.8, 5.7 Hz, 1H), 7.32 (dd, J = |
| | | | 7.9, 4.7 Hz, 1H), 7.29 (t, J = 9.1 Hz, 1H), 6.91 (d, J = 4.1 Hz, 1H), |
| | | | 3.93-4.01 (m, 1H), 3.23-3.30 (m, 3H), 3.19 (s, 3H), 1.89-1.96 |
| | | | (m, 2H) |
| 72 | B | 555.5 | ¹H NMR (DMSO-d₆) δ: 10.38 (s, 1H), 9.76 (s, 1H), 9.70 (s, 1H), 9.65 |
| | | | (s, 1H), 8.73 (d, 8.2 Hz, 1H), 8.65 (s, 1H), 7.83 (d, J = 7.9 Hz, 1H), |
| | | | 7.46-7.60 (m, 1H), 7.39-7.46 (m, 1H), 7.23-7.39 (m, 1H), 3.97 |
| | | | (m, 1H), 3.26-3.37 (m, 3H), 3.22-3.26 (m, 1H), 3.20 (s, 3H), 1.88- |
| | | | 2.00 (m, 2H) |
| 73 | C | 538.2 | ¹H NMR (DMSO-d₆) δ: 10.36 (br s, 1H), 9.69 (s, 1H), 9.64 (s, 1H), |
| | | | 8.72 (d, J = 7.8 Hz, 1H), 8.63 (s, 1H), 7.83 (d, J = 7.8 Hz, 1H), 7.53 |
| | | | (td, J = 9.0, 5.9 Hz, 1H), 7.45-7.51 (m, 1H), 7.41 (td, J = 7.8, 1.2 |
| | | | Hz, 1H), 7.30 (td, J = 9.4, 1.2 Hz, 1H), 3.37 (dd, J = 9.4, 7.0 Hz, |
| | | | 1H), 3.19 (td, J = 9.0, 7.0 Hz, 1H), 2.73 (dd, J = 9.0, 8.2 Hz, 1H), |
| | | | 2.22 (dq, J = 14.7, 7.2 Hz, 1H), 1.91-2.03 (m, 1H), 1.44 (dq, J = |
| | | | 12.3, 8.4 Hz, 1H), 0.96 (d, J = 6.7 Hz, 3H) |
| 74 | C | 536.1 | ¹H NMR (DMSO-d₆) δ: 10.37 (br s, 1H), 9.70 (s, 1H), 9.65 (s, 1H), |
| | | | 8.73 (d, J = 8.2 Hz, 1H), 8.65 (s, 1H), 7.83 (d, J = 7.8 Hz, 1H), 7.46- |
| | | | 7.53 (m, 1H), 7.39-7.45 (m, 1H), 7.30 (t, J = 8.8 Hz, 1H), 3.29 |
| | | | (br.s, 4H), 1.55 (dd, J = 7.2, 3.3 Hz, 2H), 0.58 (td, J = 7.7, 4.9 Hz, |
| | | | 1H), 0.23 (q, J = 4.3 Hz, 1H) |
| 75 | B | 572.1 | ¹H NMR (DMSO-d₆) δ: 10.36 (br s, 1H), 9.70 (s, 1H), 9.64 (s, 1H), |
| | | | 8.73 (d, J = 8.2 Hz, 1H), 8.64 (s, 1H), 7.83 (d, J = 7.8 Hz, 1H), 7.46- |
| | | | 7.60 (m, 2H), 7.38-7.45 (m, 1H), 7.29 (t, J = 8.8 Hz, 1H), 3.56- |
| | | | 3.75 (m, 2H), 3.41 (dd, J = 9.6, 7.6 Hz, 1H), 3.16-3.26 (m, 1H), |
| | | | 3.01 (dd, J = 9.8, 7.0 Hz, 1H), 2.57 (dt, J = 14.6, 7.4 Hz, 1H), 1.92- |
| | | | 2.09 (m, 1H), 1.68 (dq, J = 12.6, 7.9 Hz, 1H) |
| 76 | C | 552.2 | ¹H NMR (DMSO-d₆) δ: 10.36 (br s, 1H), 9.70 (s, 1H), 9.64 (s, 1H), |
| | | | 8.73 (d, J = 8.2 Hz, 1H), 8.62 (s, 1H), 7.83 (d, J = 7.8 Hz, 1H), 7.45- |
| | | | 7.59 (m, 2H), 7.37-7.45 (m, 1H), 7.29 (t, J = 9.0 Hz, 1H), 3.38 |
| | | | (dd, J = 9.4, 7.4 Hz, 1H), 3.17 (td, J = 9.4, 6.7 Hz, 1H), 2.77 (t, J = |
| | | | 9.0 Hz, 1H), 1.89-2.11 (m, 2H), 1.38-1.53 (m, 1H), 1.25-1.38 |
| | | | (m, 2H), 0.83 (t, J = 7.4 Hz, 3H) |
| 77 | C | 524.1 | ¹H NMR (DMSO-d₆) δ: 10.37 (s, 1H), 9.73 (s, 1H), 9.70 (s, 1H), 9.64 |
| | | | (s, 1H), 8.72 (d, J = 7.8 Hz, 1H), 8.64 (s, 1H), 7.83 (d, J = 7.8 Hz, |
| | | | 1H), 7.54 (td, J = 9.0, 5.9 Hz, 1H), 7.46-7.52 (m, 1H), 7.38-7.45 |
| | | | (m, 1H), 7.32 (td, J = 9.2, 1.6 Hz, 1H), 3.11-3.25 (m, 4H), 1.81 (dt, |
| | | | J = 6.5, 3.4 Hz, 4H) |
| 78 | C | 590.1 | ¹H NMR (DMSO-d₆) δ: 10.36 (s, 1H), 9.70 (s, 1H), 9.64 (s, 1H), 8.73 |
| | | | (d, J = 7.8 Hz, 1H), 8.65 (s, 1H), 7.83 (d, J = 7.8 Hz, 1H), 7.46- |
| | | | 7.57 (m, 2H), 7.39-7.45 (m, 1H), 7.30 (t, J = 9.0 Hz, 1H), 4.79- |
| | | | 4.91 (m, 1H), 3.49 (dd, J = 11.2, 4.9 Hz, 1H), 3.34-3.39 (m, 1H), |
| | | | 3.26-3.30 (m, 2H), 2.08-2.21 (m, 1H), 1.94-2.06 (m, 1H) |
| 79 | A | 594.1 | ¹H NMR (DMSO-d₆) δ: 10.45 (br s, 1H), 10.27 (s, 1H), 9.64 (s, 1H), |
| | | | 9.39 (s, 1H), 8.55 (s, 1H), 7.84 (dd, J = 8.2, 0.9 Hz, 1H), 7.63 (t, J = |
| | | | 8.8 Hz, 1H), 7.22-7.39 (m, 2H), 7.13 (t, J = 7.9 Hz, 1H), 7.07 (dd, |
| | | | J = 12.5, 2.4 Hz, 1H), 6.90 (dd, J = 8.9, 2.6 Hz, 1H), 6.58 (dd, J = |
| | | | 7.8, 1.0 Hz, 1H), 5.50 (br s, 2H), 3.83 (s, 3H) |
| 80 | A | 614.1 | ¹H NMR (DMSO-d₆) δ: 10.76 (br s, 1H), 10.26 (s, 1H), 9.64 (s, 1H), |
| | | | 9.38 (s, 1H), 8.56 (s, 1H), 7.95 (dd, J = 7.9, 1.1 Hz, 1H), 7.91 (dd, |
| | | | J = 7.9, 1.6 Hz, 1H), 7.84 (d, J = 8.3 Hz, 1H), 7.54 (t, J = 8.1 Hz, |
| | | | 1H), 7.20-7.39 (m, 2H), 7.12 (t, J = 7.9 Hz, 1H), 6.57 (dd, J = 7.8, |
| | | | 1.0 Hz, 1H), 5.50 (br s, 2H) |
| 81 | A | 599.0 | ¹H NMR (DMSO-d₆) δ: 10.77 (br s, 1H), 10.30 (s, 1H), 9.68 (s, 1H), |
| | | | 9.60 (s, 1H), 8.70 (d, J = 7.8 Hz, 1H), 8.59 (s, 1H), 7.97 (dd, J = 8.1, |
| | | | 1.5 Hz, 1H), 7.91 (dd, J = 7.9, 1.6 Hz, 1H), 7.82 (dd, J = 7.2, 0.9 |
| | | | Hz, 1H), 7.55 (t, J = 8.1 Hz, 1H), 7.48 (td, J = 7.3, 1.2 Hz, 1H), 7.42 |
| | | | (dd, J = 7.8, 1.5 Hz, 1H), 7.21-7.38 (m, 2H) |
| 82 | A | 578.1 | ¹H NMR (DMSO-d₆) δ: 10.55 (br s, 1H), 10.26 (s, 1H), 9.64 (s, 1H), |
| | | | 9.38 (s, 1H), 8.56 (s, 1H), 7.84 (dd, J = 8.1, 0.7 Hz, 1H), 7.62 (d, J = |
| | | | 7.3 Hz, 1H), 7.50 (td, J = 9.5, 1.2 Hz, 1H), 7.42 (dd, J = 8.1, 5.6 |
| | | | Hz, 1H), 7.21-7.39 (m, 2H), 7.12 (t, J = 7.9 Hz, 1H), 6.57 (dd, J = |
| | | | 7.8, 1.0 Hz, 1H), 5.50 (br s, 2H), 2.51 (s, 3H) |
| 83 | A | 563.1 | ¹H NMR (DMSO-d₆) δ: 10.55 (br s, 1H), 10.29 (s, 1H), 9.68 (s, 1H), |
| | | | 9.61 (s, 1H), 8.70 (d, J = 7.6 Hz, 1H), 8.59 (s, 1H), 7.76-7.88 (m, |
| | | | 1H), 7.62 (d, J = 7.8 Hz, 1H), 7.17-7.56 (m, 6H), 2.51 (s, 3H) |
| 93 | A | 579.1 | ¹H NMR (DMSO-d₆) δ: 10.51 (s, 1H), 10.10 (s, 1H), 9.71 (s, 1H), |
| | | | 8.61 (s, 1H), 7.79 (d, J = 8.2 Hz, 1H), 7.62 (d, J = 7.8 Hz, 1H), 7.49 |
| | | | (t, J = 9.0 Hz, 1H), 7.41 (td, J = 8.2, 5.9 Hz, 1H), 7.39 (t, J = 8.2 Hz, |
| | | | 1H), 7.32 (td, J = 8.7, 6.1 Hz, 1H), 7.24 (t, J = 9.4 Hz, 1H), 6.62 (d, |
| | | | J = 7.4 Hz, 1H), 6.36 (s, 2H), 2.50 (br s, 3H) |
| 94 | A | 615.0 | ¹H NMR (DMSO-d₆) δ: 10.73 (br s, 1H), 10.11 (s, 1H), 9.71 (s, 1H), |
| | | | 8.61 (s, 1H), 7.95 (d, J = 8.2 Hz, 1H), 7.90 (dd, J = 7.8, 1.2 Hz, 1H), |
| | | | 7.80 (d, J = 8.2 Hz, 1H), 7.54 (t, J = 8.0 Hz, 1H), 7.39 (t, J = 7.8 Hz, |
| | | | 1H), 7.27-7.36 (m, 1H), 7.24 (t, J = 9.2 Hz, 1H), 6.62 (d, J = 7.8 |
| | | | Hz, 1H), 6.36 (s, 2H) |
| 95 | F | 699.1 | ¹H NMR (DMSO-d₆) δ: 10.56 (br s, 1H), 10.46 (br s, 1H), 9.68 (s, |
| | | | 1H), 9.35 (s, 1H), 8.96 (d, J = 8.2 Hz, 1H), 8.58 (s, 1H), 7.96 (d, J = |
| | | | 7.8 Hz, 1H), 7.62 (d, J = 7.8 Hz, 1H), 7.53 (dd, J = 9.0, 7.8 Hz, 1H), |
| | | | 7.28-7.48 (m, 4H), 7.17-7.26 (m, 1H), 2.74 (s, 6H) |
| 96 | F | 724.2 | ¹H NMR (DMSO-d₆) δ: 10.47 (s, 1H), 9.68 (s, 1H), 9.33 (s, 1H), 8.96 |
| | | | (d, J = 8.2 Hz, 1H), 8.58 (s, 1H), 7.93 (d, J = 7.8 Hz, 1H), 7.62 |
| | | | (d, J = 7.4 Hz, 1H), 7.51-7.56 (m, 1H), 7.30-7.49 (m, 5H), 7.19- |
| | | | 7.27 (m, 1H), 3.07 (br s, 4H), 2.37-2.43 (m, 4H), 2.13 (s, 3H) |
| 97 | E | 633.3 | ¹H NMR (DMSO-d₆) δ: 10.56 (br s, 1H), 10.28 (s, 1H), 9.63 (s, 1H), |
| | | | 9.07 (s, 1H), 8.87 (d, J = 8.5 Hz, 1H), 8.54 (s, 1H), 7.72 (d, J = 7.8 |
| | | | Hz, 1H), 7.62 (d, J = 7.8 Hz, 1H), 7.38-7.50 (m, 3H), 7.30-7.35 |
| | | | (m, 2H), 7.24 (t, J = 8.7 Hz, 1H), 3.12 (br s, 6H), 2.50 (s, 3H). |
| 98 | E | 710.2 | ¹H NMR (DMSO-d₆) δ: 10.57 (br s, 1H), 10.27 (s, 1H), 9.63 (s, 1H), |
| | | | 9.12 (s, 1H), 8.90 (d, J = 8.0 Hz, 1H), 8.57 (br s, 2H), 8.55 (s, 1H), |
| | | | 7.77 (d, J = 7.5 Hz, 1H), 7.62 (d, J = 7.8 Hz, 1H), 7.25-7.50 (m, |
| | | | 8H), 4.81 (s, 2H), 3.13 (br s, 3H), 2.50 (s, 3H). |
| 99 | E | 688.2 | ¹H NMR (DMSO-d₆) δ: 10.33 (s, 1H), 9.63 (s, 1H), 9.09 (s, 1H), 8.88 |
| | | | (d, J = 8.2 Hz, 1H), 8.54 (s, 1H), 7.67 (d, J = 7.8 Hz, 1H), 7.62 (d, J = |
| | | | 7.8 Hz, 1H), 7.42-7.48 (m, 2H), 7.28-7.41 (m, 3H), 7.21 (t, J = |
| | | | 9.0 Hz, 1H), 3.68 (br s, 4H), 2.50 (s, 3H), 2.39 (br s, 4H), 2.22 (s, |
| | | | 3H). |
| 100 | E | 675.2 | ¹H NMR (DMSO-d₆) δ: 10.57 (s, 1H), 10.32 (s, 1H), 9.64 (s, 1H), |
| | | | 9.11 (s, 1H), 8.89 (d, J = 8.5 Hz, 1H), 8.54 (s, 1H), 7.71 (d, J = 7.8 |
| | | | Hz, 1H), 7.62 (d, J = 7.8 Hz, 1H), 7.41-7.49 (m, 2H), 7.29-7.40 |
| | | | (m, 3H), 7.23 (t, J = 8.7 Hz, 1H), 3.65 (br s, 8H), 2.50 (s, 3H). |
| 101 | E | 663.4 | ¹H NMR (DMSO-d₆) δ: 10.56 (s, 1H), 10.30 (s, 1H), 9.63 (s, 1H), |
| | | | 9.05 (s, 1H), 8.88 (d, J = 8.5 Hz, 1H), 8.54 (s, 1H), 7.69 (br s, 1H), |
| | | | 7.62 (d, J = 7.8 Hz, 1H), 7.49 (t, J = 8.7 Hz, 1H), 7.39-7.45 (m, |
| | | | 2H), 7.31-7.37 (m, 2H), 7.26 (t, J = 9.1 Hz, 1H), 4.80 (br s, 1H), |
| | | | 3.58-3.66 (m, 4H), 3.13 (s, 3H), 2.51 (s, 3H). |
| 102 | E | 689.5 | ¹H NMR (DMSO-d₆) δ: 10.56 (s, 1H), 10.35 (s, 1H), 9.63 (s, 1H), |
| | | | 9.08 (s, 1H), 8.88 (d, J = 8.5 Hz, 1H), 8.54 (s, 1H), 7.63 (dd, J = |
| | | | 12.0, 7.9 Hz, 2H), 7.50 (t, J = 8.7 Hz, 1H), 7.39-7.46 (m, 2H), 7.31- |
| | | | 7.37 (m, 2H), 7.26 (t, J = 9.4 Hz, 1H), 4.82 (d, J = 4.1 Hz, 1H), |
| | | | 3.77 (td, J = 8.1, 4.3 Hz, 1H), 3.27-3.33 (m, 4H), 2.50 (s, 3H), 1.78 |
| | | | (s, 2H), 1.39 (s, 2H). |
| 103 | E | 689.5 | ¹H NMR (DMSO-d₆) δ: 10.56 (s, 1H), 10.36 (s, 1H), 9.63 (s, 1H), |
| | | | 9.08 (s, 1H), 8.88 (d, J = 8.5 Hz, 1H), 8.54 (s, 1H), 7.63 (dd, J = |
| | | | 11.4, 7.8 Hz, 2H), 7.39-7.51 (m, 3H), 7.30-7.36 (m, 2H), 7.25 (t, |
| | | | J = 8.5 Hz, 1H), 4.86-4.99 (m, 1H), 3.75-3.91 (m, 1H), 3.50 (br |
| | | | s, 1H), 3.31 (s, 1H), 3.23 (br s, 1H), 2.82-3.11 (m, 1H), 2.50 (s, |
| | | | 3H), 1.88-1.90 (m, 1H), 1.75 (br s, 1H), 1.42-1.45 (m, 2H). |
| 104 | E | 689.3 | ¹H NMR (DMSO-d₆) δ: 9.92 (br s, 1H), 9.68 (s, 1H), 8.90 (d, J = 8.5 |
| | | | Hz, 1H), 8.59 (s, 1H), 8.00 (d, J = 8.2 Hz, 1H), 7.69 (t, J = 7.8 Hz, |
| | | | 1H), 7.63 (d, J = 7.8 Hz, 1H), 7.49 (t, J = 7.4 Hz, 1H), 7.45 (d, J = |
| | | | 8.9 Hz, 1H), 7.39 (td, J = 8.0, 5.9 Hz, 1H), 7.29 (td, J = 8.7, 5.7 Hz, |
| | | | 1H), 7.19 (t, J = 8.5 Hz, 1H), 3.85 (br s, 2H), 3.79 (br s, 2H), 2.50 |
| | | | (br s, 3H), 2.48 (br s, 2H), 2.08 (br s, 2H), 2.25 (s, 3H). |
| 105 | E | 634.3 | ¹H NMR (DMSO-d₆) δ: 10.52 (br s, 1H), 9.93 (s, 1H), 9.67 (s, 1H), |
| | | | 8.89 (d, J = 8.5 Hz, 1H), 8.58 (s, 1H), 8.02 (d, J = 8.0 Hz, 1H), 7.68 |
| | | | (td, J = 7.1, 1.0 Hz, 1H), 7.63 (d, J = 7.8 Hz, 1H), 7.45-7.50 (m, |
| | | | 2H), 7.40 (td, J = 7.8, 5.7 Hz, 1H), 7.31 (td, J = 8.9, 5.9 Hz, 1H), |
| | | | 7.22 (t, J = 8.9 Hz, 1H), 3.33 (s, 3H), 3.14 (s, 3H), 2.50 (s, 3H). |
| 106 | E | 711.2 | ¹H NMR (DMSO-d₆) δ: 10.52 (br s, 1H), 9.93 (s, 0.6H), 9.78 (s, |
| | | | 0.4H), 9.69 (s, 0.6H), 9.65 (s, 0.4H), 8.92 (d, J = 8.7 Hz, 1H), 8.88 |
| | | | (d, J = 8.2 Hz, 1H), 8.59 (t, J = 5.0 Hz, 2H), 8.47 (d, J = 5.7 Hz, 1H), |
| | | | 8.10 (t, J = 8.0 Hz, 1H), 7.69 (dd, J = 15.8, 8.7 Hz, 1H), 7.63 (d, J = |
| | | | 7.8 Hz, 1H), 7.46-7.53 (m, 2H), 7.38-7.43 (m, 3H), 7.29-7.35 |
| | | | (m, 1H), 7.22-7.27 (m, 1H), 5.14 (s, 0.8H), 4.87 (s, 1.2H), 3.38 (s, |
| | | | 1.8H), 3.11 (s, 1.2H), 2.50 (s, 3H). |
| 107 | E | 664.3 | ¹H NMR (DMSO-d₆) δ: 10.54 (br s, 1H), 9.95 (d, J = 6.2 Hz, 1H), |
| | | | 9.68 (d, J = 2.7 Hz, 1H), 8.89 (t, J = 9.8 Hz, 1H), 8.59 (d, J = 2.1 |
| | | | Hz, 1H), 8.01 (d, J = 8.2 Hz, 1H), 7.69 (t, J = 7.8 Hz, 1H), 7.62 (d, |
| | | | J = 7.8 Hz, 1H), 7.46-7.53 (m, 2H), 7.39-7.44 (m, 1H), 7.30- |
| | | | 7.36 (m, 1H), 7.26 (t, J = 9.1 Hz, 1H), 4.90-5.06 (m, 1H), 3.83 (t, |
| | | | J = 5.5 Hz, 1H), 3.70-3.73 (m, 1H), 3.65-3.68 (m, 1H), 3.64 (t, J = |
| | | | 5.3 Hz, 1H), 3.34 (s, 1H), 3.14 (s, 2H), 2.50 (s, 3H). |
| 108 | E | 690.3 | ¹H NMR (DMSO-d₆) δ: 10.54 (br s, 1H), 9.95 (s, 1H), 9.68 (s, 1H), |
| | | | 8.89 (d, J = 8.5 Hz, 1H), 8.58 (s, 1H), 7.96 (d, J = 8.0 Hz, 1H), 7.69 |
| | | | (ddd, J = 8.5, 7.1, 0.9 Hz, 1H), 7.62 (d, J = 7.5 Hz, 1H), 7.49 (q, J = |
| | | | 7.2 Hz, 2H), 7.41 (td, J = 8.0, 5.5 Hz, 1H), 7.32 (td, J = 8.7, 5.9 Hz, |
| | | | 1H), 7.25 (dd, J = 9.1, 0.7 Hz, 1H), 4.82 (br s, 1H), 4.17 (dt, J = |
| | | | 12.3, 4.3 Hz, 1H), 4.02 (dt, J = 12.6, 3.7 Hz, 1H), 3.80 (tt, J = 8.1, |
| | | | 3.8 Hz, 1H), 3.47 (ddd, J = 13.2, 9.7, 3.2 Hz, 2H), 2.50 (s, 3H), 1.88- |
| | | | 1.91 (m, 1H), 1.76-1.79 (m, 1H), 1.35-1.52 (m, 2H). |
| 109 | E | 690.3 | ¹H NMR (DMSO-d₆) δ: 10.54 (br s, 1H), 9.94 (s, 1H), 9.67 (d, J = |
| | | | 3.2 Hz, 1H), 8.89 (dd, J = 8.5, 4.1 Hz, 1H), 8.58 (d, J = 1.4 Hz, 1H), |
| | | | 7.96 (d, J = 8.0 Hz, 1H), 7.69 (t, J = 7.7 Hz, 1H), 7.63 (d, J = 7.8 |
| | | | Hz, 1H), 7.48 (t, J = 7.9 Hz, 2H), 7.40 (td, J = 7.8, 6.2 Hz, 1H), 7.31 |
| | | | (td, J = 8.7, 5.7 Hz, 1H), 7.22 (t, J = 8.7 Hz, 1H), 5.10 (d, J = 4.1 |
| | | | Hz, 0.5H), 4.88 (d, J = 4.1 Hz, 0.5H), 4.30 (dd, J = 12.5, 3.5 Hz, |
| | | | 0.5H), 3.97 (d, J = 13.3 Hz, 0.5H), 3.89 (dd, J = 12.8, 2.7 Hz, 1H), |
| | | | 3.63 (dt, J = 8.2, 4.1 Hz, 0.5), 3.55 (br s, 0.5H), 3.45-3.50 (m, 1H), |
| | | | 3.46 (dd, J = 12.8, 7.5 Hz, 0.5H), 3.04 (dd, J = 12.3, 8.7 Hz, 0.5H), |
| | | | 2.50 (s, 3H), 1.70-1.91 (m, 2H), 1.41-1.53 (m, 2H). |
| 110 | E | 676.3 | ¹H NMR (DMSO-d₆) δ: 10.54 (br s, 1H), 9.93 (s, 1H), 9.68 (s, 1H), |
| | | | 8.90 (d, J = 8.7 Hz, 1H), 8.59 (s, 1H), 8.05 (d, J = 8.2 Hz, 1H), 7.70 |
| | | | (ddd, J = 8.2, 7.0, 0.9 Hz, 1H), 7.63 (d, J = 7.8 Hz, 1H), 7.50 (t, J = |
| | | | 7.5 Hz, 2H), 7.40 (dd, J = 13.5, 7.8 Hz, 1H), 7.31 (dd, J = 14.6, 8.9 |
| | | | Hz, 1H), 7.22 (t, J = 8.0 Hz, 1H), 3.92-3.94 (m, 2H), 3.78-3.80 |
| | | | (m, 2H), 3.74-3.76 (m, 2H), 3.63-3.66 (m, 2H), 2.50 (s, 3H). |
| 111 | A | 608.3 | ¹H NMR (DMSO-d₆) δ: 10.37 (br s, 1H), 10.25 (s, 1H), 9.64 (s, 1H), |
| | | | 9.38 (s, 1H), 8.55 (s, 1H), 7.84 (d, J = 7.8 Hz, 1H), 7.58 (d, J = 9.0 |
| | | | Hz, 1H), 7.28-7.36 (m, 1H), 7.20-7.27 (m, 1H), 7.13 (dd, J = 9.0 |
| | | | Hz, 2H), 6.57 (d, J = 7.8 Hz, 1H), 5.50 (s, 2H), 3.88 (s, 3H). |
| 112 | A | 624.3 | ¹H NMR (DMSO-d₆) δ: 10.44 (br s, 1H), 10.25 (s, 1H), 9.64 (s, 1H), |
| | | | 9.38 (s, 1H), 8.55 (s, 1H), 7.84 (d, J = 7.8 Hz, 1H), 7.77 (d, J = 9.0 |
| | | | Hz, 1H), 7.29-7.37 (m, 1H), 7.24 (dd, J = 11.0, 9.0 Hz, 1H), 7.12 |
| | | | (dd, J = 9.0, 7.0 Hz, 2H), 6.57 (d, J = 7.8 Hz, 1H), 5.50 (br s, 2H), |
| | | | 3.91 (s, 3H), 2.67 (s, 3H). |
| 113 | F | 746.1 | ¹H NMR (DMSO-d₆) δ: 10.56 (br s, 1H), 10.45 (s, 1H), 9.69 (s, 1H), |
| | | | 9.44 (s, 1H), 8.98 (d, J = 8.2 Hz, 1H), 8.50-8.61 (m, 3H), 8.00 |
| | | | (d, J = 7.8 Hz, 1H), 7.61 (d, J = 7.4 Hz, 1H), 7.55 (dd, J = 9.4, 7.4 |
| | | | Hz, 1H), 7.27-7.50 (m, 6H), 7.15-7.25 (m, 1H), 4.35 (s, 2H), 2.74 |
| | | | (s, 3H) |
| 114 | A | 640.4 | ¹H NMR (DMSO-d₆) δ: 10.56 (br s, 1H), 10.31 (s, 1H), 9.71 (s, 1H), |
| | | | 9.66 (s, 1H), 9.24 (br s, 1H), 8.80 (d, J = 8.2 Hz, 1H), 8.57-8.66 |
| | | | (m, 2H), 8.43 (dt, J = 8.1, 1.8 Hz, 1H), 7.66-7.73 (m, 1H), 7.54- |
| | | | 7.65 (m, 3H), 7.45-7.53 (m, 1H), 7.23-7.45 (m, 3H) |
| 115 | A | 609.4 | ¹H NMR (400 MHz, DMSO-d₆) δ: 10.44 (br s, 1H), 10.28 (s, 1H), |
| | | | 9.68 (s, 1H), 9.61 (s, 1H), 8.70 (d, J = 7.8 Hz, 1H), 8.57 (s, 1H), |
| | | | 7.82 (d, J = 7.4 Hz, 1H), 7.78 (d, J = 9.0 Hz, 1H), 7.48 (ddd, J = 8.2, |
| | | | 7.0, 1.2 Hz, 1H), 7.41 (td, J = 7.8, 1.2 Hz, 1H), 7.27-7.36 (m, 1H), |
| | | | 7.16-7.26 (m, 1H), 7.12 (d, J = 9.4 Hz, 1H), 3.90 (s, 3H), 2.67 (s, |
| | | | 3H) |
| 116 | A | 593.4 | ¹H NMR (400 MHz, DMSO-d₆) δ: 10.38 (br s, 1H), 10.27 (s, 1H), |
| | | | 9.67 (s, 1H), 9.62 (s, 1H), 8.70 (d, J = 8.2 Hz, 1H), 8.58 (s, 1H), |
| | | | 7.82 (d, J = 7.4 Hz, 1H), 7.59 (dd, J = 9.0, 0.8 Hz, 1H), 7.48 (ddd, J = |
| | | | 7.8, 7.1, 0.8 Hz, 1H), 7.41 (td, J = 7.8, 1.2 Hz, 1H), 7.24-7.36 |
| | | | (m, 1H), 7.14-7.24 (m, 1H), 7.10 (t, J = 8.6 Hz, 1H), 3.87 (s, 3H), |
| | | | 2.49 (br s, 3H) |
| 117 | A | 610.2 | ¹H NMR (400 MHz, DMSO-d₆) δ: 10.41 (br s, 1H), 10.20 (s, 1H), |
| | | | 9.76 (s, 1H), 8.79 (d, J = 8.6 Hz, 1H), 8.63 (s, 1H), 8.27 (d, J = 8.6 |
| | | | Hz, 1H), 7.79 (ddd, J = 7.8, 7.0, 0.8 Hz, 1H), 7.78 (d, J = 9.0 Hz, |
| | | | 1H), 7.62 (ddd, J = 8.2, 7.4, 0.8 Hz, 1H), 7.32 (td, J = 8.6, 6.3 Hz, |
| | | | 1H), 7.21 (br. t, J = 9.0 Hz, 1H), 7.13 (d, J = 8.6 Hz, 1H), 3.91 (s, |
| | | | 3H), 2.66 (s, 3H) |
| 118 | A | 594.4 | ¹H NMR (400 MHz, DMSO-d₆) δ: 10.35 (br s, 1H), 10.21 (s, 1H), |
| | | | 9.76 (s, 1H), 8.79 (d, J = 8.2 Hz, 1H), 8.63 (s, 1H), 8.27 (d, J = 8.2 |
| | | | Hz, 1H), 7.79 (ddd, J = 8.2, 7.4, 0.8 Hz, 1H), 7.63 (ddd, J = 7.8, 7.0, |
| | | | 0.8 Hz, 1H), 7.59 (dd, J = 9.0, 1.2 Hz, 1H), 7.31 (td, J = 8.4, 6.3 Hz, |
| | | | 1H), 7.21 (t, J = 8.6 Hz, 1H), 7.13 (t, J = 8.6 Hz, 1H), 3.88 (s, 3H), |
| | | | 2.48 (d, J = 2.7 Hz, 3H) |
| 119 | F | 772.4 | ¹H NMR (400 MHz, DMSO-d₆) δ: 10.44 (br s, 1H), 9.67 (s, 1H), 9.36 |
| | | | (s, 1H), 8.95 (d, J = 8.2 Hz, 1H), 8.56 (s, 1H), 8.14 (s, 1H), 7.96 (d, |
| | | | J = 7.8 Hz, 1H), 7.78 (d, J = 9.0 Hz, 1H), 7.53 (ddd, J = 8.2, 7.4, 0.8 |
| | | | Hz, 1H), 7.44 (ddd, J = 7.8, 7.0, 0.8 Hz, 1H), 7.31 (td, J = 8.8, 5.9 |
| | | | Hz, 1H), 7.17 (t, J = 9.0 Hz, 1H), 7.10 (d, J = 9.0 Hz, 1H), 3.90 (s, |
| | | | 3H), 3.16-3.22 (m, 4H), 2.82 (s, 3H), 2.67 (s, 3H), 2.21 (s, 6H) |
| 120 | F | 756.4 | ¹H NMR (400 MHz, DMSO-d₆) δ: 10.45 (br s, 1H), 9.67 (s, 1H), 9.36 |
| | | | (s, 1H), 8.96 (d, J = 8.6 Hz, 1H), 8.56 (s, 1H), 8.14 (s, 1H), 7.96 (d, |
| | | | J = 7.8 Hz, 1H), 7.59 (d, J = 8.6 Hz, 1H), 7.53 (t, J = 7.8 Hz, 1H), |
| | | | 7.44 (t, J = 7.4 Hz, 1H), 7.30 (td, J = 8.7, 5.7 Hz, 1H), 7.17 (t, J = |
| | | | 9.0 Hz, 1H), 7.09 (t, J = 8.6 Hz, 1H), 3.87 (s, 3H), 3.19 (t, J = 6.7 |
| | | | Hz, 4H), 2.82 (s, 3H), 2.47 (d, J = 6.7 Hz, 3H), 2.18 (s, 6H) |
| 121 | A | 564.3 | ¹H NMR (400 MHz, DMSO-d₆) δ: 10.52 (br s, 1H), 10.20 (s, 1H), |
| | | | 9.77 (s, 1H), 8.79 (d, J = 8.2 Hz, 1H), 8.65 (s, 1H), 8.27 (d, J = 8.2 |
| | | | Hz, 1H), 7.80 (t, J = 7.6 Hz, 1H), 7.64 (t, J = 3.5 Hz, 1H), 7.62 (t, J = |
| | | | 3.5 Hz, 1H), 7.49 (t, J = 8.8 Hz, 1H), 7.41 (td, J = 7.6, 6.3 Hz, 1H), |
| | | | 7.28-7.36 (m, 1H), 7.24 (t, J = 9.0 Hz, 1H), 2.49-2.50 (m, 3H) |
| 122 | F | 762.4 | ¹H NMR (400 MHz, DMSO-d₆) δ: 10.41 (br s, 1H), 9.67 (s, 1H), 9.39 |
| | | | (s, 1H), 8.96 (d, J = 8.6 Hz, 1H), 8.58 (s, 1H), 8.14 (s, 1H), 7.97 (d, |
| | | | J = 7.8 Hz, 1H), 7.92 (d, J = 7.8 Hz, 1H), 7.80 (d, J = 7.8 Hz, 1H), |
| | | | 7.53 (t, J = 7.8 Hz, 1H), 7.45 (td, J = 7.7, 2.9 Hz, 2H), 7.23 (td, J = |
| | | | 8.8, 6.3 Hz, 1H), 7.06 (t, J = 9.0 Hz, 1H), 3.24 (t, J = 6.7 Hz, 2H), |
| | | | 2.82 (s, 3H), 2.69 (t, J = 6.1 Hz, 2H), 2.35 (s, 6H) |
| 123 | A | 617.2 | ¹H NMR (400 MHz, DMSO-d₆) δ: 10.78 (br s, 1H), 10.30 (s, 1H), |
| | | | 9.68 (s, 1H), 9.66 (s, 1H), 8.61 (s, 1H), 8.54 (d, J = 8.2 Hz, 1H), |
| | | | 7.92 (d, J = 7.8 Hz, 1H), 7.85 (br. d, J = 6.3 Hz, 1H), 7.42-7.53 (m, |
| | | | J = 8.2, 8.2, 4.7 Hz, 2H), 7.25 (d, J = 8.2 Hz, 1H), 7.27 (d, J = 8.2 |
| | | | Hz, 1H), 7.12 (br s, 1H) |
| 124 | A | 580.2 | ¹H NMR (400 MHz, DMSO-d₆) δ: 10.58 (s, 1H), 10.21 (s, 1H), 9.77 |
| | | | (s, 1H), 8.79 (d, J = 8.2 Hz, 1H), 8.65 (s, 1H), 8.27 (d, J = 8.2 Hz, |
| | | | 1H), 7.77-7.83 (m, 2H), 7.75 (d, J = 8.2 Hz, 1H), 7.63 (t, J = 7.4 |
| | | | Hz, 1H), 7.39 (t, J = 8.0 Hz, 1H), 7.33 (td, J = 8.6, 5.9 Hz, 1H), 7.25 |
| | | | (t, J = 9.0 Hz, 1H), 2.66 (s, 3H) |
| 125 | F | 742.4 | ¹H NMR (400 MHz, DMSO-d₆) δ: 10.62 (s, 1H), 10.44 (s, 1H), 9.70 |
| | | | (s, 1H), 9.46 (br s, 1H), 9.39 (s, 1H), 8.98 (d, J = 8.2 Hz, 1H), 8.60 |
| | | | (s, 1H), 8.01 (d, J = 7.8 Hz, 1H), 7.77 (t, J = 7.4 Hz, 2H), 7.56 (t, J = |
| | | | 7.8 Hz, 1H), 7.47 (t, J = 7.6 Hz, 1H), 7.39 (t, J = 8.0 Hz, 1H), 7.31- |
| | | | 7.37 (m, 1H), 7.29 (t, J = 9.0 Hz, 1H), 3.41 (t, J = 5.5 Hz, 2H), 3.36 |
| | | | (t, J = 4.7 Hz, 2H), 2.88 (d, J = 3.5 Hz, 6H), 2.83 (s, 3H), 2.68 (s, |
| | | | 3H) |
| 126 | F | 726.4 | ¹H NMR (400 MHz, DMSO-d₆) δ: 10.45 (br s, 1H), 9.67 (s, 1H), 9.37 |
| | | | (s, 1H), 8.96 (d, J = 8.6 Hz, 1H), 8.58 (s, 1H), 7.97 (d, J = 7.8 Hz, |
| | | | 1H), 7.63 (d, J = 7.4 Hz, 1H), 7.53 (t, J = 7.4 Hz, 1H), 7.45 (d, J = |
| | | | 7.8 Hz, 1H), 7.43 (dd, J = 6.7, 3.5 Hz, 1H), 7.38 (dd, J = 7.8, 5.5 |
| | | | Hz, 1H), 7.31 (td, J = 8.8, 5.9 Hz, 1H), 7.19 (t, J = 9.0 Hz, 1H), 3.23 |
| | | | (t, J = 6.5 Hz, 2H), 2.82 (s, 3H), 2.66 (t, J = 6.1 Hz, 2H), 2.51 (d, J = |
| | | | 2.3 Hz, 3H), 2.33 (s, 6H) |
| 127 | A | 579.2 | ¹H NMR (400 MHz, DMSO-d₆) δ: 10.61 (br s, 1H), 10.27 (s, 1H), |
| | | | 9.67 (s, 1H), 9.62 (s, 1H), 8.71 (d, J = 8.6 Hz, 1H), 8.59 (s, 1H), |
| | | | 7.81 (dd, J = 10.2, 8.2 Hz, 2H), 7.70 (br s, 1H), 7.48 (ddd, J = 8.2, |
| | | | 7.4, 0.8 Hz, 1H), 7.41 (ddd, J = 8.2, 7.0, 1.2 Hz, 1H), 7.36 (t, J = 8.2 |
| | | | Hz, 1H), 7.30 (br s, 1H), 7.19 (br s, 1H), 2.67 (s, 3H) |
| 128 | H | 766.2 | ¹H NMR (400 MHz, DMSO-d₆) δ: 10.77 (br s, 1H), 10.23 (s, 1H), |
| | | | 9.66 (s, 1H), 9.43 (s, 1H), 8.58 (s, 1H), 8.26 (d, J = 8.3 Hz, 1H), |
| | | | 7.94 (d, J = 7.9 Hz, 1H), 7.91 (dd, J = 7.9, 1.4 Hz, 1H), 7.53 (t, J = |
| | | | 8.0 Hz, 1H), 7.28-7.39 (m, 2H), 7.25 (t, J = 9.0 Hz, 1H), 6.84 (d, J = |
| | | | 7.9 Hz, 1H), 6.00 (s, 1H), 4.22 (d, J = 11.9 Hz, 2H), 3.09 (dd, J = |
| | | | 12.1, 10.8 Hz, 2H), 1.94 (td, J = 12.7, 4.4 Hz, 2H), 1.80 (d, J = 12.4 |
| | | | Hz, 2H) |
| 129 | H | 738.4 | ¹H NMR (400 MHz, DMSO-d₆) δ: 10.77 (br s, 1H), 10.22 (s, 1H), |
| | | | 9.65 (s, 1H), 9.40 (s, 1H), 8.58 (s, 1H), 8.23 (d, J = 8.1 Hz, 1H), |
| | | | 7.95 (d, J = 7.9 Hz, 1H), 7.91 (dd, J = 8.0, 1.5 Hz, 1H), 7.53 (t, J = |
| | | | 8.0 Hz, 1H), 7.34 (td, J = 8.8, 5.8 Hz, 1H), 7.29 (t, J = 8.1 Hz, 1H), |
| | | | 7.26 (t, J = 9.0 Hz, 1H), 6.79 (d, J = 7.9 Hz, 1H), 3.77 (t, J = 6.8 Hz, |
| | | | 2H), 3.57-3.68 (m, 2H), 3.51 (ddd, J = 11.9, 7.7, 4.3 Hz, 2H), 1.91 |
| | | | (quin, J = 6.9 Hz, 2H), 1.66-1.82 (m, 6H) |
| 130 | H | 712.2 | ¹H NMR (400 MHz, DMSO-d₆) δ: 10.77 (br s, 1H), 10.22 (s, 1H), |
| | | | 9.65 (s, 1H), 9.39 (s, 1H), 8.57 (s, 1H), 8.21 (d, J = 8.1 Hz, 1H), |
| | | | 7.95 (d, J = 8.0 Hz, 1H), 7.91 (dd, J = 8.0, 1.4 Hz, 1H), 7.54 (t, J = |
| | | | 8.1 Hz, 1H), 7.34 (td, J = 8.9, 6.1 Hz, 1H), 7.19-7.31 (m, 2H), 6.79 |
| | | | (d, J = 8.0 Hz, 1H), 4.30 (s, 1H), 3.76 (dt, J = 12.0, 4.5 Hz, 2H), 3.36- |
| | | | 3.45 (m, 2H), 1.58-1.78 (m, 4H), 1.20 (s, 3H) |
| 131 | H | 712.2 | ¹H NMR (400 MHz, DMSO-d₆) δ: 10.77 (br s, 1H), 10.23 (s, 1H), |
| | | | 9.65 (s, 1H), 9.40 (s, 1H), 8.58 (s, 1H), 8.23 (d, J = 8.1 Hz, 1H), |
| | | | 7.95 (d, J = 8.0 Hz, 1H), 7.91 (dd, J = 8.0, 1.5 Hz, 1H), 7.54 (t, J = |
| | | | 8.0 Hz, 1H), 7.34 (td, J = 8.8, 6.0 Hz, 1H), 7.20-7.31 (m, 2H), 6.78 |
| | | | (d, J = 7.9 Hz, 1H), 3.97 (dt, J = 12.4, 3.8 Hz, 2H), 3.40 (spt, J = 4.1 |
| | | | Hz, 1H), 3.30 (s, 3H), 3.13 (ddd, J = 12.3, 9.8, 2.5 Hz, 2H), 1.94- |
| | | | 2.11 (m, 2H), 1.64 (dtd, J = 12.5, 9.2, 3.5 Hz, 2H) |
| 132 | H | 707.2 | ¹H NMR (400 MHz, DMSO-d₆) δ: 10.77 (br s, 1H), 10.23 (s, 1H), |
| | | | 9.66 (s, 1H), 9.42 (s, 1H), 8.58 (s, 1H), 8.27 (d, J = 8.1 Hz, 1H), |
| | | | 7.95 (d, J = 8.0 Hz, 1H), 7.91 (dd, J = 7.9, 1.4 Hz, 1H), 7.54 (t, J = |
| | | | 8.0 Hz, 1H), 7.29-7.40 (m, 2H), 7.26 (t, J = 9.0 Hz, 1H), 6.82 (d, J = |
| | | | 7.9 Hz, 1H), 3.66-3.86 (m, 2H), 3.38 (ddd, J = 11.8, 8.3, 2.9 Hz, |
| | | | 2H), 3.12 (tt, J = 8.2, 4.1 Hz, 1H), 2.09 (ddd, J = 9.3, 6.2, 2.8 Hz, |
| | | | 2H), 1.87-2.02 (m, 2H) |
| 133 | H | 726.2 | ¹H NMR (400 MHz, DMSO-d₆) δ: 10.77 (br s, 1H), 10.23 (s, 1H), |
| | | | 9.65 (s, 1H), 9.39 (s, 1H), 8.57 (s, 1H), 8.21 (d, J = 8.1 Hz, 1H), |
| | | | 7.95 (d, J = 8.0 Hz, 1H), 7.91 (dd, J = 7.9, 1.4 Hz, 1H), 7.54 (t, J = |
| | | | 8.0 Hz, 1H), 7.34 (td, J = 8.4, 6.0 Hz, 1H), 7.21-7.31 (m, 2H), 6.79 |
| | | | (d, J = 7.9 Hz, 1H), 4.08 (s, 1H), 3.81-3.95 (m, 2H), 3.25 (td, J = |
| | | | 11.9, 2.8 Hz, 2H), 1.68 (td, J = 12.5, 4.0 Hz, 2H), 1.56-1.64 (m, |
| | | | 2H), 1.46 (q, J = 7.4 Hz, 2H), 0.89 (t, J = 7.4 Hz, 3H) |
| 134 | H | 672.2 | ¹H NMR (400 MHz, DMSO-d₆) δ: 10.16 (brs, 1H), 9.63 (s, 1H), 9.44 |
| | | | (s, 1H), 8.58 (s, 1H), 8.14 (s, 1H), 8.09 (d, J = 8.00 Hz, 1H), 7.80- |
| | | | 7.98 (m, 1H), 7.42 (br s, 1H), 7.25 (t, J = 8.13 Hz, 1H), 6.61 (d, J = |
| | | | 8.25 Hz, 1H), 6.52 (s, 1H), 4.76 (t, J = 5.07 Hz, 1H), 3.93 (t, J = 6.19 |
| | | | Hz, 2H), 3.69 (q, J = 5.92 Hz, 2H), 3.13 (s, 3H) |
| 135 | See text | 665.2 | ¹H NMR (400 MHz, DMSO-d₆) δ: 10.74 (br s, 1H), 10.19 (s, 1H), |
| | | | 9.77 (s, 1H), 8.76 (d, J = 8.26 Hz, 1H), 8.66 (s, 1H), 7.84-7.99 (m, |
| | | | 2H), 7.73-7.84 (m, 1H), 7.69 (d, J = 7.25 Hz, 1H), 7.53 (t, J = 8.07 |
| | | | Hz, 1H), 7.32 (d, J = 6.13 Hz, 1H), 7.22 (br s, 1H), 2.04-2.11 (m, |
| | | | 2H), 1.91-2.02 (m, 2H) |
| 136 | I | 727.4 | ¹H NMR (400 MHz, DMSO-d₆) δ: 10.74 (s, 1H), 10.13 (s, 1H), 9.74 |
| | | | (s, 1H), 8.63 (s, 1H), 8.08 (d, J = 8.13 Hz, 1H), 7.95 (d, J = 8.00 Hz, |
| | | | 1H), 7.91 (dd, J = 1.19, 7.94 Hz, 1H), 7.53 (dt, J = 6.00, 7.94 Hz, |
| | | | 2H), 7.29-7.44 (m, 1H), 7.07-7.29 (m, 1H), 6.84 (d, J = 8.00 Hz, |
| | | | 1H), 4.06-4.29 (m, 3H), 3.37-3.58 (m, 2H), 1.56-1.81 (m, 4H), |
| | | | 1.47 (q, J = 7.34 Hz, 2H), 0.89 (t, J = 7.44 Hz, 3H) |
| 137 | See text | 674.2 | ¹H NMR (400 MHz, DMSO-d₆) δ: 10.74 (br s, 1H), 10.20 (s, 1H), |
| | | | 9.68-9.85 (m, 1H), 8.50-8.71 (m, 1H), 8.30 (d, J = 8.38 Hz, 1H), |
| | | | 7.80-8.03 (m, 2H), 7.66 (t, J = 8.13 Hz, 1H), 7.53 (t, J = 8.00 Hz, |
| | | | 1H), 7.31 (d, J = 6.00 Hz, 1H), 7.22 (br s, 1H), 7.09 (d, J = 7.88 Hz, |
| | | | 1H), 4.48 (dd, J = 3.63, 5.25 Hz, 2H), 3.84 (dd, J = 3.63, 5.25 Hz, |
| | | | 2H), 3.35-3.46 (m, 3H) |
| 138 | I | 739.3 | ¹H NMR (400 MHz, DMSO-d₆) δ: 10.74 (s, 1H), 10.14 (s, 1H), 9.68- |
| | | | 9.75 (m, 1H), 8.63 (s, 1H), 8.10 (d, J = 8.25 Hz, 1H), 7.95 (d, J = |
| | | | 8.00 Hz, 1H), 7.90 (dd, J = 1.38, 8.00 Hz, 1H), 7.53 (dt, J = 4.06, |
| | | | 8.04 Hz, 2H), 7.28-7.38 (m, 1H), 7.05-7.28 (m, 1H), 6.84 (d, J = |
| | | | 8.00 Hz, 1H), 3.86-4.13 (m, 2H), 3.79 (t, J = 6.75 Hz, 2H), 3.70 |
| | | | (ddd, J = 3.94, 8.54, 12.54 Hz, 2H), 1.85-2.02 (m, 2H), 1.68-1.84 |
| | | | (m, 6H) |
| 139 | I | 728.2 | ¹H NMR (400 MHz, DMSO-d₆) δ: 10.10 (br s, 1H), 9.68-9.76 (m, |
| | | | 1H), 8.62-8.68 (m, 1H), 8.19 (d, J = 7.88 Hz, 1H), 8.13 (s, 1H), |
| | | | 7.91 (dd, J = 1.25, 7.88 Hz, 1H), 7.87 (d, J = 8.13 Hz, 1H), 7.56 (t, |
| | | | J = 8.07 Hz, 1H), 7.49 (t, J = 8.00 Hz, 1H), 7.20-7.33 (m, 1H), 7.12 |
| | | | (br s, 1H), 6.84 (d, J = 8.00 Hz, 1H), 4.59 (br s, 1H), 3.79 (br s, 4H), |
| | | | 3.61 (br s, 2H), 2.82 (br s, 4H), 2.61 (br s, 2H) |
| 140 | H | 686.2 | ¹H NMR (400 MHz, DMSO-d₆) δ: 10.76 (br s, 1H), 10.19 (s, 1H), |
| | | | 9.64 (s, 1H), 9.37 (s, 1H), 8.51-8.59 (m, 1H), 8.02 (d, J = 8.13 Hz, |
| | | | 1H), 7.91 (d, J = 7.88 Hz, 2H), 7.51 (br s, 1H), 7.09-7.38 (m, 3H), |
| | | | 6.32 (d, J = 7.88 Hz, 1H), 4.75 (d, J = 6.00 Hz, 1H), 4.63 (d, J = |
| | | | 6.00 Hz, 1H), 4.28 (t, J = 7.94 Hz, 2H), 3.99 (dd, J = 5.63, 7.88 Hz, |
| | | | 2H), 3.12 (td, J = 6.11, 19.17 Hz, 1H) |
| 141 | G | 690.2 | ¹H NMR (400 MHz, DMSO-d₆) δ: 10.78 (br s, 1H), 10.55 (s, 1H), |
| | | | 9.68 (s, 1H), 9.43 (s, 1H), 8.96 (d, J = 8.38 Hz, 1H), 8.58 (s, 1H), |
| | | | 7.81-8.02 (m, 3H), 7.54 (dt, J = 4.75, 7.69 Hz, 2H), 7.46 (t, J = |
| | | | 7.50 Hz, 1H), 7.31-7.40 (m, 1H), 7.26 (t, J = 9.13 Hz, 1H), 3.40 (q, |
| | | | J = 7.30 Hz, 2H), 1.22 (t, J = 7.38 Hz, 3H) |
| 142 | G | 746.2 | ¹H NMR (400 MHz, DMSO-d₆) δ: 10.78 (s, 1H), 10.54 (s, 1H), 9.69 |
| | | | (s, 1H), 9.42 (s, 1H), 8.97 (d, J = 8.38 Hz, 1H), 8.58 (s, 1H), 7.83- |
| | | | 8.03 (m, 3H), 7.50-7.62 (m, 2H), 7.41-7.50 (m, 1H), 7.30-7.41 |
| | | | (m, 1H), 7.27 (d, J = 8.88 Hz, 1H), 3.91 (dd, J = 4.00, 11.13 Hz, |
| | | | 2H), 3.58 (tt, J = 3.60, 11.91 Hz, 1H), 3.29 (br s, 1H), 1.90 (d, J = |
| | | | 10.76 Hz, 2H), 1.68 (dq, J = 4.57, 12.32 Hz, 2H) |
| 143 | G | 753.2 | ¹H NMR (400 MHz, DMSO-d₆) δ: 10.78 (br s, 1H), 10.53 (s, 1H), |
| | | | 9.68 (s, 1H), 9.34 (s, 1H), 8.94 (d, J = 8.50 Hz, 1H), 8.58 (s, 1H), |
| | | | 8.47 (d, J = 5.88 Hz, 2H), 7.96 (d, J = 8.00 Hz, 1H), 7.91 (dd, J = |
| | | | 1.38, 8.00 Hz, 1H), 7.62 (d, J = 8.00 Hz, 1H), 7.51 (t, J = 7.50 Hz, |
| | | | 1H), 7.54 (t, J = 8.07 Hz, 1H), 7.30-7.42 (m, 2H), 7.15-7.30 (m, |
| | | | 3H), 4.86 (s, 2H) |
| 144 | G | 760.2 | ¹H NMR (400 MHz, DMSO-d₆) δ: 10.78 (s, 1H), 10.54 (s, 1H), 9.69 |
| | | | (s, 1H), 9.43 (S, 1H), 8.96 (d, J = 8.51 Hz, 1H), 8.58 (s, 1H), 7.95 |
| | | | (d, J = 7.75 Hz, 2H), 7.91 (dd, J = 1.31, 7.94 Hz, 1H), 7.51-7.60 |
| | | | (m, 2H), 7.42-7.51 (m, 1H), 7.35 (d, J = 6.63 Hz, 1H), 7.26 (br s, |
| | | | 1H), 3.65-3.83 (m, 2H), 3.40 (d, J = 6.25 Hz, 2H), 3.22-3.28 (m, |
| | | | 2H), 2.11-2.29 (m, 1H), 1.67-1.88 (m, 2H), 1.30-1.45 (m, 2H) |
| 145 | H | 725.2 | ¹H NMR (400 MHz, DMSO-d₆) δ: 10.16 (br s, 1H), 9.63 (s, 1H), 9.47 |
| | | | (s, 1H), 8.59 (S, 1H), 8.27 (d, J = 8.25 Hz, 1H), 7.93 (dd, J = 1.38, |
| | | | 7.88 Hz, 1H), 7.73 (d, J = 7.75 Hz, 1H), 7.41 (t, J = 7.94 Hz, 1H), |
| | | | 7.31 (t, J = 8.07 Hz, 1H), 7.16 (dt, J = 6.07, 9.04 Hz, 1H), 6.94 (t, J = |
| | | | 9.01 Hz, 1H), 6.80 (d, J = 8.00 Hz, 1H), 4.41 (d, J = 12.13 Hz, |
| | | | 2H), 2.90-3.21 (m, 2H), 2.82 (t, J = 11.63 Hz, 2H), 2.65 (s, 6H), |
| | | | 2.01-2.13 (m, 2H), 1.70-1.87 (m, 2H) |
| 146 | H | 727.2 | ¹H NMR (400 MHz, DMSO-d₆) δ: 10.20 (s, 1H), 9.64 (s, 1H), 9.45 |
| | | | (s, 1H), 8.59 (s, 1H), 8.28 (d, J = 8.13 Hz, 1H), 7.92 (dd, J = 1.38, |
| | | | 7.88 Hz, 1H), 7.84 (d, J = 7.88 Hz, 1H), 7.47 (t, J = 7.94 Hz, 1H), |
| | | | 7.32 (t, J = 8.13 Hz, 1H), 7.16-7.28 (m, 1H), 7.10 (t, J = 8.82 Hz, |
| | | | 1H), 6.78 (d, J = 8.00 Hz, 1H), 3.54-3.69 (m, 6H), 2.85 (br s, 4H), |
| | | | 2.60-2.73 (m, 2H) |
| 147 | H | 698.3 | ¹H NMR (400 MHz, DMSO-d₆) δ: 10.77 (s, 1H), 10.27 (s, 1H), 9.65 |
| | | | (s, 1H), 9.39 (s, 1H), 8.57 (s, 1H), 7.94 (d, J = 8.13 Hz, 1H), 7.81- |
| | | | 7.93 (m, 2H), 7.53 (t, J = 8.07 Hz, 1H), 7.29-7.41 (m, 1H), 7.25 (br |
| | | | s, 1H), 7.21 (t, J = 8.07 Hz, 1H), 6.52 (d, J = 8.88 Hz, 1H), 5.99 (t, |
| | | | J = 5.94 Hz, 1H), 3.71-3.86 (m, 2H), 3.64 (q, J = 7.75 Hz, 1H), |
| | | | 3.54 (dd, J = 5.32, 8.44 Hz, 1H), 3.25 (t, J = 6.63 Hz, 2H), 2.60- |
| | | | 2.75 (m, 1H), 1.91-2.13 (m, 1H), 1.67 (dd, J = 5.57, 12.82 Hz, 1H) |
| 148 | A | 559.2 | ¹H NMR (400 MHz, DMSO-d₆) δ: 10.28 (s, 1H), 9.67 (s, 1H), 9.62 |
| | | | (s, 1H), 8.70 (d, J = 8.13 Hz, 1H), 8.60 (s, 1H), 7.79 (d, J = 7.88 Hz, |
| | | | 1H), 7.82 (d, J = 7.88 Hz, 1H), 7.54 (d, J = 6.88 Hz, 1H), 7.37-7.52 |
| | | | (m, 3H), 7.33 (t, J = 7.44 Hz, 1H), 7.04-7.30 (m, 2H), 3.05 (q, J = |
| | | | 7.38 Hz, 2H), 1.21 (t, J = 7.44 Hz, 3H) |
| 149 | H | 686.4 | ¹H NMR (400 MHz, DMSO-d₆) δ: 10.41 (br s, 1H), 10.22 (s, 1H), |
| | | | 9.65 (s, 1H), 9.40 (s, 1H), 8.59 (s, 1H), 8.21 (d, J = 8.13 Hz, 1H), |
| | | | 7.78 (d, J = 7.88 Hz, 1H), 7.58 (t, J = 7.50 Hz, 1H), 7.47 (d, J = 7.63 |
| | | | Hz, 1H), 7.35 (t, J = 7.57 Hz, 1H), 7.17-7.32 (m, 3H), 6.79 (d, J = |
| | | | 8.00 Hz, 1H), 4.08 (s, 1H), 3.89 (d, J = 11.88 Hz, 2H), 3.16-3.29 |
| | | | (m, 2H), 3.04 (q, J = 7.38 Hz, 2H), 1.53-1.77 (m, 4H), 1.46 (q, J = |
| | | | 7.30 Hz, 2H), 1.21 (t, J = 7.44 Hz, 3H), 0.89 (t, J = 7.38 Hz, 3H) |
| 150 | A | 611.2 | ¹H NMR (400 MHz, DMSO-d₆) δ: 10.85 (br s, 1H), 10.30 (s, 1H), |
| | | | 9.68 (s, 1H), 9.63 (s, 1H), 8.70 (d, J = 8.00 Hz, 1H), 8.57 (s, 1H), |
| | | | 7.82 (d, J = 7.88 Hz, 1H), 7.72 (br s, 1H), 7.22-7.51 (m, 6H) |
| 151 | H | 738.4 | ¹H NMR (400 MHz, DMSO-d6) δ: 10.85 (br s, 1H), 10.23 (s, 1H), |
| | | | 9.65 (s, 1H), 9.41 (s, 1H), 8.56 (s, 1H), 8.21 (d, J = 8.26 Hz, 1H), |
| | | | 7.73 (d, J = 6.00 Hz, 1H), 7.42-7.51 (m, 1H), 7.32-7.42 (m, 2H), |
| | | | 7.29 (t, J = 8.07 Hz, 2H), 6.79 (d, J = 8.00 Hz, 1H), 4.08 (s, 1H), |
| | | | 3.89 (d, J = 11.88 Hz, 2H), 3.18-3.29 (m, 2H), 1.52-1.76 (m, 4H), |
| | | | 1.46 (q, J = 7.34 Hz, 2H), 0.89 (t, J = 7.44 Hz, 3H) |
| 152 | A | 579.3 | ¹H NMR (400 MHz, DMSO-d₆) δ: 10.53 (br s, 1H), 10.28 (s, 1H), |
| | | | 9.68 (s, 1H), 9.62 (s, 1H), 8.71 (d, J = 8.13 Hz, 1H), 8.60 (s, 1H), |
| | | | 7.81 (t, J = 8.69 Hz, 2H), 7.62 (br s, 1H), 7.45-7.54 (m, 1H), 7.41 |
| | | | (t, J = 7.57 Hz, 2H), 7.26 (br s, 2H), 2.40 (s, 3H) |
| 153 | H | 706.3 | ¹H NMR (400 MHz, DMSO-d₆) δ: 10.55 (br s, 1H), 10.22 (s, 1H), |
| | | | 9.65 (s, 1H), 9.23-9.49 (m, 1H), 8.58 (s, 1H), 8.21 (d, J = 8.13 Hz, |
| | | | 1H), 7.79 (d, J = 7.75 Hz, 1H), 7.64 (d, J = 7.50 Hz, 1H), 7.39 (t, J = |
| | | | 7.75 Hz, 1H), 7.28 (t, J = 8.00 Hz, 2H), 7.11-7.25 (m, 1H), 6.79 |
| | | | (d, J = 8.00 Hz, 1H), 4.08 (s, 1H), 3.89 (d, J = 11.76 Hz, 2H), 3.17- |
| | | | 3.29 (m, 2H), 2.40 (s, 3H), 1.54-1.82 (m, 4H), 1.46 (q, J = 7.38 |
| | | | Hz, 2H), 0.89 (t, J = 7.44 Hz, 3H) |
| 154 | A | 581.2 | ¹H NMR (400 MHz, DMSO-d₆) δ: 10.59 (br s, 1H), 10.28 (s, 1H), |
| | | | 9.68 (s, 1H), 9.61 (s, 1H), 8.70 (d, J = 8.13 Hz, 1H), 8.58 (s, 1H), |
| | | | 7.82 (d, J = 7.88 Hz, 1H), 7.64 (dd, J = 5.07, 8.57 Hz, 1H), 7.37- |
| | | | 7.54 (m, 3H), 7.27-7.37 (m, 1H), 7.23 (br s, 1H), 2.54-2.59 (m, |
| | | | 3H) |
| 155 | H | 708.4 | ¹H NMR (400 MHz, DMSO-d₆) δ: 10.58 (br s, 1H), 10.21 (s, 1H), |
| | | | 9.65 (s, 1H), 9.39 (s, 1H), 8.57 (s, 1H), 8.21 (d, J = 8.13 Hz, 1H), |
| | | | 7.63 (dd, J = 4.88, 8.63 Hz, 1H), 7.45 (q, J = 8.80 Hz, 1H), 7.18- |
| | | | 7.39 (m, 3H), 6.79 (d, J = 8.00 Hz, 1H), 4.08 (s, 1H), 3.89 (d, J = |
| | | | 11.76 Hz, 2H), 3.17-3.29 (m, 2H), 2.56 (d, J = 2.25 Hz, 3H), 1.64- |
| | | | 1.75 (m, 2H), 1.55-1.64 (m, 2H), 1.46 (q, J = 7.25 Hz, 2H), 0.89 |
| | | | (t, J = 7.44 Hz, 3H) |
| 156 | A | 633.2 | ¹H NMR (400 MHz, DMSO-d₆) δ: 10.75 (br s, 1H), 10.32 (s, 1H), |
| | | | 9.68 (s, 1H), 9.61 (s, 1H), 8.70 (d, J = 8.0 Hz, 1H), 8.60 (s, 1H), |
| | | | 8.10 (d, J = 8.0 Hz, 1H), 8.03 (d, J = 8.0 Hz, 1H), 7.86 (t, J = 8.1 |
| | | | Hz, 1H), 7.82 (d, J = 7.9 Hz, 1H), 7.48 (t, J = 7.6 Hz, 1H), 7.41 (t, J = |
| | | | 7.7 Hz, 1H), 7.36 (dd, J = 8.3, 6.0 Hz, 1H), 7.30 (t, J = 9.3 Hz, 1H) |
| 157 | A | 605.2 | ¹H NMR (400 MHz, DMSO-d₆) δ: 11.05 (br s, 1H), 10.35 (s, 1H), |
| | | | 9.69 (s, 1H), 9.64 (s, 1H), 8.71 (d, J = 8.1 Hz, 1H), 8.62 (s, 1H), |
| | | | 8.21 (s, 1H), 7.83 (d, J = 7.8 Hz, 1H), 7.49 (t, J = 7.9 Hz, 1H), 7.42 |
| | | | (t, J = 7.6 Hz, 1H), 7.35-7.40 (m, 1H), 7.32 (t, J = 9.3 Hz, 1H) |
| 158 | H | 760.2 | ¹H NMR (400 MHz, DMSO-d₆) δ: 10.75 (br s, 1H), 10.25 (s, 1H), |
| | | | 9.66 (s, 1H), 9.40 (s, 1H), 8.59 (s, 1H), 8.21 (d, J = 8.3 Hz, 1H), |
| | | | 8.10 (d, J = 8.0 Hz, 1H), 8.02 (d, J = 8.3 Hz, 1H), 7.86 (t, J = 8.0 |
| | | | Hz, 1H), 7.32-7.42 (m, 1H), 7.21-7.32 (m, 2H), 6.79 (d, J = 8.0 |
| | | | Hz, 1H), 4.09 (s, 1H), 3.89 (dt, J = 11.9, 3.9 Hz, 2H), 3.25 (td, J = |
| | | | 11.0, 2.6 Hz, 2H), 1.68 (td, J = 12.0, 4.0 Hz, 2H), 1.54-1.64 (m, |
| | | | 2H), 1.46 (q, J = 7.5 Hz, 2H), 0.89 (t, J = 7.4 Hz, 3H) |
| 159 | H | 732.2 | ¹H NMR (400 MHz, DMSO-d₆) δ: 11.04 (br s, 1H), 10.27 (s, 1H), |
| | | | 9.66 (s, 1H), 9.41 (s, 1H), 8.60 (s, 1H), 8.15-8.29 (m, 2H), 7.34- |
| | | | 7.43 (m, 1H), 7.24-7.34 (m, 2H), 6.79 (d, J = 8.0 Hz, 1H), 4.09 (s, |
| | | | 1H), 3.89 (dt, J = 11.6, 3.8 Hz, 2H), 3.25 (td, J = 11.5, 3.0 Hz, 2H), |
| | | | 1.68 (td, J = 12.0, 3.6 Hz, 2H), 1.55-1.64 (m, 2H), 1.46 (q, J = 7.3 |
| | | | Hz, 2H), 0.89 (t, J = 7.4 Hz, 3H) |

TABLE 4

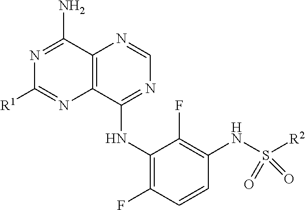

| | | | HCT116 | SW480 | Kinase | Kinase |
|---|---|---|---|---|---|---|
| | | | PERK IC₅₀ | PERK IC₅₀ | IC₅₀ | IC₅₀ |
| | | | (μM) | (μM) | BRAF | CRAF |
| Example | R¹ | R² | (Yₘᵢₙ %) | (Yₘᵢₙ %) | (nM) | (nM) |
| 84 | C3 | B1 | ++ (8) | ++ (10) | | |
| 85 | C5 | B1 | + (8) | ++ (4) | | |
| 86 | C5 | B12 | ++ (8) | ++ (3) | | |
| 87 | C3 | B12 | ++ (2) | +++ (11) | | |
| 88 | C7 | B12 | ++ (3) | ++ (2) | * | § |

For pERK assays, + denotes a 10-30 μM IC₅₀ range, ++ denotes a 1-10 μM IC₅₀ range, +++ denotes a 0.5-1 μM IC₅₀ range and ++++ denotes an IC₅₀ <0.5 μM. The % Yₘᵢₙ value indicates the lowest value of each IC₅₀ curve. Compounds that exhibit IC₅₀ curves with Yₘᵢₙ values above −20% are considered to display minimal or no induction and do not cause detectable paradoxical activation of the pathway. For the BRAF biochemical kinase assay, * denotes an IC₅₀ >10 nM, ** denotes a 1-10 nM IC₅₀ range and *** denotes an IC₅₀ <1 nM. For the CRAF biochemical kinase assay, § denotes an IC₅₀ >50 nM, §§ denotesa 10-50 nM IC₅₀ range and $$$ denotes an IC₅₀ <10 nM.

### Characterization of Compounds in Table 4

| | HRMS | |
|---|---|---|
| | m/z | |
| Example | (MH⁺) | ¹H NMR (400 MHz) |
| 84 | 547.2 | ¹H NMR (DMSO-d₆) δ: 10.09 (br s, 1H), 9.01 (s, 1H), 8.06 (s, 1H), 7.60- |
| | | 7.73 (m, 3H), 7.45 (br s, 1H), 7.11-7.24 (m, 2H), 7.04-7.11 (m, 2H), |
| | | 5.42 (dt, J = 54.0, 3.1 Hz, 1H), 3.83-3.92 (m, 1H), 3.81 (s, 3H), 3.66 |
| | | (ddd, J = 40.3, 14.1, 3.5 Hz, 1H), 3.53 (td, J = 10.9, 7.2 Hz, 1H), 2.05- |
| | | 2.28 (m, 2H) |
| 85 | 554.2 | ¹H NMR (DMSO-d₆) δ: 10.13 (br s, 1H), 9.54 (s, 1H), 8.59 (br s, 1H), 8.40 |
| | | (br s, 1H), 8.36 (s, 1H), 8.24 (br s, 1H), 7.63-7.72 (m, 2H), 7.15-7.29 |
| | | (m, 2H), 7.04-7.13 (m, 2H), 3.81 (s, 3H), 2.53 (s, 3H), 2.10 (s, 3H) |
| 86 | 568.2 | ¹H NMR (DMSO-d₆) δ: 10.15 (s, 1H), 9.55 (s, 1H), 8.60 (br s, 1H), 8.40 |
| | | (s, 1H), 8.35 (s, 1H), 8.24 (s, 1H), 7.67 (d, J = 9.0 Hz, 1H), 7.24 (td, J = |
| | | 8.6, 5.9 Hz, 1H), 7.17 (t, J = 9.0 Hz, 1H), 6.95 (d, J = 2.3 Hz, 1H), 6.86 |
| | | (dd, J = 8.8, 2.5 Hz, 1H), 3.79 (s, 3H), 2.57 (s, 3H), 2.53 (s, 3H), 2.10 (s, |
| | | 3H) |
| 87 | 561.2 | ¹H NMR (DMSO-d₆) δ: 10.11 (s, 1H), 9.02 (s, 1H), 8.05 (s, 1H), 7.67 (d, |
| | | J = 9.0 Hz, 1H), 7.65 (br s, 1H), 7.44 (br s, 1H), 7.19 (td, J = 9.0, 5.9 Hz, |
| | | 1H), 7.12 (t, J = 9.4 Hz, 1H), 6.94 (d, J = 2.3 Hz, 1H), 6.85 (dd, J = 8.8, |
| | | 2.5 Hz, 1H), 3.79-3.91 (m, 2H), 3.79 (s, 3H), 3.66 (ddd, J = 39.9, 14.1, |
| | | 3.5 Hz, 1H), 3.53 (td, J = 10.7, 7.2 Hz, 1H), 2.57 (s, 3H), 2.14-2.30 (m, |
| | | 2H) |
| 88 | 590.2 | ¹H NMR (DMSO-d₆) δ: 10.18 (s, 1H), 9.76 (s, 1H), 9.53 (s, 1H), 8.83 (d, |
| | | J = 8.2 Hz, 1H), 8.66 (s, 1H), 8.39 (br s, 1H), 8.38 (s, 1H), 7.75 (d, J = |
| | | 7.4 Hz, 1H), 7.68 (d, J = 8.6 Hz, 1H), 7.40 (t, J = 7.0 Hz, 1H), 7.35 (t, J = |
| | | 7.0 Hz, 1H), 7.27 (td, J = 8.6, 5.5 Hz, 1H), 7.21 (t, J = 9.4 Hz, 1H), 6.96 |
| | | (d, J = 2.3 Hz, 1H), 6.87 (dd, J = 8.8, 2.5 Hz, 1H), 3.79 (s, 3H), 2.59 (s, |
| | | 3H) |

TABLE 5

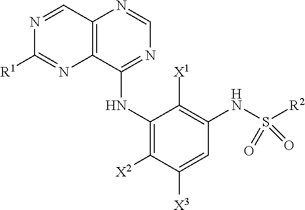

| | | | | HCT116 | SW480 | Kinase | Kinase |
|---|---|---|---|---|---|---|---|
| | | | | PERK | PERK | IC₅₀ | IC₅₀ |
| Exam- | | | X¹/X²/ | IC₅₀ | IC₅₀ | (nM) | (nM) |
| ple | R¹ | R² | X³ | (μM) | (μM) | BRAF | CRAF |
| 89 | C1 | B1 | Cl/F/H | + (−5) | + (3.5) | | |
| 160 | C7 | B41 | F/F/F | ++++ (−9) | | ** | §§§ |
| 161 | C22 | B41 | F/F/F | ++ (−2.7) | | ** | §§§ |
| 162 | C438 | B41 | F/F/F | ++++ (−9) | | ** | §§§ |
| 163 | C416 | B41 | F/F/F | ++++ (−14) | | ** | §§§ |

For pERK assays, + denotes a 10-30 μM IC₅₀ range, ++ denotes a 1-10 μM IC₅₀ range, +++ denotes a 0.5-1 μM IC₅₀ range and ++++ denotes an IC₅₀ <0.5 μM. The % Yₘᵢₙ value indicates the lowest value of each IC₅₀ curve. Compounds that exhibit IC₅₀ curves with Yₘᵢₙ values above −20% are considered to display minimal or no induction and do not cause detectable paradoxical activation of the pathway. For the BRAF biochemical kinase assay, * denotes an IC₅₀ >10 nM, ** denotes a 1-10 nM IC₅₀ range and ** denotes an IC₅₀ <1 nM. For the CRAF biochemical kinase assay, § denotes an IC₅₀ >50 nM, §§ denotes a 10-50 nM IC₅₀ range and §§§ denotes an IC₅₀ <10 nM.

### Characterization of the Compound in Table 5

| | HRMS | |
|---|---|---|
| | m/z | |
| Example | (MH⁺) | ¹H NMR (400 MHz) |
| 89 | 507.1 | ¹H NMR (DMSO-d₆) δ: 10.14 (s, 1H), 9.99 (br s, 1H), 9.29 (s, 1H), 8.49 (s, |
| | | 1H), 7.68 (d, J = 9.0 Hz, 2H), 7.36 (t, J = 9.0 Hz, 1H), 7.20-7.32 (m, 1H), 7.09 |
| | | (d, J = 8.6 Hz, 2H), 3.82 (s, 3H), 2.71 (br s, 3H) |
| 160 | 619.2 | ¹H NMR (400 MHz, DMSO-d₆) δ: 11.08 (br s, 1H), 10.42 (s, 1H), 9.71 (s, 1H), |
| | | 9.60 (s, 1H), 8.70 (d, J = 8.1 Hz, 1H), 8.63 (s, 1H), 7.97 (d, J = 7.9 Hz, 2H), |
| | | 7.83 (d, J = 7.9 Hz, 1H), 7.45-7.60 (m, 3H), 7.39-7.45 (m, 1H) |
| 161 | 618.2 | ¹H NMR (400 MHz, DMSO-d₆) δ: 11.04 (br s, 1H), 10.35 (s, 1H), 9.79 (s, 1H), |
| | | 8.76 (d, J = 8.3 Hz, 1H), 8.68 (s, 1H), 8.28 (d, J = 8.3 Hz, 1H), 7.96 (d, J = 8.0 |
| | | Hz, 2H), 7.80 (dd, J = 7.7 Hz, 1H), 7.63 (dd, J = 7.6 Hz, 1H), 7.48-7.58 (m, |
| | | 2H) |
| 162 | 745.3 | ¹H NMR (400 MHz, DMSO-d₆) δ: 11.07 (br s, 1H), 10.33 (s, 1H), 9.67 (s, 1H), |
| | | 9.38 (s, 1H), 8.61 (s, 1H), 8.20 (d, J = 8.1 Hz, 1H), 7.89-8.00 (m, 2H), 7.46- |
| | | 7.58 (m, 2H), 7.29 (dd, J = 8.1 Hz, 1H), 6.79 (d, J = 8.0 Hz, 1H), 4.08 (s, 1H), |
| | | 3.84-3.93 (m, 2H), 3.21-3.30 (m, 2H), 1.56-1.73 (m, 4H), 1.46 (q, J = 7.3 |
| | | Hz, 2H), 0.89 (t, J = 7.4 Hz, 3H) |
| 163 | 718.2 | ¹H NMR (400 MHz, DMSO-d₆) δ: 11.07 (br s, 1H), 10.33 (s, 1H), 9.67 (s, 1H), |
| | | 9.39 (s, 1H), 8.62 (s, 1H), 8.22 (d, J = 8.1 Hz, 1H), 7.89-8.01 (m, 2H), 7.45- |
| | | 7.59 (m, 2H), 7.29 (dd, J = 8.1 Hz, 1H), 6.77 (d, J = 8.1 Hz, 1H), 4.85 (br s, |
| | | 1H), 4.20 (dd, J = 11.1, 2.8 Hz, 1H), 4.06 (d, J = 11.8 Hz, 1H), 3.73 (br s, 1H), |
| | | 2.74-2.85 (m, 1H), 2.59 (t, J = 10.3 Hz, 1H), 1.98 (dd, J = 11.7, 3.4 Hz, 1H), |
| | | 1.76-1.87 (m, 1H), 1.61-1.74 (m, 1H), 1.20-1.37 (m, 1H) |

Numerous modifications could be made to any of the embodiments described above without departing from the scope of the present invention. Any references, patents or scientific literature documents referred to in the present document are incorporated herein by reference in their entirety for all purposes.

1. A compound of Formula I: 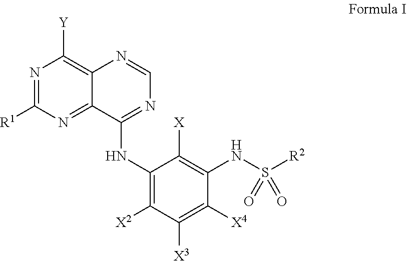
wherein:
R¹ is selected from substituted or unsubstituted OR³, SR³, NH₂, NHR³, N(R³)₂, C₃₋₈cycloalkyl, C₄₋₈heterocycloalkyl, C₆₋₁₀aryl and C₅₋₁₀heteroaryl;
R² is selected from substituted C₆aryl and C₅₋₁₀heteroaryl, substituted or unsubstituted C₄₋₈heterocycloalkyl, and N(R³)₂;
R³ is independently in each occurrence selected from substituted or unsubstituted C₁₋₈alkyl, C₃₋₈cycloalkyl, C₄₋₈heterocycloalkyl, C₆₋₁₀aryl and C₅₋₁₀heteroaryl;
X¹ is halo or an electron-withdrawing group;
X² is selected from H, halo, and an electron-withdrawing group;
X³ and X⁴ are each selected from H, halo, an electron-withdrawing group, C₁₋₃alkyl, C₃₋₄cycloalkyl, and OC₁₋₃alkyl;
Y is selected from H, halo, CN, OH, OC₁₋₈alkyl, NH₂, NHC₁₋₈alkyl, N(C₁₋₈alkyl)₂, and a substituted or unsubstituted C₁₋₈alkyl;
or a pharmaceutically acceptable salt or solvate thereof;
provided that the compound is other than:

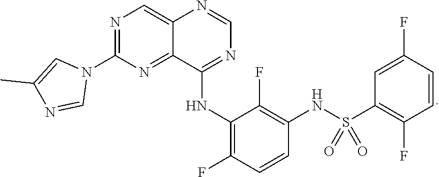 [SMILES: Cc1cn(-c2ncc3ncnc(Nc4c(F)ccc(NS(=O)(=O)c5cc(F)ccc5F)c4F)c3n2)cn1]

2-3. (canceled)

4. The compound of claim 1, wherein R² is a group of the formula: 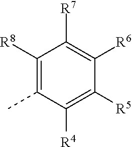
wherein:
R⁴ is selected from H, F, Cl, Br, CN, and a substituted or unsubstituted C₁₋₃alkyl, C₃₋₄cycloalkyl or OC₁₋₃alkyl;
R⁵ is selected from H, F, Cl, CN, and a substituted or unsubstituted C₁₋₃alkyl, C₃₋₄cycloalkyl or OC₁₋₃alkyl;
R⁶ is selected from H, F, Cl, Br, NO₂, NH₂, and a substituted or unsubstituted C₁₋₃alkyl, C₃₋₄ cycloalkyl or OC₁₋₃alkyl;
R⁷ is selected from H, F, Cl, and a substituted or unsubstituted C₁₋₃alkyl;
R⁸ is selected from H, F, and a substituted or unsubstituted C₁₋₃alkyl;
or R⁴ and R⁵ or R⁵ and R⁶ are taken together with their adjacent carbon atoms to form a substituted or unsubstituted carbocycle or heterocycle provided that the heterocycle is not a benzoxazolinone; and
(---) represents a bond;
wherein when R⁴ is H or F, then at least one of R⁵, R⁶, R⁷ or R⁸ is other than H or F; and
wherein when R⁵ is CN, then at least one of R⁴, R⁶, R⁷ or R⁸ is other than H.

5-16. (canceled)

17. The compound of claim 1, wherein R² is a group of the formula: 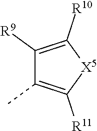
wherein:
X⁵ is selected from NH, NC₁₋₃alkyl, NC₃₋₄cycloalkyl, O and S;
R⁹, R¹⁰, R¹¹ are each independently selected from H, F, Cl, CN, and a substituted or unsubstituted C₁₋₃alkyl, C₃₋₄cycloalkyl, C(O)OC₁₋₃alkyl or OC₁₋₃alkyl, provided that one of R⁹ and R¹¹ is H and the other is different from H; and
(---) represents a bond.

18. The compound of claim 1, wherein R² is a group of the formula: 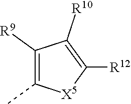
wherein:
X⁵ is selected from NH, NC₁₋₃alkyl, NC₃₋₄cycloalkyl, O and S;
R⁹ is selected from F, Cl, CN, and a substituted or unsubstituted C₁₋₃alkyl, C₃₋₄cycloalkyl, C(O)OC₁₋₃alkyl or OC₁₋₃alkyl;
R¹⁰ and R¹² are each independently selected from H, F, Cl, CN, and a substituted or unsubstituted C₁₋₃alkyl, C₃₋₄cycloalkyl, C(O)OC₁₋₃alkyl or OC₁₋₃alkyl; and
(---) represents a bond.

19-22. (canceled)

23. The compound of claim 1, wherein R² is a group of the formula: 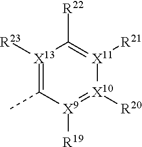
wherein:
X⁹, X¹⁰, X¹¹, X¹², and X¹³ are independently selected from N and C, wherein at least one and at most two of X⁹, X¹⁰, X¹¹, X¹², and X¹³ are N; and
R¹⁹, R²⁰, R²¹, R²² and R²³ are selected from H, F, Cl, Br, CN, NO₂, NH₂, and a substituted or unsubstituted C₁₋₃alkyl, C₃₋₄cycloalkyl or OC₁₋₃alkyl, or are absent when their attached X⁹, X¹⁰, X¹¹, X¹², or X¹³ is N;
wherein at least one of X⁹ and X¹³ is not N; and
wherein when one of X⁹ and X¹³ is N, then the other is not N or CH.

24. The compound of claim 1, wherein R² is a group of the formula: 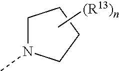
wherein:
R¹³ is independently in each occurrence selected from F, Cl, and a substituted or unsubstituted C₁₋₃alkyl, C₃₋₄cycloalkyl, or C₁₋₃alkoxy;
n is an integer selected from 0 to 8; or
is between 2 and 8 and two R¹⁸ are taken together with their adjacent carbon atoms to form a C₃₋₄cycloalkyl; and
(---) represents a bond.

25-37. (canceled)

38. The compound of claim 1, wherein R¹ is a substituted or unsubstituted group selected from imidazolyl, pyrazolyl, triazolyl, indolyl, indazolyl, benzimidazolyl, benzotriazolyl, pyrrolopyridinyl (e.g. pyrrolo[3,2-b]pyridinyl or pyrrolo[3,2-c]pyridinyl), pyrazolopyridinyl (e.g. pyrazolo[1,5-a]pyridinyl), purinyl, and imidazopyrazinyl (e.g. imidazo[4,5-b]pyrazinyl).

39-42. (canceled)

43. The compound of claim 1, wherein R¹ is substituted with at least one substituent selected from OH, halo, CN, NO₂, C₁₋₆alkyl, C₂₋₆alkenyl, C₂₋₆alkynyl, OC₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂;
wherein:
R¹⁴ is independently in each occurrence selected from H, C₁₋₆alkyl, C₂₋₆alkenyl, C₂₋₆alkynyl, C₃₋₁₀cycloalkyl, C₄₋₁₀heterocycloalkyl, C₆aryl, and C₅₋₁₀heteroaryl, or two R¹⁴ are taken together with their adjacent nitrogen atom to form a C₄₋₁₀heterocycloalkyl group;
R¹⁵ is independently in each occurrence selected from C₁₋₆alkyl, C₂₋₆alkenyl, C₂₋₆alkynyl, C₃₋₁₀cycloalkyl, C₆aryl, and C₅₋₁₀heteroaryl; and
R¹⁶ is independently in each occurrence selected from H, C₁₋₆alkyl, C₂₋₆alkenyl, C₂₋₆alkynyl, C₃₋₁₀cycloalkyl, C₆aryl, and C₅₋₁₀heteroaryl;
wherein said alkyl, alkenyl, alkynyl, cycloalkyl, heterocycloalkyl, aryl or heteroaryl group is optionally further substituted.

44. The compound of claim 1, wherein R¹ is a group of the formula: 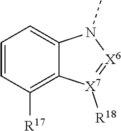
wherein:
R¹⁷ is selected from H, OH, halo, CN, NO₂, C₁₋₆alkyl, C₂₋₆alkenyl, C₂₋₆alkynyl, OC₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂;
X⁶ is N or CH; and
X⁷ is N and R¹⁸ is absent; or
X⁷ is C and R¹⁸ is selected from C₁₋₆alkyl, C₂₋₆alkenyl, C₂₋₆alkynyl, OC₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂;
wherein
R¹⁴ is independently in each occurrence selected from H, C₁₋₆alkyl, C₂₋₆alkenyl, C₂₋₆alkynyl, C₃₋₁₀cycloalkyl, C₄₋₁₀heterocycloalkyl, C₆aryl, and C₅₋₁₀heteroaryl, or two R¹⁴ are taken together with their adjacent nitrogen atom to form a C₄₋₁₀heterocycloalkyl group;
R¹⁵ is independently in each occurrence selected from C₁₋₆alkyl, C₂₋₆alkenyl, C₂₋₆alkynyl, C₃₋₁₀cycloalkyl, C₆aryl, and C₅₋₁₀heteroaryl; and
R¹⁶ is independently in each occurrence selected from H, C₁₋₆alkyl, C₂₋₆alkenyl, C₂₋₆alkynyl, C₃₋₁₀cycloalkyl, C₆aryl, and C₅₋₁₀heteroaryl;
wherein said alkyl, alkenyl, alkynyl, cycloalkyl, heterocycloalkyl, or heteroaryl is optionally further substituted; and
wherein (---) represents a bond.

45-61. (canceled)

62. The compound of claim 1, wherein R¹ is selected from: 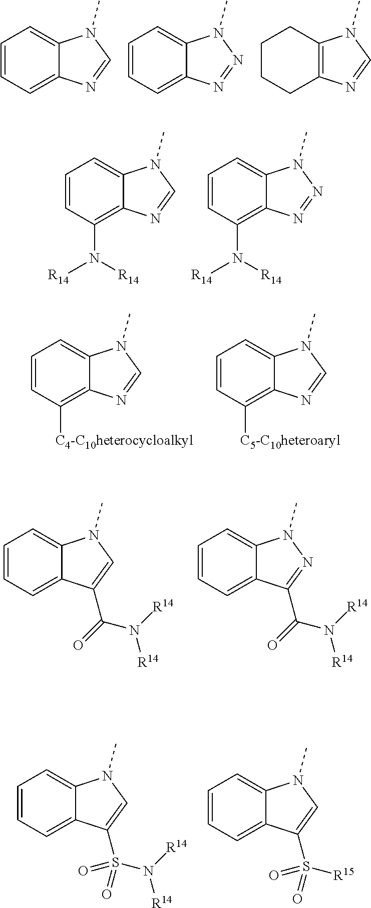
wherein R¹⁴ is as defined herein and (---) represents a bond.

63. (canceled)

64. The compound of claim 1, wherein R¹ is a group of the formula: 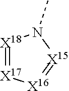
wherein:
X¹⁵, X¹⁶, X¹⁷, and X¹⁸ are independently selected from O, N, S, and CR¹⁷, wherein R¹⁷ is as previously defined;
wherein at most two of X¹⁵, X¹⁶, X¹⁷, and X¹⁸ are O, N, or S.

65-75. (canceled)

76. The compound of claim 1, wherein said compound is of Formula II: 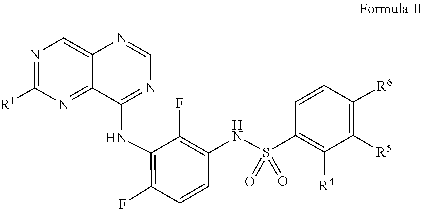
wherein R¹, R⁴, R⁵, and R⁶ are each independently as defined herein;
or said compound is of Formula IV: 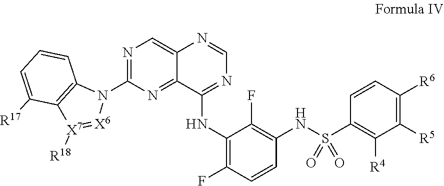
wherein X⁶, X⁷, R⁴, R⁵, R⁶, R¹⁷, and R¹⁸ are each independently as previously defined;
or said compound is of Formula V: 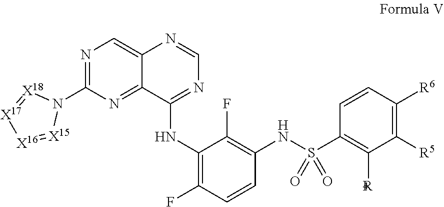
wherein R⁴, R⁵, R⁶, X¹⁵, X¹⁶, X¹⁷, and X¹⁸ are each independently as previously defined;
or said compound is of Formula III: 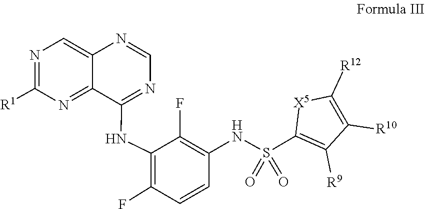
wherein R¹, R⁹, R¹⁰, R¹², and X⁵ are each independently as previously defined;
or said compound is of Formula VI: 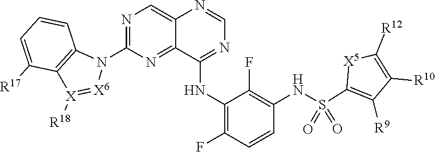
wherein R⁹, R¹⁰, R¹², R¹⁷, R¹⁸, X⁵, X⁶, and X⁷, are each independently as previously defined;
or said compound is of Formula VII: 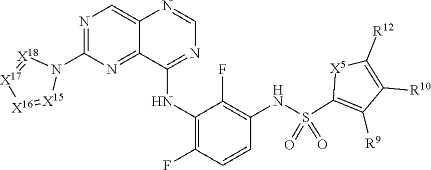
wherein R⁹, R¹⁰, R¹², X⁵, X¹⁵, X¹⁶, X¹⁷ and X¹⁸ are each independently as previously defined.

77-81. (canceled)

82. The compound of claim 1, wherein said compound is selected from Examples 1 to 163 as defined herein, or a salt and/or solvate thereof.

83-84. (canceled)

85. A pharmaceutical composition comprising a compound as defined in claim 1, together with a pharmaceutically acceptable carrier, diluent or excipient.

86-94. (canceled)

95. A method for the treatment of a disease or disorder selected from a proliferative disease or disorder, a developmental anomaly caused by dysregulation of the RAS-ERK signaling cascade (RASopathies), or an inflammatory disease or an immune system disorder, comprising administering a compound as defined in claim 1 to a subject in need thereof.

96-103. (canceled)

104. A method for inhibiting abnormal proliferation of cells, comprising contacting the cells with a compound as defined in claim 1.

105-112. (canceled)
